(19) United States
(12) Patent Application Publication
(10) Pub. No.: US 2024/0401019 A1
(43) Pub. Date: Dec. 5, 2024

(54) RECOMBINANT OLIVETOLIC ACID CYCLASE POLYPEPTIDES ENGINEERED FOR ENHANCED BIOSYNTHESIS OF CANNABINOIDS

(71) Applicants: WILLOW BIOSCIENCES, INC., Calgary (CA); EPIMERON USA, INC., Sunnyvale, CA (US)

(72) Inventors: Trish CHOUDHARY, Belmont, CA (US); Xueyang FENG, Fremont, CA (US); Thanh NGUYEN, Fremont, CA (US); Matthew WORKENTINE, Calgary (CA)

(73) Assignees: WILLOW BIOSCIENCES, INC., Calgary (CA); EPIMERON USA, INC., Sunnyvale, CA (US)

(21) Appl. No.: 18/660,840

(22) Filed: May 10, 2024

Related U.S. Application Data
(63) Continuation of application No. 18/338,242, filed on Jun. 20, 2023, now Pat. No. 12,018,301.
Application No. 18/338,242 is a continuation of application No. PCT/US2022/075170, filed on Aug. 18, 2022.
(60) Provisional application No. 63/341,996, filed on May 13, 2022.
(60) Provisional application No. 63/320,421, filed on Mar. 16, 2022.
(60) Provisional application No. 63/235,087, filed on Aug. 19, 2021.

(51) Int. Cl. C12N 9/88; C12N 15/63; C12P 17/18

(52) U.S. Cl. CPC C12N 9/88; C12N 15/63; C12P 17/182; C12Y 404/01026

(57) ABSTRACT

The present disclosure relates to recombinant polypeptides that have olivetolic acid cyclase activity, nucleic acids encoding these recombinant polypeptides, recombinant host cells that produce these recombinant polypeptides, and compositions comprising the recombinant polypeptides, nucleic acids, and/or recombinant host cells. The present disclosure also relates to uses of these recombinant polypeptides, nucleic acids encoding them, and recombinant host cells comprising them, in methods for the preparation of cannabinoids and cannabinoid precursors.

### CROSS REFERENCE TO RELATED APPLICATIONS

This application is continuation of U.S. application Ser. No. 18/338,242, filed Jun. 20, 2023, which is a continuation of International Application Number PCT/US2022/075710, filed Aug. 18, 2022, which claims priority of U.S. Provisional Patent Application No. 63/341,996, filed May 13, 2022, U.S. Provisional Patent Application No. 63/320,421, filed Mar. 16, 2022, and U.S. Provisional Patent Application No. 63/235,087, filed Aug. 19, 2021, the entirety of each of which is hereby incorporated by reference herein.

### FIELD

The present disclosure relates to engineered genes encoding recombinant polypeptides having olivetolic acid cyclase (OAC) activity and the use of these genes and polypeptides in recombinant host cell and in vitro systems for the production of cannabinoid compounds.

### REFERENCE TO SEQUENCE LISTING

The official copy of the Sequence Listing is submitted concurrently with the specification via USPTO Patent Center as an WIPO Standard ST.26 formatted XML file with file name “13421-015WO1.xml”, a creation date of Aug. 15, 2022, and a size of 1,289,937 bytes. This Sequence Listing filed via USPTO Patent Center is part of the specification and is incorporated in its entirety by reference herein.

### BACKGROUND

Cannabinoids are a class of compounds that act on endocannabinoid receptors and include the phytocannabinoids naturally produced by Cannabis sativa. Cannabinoids include the more prevalent and well-known compounds, Δ⁹-tetrahydrocannabinol (THC), cannabidiol (CBD), as well as 80 or more less prevalent cannabinoids, cannabinoid precursors, related metabolites, and synthetically produced derivative compounds. Cannabinoids are increasingly used to treat a range of diseases and conditions such as multiple sclerosis and chronic pain. Current large-scale production of cannabinoids for pharmaceutical or other use is through extraction from plants. These plant-based production processes, however, have several challenges including susceptibility of the plants to inconsistent production caused by variance in biotic and abiotic factors, difficulty reproducing identical cannabinoid accumulation profiles, and difficulty in producing a single cannabinoid compound with purity high enough for pharmaceutical applications. While some cannabinoids can be produced as a single pure product via chemical synthesis, these processes have proven very costly and too costly for large-scale production.

More economical biosynthetic approaches to cannabinoid production are being developed using microbial hosts. These processes have the potential to be robust, scalable, and capable of producing single cannabinoid compound with higher purity compared to other current processes. Several biosynthetic systems for cannabinoid compound have been reported (see e.g., WO2019071000, WO2018200888, WO2018148849, WO2019014490, US20180073043, US20180334692, and WO2019046941). These biosynthetic systems typically incorporate a four enzyme pathway derived from Cannabis sativa including: (1) an acyl activating enzyme (AAE) of class E.C. 6.2.1.1; (2) an olivetol synthase (OLS) of class E.C. 2.3.1.206; (3) an olivetolic acid cyclase (OAC) of class E.C. 4.4.1.26, and (4) a prenyltransferase (PT) of class E.C. 2.5.1.102. In C. sativa this four enzymes cannabinoid pathway is capable of carrying out the conversion of a hexanoic acid (HA) starting compound to the cannabinoid precursor compound, olivetolic acid (OA), followed by the prenylation of OA with geranyl pyrophosphate (GPP) to provide the cannabinoid, cannabigerolic acid (CBGA). A recombinant version of this pathway in microbial hosts has been shown to be capable of producing OA and CBGA to some extent, but are not efficient in the production of these compounds, or the downstream cannabinoid compounds, cannabidiolic acid (CBDA), or Δ⁹-tetrahydrocannabinolic acid (THCA).

There exists a need for improved recombinant genes encoding cannabinoid pathway enzymes (such as OAC) that when integrated in recombinant host cell systems enhance the biosynthetic production of cannabinoid precursors, and cannabinoids, such as OA, CBGA, CBDA, and THCA, and the rare precursors and rare cannabinoids such as DA, CBGVA, CBDVA, and THCVA.

### SUMMARY

The present disclosure relates generally to engineered genes encoding recombinant polypeptides with olivetolic acid cyclase (OAC) activity, and the use of these engineered genes in recombinant host cell systems for the enhanced biosynthetic production of cannabinoids and cannabinoid precursor compounds. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a recombinant polypeptide having olivetolic acid cyclase (OAC) activity, wherein the polypeptide comprises an amino acid sequence of at least 80% identity to SEQ ID NO: 6 or 20, and an amino acid residue difference as compared to SEQ ID NO: 6 or 20 at one or more positions selected from: A2, L6, V8, L9, K10, F11, K12, E14, T16, E17, A18, E21, E22, F23, K25, T26, Y27, V28, N29, V31, I33, A36, V40, Y41, K44, D45, V46, T47, Q48, K49, N50, E52, E53, Y55, T56, H57, 158, T62, T62, E64, V66, T68, Q70, D71, I74, P76, A77, H78, G80, G82, D83, V84, Y85, R86, S87, F88, E90, K91, I94, Y97, T98, and R100.

In at least one embodiment, the polypeptide comprises an amino acid sequence of at least 80% identity to SEQ ID NO: 6, and an amino acid residue difference as compared to SEQ ID NO: 6 at each of a combination of six positions, wherein the combination of six positions are selected from the combinations listed in Table 4.

In at least one embodiment, the polypeptide comprises amino acid residue differences are selected from: A2G, A2S, A2P, A2V, L6F, V8I, L9A, L9F, L9G, L9I, L9M, L9S, L9V, K10A, F11L, K12L, K12N, K12Q, K12V, E14G, T16P, T16Q, E17G, A18E, A18S, E21L, E21V, E22L, F23I, K25D, K25G, K25E, K25N, K25R, K25S, T26A, T26N, Y27F, V28C, N29D, N29G, V31A, V31E, V31M, V31S, I33D, I33E, I33V, A36E, A36F, A36L, A36Q, A36S, V40A, V40G, Y41E, Y41Q, Y41S, Y41T, K44P, D45V, V46I, V46L, T47A, T47G, T47S, T47S, Q48C, Q48H, Q48M, Q48P, K49A, K49C, K49G, K49H, K49L, K49N, K49P, K49R, K49S, K49T, K49V, N50Y, E52Q, E52R, E52S, E53A, E53F, E53H, E53L, E53R, E53S, E53V, Y55W, T56S, H57G, I58C, I58V, T62C, T62G, E64D, E64K, V66I, V66L, E67S, T68A, T68C, T68E, T68G, T68H, T68M, T68Q, T68S, Q70A, Q70K, D71G, I74G, I74H, I74K, I74L, I74M, I74N, I74Q, I74R, I74S, I74T, I74V, P76V, A77E, H78P, G80K, G82A, G82R, D83K, D83R, V84I, V84M, Y85F, R86S, S87H, S87K, S87P, F88W, F88Y, E90D, K91E, I94K, Y97F, T98V, R100A, and R100G.

In at least one embodiment, the polypeptide comprises a combination of amino acid differences selected from any of the combinations listed in Table 5, and/or any combination present in a polypeptide listed in Tables 5, 7, 8, 9, 10, 13, 14, and/or 15, as disclosed herein.

In at least one embodiment, the polypeptide comprises an amino acid sequence of at least 80%, at least 85%, at least 90%, at least 95%, at least 97%, at least 98%, or at least 99% identity to a sequence selected from the group consisting of even-numbered SEQ ID NOs: 22 to 890.

In at least one embodiment, the olivetolic acid cyclase activity of the polypeptide as compared to the CsOAC polypeptide consisting of SEQ ID NO: 6 or 20 at least 0.2-fold, at least 0.4-fold, at least 0.6-fold, at least 0.8-fold, at least 1.0-fold, at least 1.2-fold, at least 1.4-fold, at least 1.8-fold, at least 1.6-fold, at least 2-fold, at least 4-fold, or more. In at least one embodiment, the olivetolic acid cyclase activity of the polypeptide is measured as the rate of conversion of the substrate 3,5,7-trioxododecanoyl-CoA (compound (2)) to olivetolic acid (compound (1)); optionally, under reaction conditions of pH 7 and 30C.

In at least one embodiment, the olivetolic acid cyclase activity of the polypeptide when expressed in a recombinant host cell comprising a pathway capable of producing 3,5,7-trioxododecanoyl-CoA (compound (2)) results in a titer of olivetolic acid (compound (1)) produced by the cell that is relative to a control cell expressing the CsOAC polypeptide of SEQ ID NO: 6 or 20 at least 0.2-fold, at least 0.4-fold, at least 0.6-fold, at least 0.8-fold, at least 1.0-fold, at least 1.2-fold, at least 1.4-fold, at least 1.8-fold, at least 1.6-fold, at least 2-fold, at least 4-fold, or more.

In at least one embodiment, the present disclosure also provides a polynucleotide encoding a recombinant polypeptide having olivetolic acid cyclase activity of the present disclosure. In at least one embodiment, the polynucleotide comprises: (a) a sequence of at least 80%, at least 85%, at least 90%, at least 95%, at least 97%, at least 98%, or at least 99% identity to a sequence selected from the group consisting of odd-numbered SEQ ID NOs: 21 to 889; or (b) a codon degenerate sequence of a sequence selected from the group consisting of odd-numbered SEQ ID NOs: 21 to 889.

In at least one embodiment, the present disclosure also provides an expression vector comprising a polynucleotide encoding a recombinant polypeptide having olivetolic acid cyclase activity of the present disclosure, optionally wherein, the expression vector comprises a control sequence.

In at least one embodiment, the present disclosure also provides a recombinant host cell comprising: (a) a polynucleotide encoding a recombinant polypeptide having olivetolic acid cyclase activity of the present disclosure, or (b) an expression vector comprising a polynucleotide encoding a recombinant polypeptide having olivetolic acid cyclase activity of the present disclosure.

In at least one embodiment, the present disclosure provides a method for preparing a recombinant polypeptide having olivetolic acid cyclase activity of the present disclosure wherein the method comprises culturing a recombinant host cell of the present disclosure and isolating the polypeptide from the cell.

In at least one embodiment, the present disclosure provides a method for preparing a recombinant polypeptide having olivetolic acid cyclase activity comprising:
(a) transforming a host cell with an expression vector comprising a polynucleotide encoding a recombinant polypeptide having olivetolic acid cyclase activity of the present disclosure;
(b) culturing said transformed host cell under conditions whereby said recombinant polypeptide is produced by said host cell; and
(c) recovering said recombinant polypeptide from said host cells.

In at least one embodiment, the present disclosure also provides a recombinant host cell comprising a nucleic acid encoding a recombinant polypeptide having olivetolic acid cyclase activity of the present disclosure.

In at least one embodiment of the recombinant host cell, the host cell further comprises a pathway of enzymes capable of producing a cannabinoid precursor; optionally, wherein the cannabinoid precursor is divarinic acid (DA) or olivetolic acid (OA).

In at least one embodiment of the recombinant host cell, the host cell further comprises a pathway of enzymes capable of producing a tetraketide cannabinoid precursor; optionally, wherein the tetraketide cannabinoid precursor is 3,5,7-trioxododecanoyl-CoA. In at least one embodiment, the pathway comprises enzymes capable of converting hexanoic acid (HA) to 3,5,7-trioxododecanoyl-CoA.

In at least one embodiment of the recombinant host cell, the pathway comprises enzymes capable of catalyzing reactions (i)-(ii):

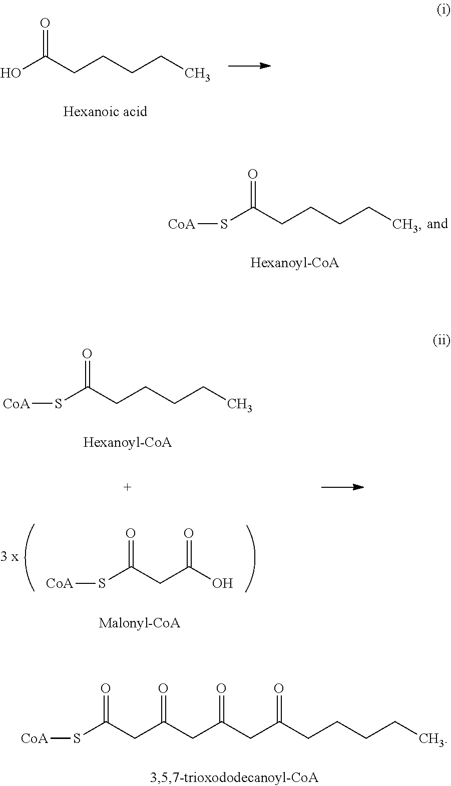

In at least one embodiment, the pathway comprises at least the enzymes AAE, and OLS; optionally, wherein the enzymes AAE, and OLS, have an amino acid sequence of at least 90% identity to SEQ ID NO: 2 (AAE), and SEQ ID NO: 4 (OLS), respectively.

In at least one embodiment of the recombinant host cell, the host cell further comprises a nucleic acid encoding an enzyme capable of catalyzing the conversion of OA to CBGA. In at least one embodiment, the pathway comprises an enzyme capable of catalyzing reaction (iv):

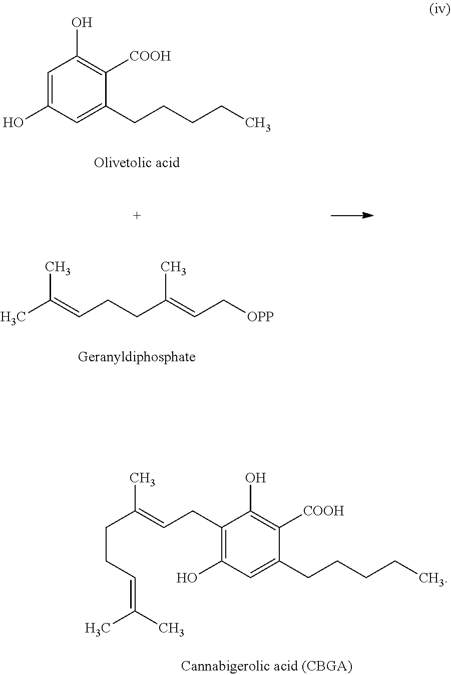

In at least one embodiment, the host cell further comprises a nucleic acid encoding a prenyltransferase; optionally, wherein the prenyltransferase has an amino acid sequence of at least 90% identity to SEQ ID NO: 8 or 10.

In at least one embodiment of the recombinant host cell, the host cell further comprises a nucleic acid encoding an enzyme capable of catalyzing the conversion of CBGA to Δ⁹-THCA. CBDA, and/or CBCA; optionally, wherein the host cell further comprises a nucleic acid encoding an enzyme capable of catalyzing a reaction (v), (vi), and/or (vii):

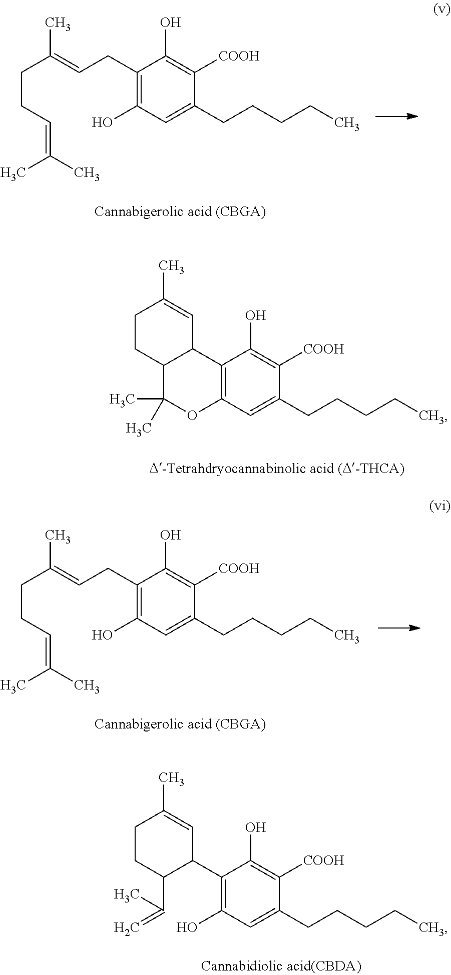

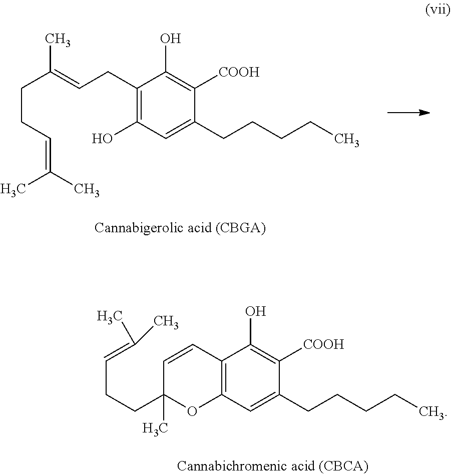 [SMILES: CCCCCc1cc(O)c(C/C=C(\C)CCC=C(C)C)c(O)c1C(=O)O ; CCCCCc1cc2c(c(O)c1C(=O)O)C=CC(C)(CCC=C(C)C)O2]

In at least one embodiment of the recombinant host cell, the host cell further comprises a nucleic acid encoding THCA synthase, CBDA synthase, and/or CBCA synthase; optionally, wherein the CBDA synthase has an amino acid sequence of at least 90% identity to SEQ ID NO: 12 or 14; and the THCA synthase having an amino acid sequence of at least 90% identity to SEQ ID NO: 16 or 18.

In at least one embodiment of the recombinant host cell, the host cell is capable of producing a cannabinoid selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC). Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment of the recombinant host cell, the host cell comprises a pathway capable of producing CBGA, and the production of CBGA is at least 0.2-fold, at least 0.4-fold, at least 0.6-fold, at least 0.8-fold, at least 1.0-fold, at least 1.2-fold, at least 1.4-fold, at least 1.8-fold, at least 1.6-fold, at least 2-fold, at least 4-fold, or more, relative to a control recombinant host cell comprising a pathway with the recombinant polypeptide having olivetolic acid cyclase activity replaced by a polypeptide of SEQ ID NO: 6 or 20.

In at least one embodiment of the recombinant host cell, the source of the host cell is selected from Saccharomyces cerevisiae, Yarrowia lipolytica, Pichia pastoris, and Escherichia coli.

In at least one embodiment, the present disclosure also provides a method for producing a cannabinoid or a cannabinoid precursor comprising: (a) culturing in a suitable medium a recombinant host cell of the present disclosure; and (b) recovering the produced cannabinoid or cannabinoid precursor. In at least one embodiment, the method further comprises contacting a cell-free extract of the culture with a biocatalytic reagent or chemical reagent.

In at least one embodiment, the present disclosure also provides a method for preparing a compound of structural formula (I)

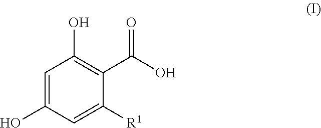

wherein, R¹ is C1-C7 alkyl, the method comprising contacting under suitable reactions conditions a compound of structural formula (II)

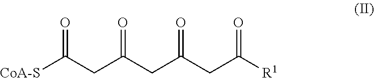

wherein, R¹ is C1-C7 alkyl, and a recombinant polypeptide have olivetolic acid cyclase activity of the present disclosure. In at least one embodiment: (a) the compound of structure formula (I) is olivetolic acid (OA) and the compound of structural formula (II) is 3,5,7-trioxododecanoyl -CoA; or (b) the compound of structure formula (I) is divarinic acid (DA) and the compound of structural formula (II) is 3,5,7-trioxodecanoyl-CoA acid.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary four enzyme pathway capable of converting hexanoic acid (HA) to the cannabinoid precursor, olivetolic acid (OA), and then further converting OA to the cannabinoid, cannabigerolic acid (CBGA). The four enzymes catalyzing the steps in the biosynthetic pathway are AAE, OLS, OAC, and PT.

FIG. 2 depicts three exemplary two step pathways for converting the cannabinoid, CBGA, to one or more of the cannabinoids, Δ⁹-THCA, CBDA, and/or CBCA, and then, optionally, further converting them to the decarboxylated cannabinoids, Δ⁹-THC, CBD, and/or CBC. The first conversion from CBGA to Δ⁹-THCA, CBDA, and/or CBCA can be catalyzed by a cannabinoid synthase, CBDA synthase (CBDAS), THCA synthase (THCAS) and/or CBCA synthase (CBCAS), respectively. As described elsewhere herein, in some embodiments the single cannabinoid synthase (e.g., CBDAS) is capable of catalyzing not only the conversion of CBGA to its preferred product (e.g., CBDAS preferentially converts CBGA to CBDA), but also converts CBGA to one or both of the other cannabinoid acid products, typically in lesser amounts.

FIG. 3 depicts an exemplary four enzyme pathway capable of converting butyric acid (BA) to the rare cannabinoid precursor, divarinic acid (DA), and then further converting DA to the rare cannabinoid, cannabigerovarinic acid (CBGVA). The four enzymes catalyzing the steps in the biosynthetic pathway are AAE, OLS, OAC, and PT.

FIG. 4 depicts three exemplary two step pathways for converting the rare cannabinoid, CBGVA, to one or more of the rare cannabinoids, Δ⁹-THCVA, CBDVA, and/or CBCVA, and then, optionally, further converting them to the decarboxylated cannabinoids, Δ⁹-THCV, CBDV, and/or CBCV. The first conversion from CBGVA to Δ⁹-THCVA, CBDVA, and/or CBCVA can be catalyzed by a single cannabinoid synthase, CBDAs, THCAs and/or CBCAs, respectively. As described elsewhere herein, in some embodiments the single cannabinoid synthase (e.g., CBDAs) is capable of catalyzing not only the conversion of CBGVA to its preferred product (e.g., CBDAs preferentially converts CBGVA to CBDVA), but also converts CBGVA to one or both of the other cannabinoid acid products, typically in lesser amounts.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor, and is intended to include the endocannabinoid compounds that are produced naturally in animals, the phytocannabinoid compounds produced naturally in cannabis plants, and the synthetic cannabinoids compounds. Cannabinoids as referenced in the present disclosure include, but are not limited to, the exemplary naturally occurring and synthetic cannabinoid product compounds shown below in Table 1 (below).

TABLE 1
Exemplary cannabinoid product compounds

| | Abbrev. | |
|---|---|---|
| Compound Name | Name | Chemical Structure |
| cannabigerolic acid | CBGA | 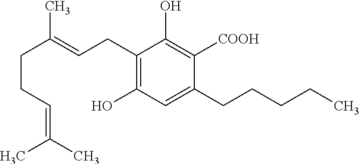 [SMILES: CCCCCc1cc(O)c(C/C=C(\C)CCC=C(C)C)c(O)c1C(=O)O] |
| cannabigerol | CBG | 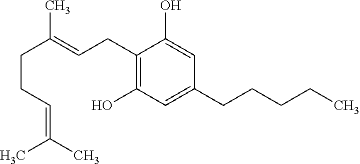 [SMILES: CCCCCc1cc(O)c(C/C=C(\C)CCC=C(C)C)c(O)c1] |
| Δ⁹-tetrahydrocannabinolic acid | Δ⁹-THCA | 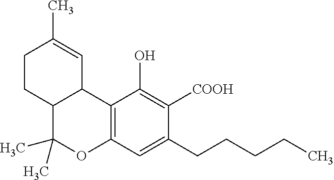 [SMILES: CCCCCc1cc2c(c(O)c1C(=O)O)C1C=C(C)CCC1C(C)(C)O2] |
| Δ⁹-tetrahydrocannabinol | Δ⁹-THC | 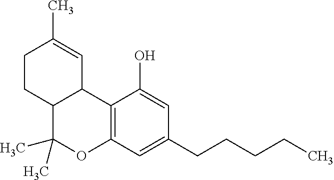 [SMILES: CCCCCc1cc(O)c2c(c1)OC(C)(C)C1CCC(C)=CC21] |
| Δ⁸-tetrahydrocannabinolic acid | Δ⁸-THCA | 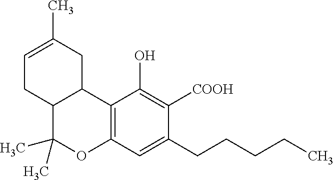 [SMILES: CCCCCc1cc2c(c(O)c1C(=O)O)C1CC(C)=CCC1C(C)(C)O2] |
| Δ⁸-tetrahydrocannabinol | Δ⁸-THC | 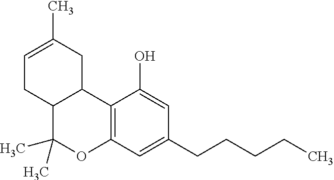 [SMILES: CCCCCc1cc(O)c2c(c1)OC(C)(C)C1CC=C(C)CC21] |
| cannabidiolic acid | CBDA | 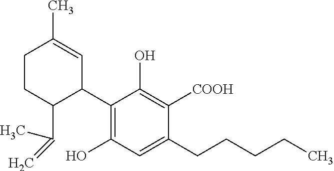 [SMILES: C=C(C)C1CCC(C)=CC1c1c(O)cc(CCCCC)c(C(=O)O)c1O] |
| cannabidiol | CBD | 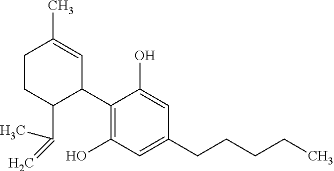 [SMILES: C=C(C)C1CCC(C)=CC1c1c(O)cc(CCCCC)cc1O] |
| cannabichromenic acid | CBCA | 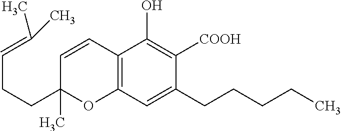 [SMILES: CCCCCc1cc2c(c(O)c1C(=O)O)C=CC(C)(CCC=C(C)C)O2] |
| cannabichromene | CBC | 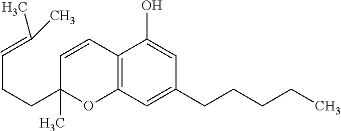 [SMILES: CCCCCc1cc(O)c2c(c1)OC(C)(CCC=C(C)C)C=C2] |
| cannabinolic acid | CBNA | 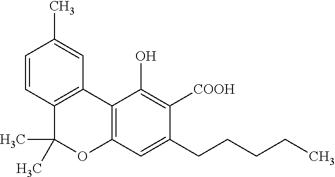 [SMILES: CCCCCc1cc2c(c(O)c1C(=O)O)-c1cc(C)ccc1C(C)(C)O2] |
| cannabinol | CBN | 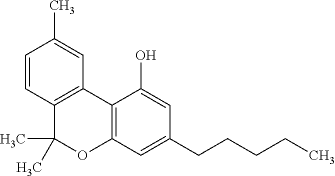 [SMILES: CCCCCc1cc(O)c2c(c1)OC(C)(C)c1ccc(C)cc1-2] |
| cannabidivarinic acid | CBDVA | 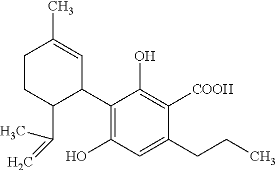 [SMILES: C=C(C)C1CCC(C)=CC1c1c(O)cc(CCC)c(C(=O)O)c1O] |
| cannabidivarin | CBDV | 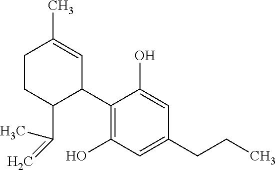 [SMILES: C=C(C)C1CCC(C)=CC1c1c(O)cc(CCC)cc1O] |
| Δ⁹-tetrahydrocannabivarinic acid | Δ⁹- THCVA | 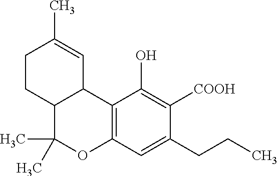 [SMILES: CCCc1cc2c(c(O)c1C(=O)O)C1C=C(C)CCC1C(C)(C)O2] |
| Δ⁹-tetrahydrocannabivarin | Δ⁹-THCV | 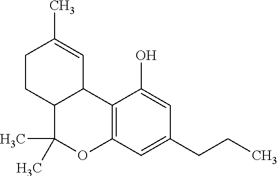 [SMILES: CCCc1cc(O)c2c(c1)OC(C)(C)C1CCC(C)=CC21] |
| cannabidibutolic acid | CBDBA | 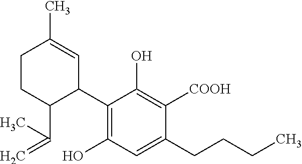 [SMILES: C=C(C)C1CCC(C)=CC1c1c(O)cc(CCCC)c(C(=O)O)c1O] |
| cannabidibutol | CBDB | 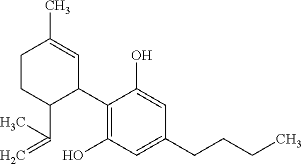 [SMILES: C=C(C)C1CCC(C)=CC1c1c(O)cc(CCCC)cc1O] |
| Δ⁹- tetrahydrocannabutolic acid | Δ⁹-THCBA | 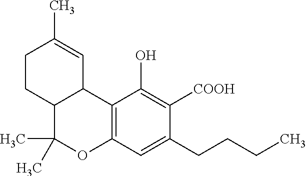 [SMILES: CCCCc1cc2c(c(O)c1C(=O)O)C1C=C(C)CCC1C(C)(C)O2] |
| Δ⁹-tetrahydrocannabutol | Δ⁹-THCB | 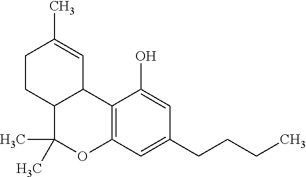 [SMILES: CCCCc1cc(O)c2c(c1)OC(C)(C)C1CCC(C)=CC21] |
| cannabigerophorolic acid | CBGPA | 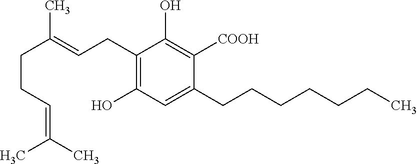 [SMILES: CCCCCCCc1cc(O)c(C/C=C(\C)CCC=C(C)C)c(O)c1C(=O)O] |
| cannabigerophorol | CBGP | 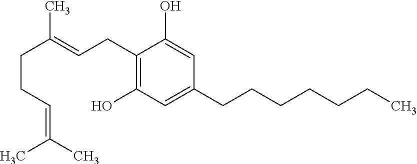 [SMILES: CCCCCCCc1cc(O)c(C/C=C(\C)CCC=C(C)C)c(O)c1] |
| cannabidiphorolic acid | CBDPA | 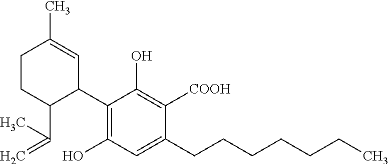 [SMILES: C=C(C)C1CCC(C)=CC1c1c(O)cc(CCCCCCC)c(C(=O)O)c1O] |
| cannabidiphorol | CBDP | 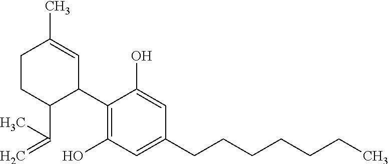 [SMILES: C=C(C)C1CCC(C)=CC1c1c(O)cc(CCCCCCC)cc1O] |
| Δ⁹- tetrahydrocannabiphorolic acid | Δ⁹- THCPA | 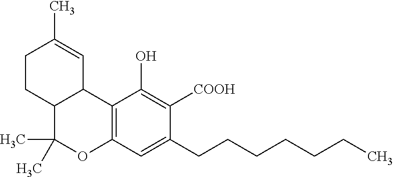 [SMILES: CCCCCCCc1cc2c(c(O)c1C(=O)O)C1C=C(C)CCC1C(C)(C)O2] |
| Δ⁹-tetrahydrocannabiphorol | Δ⁹-THCP | 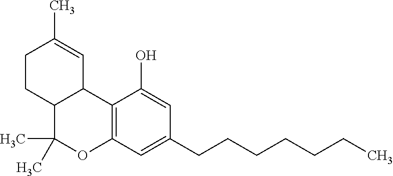 [SMILES: CCCCCCCc1cc(O)c2c(c1)OC(C)(C)C1CCC(C)=CC21] |
| cannabichromevarinic acid | CBCVA | 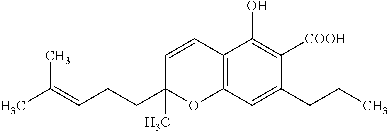 [SMILES: CCCc1cc2c(c(O)c1C(=O)O)C=CC(C)(CCC=C(C)C)O2] |
| cannabichromevarin | CBCV | 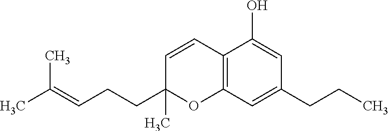 [SMILES: CCCc1cc(O)c2c(c1)OC(C)(CCC=C(C)C)C=C2] |
| cannabigerovarinic acid | CBGVA | 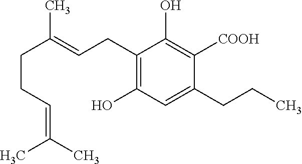 [SMILES: CCCc1cc(O)c(C/C=C(\C)CCC=C(C)C)c(O)c1C(=O)O] |
| cannabigerovarin | CBGV | 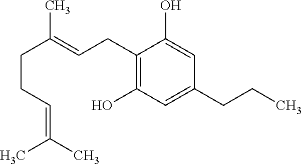 [SMILES: CCCc1cc(O)c(C/C=C(\C)CCC=C(C)C)c(O)c1] |
| cannabicyclolic acid | CBLA | 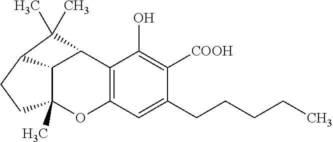 |
| cannabicyclol | CBL | 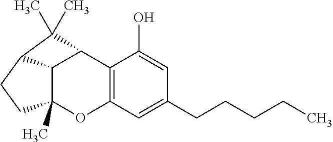 |
| cannabielsoinic acid | CBEA | 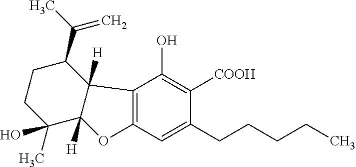 [SMILES: C=C(C)[C@@H]1CC[C@](C)(O)[C@H]2Oc3cc(CCCCC)c(C(=O)O)c(O)c3[C@@H]12] |
| cannabielsoin | CBE | 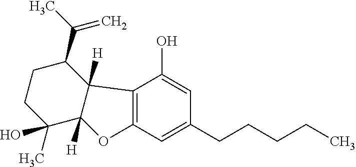 [SMILES: C=C(C)[C@@H]1CC[C@](C)(O)[C@H]2Oc3cc(CCCCC)cc(O)c3[C@@H]12] |
| cannabicitranic acid | CBTA | 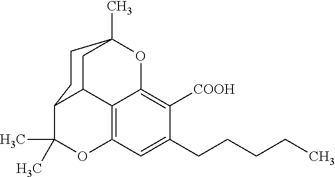 [SMILES: CCCCCc1cc2c3c(c1C(=O)O)OC1(C)CCC(C3C1)C(C)(C)O2] |
| cannabicitran | CBT | 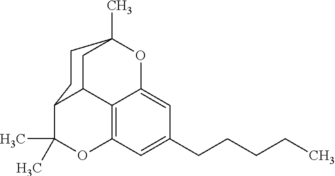 [SMILES: CCCCCc1cc2c3c(c1)OC(C)(C)C1CCC(C)(CC31)O2] |

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” refers to a pathway that can convert a cannabinoid precursor molecule, such as hexanoic acid, into a cannabinoid molecule, such as cannabigerolic acid (CBGA). For example, the four enzymes AAE, OLS, OAC, and PT which convert hexanoic acid to CBGA, form a pathway capable of producing a cannabinoid.

“Cannabinoid precursor” as used herein refers to a compound capable of being converted into a cannabinoid by a pathway capable producing a cannabinoid. Cannabinoid precursors as referenced in the present disclosure include, but are not limited to, the exemplary naturally occurring and synthetic cannabinoid precursors with varying alkyl carbon chain lengths summarized in Table 2 (below).

TABLE 2
Exemplary cannabinoid precursor compounds

| | Abbrev. | |
|---|---|---|
| Compound Name | Name | Chemical Structure |
| Orcinolic acid (2,4-dihydroxy-6- methylbenzoic acid) | OrcA | 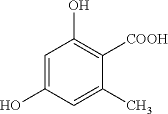 |
| Divarinic acid (2,4-dihydroxy-6- propylbenzoic acid) | DA | 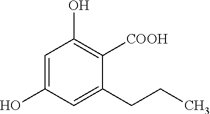 |
| Butolic acid (2-butyl-4,6- dihydroxybenzoic acid) | BA | 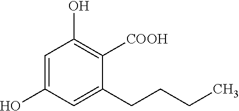 |
| Olivetolic acid (2,4-dihydroxy-6- pentylbenzoic acid) | OA | 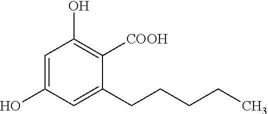 [SMILES: CCCCCc1cc(O)cc(O)c1C(=O)O] |
| 2-hexyl-4,6- dihydroxybenzoic acid | DHBA | 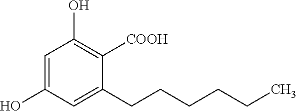 [SMILES: CCCCCCc1cc(O)cc(O)c1C(=O)O] |
| Sphaerophorolic acid (2-heptyl-4,6- dihydroxybenzoic acid) | PA | 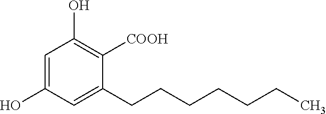 [SMILES: CCCCCCCc1cc(O)cc(O)c1C(=O)O] |

“Conversion” as used herein refers to the enzymatic conversion of a substrate(s) to a corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine, and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon degenerate” describes a nucleotide sequence that has one or more different codons relative to the reference nucleotide sequence but which encodes a polypeptide that is identical to the polypeptide encoded by a reference nucleotide sequence. The different codons between the nucleotide sequence and the reference nucleotide sequence are called “synonyms” or “synonymous” codons in that they use different triplets of nucleotides to encode the same amino acid in a polypeptide.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several different “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; Mcinerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N =−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

Engineered Genes Encoding Recombinant Polypeptides with OAC Activity

The present disclosure provides engineered genes that encode recombinant polypeptides having olivetolic acid cyclase (OAC) activity, a carbon-sulfur lyase enzyme of class E.C. 4.4.1.26. When integrated into a recombinant host cell (e.g., S. cerevisiae) having a pathway capable of producing a tetraketide-CoA, such as 3,5,7-trioxododecanoyl-CoA, the presence of an engineered OAC gene expressing the recombinant polypeptides can result in the production of the cyclized aromatic cannabinoid precursor product, such as olivetolic acid (OA) with an enhanced yield. In at least one embodiment, when an engineered gene of the present disclosure is integrated in a recombinant host cell capable of producing the C-12 tetraketide-CoA compound, 3,5,7-trioxododecanoyl-CoA, the cyclized aromatic product OA, is produced by the host cell in greater yield relative to a comparable recombinant host cell integrated with a codon-optimized version of the gene encoding the wild-type Cannabis sativa OAC polypeptide of SEQ ID NO: 6 or 20.

The activity of the CsOAC polypeptide in the cannabinoid pathway of C. sativa is the cyclization of the C-12 tetraketide-CoA substrate, 3,5,7-trioxododecanoyl-CoA (compound (2)) to form the cannabinoid precursor product, olivetolic acid (compound (1)), as shown in Scheme 1.

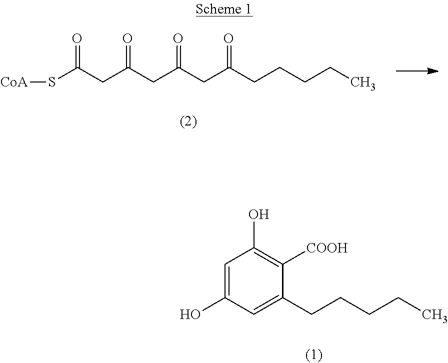

The engineered genes encoding recombinant polypeptides of the present disclosure exhibit the exemplary OAC activity of Scheme 1 when incorporated and expressed in a recombinant host cell comprising a pathway that produces the tetraketide-CoA cannabinoid precursor. Specifically, the recombinant polypeptides have OAC activity capable of hydrolyzing the CoA-thiol of 3,5,7-trioxododecanoyl-CoA (compound (2)) and cyclizing the tetraketide to form the cannabinoid precursor product, OA (compound (1)). The OAC activity resulting in the conversion of the tetraketide-CoA cannabinoid precursor substrate (e.g., compound (2)) to the cannabinoid precursor product (e.g., compound (1)) as in Scheme 1, when carried out by the engineered genes of the present disclosure integrated in a recombinant host cell results in a yield of OA the is comparable to or increased relative to a control recombinant host cell strain integrated with the yeast codon-optimized genes of either SEQ ID NO: 5 or 19 that encode the wild-type CsOAC polypeptide of SEQ ID NO: 6 or 20. Without intending to be bound by any particular theory or mechanism, the altered yield of the cyclized cannabinoid precursor product is correlated with the one or more residue differences in recombinant polypeptides of the present disclosure, as compared to the CsOAC amino acid sequence of SEQ ID NO: 6 or 20. Exemplary engineered genes and encoded recombinant polypeptides with OAC activity that exhibit the unexpected and surprising technical effect of comparable or increased cannabinoid or cannabinoid precursor yield when integrated in a recombinant host cell are summarized in Table 3 below (as well as in the following Examples and the accompanying Sequence Listing).

TABLE 3
Recombinant polypeptides with olivetolic acid cyclase (OAC) activity

| | NT | AA |
|---|---|---|
| | SEQ ID | SEQ ID |
| aa differences relative to CsOAC¹ | NO: | NO: |
| T16Q | 21 | 22 |
| P76V | 23 | 24 |
| L9V | 25 | 26 |
| K44P | 27 | 28 |
| I74S | 29 | 30 |
| H57G | 31 | 32 |
| K49R | 33 | 34 |
| E14G | 35 | 36 |
| D71G | 37 | 38 |
| K91E | 39 | 40 |
| L9V, E21V | 41 | 42 |
| T26N, K49R, D71G, K91E | 43 | 44 |
| K12L | 45 | 46 |
| A18S | 47 | 48 |
| L9V, E14G, T16Q, V40G, K49R, | 49 | 50 |
| D71G, 174M, K91E | | |
| E17G, K44P | 51 | 52 |
| I94K | 53 | 54 |
| E64K | 55 | 56 |
| I33E, K49R, D71G, K91E | 57 | 58 |
| L9V, E21V, G82A | 59 | 60 |
| A77E | 61 | 62 |
| L9V, E14G, T16Q, K49R, D71G, K91E | 63 | 64 |
| T62G | 65 | 66 |
| F11L | 67 | 68 |
| S87K | 69 | 70 |
| D83R | 71 | 72 |
| L9V, E14G, T16Q, K49R, D71G, V84M, K91E | 73 | 74 |
| G82R | 75 | 76 |
| K25R, K49R, D71G, K91E | 77 | 78 |
| L9V, E14G, T16Q, A36E, K49R, D71G, K91E | 79 | 80 |
| K49R, D71G, K91E | 81 | 82 |
| L9V, E14G, T16Q, K49R, D71G, K91E | 83 | 84 |
| L9V, E14G, K49R, D71G, K91E | 85 | 86 |
| L9V, E14G, K49R, H57G, D71G, K91E | 87 | 88 |
| K25S, K49R, D71G, K91E | 89 | 90 |
| L9V, E14G, T16Q, K49R, D71G, K91E | 91 | 92 |
| L9V, E14G, K49R, D71G, K91E, Y97F | 93 | 94 |
| L9V, E14G, K49R, D71G, K91E, Y55W | 95 | 96 |
| L9I, E14G, K49R, D71G, K91E | 97 | 98 |
| L9V, E14G, K49R, D71G, K91E, V66L | 99 | 100 |
| L9V, E14G, V31S, K49R, D71G, K91E | 101 | 102 |
| L9V, E14G, K49R, D71G, K91E, V31M | 103 | 104 |
| L9V, E14G, K49R, D71G, K91E, V31E | 105 | 106 |
| L9V, E14G, K49R, D71G, K91E, V28C | 107 | 108 |
| L9V, E14G, K49R, D71G, K91E, T68S | 109 | 110 |
| L9V, E14G, K49R, D71G, K91E, T68Q | 111 | 112 |
| L9V, E14G, K49R, D71G, K91E, T68M | 113 | 114 |
| L9V, E14G, K49R, D71G, K91E, T68G | 115 | 116 |
| L9V, E14G, K49R, D71G, K91E, T68E | 117 | 118 |
| L9V, E14G, K49R, D71G, K91E, T68A | 119 | 120 |
| L9V, E14G, K49R, D71G, K91E, N29G | 121 | 122 |
| L9V, E14G, K49R, D71G, K91E, L6F | 123 | 124 |
| L9V, E14G, K49R, D71G, K91E, K25G, H78P | 125 | 126 |
| L9V, E14G, K49R, D71G, K91E, I74V | 127 | 128 |
| L9V, E14G, K49R, D71G, K91E, I74T | 129 | 130 |
| L9V, E14G, K49R, D71G, K91E, I74M | 131 | 132 |
| L9V, E14G, K49R, D71G, K91E, I74L | 133 | 134 |
| L9V, E14G, K49R, D71G, K91E, I74G | 135 | 136 |
| L9V, E14G, K49R, D71G, K91E, I33V | 137 | 138 |
| L9V, E14G, K49R, D71G, K91E, I33D | 139 | 140 |
| L9V, E14G, K49R, D71G, K91E, E64D | 141 | 142 |
| L9V, E14G, K49R, D71G, K91E, E53S | 143 | 144 |
| L9V, E14G, K49R, D71G, K91E, E53R, V84I | 145 | 146 |
| L9V, E14G, K49R, D71G, K91E, E53R | 147 | 148 |
| L9V, E14G, K49R, D71G, K91E, E53L | 149 | 150 |
| L9V, E14G, K49R, D71G, K91E, E53H | 151 | 152 |
| L9V, E14G, K49R, D71G, K91E, E53F | 153 | 154 |
| L9V, E14G, K49R, D71G, K91E, E53A | 155 | 156 |
| L9V, E14G, K49R, D71G, K91E, E52R | 157 | 158 |
| L9V, E14G, K49R, D71G, K91E, E52Q | 159 | 160 |
| L9V, E14G, K49R, D71G, K91E, D45V | 161 | 162 |
| L9V, E14G, K49R, D71G, K91E, A2G, I74N | 163 | 164 |
| L9V, E14G, K49R, E53S, D71G, G80K, K91E | 165 | 166 |
| L9V, E14G, K49R, E53S, T68H, D71G, K91E | 167 | 168 |
| L9V, E14G, K49R, E53S, D71G, I74S, K91E | 169 | 170 |
| L9V, E14G, K25D, K49R, E53S, D71G, K91E | 171 | 172 |
| L9V, E14G, K49R, E53S, D71G, S87H, K91E | 173 | 174 |
| L9V, E14G, K49R, E53S, T68C, D71G, K91E | 175 | 176 |
| L9V, E14G, K49R, E53S, D71G, F88Y, K91E | 177 | 178 |
| L9V, E14G, K49R, E53S, D71G, I74K, K91E | 179 | 180 |
| L9V, E14G, Y41Q, K49R, E53S, D71G, K91E | 181 | 182 |
| L9V, E14G, Y41T, K49R, E53S, D71G, K91E | 183 | 184 |
| L9V, E14G, K49R, E53S, T68G, D71G, K91E | 185 | 186 |
| L9V, E14G, K49R, E53S, D71G, D83R, K91E | 187 | 188 |
| L9V, E14G, Y41S, K49R, E53S, D71G, K91E | 189 | 190 |
| L9V, E14G, E21L, K49R, D71G, K91E | 191 | 192 |
| L9V, E14G, K49R, E52S, D71G, K91E | 193 | 194 |
| L9V, E14G, K49R, D71G, D83K, K91E | 195 | 196 |
| L9V, E14G, K49R, D71G, K91E, T98V | 197 | 198 |
| L9V, E14G, K49R, D71G, 174R, K91E | 199 | 200 |
| A2V, L9V, E14G, K49R, D71G, K91E | 201 | 202 |
| L9V, E14G, K49R, T68C, D71G, K91E | 203 | 204 |
| L9V, E14G, K49R, D71G, G82A, K91E | 205 | 206 |
| L9V, E14G, K49R, T68A, D71G, K91E | 207 | 208 |
| L9V, E14G, T47S, K49R, D71G, 174Q, K91E | 209 | 210 |
| L9V, E14G, T47G, K49R, D71G, K91E | 211 | 212 |
| L9V, E14G, K49R, D71G, F88W, K91E | 213 | 214 |
| L9V, E14G, V31A, K49R, D71G, K91E | 215 | 216 |
| A2S, L9V, E14G, K49R, D71G, K91E | 217 | 218 |
| L9V, E14G, A18E, K49R, D71G, K91E | 219 | 220 |
| L9V, E14G, K49R, D71G, R86S, K91E | 221 | 222 |
| L9V, E14G, K49R, D71G, I74H, K91E | 223 | 224 |
| L9V, E14G, K49R, D71G, I74Q, K91E | 225 | 226 |
| L9V, E14G, T47S, K49R, D71G, K91E | 227 | 228 |
| L9V, E14G, K49R, D71G, I74N, K91E | 229 | 230 |
| L9V, E14G, V46L, K49R, E53S, D71G, K91E | 231 | 232 |
| A2G, L9I, K25R | 233 | 234 |
| L9I, E53S, I94K | 235 | 236 |
| A36E | 237 | 238 |
| E53H, E64D | 239 | 240 |
| L9I, K25R, A36E, E53H, I94K | 241 | 242 |
| A2G, L9I, K25R, E53S, E64D, I94K | 243 | 244 |
| K25S, A36E | 245 | 246 |
| L9I, T16Q, K25R, E53S, E64D | 247 | 248 |
| K25S, I94K | 249 | 250 |
| A2G, L9I, K25G | 251 | 252 |
| E53A | 253 | 254 |
| A2G, L9V, T16P | 255 | 256 |
| E53A, E64D, I94K | 257 | 258 |
| L9I, K25S, A36E, E64D, I94K | 259 | 260 |
| L9I, K25S, A36E, E53R, I94K | 261 | 262 |
| K25E, A36E, E64D, I94K | 263 | 264 |
| K25R, E64D | 265 | 266 |
| K25R, A36E, E53S, E64D, I94K | 267 | 268 |
| A2G, L9I, A36E, E53S, E64D, I94K | 269 | 270 |
| A2G, L9V, Y27F, E52R, V66L, V84M | 271 | 272 |
| L9V, V66L | 273 | 274 |
| L9V, Y27F, V84M | 275 | 276 |
| A2G, L9V | 277 | 278 |
| L9I, Y27F, E53S, V66L, V84M | 279 | 280 |
| V66L, V84M | 281 | 282 |
| L9V, E52Q, V66L, P76V | 283 | 284 |
| L9I, Y27F, V66L, V84I | 285 | 286 |
| A2G, L9V, Y27F, V66L | 287 | 288 |
| Y27F | 289 | 290 |
| A2G, L9V, V66L, V84M | 291 | 292 |
| L9V, T26A, E52Q, P76V | 293 | 294 |
| L9V, E52R, V84M | 295 | 296 |
| L9V, Y27F, V66L, V84M | 297 | 298 |
| L9I, N29G, T62G, T68E, I94K | 299 | 300 |
| L9I, E14G, K44P, T68E, I94K | 301 | 302 |
| E14G, T68S, I94K | 303 | 304 |
| E14G, T68A, I94K | 305 | 306 |
| T62G, T68G, I94K | 307 | 308 |
| L9V, E14G, T68E, I94K | 309 | 310 |
| L9V, I94K | 311 | 312 |
| L9V, N29G, T68E, I94K | 313 | 314 |
| A2G, T16P, V31M, K49R, S87K | 315 | 316 |
| V31E, F63L, S87K | 317 | 318 |
| A2G, K49R | 319 | 320 |
| V31M, K49R, S87K | 321 | 322 |
| A2G, V31S, K49R, K91E | 323 | 324 |
| V31E, K49R, S87K | 325 | 326 |
| T16P, V31M, K49R | 327 | 328 |
| L9V, E14G, V31S, K49R, K91E | 329 | 330 |
| K12L, V31M, K49R | 331 | 332 |
| L9V, K91E | 333 | 334 |
| L9I, E14G, V40A, K49R | 335 | 336 |
| L9V, K49R, D71G, K91E | 337 | 338 |
| L9I, E14G, Y27F, D71G, K91E | 339 | 340 |
| E14G, K49R, D71G, K91E | 341 | 342 |
| L9I, Y27F, K91E | 343 | 344 |
| K49R, Y85F, K91E | 345 | 346 |
| L9I, E14G, D71G, K91E | 347 | 348 |
| L9V, K49R, K91E | 349 | 350 |
| L9I, E14G, K49R, D71G | 351 | 352 |
| E14G, Y27F, K49R | 353 | 354 |
| L9V, T16Q, K49R, K91E | 355 | 356 |
| L9I, K49R, K91E | 357 | 358 |
| L9V, E14G, Y27F, K49R, K91E | 359 | 360 |
| K49R, K91E | 361 | 362 |
| L9I, E14G, D71G | 363 | 364 |
| E14G, K49R, K91E | 365 | 366 |
| K49R, E64D, I74T | 367 | 368 |
| K12L, Y27F, K49R, I74M | 369 | 370 |
| A2G, Y27F, E64K, I74V | 371 | 372 |
| Y27F, K49R, E64D, I74V | 373 | 374 |
| Y27F, K49R, E64D, I74T | 375 | 376 |
| A2G, K49R, E64D, I74M | 377 | 378 |
| A2G, K49R, E64D, I74N | 379 | 380 |
| K49R, E64K, I74N | 381 | 382 |
| L9V, E14G, K25A, Q48P, I74N | 383 | 384 |
| K12L, K49R, I74V | 385 | 386 |
| Y27F, K49R, I74V | 387 | 388 |
| K49R, I74S | 389 | 390 |
| A2G, K12L, K49R, I74N | 391 | 392 |
| L9V, E14G, T47A, E64D, I74V | 393 | 394 |
| K12L, K49R, E64D, I74S | 395 | 396 |
| Y27F, K49R, I74N | 397 | 398 |
| A2G, E64K, I74M | 399 | 400 |
| V8I, L9I, I33V, K49R, T56S, D71T | 401 | 402 |
| V8I, L9I, I33D, K49R, T56S, I58V | 403 | 404 |
| V8I, L9I, K12N, K49R, Y55W, T56S | 405 | 406 |
| V8I, L9I, K12V, I33V, T56S, E64D | 407 | 408 |
| L9I, K12V, I33V, T56S, 158V, E64D | 409 | 410 |
| A2P, V81, L9I, E64D, T68S, R100G | 411 | 412 |
| V8I, K12V, T56S, I58V, E64D, Y97F | 413 | 414 |
| V31M, K49R, I58V, T62C, E64D, T68A | 415 | 416 |
| V8I, K49R, I58V, E64D, T68M, I74M | 417 | 418 |
| A36P, I58V, E64D, T68G, Q70K, I74M | 419 | 420 |
| E14G, A36Q, K49R, I58V, E64D, Q70K | 421 | 422 |
| A2P, V8I, L9I, I33V, E64D, I94K | 485 | 486 |
| V8I, L9I, A36S, Y55W, E64D, I94K | 487 | 488 |
| V8I, L9I, I33V, K49R, I58V, T62C | 489 | 490 |
| V8I, L9I, I33V, K49R, I58V, T62C | 491 | 492 |
| V8I, L9I, I33D, K49R, T56S, D71T | 493 | 494 |
| V8I, L9I, K12N, I33V, K49R, D71T | 495 | 496 |
| V8I, L9I, I33V, K49R, I58V, R100G | 497 | 498 |
| V8I, L9I, K12N, I33V, K49R, D71T | 499 | 500 |
| V8I, L9I, K49R, Y55W, I58V, R100G | 501 | 502 |
| V8I, L9I, I33V, K49R, T56S, R100G | 503 | 504 |
| V8I, L9I, I33V, A36L, K49R, I58V | 505 | 506 |
| L9I, I33V, A36Q, I58V, E64D, I94K | 507 | 508 |
| V8I, L9I, K10A, I33V, T56S, I58V | 509 | 510 |
| L9I, I33V, K49R, T56S, I58V, R100G | 511 | 512 |
| V8I, L9I, K12N, I33V, K49R, D71T | 513 | 514 |
| L9I, I33V, A36Q, I58V, E64D, I94K | 515 | 516 |
| V8I, L9I, K10A, N29D, I33V, I58V | 517 | 518 |
| L9I, I33V, K49R, T56S, I58V, R100G | 519 | 520 |
| V8I, L9I, I33D, T56S, I58V, E64D | 521 | 522 |
| V8I, L9I, I33D, K49R, T56S, D71T | 523 | 524 |
| V8I, L9I, I33D, K49R, T56S, D71T | 525 | 526 |
| L9I, A36Q, Y55W, I58V, E64D, I94K | 527 | 528 |
| V8I, L9I, I33V, T56S, I58V, Q70K | 529 | 530 |
| V8I, L9I, K49R, Y55W, T56S, R100G | 531 | 532 |
| V8I, L9I, I33D, K49R, T56S, D71T | 533 | 534 |
| V8I, L9I, K10A, N29D, I33V, T56S | 535 | 536 |
| V8I, L9I, K10A, N29D, I33V, T56S | 537 | 538 |
| A2P, V8I, L9I, Y55W, E64D, I94K | 539 | 540 |
| L9I, A36Q, Y55W, I58V, E64D, I94K | 541 | 542 |
| V8I, L9I, I33V, T56S, I58V, R100G | 543 | 544 |
| V8I, L9I, Y55W, T56S, I58V, R100G | 545 | 546 |
| L9I, A36Q, Y55W, I58V, E64D, I94K | 547 | 548 |
| V8I, L9I, K12N, T16Q, K49R, T56S | 549 | 550 |
| V8I, L9I, K49R, Y55W, I58V, T62C | 551 | 552 |
| A2P, V8I, L9I, T16Q, E64D, I94K | 553 | 554 |
| V8I, L9I, I33D, T56S, I58V, E64D | 555 | 556 |
| V8I, L9I, T16Q, A36F, K49R, I58V | 557 | 558 |
| V8I, I33V, K49R, T56S, I58V, R100G | 559 | 560 |
| V8I, L9I, T16Q, T56S, I58V, Q70K | 561 | 562 |
| V8I, L9I, T16Q, T56S, I58V, R100G | 563 | 564 |
| V8I, L9I, K10A, N29D, I33V, T56S | 565 | 566 |
| V8I, L9I, K10A, N29D, I33V, T56S | 567 | 568 |
| L9I, I33V, K49R, T56S, I58V, R100G | 569 | 570 |
| V8I, L9I, Y55W, T56S, I58V, R100G | 571 | 572 |
| L9I, A36Q, Y55W, I58V, E64D, I94K | 573 | 574 |
| V8I, L9I, K12V, I33V, T56S, I58V | 575 | 576 |
| V8I, L9I, T16Q, A36F, K49R, I58V | 577 | 578 |
| V8I, L9I, K12N, I33V, T56S, D71T | 579 | 580 |
| V8I, L9I, K49R, Y55W, T56S, R100G | 581 | 582 |
| V8I, L9I, I33V, A36S, T62C, E64D | 583 | 584 |
| V8I, K49R, Y55W, T56S, I58V, R100G | 585 | 586 |
| L9I, A36Q, I58V, E64D, T68S, I94K | 587 | 588 |
| V8I, L9I, T56S, I58V, T68S, R100G | 589 | 590 |
| V8I, L9I, K12V, T56S, E64D, T68S | 591 | 592 |
| L9I, A36Q, I58V, E64D, I94K, Y97F | 593 | 594 |
| V8I, L9I, V28C, K49R, T56S, I58V | 595 | 596 |
| V8I, L9I, K12V, I58V, E64D, T68G | 597 | 598 |
| V8I, L9I, A36S, E64D, I94K, Y97F | 599 | 600 |
| V8I, L9I, K12N, K49R, T68G, D71T | 601 | 602 |
| V8I, L9I, K49R, I58V, T62C, Y97F | 603 | 604 |
| V8I, L9I, V28C, K49R, T56S, D71T | 605 | 606 |
| V8I, L9I, K49R, I58V, T62C, Y97F | 607 | 608 |
| V8I, L9I, K10A, N29D, I58V, T68G | 609 | 610 |
| V8I, L9I, K49R, I58V, T68S, R100G | 611 | 612 |
| L9I, K49R, T56S, I58V, T68G, R100G | 613 | 614 |
| V8I, L9I, K12V, T56S, E64D, T68S | 615 | 616 |
| V8I, L9I, K10A, N29D, I58V, T68G | 617 | 618 |
| V8I, L9I, A36S, E64D, I94K, Y97F | 619 | 620 |
| V8I, L9I, K12N, K49R, T56S, Y97F | 621 | 622 |
| V8I, L9I, K12V, I58V, E64D, T68G | 623 | 624 |
| V8I, L9I, K12V, I58V, E64D, Y97F | 625 | 626 |
| V8I, L9I, T56S, I58V, T68S, R100G | 627 | 628 |
| V8I, L9I, K49R, I58V, T62C, T68G | 629 | 630 |
| V8I, L9I, K12V, T56S, E64D, T68S | 631 | 632 |
| V8I, K49R, T56S, I58V, T68S, R100G | 633 | 634 |
| V8I, L9I, T56S, I58V, T68G, Q70K | 635 | 636 |
| V8I, L9I, K49R, I58V, T62C, T68G | 637 | 638 |
| V8I, L9I, K12V, T56S, I58V, T68G | 639 | 640 |
| V8I, L9I, K12V, T56S, I58V, T68S | 641 | 642 |
| V8I, L9I, A36F, K49R, I58V, T68S | 643 | 644 |
| V8I, L9I, K49R, I58V, T62C, Y97F | 645 | 646 |
| V8I, L9I, A36F, K49R, I58V, Y97F | 647 | 648 |
| V8I, L9I, K12N, K49R, T68G, D71T | 649 | 650 |
| V8I, L9I, K12V, T56S, E64D, T68S | 651 | 652 |
| V8I, L9I, K49R, I58V, Y97F, R100G | 653 | 654 |
| V8I, L9I, A36F, K49R, I58V, Y97F | 655 | 656 |
| V8I, L9I, K12V, T56S, E64D, T68S | 657 | 658 |
| V8I, L9I, K10A, N29D, T56S, T68S | 659 | 660 |
| V8I, L9I, V28C, K49R, T56S, I58V | 661 | 662 |
| V8I, L9I, K10A, T56S, I58V, Y97F | 663 | 664 |
| V8I, L9I, K12V, E14G, K49R, E67S | 665 | 666 |
| L9I, A36S, K49R, E64D, T68M, I74M | 667 | 668 |
| L9I, Y41E, K49R, I58V, V66I, T68E | 669 | 670 |
| V8I, L9I, K12V, E14G, K49R, E67S | 671 | 672 |
| V8I, L9I, K12N, T16Q, K49R, I58V | 673 | 674 |
| V8I, L9I, K12V, E14G, K49R, E67S | 675 | 676 |
| V8I, L9I, K25N, K49R, I58V, T68S | 677 | 678 |
| V8, L9I, K49R, T56S, V66I, T68M | 679 | 680 |
| V8I, L9I, K12V, E14G, K49R, E67S | 681 | 682 |
| V8I, L9I, K25N, A36P, T68G, I74M | 683 | 684 |
| L9I, Y41E, K49R, I58V, V66I, T68E | 685 | 686 |
| V8I, L9I, K49R, E64D, Y97F, R100G | 687 | 688 |
| V8I, L9I, K12V, E14G, K49R, E67S | 689 | 690 |
| V8I, L9I, I33V, I58V, E64D, R100G | 691 | 692 |
| L9I, A36S, K49R, E64D, T68M, I74M | 693 | 694 |
| V8I, L9I, K49R, E64D, Y97F, R100G | 695 | 696 |
| V8I, L9I, Y41E, I58V, E64D, T68A | 697 | 698 |
| V8I, K12Q, V31M, V66I, T68S, I74M | 699 | 700 |
| V8I, L9I, Y41E, I58V, E64D, T68A | 701 | 702 |
| L9I, E14G, I58V, T62C, E64D, V66I | 703 | 704 |
| V8I, L9I, K49R, T56S, V66I, T68M | 705 | 706 |
| V8I, L9I, K25N, A36P, T68G, I74M | 707 | 708 |
| V8I, L9I, K25N, K49R, I58V, T68S | 709 | 710 |
| L9I, T16Q, K49R, I58V, E64D, R100G | 711 | 712 |
| V8I, K12Q, V31M, V66I, T68S, I74M | 713 | 714 |
| V8I, K12Q, I33V, K49R, I58V, I74M | 715 | 716 |
| V8I, L9I, K49R, E64D, Y97F, R100G | 717 | 718 |
| L9I, T16Q, K49R, I58V, E64D, R100G | 719 | 720 |
| L9I, E14G, I58V, T62C, E64D, V66I | 721 | 722 |
| V8I, L9I, K49R, E64D, Y97F, R100G | 723 | 724 |
| V8I, K12N, I33V, I58V, E64D, V66I | 725 | 726 |
| A2P, L9I, K25N, V31M, K49R, Y55W | 727 | 728 |
| V8I, L9I, K12N, T16Q, K49R, I58V | 729 | 730 |
| L9I, A36S, K49R, E64D, T68M, I74M | 731 | 732 |
| L9I, K49R, I58V, T68A, D71T, I94K | 733 | 734 |
| L9I, E14G, I58V, T62C, E64D, V66I | 735 | 736 |
| V8I, E14G, K49R, I58V, E64D, R100A | 737 | 738 |
| L9V, E14G, K49R, E53S, D71G, K91E | 739 | 740 |
| A2S, V8I, K49R, E53V, T56S, T68S | 741 | 742 |
| V8I, K12Q, V31M, V66I, T68S, I74M | 743 | 744 |
| V8I, K12Q, V31M, V66I, T68S, I74M | 745 | 746 |
| A2P, V8I, K49R, T62C, T68E, I74M | 747 | 748 |
| V8I, L9I, K12N, T16Q, K49R, I58V | 749 | 750 |
| V8I, L9I, I33V, K49R, T62C, E64D | 751 | 752 |
| V8I, K12Q, V31M, V66I, T68S, I74M | 753 | 754 |
| L9I, E53V, I58V, E64D, T68S, I74M | 755 | 756 |
| V8I, V28C, A36S, K49R, T56S, V66I | 757 | 758 |
| V8I, K12Q, V31M, V66I, T68S, I74M | 759 | 760 |
| V8I, L9I, K49R, E64D, Y97F, R100G | 761 | 762 |
| V8I, L9I, I33V, I58V, E64D, R100G | 763 | 764 |
| V8I, E14G, K49R, I58V, E64D, R100A | 765 | 766 |
| V8I, L9I, K25N, A36P, T68G, I74M | 767 | 768 |
| V8I, V31M, E64D, T68Q, Q70K, D71T | 769 | 770 |
| L9I, K25N, A36Q, K49R, V66I, T68Q | 771 | 772 |
| L9I, K25N, A36Q, K49R, V66I, T68Q | 773 | 774 |
| V8I, K49R, E64D, T68S, E90D, I94K | 775 | 776 |
| V8I, A36S, T56S, T68S, E90D, I94K | 777 | 778 |
| L9I, K49R, T62C, E64D, T68G, I74M | 779 | 780 |
| K12N, T56S, I58V, V66I, T68A, D71T | 781 | 782 |
| L9I, I33V, Q48M, I58V, E64D, I74M | 783 | 784 |
| L9I, V31M, K49R, N50Y, T68S, I74M | 785 | 786 |
| V8I, K25N, I33V, K49R, E64D, I74M | 787 | 788 |
| L9I, A18S, K25N, K49R, I58V, S87P | 789 | 790 |
| L9I, E22L, K49R, E64D, I74M, Y97F | 791 | 792 |
| V8I, K49R, E64D, T68S, E90D, I94K | 793 | 794 |
| L9I, E22L, V46I, E64D, I74M, Y97F | 795 | 796 |
| V8I, A18S, K49R, E64D, V66I, I94K | 797 | 798 |
| L9I, E22L, K49R, E64D, I74M, Y97F | 799 | 800 |
| A2S, V8I, T56S, T62C, T68S, I74M | 801 | 802 |
| Y41E, K49R, T56S, I58V, V66I, T68A | 803 | 804 |
| K10A, K49R, I58V, E64D, V66I, T68E | 805 | 806 |
| K12N, A18S, K49R, I58V, V66I, I74M | 807 | 808 |
| K49R, I58V, T62C, T68E, D71T, I74M | 809 | 810 |
| I33V, K49R, T62C, E64D, V661, I74M | 811 | 812 |
| T16Q, K25N, A36Q, K49R, V661, I74M | 813 | 814 |
| A2P, I58V, E64D, E67S, T68Q, R100G | 815 | 816 |
| I33V, K49R, T62C, E64D, V661, I74M | 817 | 818 |
| K49R, I58V, T68A, D71T, I74M, R100G | 819 | 820 |
| K25N, K49R, I58V, E64D, V66I, T68S | 821 | 822 |
| A36Q, K49R, I58V, E64D, V66I, K91E | 823 | 824 |
| K12N, A18S, K49R, I58V, V661, I74M | 825 | 826 |
| E21V, A36Q, I58V, V66I, E67S, I74M | 827 | 828 |
| A2P, E14G, Q48C, I58V, E64D, V66I | 829 | 830 |
| A2P, K12V, T16Q, K49R, I58V, V66I | 831 | 832 |
| A2P, K12V, T16Q, K49R, I58V, V66I | 833 | 834 |
| I33V, K49R, T62C, E64D, V66I, I74M | 835 | 836 |
| A2P, V31M, 158V, T62C, E64D, T68S | 837 | 838 |
| A36P, I58V, E67S, T68S, I74M, R100G | 839 | 840 |
| F23I, K49R, V66I, T68Q, Q70A, I74M | 841 | 842 |
| E22L, V31M, A36S, V66I, T68S, I74M | 843 | 844 |
| A36S, K49R, I58C, E64D, V66I, T68S | 845 | 846 |
| T16Q, A36Q, Q48H, I58V, E64D, I74M | 847 | 848 |
| K25N, K49R, Y55W, I58V, E64D, I74M | 849 | 850 |
| A36S, Q48C, E64D, T68E, I74M, S87P | 851 | 852 |
| K49R, V66I, T68Q, D71T, I74M, I94K | 853 | 854 |
| A2P, V8I, L9V, E64D, T68S, R100G | 855 | 856 |
| A2P, V8I, L9T, E64D, T68S, R100G | 857 | 858 |
| A2P, V8I, L9C, E64D, T68S, R100G | 859 | 860 |
| A2P, V8I, L9G, E64D, T68S, R100G | 861 | 862 |
| A2P, V8I, L9A, E64D, T68S, R100G | 863 | 864 |
| A2P, V8I, L9M, E64D, T68S, R100G | 865 | 866 |
| A2P, V8I, L9F, E64D, T68S, R100G | 867 | 868 |
| A2P, V8I, L9S, E64D, T68S, R100G | 869 | 870 |
| V8I, K49G, I58V, E64D, T68M, I74M | 871 | 872 |
| V8I, K49A, I58V, E64D, T68M, I74M | 873 | 874 |
| V8I, K49H, I58V, E64D, T68M, I74M | 875 | 876 |
| V8I, K49C, I58V, E64D, T68M, I74M | 877 | 878 |
| V8I, K49T, I58V, E64D, T68M, I74M | 879 | 880 |
| V8I, K49V, I58V, E64D, T68M, I74M | 881 | 882 |
| V8I, K49S, I58V, E64D, T68M, I74M | 883 | 884 |
| V8I, K49N, I58V, E64D, T68M, I74M | 885 | 886 |
| V8I, K49P, I58V, E64D, T68M, I74M | 887 | 888 |
| V8I, K49L, I58V, E64D, T68M, I74M | 889 | 890 |

¹Amino acid differences relative to the wild-type CsOAC sequence of SEQ ID NO: 6 or 20 denoted by standard format of single letter amino acid and position number followed by substituted amino acid in single-letter-e.g., “179C”.
¹Amino acid differences relative to the wild-type CsOAC sequence of SEQ ID NO: 6 or 20 denoted by standard format of single letter amino acid and position number followed by substituted amino acid in single-letter-e.g., “179C”.

In at least one embodiment, the recombinant polypeptides having OAC activity encoded by the engineered genes of the present disclosure have one or more residue differences as compared to the wild-type CsOAC polypeptide of SEQ ID NO: 6 or 20. In some embodiments, the recombinant polypeptides have one or more residue differences at residue positions selected from A2, L6, V8, L9, K10, F11, K12, E14, T16, E17, A18, E21, E22, F23, K25, T26, Y27, V28, N29, V31, I33, A36, V40, Y41, K44, D45, V46, T47, Q48, K49, N50, E52, E53, Y55, T56, H57, I58, T62, T62, E64, V66, T68, Q70, D71, I74, P76, A77, H78, G80, G82, D83, V84, Y85, R86, S87, F88, E90, K91, I94, Y97, T98, and R100.

In at least one embodiment, the polypeptide comprises an amino acid sequence of at least 80% identity to SEQ ID NO: 6, and an amino acid residue difference as compared to SEQ ID NO: 6 at each of a combination of six positions, wherein the combination of six positions are selected from the combinations listed in Table 4.

TABLE 4
Specific Combinations of Six Positions Amino Acid Residue
Differences (relative to SEQ ID NO: 6 or 20)

| | |
|---|---|
| A2, V8, L9, E64, T68, R100 | V8, K49, I58, E64, T68, I74 |
| A2, I58, E64, E67, T68, R100 | V8, L9, T16, T56, I58, R100 |
| A2, K12, T16, K49, I58, V66 | V8, L9, T56, I58, T68, Q70 |
| A2, L9, E14, K49, D71, K91 | V8, L9, T56, I58, T68, R100 |
| A2, L9, K25, V31, K49, Y55 | V8, L9, V28, K49, T56, D71 |
| A2, V31, I58, T62, E64, T68 | V8, L9, V28, K49, T56, I58 |
| A2, V8, K49, E53, T56, T68 | V8, L9, Y41, I58, E64, T68 |
| A2, V8, K49, T62, T68, I74 | V8, L9, Y55, T56, I58, R100 |
| A2, E14, Q48, I58, E64, V66 | V8, V28, A36, K49, T56, V66 |
| A2, V8, L9, I33, E64, I94 | V8, V31, E64, T68, Q70, D71 |
| A2, V8, L9, T16, E64, I94 | L9, A18, K25, K49, I58, S87 |
| A2, V8, L9, Y55, E64, I94 | L9, A36, I58, E64, I94, Y97 |
| A2, V8, T56, T62, T68, I74 | L9, A36, I58, E64, T68, I94 |
| L6, L9, E14, K49, D71, K91 | L9, A36, K49, E64, T68, I74 |
| V8, A18, K49, E64, V66, I94 | L9, A36, Y55, I58, E64, I94 |
| V8, A36, T56, T68, E90, I94 | L9, E14, A18, K49, D71, K91 |
| V8, E14, K49, I58, E64, R100 | L9, E14, D45, K49, D71, K91 |
| V8, I33, K49, T56, I58, R100 | L9, E14, E21, K49, D71, K91 |
| V8, K12, I33, I58, E64, V66 | L9, E14, I33, K49, D71, K91 |
| V8, K12, I33, K49, I58, I74 | L9, E14, I58, T62, E64, V66 |
| V8, K12, T56, I58, E64, Y97 | L9, E14, K49, D71, D83, K91 |
| V8, K12, V31, V66, T68, I74 | L9, E14, K49, D71, F88, K91 |
| V8, K25, I33, K49, E64, I74 | L9, E14, K49, D71, G82, K91 |
| V8, K49, E64, T68, E90, I94 | L9, E14, K49, D71, I74, K91 |
| V8, L9, T16, T56, I58, Q70 | L9, E14, K49, D71, K91, T98 |
| V8, K49, T56, I58, T68, R100 | L9, E14, K49, D71, K91, Y97 |
| V8, K49, Y55, T56, I58, R100 | L9, E14, K49, D71, R86, K91 |
| V8, L9, A36, E64, I94, Y97 | L9, E14, K49, E52, D71, K91 |
| V8, L9, A36, K49, I58, T68 | L9, E14, K49, E53, D71, K91 |
| V8, L9, A36, K49, I58, Y97 | L9, E14, K49, E64, D71, K91 |
| V8, L9, A36, Y55, E64, I94 | L9, E14, K49, H57, D71, K91 |
| V8, L9, I33, A36, K49, I58 | L9, E14, K49, T68, D71, K91 |
| V8, L9, I33, A36, T62, E64 | L9, E14, K49, V66, D71, K91 |
| V8, L9, I33, I58, E64, R100 | L9, E14, K49, Y55, D71, K91 |
| V8, L9, I33, K49, I58, R100 | L9, E14, N29, K49, D71, K91 |
| V8, L9, I33, K49, I58, T62 | L9, E14, T16, K49, D71, K91 |
| V8, L9, I33, K49, T56, D71 | L9, E14, T47, K49, D71, K91 |
| V8, L9, I33, K49, T56, I58 | L9, E14, V28, K49, D71, K91 |
| V8, L9, I33, K49, T56, R100 | L9, E14, V31, K49, D71, K91 |
| V8, L9, I33, K49, T62, E64 | L9, E22, K49, E64, I74, Y97 |
| V8, L9, I33, T56, I58, E64 | L9, E22, V46, E64, I74, Y97 |
| V8, L9, I33, T56, I58, Q70 | L9, E53, I58, E64, T68, I74 |
| V8, L9, I33, T56, I58, R100 | L9, I33, A36, I58, E64, I94 |
| V8, L9, K10, I33, T56, I58 | L9, I33, K49, T56, I58, R100 |
| V8, L9, K10, N29, I33, I58 | L9, I33, Q48, I58, E64, I74 |
| V8, L9, K10, N29, I33, T56 | L9, K12, I33, T56, I58, E64 |
| V8, L9, K10, N29, I58, T68 | L9, K25, A36, K49, V66, T68 |
| V8, L9, K10, N29, T56, T68 | L9, K49, I58, T68, D71, I94 |
| V8, L9, K10, T56, I58, Y97 | L9, K49, T56, I58, T68, R100 |
| V8, L9, K12, E14, K49, E67 | L9, K49, T62, E64, T68, I74 |
| V8, L9, K12, I33, K49, D71 | L9, T16, K49, I58, E64, R100 |
| V8, L9, K12, I33, T56, D71 | L9, T16, T26, K49, D71, K91 |
| V8, L9, K12, I33, T56, E64 | L9, V31, K49, N50, T68, I74 |
| V8, L9, K12, I33, T56, I58 | L9, Y41, K49, I58, V66, T68 |
| V8, L9, K12, I58, E64, T68 | K10, K49, I58, E64, V66, T68 |
| V8, L9, K12, I58, E64, Y97 | K12, A18 K49, I58, V66, I74 |
| V8, L9, K12, K49, T56, Y97 | K12, T56, I58, V66, T68, D71 |
| V8, L9, K12, K49, T68, D71 | E14, A36, K49, I58, E64, Q70 |
| V8, L9, K12, K49, Y55, T56 | T16, A36, Q48, I58, E64, I74 |
| V8, L9, K12, T16, K49, I58 | T16, K25, A36, K49, V66, I74 |
| V8, L9, K12, T16, K49, T56 | E21, A36, I58, V66, E67, I74 |
| V8, L9, K12, T56, E64, T68 | E22, V31, A36, V66, T68, I74 |
| V8, L9, K12, T56, I58, T68 | F23, K49, V66, T68, Q70, I74 |
| V8, L9, K25, A36, T68, I74 | K25, K49, I58, E64, V66, T68 |
| V8, L9, K25, K49, I58, T68 | K25, K49, Y55, I58, E64, I74 |
| V8, L9, K49, E64, Y97, R100 | V31, K49, I58, T62, E64, T68 |
| V8, L9, K49, I58, T62, T68 | I33, K49, T62, E64, V66, I74 |
| V8, L9, K49, I58, T62, T68 | A36, I58, E64, T68, Q70, I74 |
| V8, L9, K49, I58, T62, Y97 | A36, I58, E67, T68, I74, R100 |
| V8, L9, K49, I58, T68, R100 | A36, K49, I58, E64, V66, K91 |
| V8, L9, K49, I58, Y97, R100 | A36, K49, I58, E64, V66, T68 |
| V8, L9, K49, T56, V66, T68 | A36, Q48, E64, T68, I74, S87 |
| V8, L9, K49, Y55, I58, R100 | Y41, K49, T56, I58, V66, T68 |
| V8, L9, K49, Y55, I58, T62 | K49, I58, T62, T68, D71, I74 |
| V8, L9, K49, Y55, T56, R100 | K49, I58, T68, D71, I74, R100 |
| V8, L9, T16, A36, K49, I58 | K49, V66, T68, D71, I74, I94 |

In at least one embodiment, the amino acid residue differences are selected from A2G, A2S, A2P, A2V, L6F, V8I, L9A, L9F, L9G, L9I, L9M, L9S, L9V, K10A, F11L, K12L, K12N, K12Q, K12V, E14G, T16P, T16Q, E17G, A18E, A18S, E21L, E21V, E22L, F23I, K25D, K25G, K25E, K25N, K25R, K25S, T26A, T26N, Y27F, V28C, N29D, N29G, V31A, V31E, V31M, V31S, I33D, I33E, I33V, A36E, A36F, A36L, A36Q, A36S, V40A, V40G, Y41E, Y41Q, Y41S, Y41T, K44P, D45V, V46I, V46L, T47A, T47G, T47S, T47S, Q48C, Q48H, Q48M, Q48P, K49A, K49C, K49G, K49H, K49L, K49N, K49P, K49R, K49S, K49T, K49V, N50Y, E52Q, E52R, E52S, E53A, E53F, E53H, E53L, E53R, E53S, E53V, Y55W, T56S, H57G, I58C, I58V, T62C, T62G, E64D, E64K, V66I, V66L, E67S, T68A, T68C, T68E, T68G, T68H, T68M, T68Q, T68S, Q70A, Q70K, D71G, I74G, I74H, I74K, I74L, I74M, I74N, I74Q, I74R, I74S, I74T, I74V, P76V, A77E, H78P, G80K, G82A, G82R, D83K, D83R, V84I, V84M, Y85F, R86S, S87H, S87K, S87P, F88W, F88Y, E90D, K91E, I94K, Y97F, T98V, R100A, and R100G.

It is contemplated that various combinations of the residue differences associated with comparable or increased OA production relative to the gene encoding the wild type CsOAC can be incorporated in further engineered genes to provide expression of further recombinant polypeptides having desirable functional characteristics in a recombinant host cell. In at least one embodiment, the desirable functional characteristic is increased titer produced of the cannabinoid precursor, OA, and/or the downstream cannabinoid compound, CBGA. Some exemplary combinations of residue differences are described in Tables 3, 5, 7, 8, 9, 10, 13, 14, and 15, and elsewhere herein. For example, the present disclosure provides a engineered genes encoding a recombinant polypeptide having combinations of two, three, four, five, six, or seven amino acid residue differences as compared to wild-type CsOAC (SEQ ID NO: 6 or 20) over a wide range of residue positions spanning the full length of the protein, including positions A2, L6, V8, L9, K10, F11, K12, E14, T16, E17, A18, E21, E22, F23, K25, T26, Y27, V28, N29, V31, I33, A36, V40, Y41, K44, D45, V46, T47, Q48, K49, N50, E52, E53, Y55, T56, H57, I58, T62, T62, E64, V66, T68, Q70, D71, I74, P76, A77, H78, G80, G82, D83, V84, Y85, R86, S87, F88, E90, K91, I94, Y97, T98, and R100. Combinations of amino acid residue differences relative to the wild-type CsOAC (SEQ ID NO: 6 or 20) exemplified in the recombinant OAC polypeptides of the present disclosure are provided in Table 3, and results demonstrating the OAC activity of these engineered polypeptides is provided in Tables 7, 8, 9, 10, 13, 14, and 15. Accordingly, in at least one embodiment, the engineered gene encodes a recombinant polypeptide having at least a combination of two, three, four, five, six, or seven amino acid residue differences relative to the polypeptide of SEQ ID NO: 6 or 20 selected from those combinations listed in Table 5 (below).

TABLE 5
Specific Combinations Amino Acid Residue Differences
(relative to SEQ ID NO: 6 or 20)

| | |
|---|---|
| A2G, K49R | L9V, K91E |
| A2G, L9V | L9V, V66L |
| A2G, E64K, I74M | L9V, E14G, K25A, Q48P, I74N |
| A2G, K12L, K49R, I74N | L9V, E14G, K25D, K49R, E53S, D71G, |
| | K91E |
| A2G, K49R, E64D, I74M | L9V, E14G, K25G, K49R, D71G, H78P, |
| | K91E |
| A2G, L9I, K25G | L9V, E14G, K49R, D71G, K91E |
| A2G, L9I, K25R | L9V, E14G, K49R, E53R, D71G, V84I, K91E |
| A2G, L9V, T16P | L9V, E14G, K49R, E53S, D71G, D83R, |
| | K91E |
| A2G, L9V, E14G, K49R, D71G, I74N, K91E | L9V, E14G, K49R, E53S, D71G, F88Y, |
| | K91E |
| A2G, L9V, V66L, V84M | L9V, E14G, K49R, E53S, D71G, G80K, |
| | K91E |
| A2G, L9V, Y27F, V66L | L9V, E14G, K49R, E53S, D71G, I74K, K91E |
| A2G, T16P, V31M, K49R, S87K | L9V, E14G, K49R, E53S, D71G, I74S, K91E |
| A2G, V31S, K49R, K91E | L9V, E14G, K49R, E53S, D71G, S87H, |
| | K91E |
| A2G, Y27F, E64K, I74V | L9V, E14G, K49R, E53S, T68C, D71G, |
| | K91E |
| E14G, K49R, K91E | L9V, E14G, K49R, E53S, T68G, D71G, |
| | K91E |
| E14G, K49R, D71G, K91E | L9V, E14G, K49R, E53S, T68H, D71G, |
| | K91E |
| E14G, T68A, I94K | L9V, E14G, T16Q, A36E, K49R, D71G, |
| | K91E |
| E14G, T68S, I94K | L9V, E14G, T16Q, K49R, D71G, V84M, |
| | K91E |
| E14G, Y27F, K49R | L9V, E14G, T47A, E64D, I74V |
| E17G, K44P | L9V, E14G, T47S, K49R, D71G, I74Q, K91E |
| E53A, E64D, I94K | L9V, E14G, T68E, I94K |
| E53H, E64D | L9V, E14G, V31S, K49R, K91E |
| K12L, K49R, I74V | L9V, E14G, V46L, K49R, E53S,D71G, K91E |
| K12L, K49R, E64D, I74S | L9V, E14G, Y27F, K49R, K91E |
| K12L, V31M, K49R | L9V, E14G, Y41Q, K49R, E53S, D71G, |
| | K91E |
| K12L, Y27F, K49R, I74M | L9V, E14G, Y41S, K49R, E53S, D71G, |
| | K91E |
| K25E, A36E, E64D, I94K | L9V, E14G, Y41T, K49R, E53S, D71G, |
| | K91E |
| K25R, A36E, E53S, E64D, I94K | L9V, E21V, G82A |
| K25R, E64D | L9V, E52Q, V66L, P76V |
| K25S, A36E | L9V, E52R, V84M |
| K25S, I94K | L9V, K49R, K91E |
| K49R, I74S | L9V, K49R, D71G, K91E |
| K49R, K91E | L9V, N29G, T68E, I94K |
| K49R, E64D, I74T | L9V, T16Q, K49R, K91E |
| K49R, E64K, I74N | L9V, T26A, E52Q, P76V |
| K49R, Y85F, K91E | L9V, Y27F, V84M |
| L9I, E14G, D71G | L9V, Y27F, V66L, V84M |
| L9I, E14G, D71G, K91E | T16P, V31M, K49R |
| L9I, E14G, K44P, T68E, I94K | T62G, T68G, I94K |
| L9I, E14G, K49R, D71G | V31E, F63L, S87K |
| L9I, E14G, K49R, D71G, K91E | V31E, K49R, S87K |
| L9I, E14G, V40A, K49R | V31M, K49R, S87K |
| L9I, E14G, Y27F, D71G, K91E | V66L, V84M |
| L9I, E53S, I94K | Y27F, K49R, I74N |
| L9I, K25R, A36E, E53H, I94K | Y27F, K49R, I74V |
| L9I, K25S, A36E, E53R, I94K | Y27F, K49R, E64D, I74T |
| L9I, K25S, A36E, E64D, I94K | Y27F, K49R, E64D, I74V |
| L9I, K49R, K91E | L9I, Y27F, K91E |
| L9I, N29G, T62G, T68E, I94K | L9I, Y27F, E53S, V66L, V84M |
| L9I, T16Q, K25R, E53S, E64D | L9I, Y27F, V66L, V84I |
| L9V, I94K | L9V, E21V |
| A2P, V8I, L9V, E64D, T68S, R100G | V8I, K49R, I58V, E64D, T68M, I74M |
| A2P, I58V, E64D, E67S, T68Q, R100G | V8I, L9I, T56S, I58V, T68S, R100G |
| A2P, K12V, T16Q, K49R, I58V, V66I | V8I, L9I, V28C, K49R, T56S, D71T |
| A2P, L9I, K25N, V31M, K49R, Y55W | V8I, L9I, V28C, K49R, T56S, I58V |
| A2P, V31M, I58V, T62C, E64D, T68S | V8I, L9I, Y41E, I58V, E64D, T68A |
| A2P, V8I, K49R, T62C, T68E, I74M | V8I, L9I, Y55W, T56S, I58V, R100G |
| A2P, V8I, L9A, E64D, T68S, R100G | V8I, V28C, A36S, K49R, T56S, V66I |
| A2P, V8I, L9C, E64D, T68S, R100G | V8I, V31M, E64D, T68Q, Q70K, D71T |
| A2P, V8I, L9F, E64D, T68S, R100G | L9I, A18S, K25N, K49R, I58V, S87P |
| A2P, V8I, L9G, E64D, T68S, R100G | L9I, A36Q, I58V, E64D, I94K, Y97F |
| A2P, V8I, L9I, E64D, T68S, R100G | L9I, A36Q, I58V, E64D, T68S, I94K |
| A2P, V8I, L9I, I33V, E64D, I94K | L9I, A36Q, Y55W, I58V, E64D, I94K |
| A2P, V8I, L9I, T16Q, E64D, I94K | L9I, A36S, K49R, E64D, T68M, I74M |
| A2P, V8I, L9I, Y55W, E64D, I94K | L9I, E14G, I58V, T62C, E64D, V66I |
| A2P, V8I, L9M, E64D, T68S, R100G | L9I, E22L, K49R, E64D, I74M, Y97F |
| A2P, V8I, L9S, E64D, T68S, R100G | L9I, E22L, V46I, E64D, I74M, Y97F |
| A2P, V8I, L9T, E64D, T68S, R100G | L9I, E53V, I58V, E64D, T68S, I74M |
| A2P, E14G, Q48C, I58V, E64D, V66I | L9I, I33V, A36Q, I58V, E64D, I94K |
| A2S, L9V, E14G, K49R, D71G, K91E | L9I, I33V, K49R, T56S, I58V, R100G |
| A2S, V8I, K49R, E53V, T56S, T68S | L9I, I33V, Q48M, I58V, E64D, I74M |
| A2S, V8I, T56S, T62C, T68S, I74M | L9I, K12V, I33V, T56S, I58V, E64D |
| A2V, L9V, E14G, K49R, D71G, K91E | L9I, K25N, A36Q, K49R, V66I, T68Q |
| L6F, L9V, E14G, K49R, D71G, K91E | L9I, K49R, I58V, T68A, D71T, I94K |
| V8I, A18S, K49R, E64D, V66I, I94K | L9I, K49R, T56S, I58V, T68G, R100G |
| V8I, A36S, T56S, T68S, E90D, I94K | L9I, K49R, T62C, E64D, T68G, I74M |
| V8I, E14G, K49R, I58V, E64D, R100A | L9I, T16Q, K49R, I58V, E64D, R100G |
| V8I, E14G, K49R, I58V, E64D, R100A | L9I, V31M, K49R, N50Y, T68S, I74M |
| V8I, I33V, K49R, T56S, I58V, R100G | L9I, Y41E, K49R, I58V, V66I, T68E |
| V8I, K12N, I33V, I58V, E64D, V66I | L9V, E14G, A18E, K49R, D71G, K91E |
| V8I, K12Q, I33V, K49R, I58V, I74M | L9V, E14G, D45V, K49R, D71G, K91E |
| V8I, K12Q, V3IM, V66I, T68S, I74M | L9V, E14G, E21L, K49R, D71G, K91E |
| V8I, K12V, T56S, I58V, E64D, Y97F | L9V, E14G, I33D, K49R, D71G, K91E |
| V8I, K25N, I33V, K49R, E64D, I74M | L9V, E14G, I33V, K49R, D71G, K91E |
| V8I, K49A, I58V, E64D, T68M, I74M | L9V, E14G, K49R, D71G, D83K, K91E |
| V8I, K49C, I58V, E64D, T68M, I74M | L9V, E14G, K49R, D71G, F88W, K91E |
| V8I, K49G, I58V, E64D, T68M, I74M | L9V, E14G, K49R, D71G, G82A, K91E |
| V8I, K49H, I58V, E64D, T68M, I74M | L9V, E14G, K49R, D71G, I74G, K91E |
| V8I, K49L, I58V, E64D, T68M, I74M | L9V, E14G, K49R, D71G, I74H, K91E |
| V8I, K49N, I58V, E64D, T68M, I74M | L9V, E14G, K49R, D71G, I74L, K91E |
| V8I, K49P, I58V, E64D, T68M, I74M | L9V, E14G, K49R, D71G, I74M, K91E |
| V8I, K49R, E64D, T68S, E90D, I94K | L9V, E14G, K49R, D71G, I74N, K91E |
| V8I, K49R, T56S, I58V, T68S, R100G | L9V, E14G, K49R, D71G, I74Q, K91E |
| V8I, K49R, Y55W, T56S, I58V, R100G | L9V, E14G, K49R, D71G, I74R, K91E |
| V8I, K49S, I58V, E64D, T68M, I74M | L9V, E14G, K49R, D71G, I74T, K91E |
| V8I, K49T, I58V, E64D, T68M, I74M | L9V, E14G, K49R, D71G, I74V, K91E |
| V8I, K49V, I58V, E64D, T68M, I74M | L9V, E14G, K49R, D71G, K91E, T98V |
| V8I, L9I, A36F, K49R, I58V, T68S | L9V, E14G, K49R, D71G, K91E, Y97F |
| V8I, L9I, A36F, K49R, I58V, Y97F | L9V, E14G, K49R, D71G, R86S, K91E |
| V8I, L9I, A36S, E64D, I4K, Y97F | L9V, E14G, K49R, E52Q, D71G, K91E |
| V8I, L9I, A36S, Y55W, E64D, I94K | L9V, E14G, K49R, E52R, D71G, K91E |
| V8I, L9I, I33D, K49R, T56S, D71T | L9V, E14G, K49R, E52S, D71G, K91E |
| V8I, L9I, 133D, K49R, T56S, I58V | L9V, E14G, K49R, E53A, D71G, K91E |
| V8I, L9I, I33D, T56S, I58V, E64D | L9V, E14G, K49R, E53F, D71G, K91E |
| V8I, L9I, I33V, A36L, K49R, I58V | L9V, E14G, K49R, E53H, D71G, K91E |
| V8I, L9I, I33V, A36S, T62C, E64D | L9V, E14G, K49R, E53L, D71G, K91E |
| V8I, L9I, 133V, I58V, E64D, R100G | L9V, E14G, K49R, E53R, D71G, K91E |
| V8I, L9I, I33V, K49R, I58V, R100G | L9V, E14G, K49R, E53S, D71G, K91E |
| V8I, L9I, I33V, K49R, I58V, T62C | L9V, E14G, K49R, E64D, D71G, K91E |
| V8I, L9I, I33V, K49R, T56S, D71T | L9V, E14G, K49R, H57G, D71G, K91E |
| V8I, L9I, I33V, K49R, T56S, R100G | L9V, E14G, K49R, T68A, D71G, K91E |
| V8I, L9I, I33V, K49R, T62C, E64D | L9V, E14G, K49R, T68C, D71G, K91E |
| V8I, L9I, I33V, T56S, I58V, Q70K | L9V, E14G, K49R, T68E, D71G, K91E |
| V8I, L9I, I33V, T56S, I58V, R100G | L9V, E14G, K49R, T68G, D71G, K91E |
| V8I, L9I, K10A, I33V, T56S, I58V | L9V, E14G, K49R, T68M, D71G, K91E |
| V8I, L9I, K10A, N29D, I33V, I58V | L9V, E14G, K49R, T68Q, D71G, K91E |
| V8I, L9I, K10A, N29D, I33V, T56S | L9V, E14G, K49R, T68S, D71G, K91E |
| V8I, L9I, K10A, N29D, I58V, T68G | L9V, E14G, K49R, V66L, D71G, K91E |
| V8I, L9I, K10A, N29D, T56S, T68S | L9V, E14G, K49R, Y55W, D71G, K91E |
| V8I, L9I, K10A, T56S, I58V, Y97F | L9V, E14G, N29G, K49R, D71G, K91E |
| V8I, L9I, K12N, I33V, K49R, D71T | L9V, E14G, T16Q, K49R, D71G, K91E |
| V8I, L9I, K12N, I33V, T56S, D71T | L9V, E14G, T47G, K49R, D71G, K91E |
| V8I, L9I, K12N, K49R, T56S, Y97F | L9V, E14G, T47S, K49R, D71G, K91E |
| V8I, L9I, K12N, K49R, T68G, D71T | L9V, E14G, V28C, K49R, D71G, K91E |
| V8I, L9I, K12N, K49R, Y55W, T56S | L9V, E14G, V31A, K49R, D71G, K91E |
| V8I, L9I, K12N, T16Q, K49R, I58V | L9V, E14G, V31E, K49R, D71G, K91E |
| V8I, L9I, K12N, T16Q, K49R, T56S | L9V, E14G, V31M, K49R, D71G, K91E |
| V8I, L9I, K12V, E14G, K49R, E67S | L9V, E14G, V31S, K49R, D71G, K91E |
| V8I, L9I, K12V, I33V, T56S, E64D | L9V, T16P, T26A, K49R, D71G, K91E |
| V8I, L9I, K12V, I33V, T56S, I58V | K10A, K49R, I58V, E64D, V66I, T68E |
| V8I, L9I, K12V, I58V, E64D, T68G | K12N, A18S, K49R, I58V, V66I, I74M |
| V8I, L9I, K12V, I58V, E64D, Y97F | K12N, T56S, I58V, V66I, T68A, D71T |
| V8I, L9I, K12V, T56S, E64D, T68S | E14G, A36Q, K49R, I58V, E64D, Q70K |
| V8I, L9I, K12V, T56S, I58V, T68G | T16Q, A36Q, Q48H, I58V, E64D, I74M |
| V8I, L9I, K12V, T56S, I58V, T68S | T16Q, K25N, A36Q, K49R, V66I, I74M |
| V8I, L9I, K25N, A36P, T68G, I74M | E21V, A36Q, I58V, V66I, E67S, I74M |
| V8I, L9I, K25N, K49R, I58V, T68S | E22L, V31M, A36S, V66I, T68S, I74M |
| V8I, L9I, K49R, E64D, Y97F, R100G | F23I, K49R, V66I, T68Q, Q70A, I74M |
| V8I, L9I, K49R, I58V, T62C, T68G | K25N, K49R, I58V, E64D, V66I, T68S |
| V8I, L9I, K49R, I58V, T62C, Y97F | K25N, K49R, Y55W, I58V, E64D, I74M |
| V8I, L9I, K49R, I58V, T68S, R100G | V31M, K49R, I58V, T62C, E64D, T68A |
| V8I, L9I, K49R, I58V, Y97F, R100G | I33V, K49R, T62C, E64D, V66I, I74M |
| V8I, L9I, K49R, T56S, V66I, T68M | A36P, I58V, E64D, T68G, Q70K, I74M |
| V8I, L9I, K49R, T56S, V66I, T68M | A36P, I58V, E67S, T68S, I74M, R100G |
| V8I, L9I, K49R, Y55W, I58V, R100G | A36Q, K49R, I58V, E64D, V66I, K91E |
| V8I, L9I, K49R, Y55W, I58V, T62C | A36S, K49R, I58C, E64D, V66I, T68S |
| V8I, L9I, K49R, Y55W, T56S, R100G | A36S, Q48C, E64D, T68E, I74M, S87P |
| V8I, L9I, T16Q, A36F, K49R, I58V | Y41E, K49R, T56S, I58V, V66I, T68A |
| V8I, L9I, T16Q, T56S, I58V, Q70K | K49R, I58V, T62C, T68E, D71T, 174M |
| V8I, L9I, T16Q, T56S, I58V, R100G | K49R, I58V, T68A, D71T, I74M, R100G |
| V8I, L9I, T56S, I58V, T68G, Q70K | K49R, V66I, T68Q, D71T, I74M, I94K |

Based on the correlation of recombinant polypeptide functional information provided herein with the sequence information provided in Tables 3, 7, 8, 9, 10, 13, 14, and 15, the accompanying Sequence Listing, one of ordinary skill can recognize that the present disclosure provides a range of recombinant polypeptides having OAC activity, wherein the polypeptide comprises an amino acid sequence comprising one or more of the amino acid differences or combinations of amino acid differences relative to CsOAC (SEQ ID NO: 6 or 20) disclosed in any one of SEQ ID NO: 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, and 890 (i.e., the sequences of even-numbered SEQ ID NOs: 22 to 890), and otherwise have at least 80%, at least 85% at least 90%, at least 95%, at least 97%, at least 98%, or at least 99% identity to a sequence selected from the group consisting of the even-numbered SEQ ID NOs: 22 to 890.

Thus, in at least one embodiment, a recombinant polypeptide of the present disclosure having OAC activity can have an amino acid sequence comprising one or more of the amino acid differences or sets of amino acid differences relative to CsOAC (SEQ ID NO: 6 or 20) disclosed in any one of the sequences of the even-numbered SEQ ID NOs: 22 to 890, and additionally have 1-2, 1-3, 1-4, 1-5, 1-6, 1-7, 1-8, 1-9, 1-10, 1-11, 1-12, 1-14, 1-15, 1-16, 1-18, or 1-20, residue differences at other residue positions. In some embodiments, the number of differences can be 1, 2, 3, 4, 5, 6, 7, 8, 9, 10, 11, 12, 14, 15, 16, 18, or 20 residue differences at the other residue positions.

In addition to the residue positions specified above, any of the engineered prenyltransferase polypeptides disclosed herein can further comprise other residue differences relative to the reference polypeptide of CsOAC (SEQ ID NO: 6 or 20) at other residue positions.

Residue differences at these other residue positions can provide for additional variations in the amino acid sequence without adversely affecting the ability of the recombinant polypeptide to carry out the desired biocatalytic conversion (e.g., conversion of compound (2) to compound (1). In some embodiments, the recombinant polypeptides can have additionally 1-2, 1-3, 1-4, 1-5, 1-6, 1-7, 1-8, 1-9, 1-10, 1-11, 1-12, 1-14, 1-15, 1-16, 1-18, or 1-20 residue differences at other amino acid residue positions as compared to SEQ ID NO: 6 or 20. In some embodiments, the number of differences can be 1, 2, 3, 4, 5, 6, 7, 8, 9, 10, 11, 12, 14, 15, 16, 18, or 20 residue differences at other residue positions. The residue difference at these other positions can include conservative changes or non-conservative changes. In some embodiments, the residue differences can comprise conservative substitutions and non-conservative substitutions as compared to the reference polypeptide of CsOAC (SEQ ID NO: 6 or 20).

In some embodiments, the recombinant polypeptides of the disclosure can be in the form of fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the recombinant polypeptides described herein can be used with or without fusions to other polypeptides. It is also contemplated that the recombinant polypeptides described herein are not restricted to the genetically encoded amino acids. In addition to the genetically encoded amino acids, the polypeptides described herein may be comprised, either in whole or in part, of naturally-occurring and/or synthetic non-encoded amino acids.

In another aspect, the present disclosure provides polynucleotides encoding the recombinant polypeptides having OAC activity and increased activity and/or yield as described herein. In at least one embodiment, the polynucleotide encoding a recombinant polypeptide having OAC activity comprises an amino acid sequence that is at least about 80%, 85%, 90%, 91%, 92%, 93%, 94%, 95%, 96%, 97%, 98%, 99%, or more identical to the CsOAc polypeptide sequence of SEQ ID NO: 6 or 20. In some embodiments, the polynucleotide encodes a recombinant polypeptide comprising an amino acid sequence that has the percent identity described above and has one or more amino acid residue differences as compared to CsOAC (SEQ ID NO: 6 or 20) described elsewhere herein.

In at least one embodiment, the polynucleotide has a sequence encoding a recombinant polypeptide that includes an amino acid difference relative to CsOAC (SEQ ID NO: 6 or 20), and also has one or more codon differences relative to the SEQ ID NO: 5 or SEQ ID NO: 19, which codon differences result in increased yield of the cannabinoid precursor or cannabinoid product produced by a recombinant host cell in which the polynucleotide sequence is integrated. In at least one embodiment, the polynucleotide has a sequence of at least 80% identity to SEQ ID NO: 5 or 19, and a codon difference as compared to either of SEQ ID NO: 5 or 19 at a position not encoding an amino acid residue difference relative to CsOAC (SEQ ID NO: 6 or 20).

It is also contemplated that the polynucleotides encoding the recombinant polypeptides having OAC activity as described herein, can include a combination of one or more codon differences relative to SEQ ID NO: 5 or 19, wherein at least one the codon differences encodes an amino acid difference as compared to the CsOAC polypeptide (SEQ ID NO: 6 or 20) and at least one codon difference does not encode an amino acid difference as compared to SEQ ID NO: 6 or 20. Accordingly, in at least one embodiment, the present disclosure provides a engineered polynucleotide sequence encoding a recombinant polypeptide having OAC activity, wherein the polynucleotide sequence comprises a combination of a codon differences encoding an amino acid difference.

In at least one embodiment, the polynucleotide comprises a sequence encoding an exemplary recombinant polypeptide having OAC activity as disclosed in Tables 3, 5, 6, 7, 8, 11, 12, and 13, and the accompanying Sequence Listing. In at least one embodiment, the polynucleotide comprises a sequence of at least 80%, at least 85%, at least 90%, at least 95%, at least 97%, at least 98%, or at least 99% identity to a sequence selected from the group consisting of SEQ ID NO: 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545. 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, and 889 (i.e., the sequences of odd-numbered SEQ ID NOs: 21 to 889). In at least one embodiment, the polynucleotide comprises a codon degenerate sequence of a polynucleotide sequence selected from the group consisting of the odd-numbered SEQ ID NOs: 21 to 889.

The polynucleotide sequences encoding the recombinant polypeptides of the present disclosure may be operatively linked to one or more heterologous regulatory sequences that control gene expression to create a recombinant polynucleotide capable of expressing the polypeptide. Expression constructs containing a heterologous polynucleotide encoding the recombinant polypeptide can be introduced into appropriate host cells to express the corresponding polypeptide. Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved transaminase enzymes disclosed herein. Thus, having identified a particular amino acid sequence, those skilled in the art could make any number of different nucleic acids by simply modifying the sequence of one or more codons in a way which does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein, including the amino acid sequences presented in Tables 3, 7, 8, 9, 10, 13, 14, and 15, and the accompanying Sequence Listing.

The codons can be selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. It is contemplated that all codons need not be replaced to optimize the codon usage of the recombinant polypeptide since the natural sequence will comprise preferred codons and because use of preferred codons may not be required for all amino acid residues. Consequently, codon optimized polynucleotides encoding the recombinant polypeptide may contain preferred codons at about 40%, 50%, 60%, 70%, 80%, or greater than 90% of codon positions of the full length coding region.

The present disclosure also provides an expression vector comprising a polynucleotide encoding a recombinant polypeptide having OAC activity, and one or more expression regulating regions such as a promoter, a terminator, a replication origin, or the like, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the recombinant polypeptide at such sites. Alternatively, a polynucleotide sequence of the present disclosure may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression. The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively. the vector may be one which, when introduced into the host cell, is integrated into the genome. and replicated together with the chromosome(s) into which it has been integrated. Furthermore, a single vector or plasmid or two or more vectors or plasmids which together contain the total DNA to be introduced into the genome of the host cell, or a transposon may be used. In at least one embodiment, the expression vector further comprises one or more selectable markers, which permit easy selection of transformed cells.

The present disclosure also provides host cell comprising a polynucleotide or expression vector encoding a recombinant polypeptide of the present disclosure, wherein the polynucleotide is operatively linked to one or more control sequences for expression of the polypeptide having OAC activity in the host cell. Host cells for use in expressing the polypeptides encoded by the expression vectors of the present invention are well known in the art and include but are not limited to, bacterial cells, such as E. coli, or fungal cells, such as Saccharomyces cerevisiae or Pichia pastoris, insect cells, such as Drosophila S2 and Spodoptera Sf9, animal cells, such as CHO, COS, BHK, 293, and plant cells. Appropriate culture mediums and growth conditions for the above-described host cells are well known in the art. Accordingly, in at least one embodiment, the present disclosure provides a method for producing a cannabinoid comprising: (a) culturing in a suitable medium a recombinant host cell of the present disclosure; and (b) recovering the produced cannabinoid.

### Use in Recombinant Host Cells

The engineered genes that encode recombinant polypeptides having OAC activity can be incorporated into recombinant host cells for enhanced in vivo biosynthesis of cannabinoids and cannabinoid precursors. In the context of recombinant host cells, recombinant polynucleotides corresponding to the engineered genes can be integrated into a recombinant host cell that has a heterologous pathway capable of producing a cannabinoid or cannabinoid precursor. Generally, such a heterologous pathway integrated in a recombinant host cell includes a polynucleotide sequence encoding three, four, or five linked enzymes that are capable of converting a precursor molecule, such as hexanoic acid (HA) (and associated co-substrates such as malonyl CoA) to a cannabinoid precursor molecule, such as OA, then further convert that cannabinoid precursor to a prenylated cannabinoid compound, such as CBGA, and in some cases, where a fifth synthase enzyme is encoded, to a further cannabinoid molecule, such as THCA.

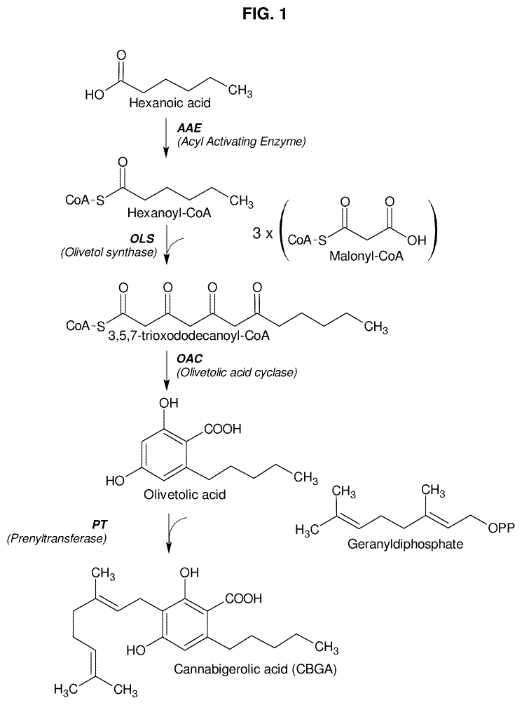

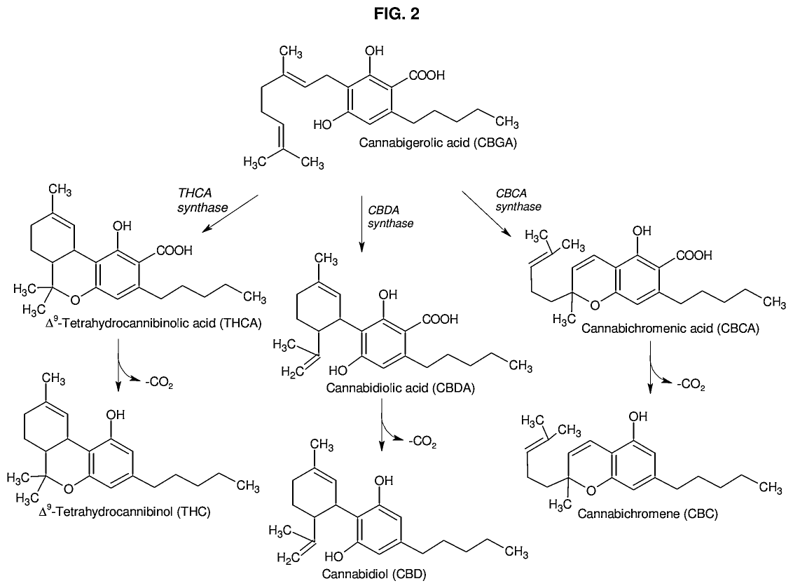

One exemplary cannabinoid pathway is depicted in FIG. 1. As shown in FIG. 1, this pathway is capable of converting hexanoic acid (HA) to the cannabinoid, cannabigerolic acid (CBGA). The pathway of FIG. 1 includes the sequence of four enzymes: (1) acyl activating enzyme (AAE), a CoA ligase enzyme of class E.C. 6.2.1.1, or a fatty acyl-CoA ligase (FACL) of class E.C.6.2.1.3 (e.g., FAA1 or FAA4); (2) olivetol synthase (OLS), a CoA synthase enzyme of class E.C. 2.3.1.206; (3) olivetolic acid cyclase (OAC), a carbon-sulfur lyase enzyme of class E.C. 4.4.1.26, and (4) prenyltransferase (PT), a transferase of class E.C. 2.5.1.102. The first two enzymes carry out the conversion of the HA starting compound to the precursor tetraketide-CoA compound, 3,5,7-trioxododecanoyl-CoA. The activity of the third enzyme, OAC, catalyzes the CoA lyase and cyclization of the tetraketide-CoA to provide the cannabinoid precursor, olivetolic acid (OA). The prenyltransferase activity of the fourth enzyme catalyzes the prenylation of OA with geranyl pyrophosphate (GPP), thereby forming the cannabinoid compound, CBGA. As illustrated by the FIG. 2, further enzymatic modification of the prenylated cannabinoid compound, CBGA, to provide cannabinoids, such as CBDA, THCA, and/or CBCA, can be carried out by including a cannabinoid synthase (e.g., CBDAS, THCAS) as a fifth enzyme in the pathway.

Exemplary cannabinoid pathway enzymes that can be introduced into a recombinant host cell to provide the pathways illustrated in FIGS. 1 and 2 include, but are not limited to, the enzymes derived from C. sativa, AAE1, OLS, OAC, PT4, CBDAS, and/or THCAS, listed in Table 6 (below), and homologs and variants of these enzymes, as described elsewhere herein.

TABLE 6
Exemplary cannabinoid pathway enzymes

| | | SEQ ID | SEQ ID |
|---|---|---|---|
| Name | Source | NO: | NO: |
| (type) | (accession) | (nt) | (aa) |
| AAE1 | Cannabis sativa | 1 | 2 |
| (acyl activating enzyme) | (AFD33345.1) | | |
| OLS | Cannabis sativa | 3 | 4 |
| (olivetol synthase) | (BAG14339.1) | | |
| CSOAC | Cannabis sativa | 5 | 6 |
| (olivetolic acid cyclase) | (AFN42527.1) | | |
| PT4 | Cannabis sativa | 7 | 8 |
| (aromatic prenyltransferase) | (DAC76710.1) | | |
| d82_PT4 | 82 aa N-term truncation of | 9 | 10 |
| (aromatic prenyltransferase) | SEQ ID NO: 8 | | |
| CBDAS | Cannabis sativa | 11 | 12 |
| (CBDA synthase) | (BAF65033.1) | | |
| d28_CBDAS | 28 aa N-term truncation of | 13 | 14 |
| (CBDA synthase) | SEQ ID NO: 12 | | |
| THCAS | Cannabis sativa | 15 | 16 |
| (THCA synthase) | (BAC41356.1) | | |
| d28_THCAS | 28 aa N-term truncation | 17 | 18 |
| (THCA synthase) | of SEQ ID NO: 16 | | |

The sequences of the exemplary cannabinoid pathway enzymes AAE1, OLS, CsOAC, PT4, CBDAS, and THCAS listed in Table 6 are naturally occurring sequences derived from the plant source, Cannabis sativa. In the recombinant host cell embodiments of the present disclosure, it is contemplated that the polynucleotide encoding the CsOAC enzyme of SEQ ID NO: 6 or 20 is replaced in the host cell by an engineered recombinant polynucleotide encoding a recombinant polypeptide having OAC activity. It is contemplated that the other heterologous cannabinoid pathway enzymes used in the recombinant host can include enzymes derived from naturally occurring sequence homologs of the Cannabis sativa enzymes, AAE1, OLS, PT4, CBDAS, THCAS, CBCAS. For example, based on the sequence, accession, and enzyme classification information provided herein, one of ordinary skill can identify known naturally occurring homologs to AAE1, OLS, PT4, CBDAS, THCAS, CBCAS, having activity in the desired biocatalytic reaction. In at least one embodiment, it is contemplated that a FACL enzyme, such as FAA1 from S. cerevisiae (UniProt entry: P30624) or FAA4 from S. cerevisiae (Uniprot entry: P47912), can be substituted for AAE1 or other AAE enzyme in a pathway.

Additionally, it is contemplated that the pathway enzymes AAE1, OLS, PT4, CBDAS, THCAS, CBCAS, or their homologs, as used in a recombinant host cell including an engineered gene of the present disclosure can include enzymes having non-naturally occurring sequences. For example, enzymes with amino acid sequences engineered to function optimally in a particular enzyme pathway, and/or optimally for production of particular cannabinoid, and/or optimally in a particular host. Methods for preparing such non-naturally occurring enzyme sequences are known in the art and include methods for enzyme engineering such as directed evolution (see, e.g., Stemmer, 1994, Proc Natl Acad Sci USA 91:10747-10751; PCT Publ. Nos. WO 95/22625, WO 97/0078, WO 97/35966, WO 98/27230, WO 00/42651, and WO 01/75767;U.S. Pat. Nos. 6,537,746; 6,117,679; 6,376,246; and 6,586,182; and U.S. Pat. Publ. Nos. 20080220990A1 and 20090312196A1; each of which is hereby incorporated by reference herein). Other modifications of cannabinoid pathway enzymes contemplated by the present disclosure include modification of the enzyme's amino acid sequence at either its N- or C-terminus by truncation or fusion. For example, in at least one embodiment of the pathway of producing a cannabinoid, versions of the AAE1, OLS, PT4, and/or CBDAS enzymes that are engineered with amino acid substitutions and/or truncated at the N- or C-terminus can be prepared using methods known in the art, and used in the compositions and methods of the present disclosure. In one embodiment, a CBDAS enzyme of SEQ ID NO: 12 that is truncated at the N-terminus by 28 amino acids to delete the native signal peptide can be used. The amino acid sequence of such a truncated CBDAS is provided herein as the d28_CBDAS enzyme of SEQ ID NO: 14. Accordingly, in at least one embodiment of the recombinant host cell, the pathway capable of producing a cannabinoid precursor or cannabinoid comprises at least enzymes having an amino acid sequence at least 90% identity to SEQ ID NO: 2 (AAE1), SEQ ID NO: 4 (OLS), SEQ ID NO: 8 (d82_PT4), and an amino acid sequence of at least 90% identity to recombinant polypeptide having OAC activity of the present disclosure as provided in Tables 3, 7, 8, 9, 10, 13, 14, and 15, and the accompanying Sequence Listing. Additionally, in at least one embodiment of the recombinant host cell, the pathway capable of producing a cannabinoid can further comprise a cannabinoid synthase of SEQ ID NO: 14 (d28_CBDAS) and/or SEQ ID NO: 18 (d28_THCAS).

The recombinant polypeptides having OAC activity encoded by the engineered genes of the present disclosure when integrated into recombinant host cells with a pathway capable of converting hexanoic acid (HA) to the C-12 tetraketide-CoA precursor, 3,5,7-trioxododecanoyl -CoA, can provide enhanced yields of the cannabinoid precursor, OA, which can be further converted to the cannabinoids, CBGA, CBDA, THCA, etc. It is contemplated that any of the engineered genes of the present disclosure that encode recombinant polypeptides having OAC activity can be incorporated into a four or five enzyme cannabinoid pathway as depicted in FIG. 1 and FIG. 2 to express the OAC activity needed for the biosynthesis of OA, and its downstream products, CBGA, CBDA, THCA, and/or CBCA

Accordingly, in at least one embodiment, the present disclosure provides a recombinant host cell comprising recombinant polynucleotides encoding a pathway capable of producing a cannabinoid, wherein the pathway comprises enzymes capable of catalyzing reactions (i)-(iv):

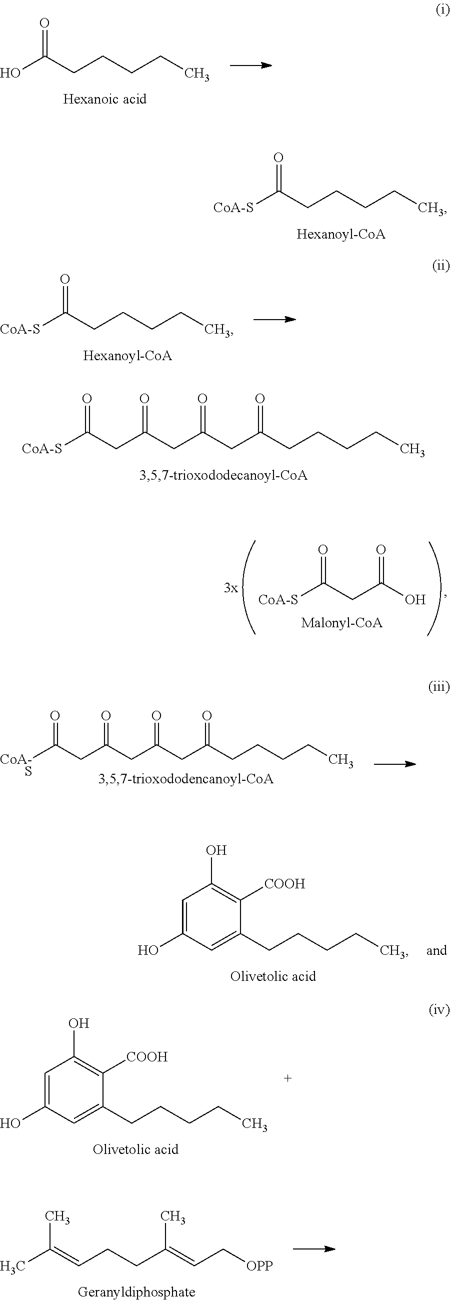

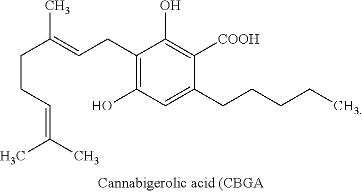 [SMILES: CCCCCc1cc(O)c(C/C=C(\C)CCC=C(C)C)c(O)c1C(=O)O]

As shown in FIG. 1, exemplary enzymes capable of catalyzing reactions (i)-(iv) are: (i) acyl activating enzyme (AAE) or fatty acyl-CoA ligase (FACL); (ii) olivetol synthase (OLS); (iii) olivetolic acid cyclase (OAC); and (iv) prenyitransferase (PT). In at least one embodiment, the OAC of the pathway of the recombinant host cell is a recombinant polypeptide having OAC activity of the present disclosure, such as an exemplary recombinant polypeptide as disclosed in Tables 3, 7, 8, 9, 10, 13, 14, and 15.

In at least one embodiment, it is contemplated that a recombinant host cell comprising a pathway comprising the two enzymes, AAE, and OLS (or the two enzymes FACL, and OLS), could modified by integrating a recombinant polynucleotide of the present disclosure to provide expression of a recombinant polypeptide with the OAC activity to convert the C-12 tetraketide -CoA precursor, 3,5,7-trioxododecanoyl-CoA, to the cannabinoid precursor, OA, thereby providing a three enzyme cannabinoid pathway as illustrated by the first three steps depicted FIG. 1 corresponding to the reactions (i)-(iii) below:

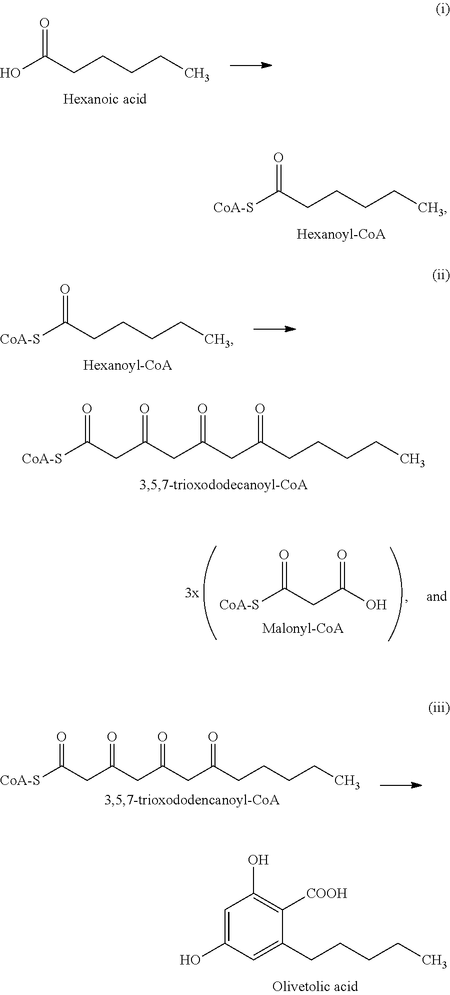

As shown in FIG. 2, the cannabinoid compound, CBGA, that is produced by the pathway of FIG. 1, can be further converted by a cannabinoid synthase to at least three other different cannabinoid compounds, Δ⁹-tetrahydrocannabinolic acid (THCA), cannabidiolic acid (CBDA), and/or cannabichromenic acid (CBCA). Accordingly, in at least one embodiment, the present disclosure provides a recombinant host cell comprising a pathway capable of converting hexanoic acid to CBGA and further comprising an enzyme capable of catalyzing the conversion of (v) CBGA to Δ⁹-THCA; (vi) CBGA to CBDA; and/or (vii) CBGA to CBCA. Thus, in at least one embodiment, the recombinant host cell comprises pathway capable of converting hexanoic acid to CBGA further comprises further comprises enzymes capable of catalyzing a reaction (v), (vi), and/or (vii):

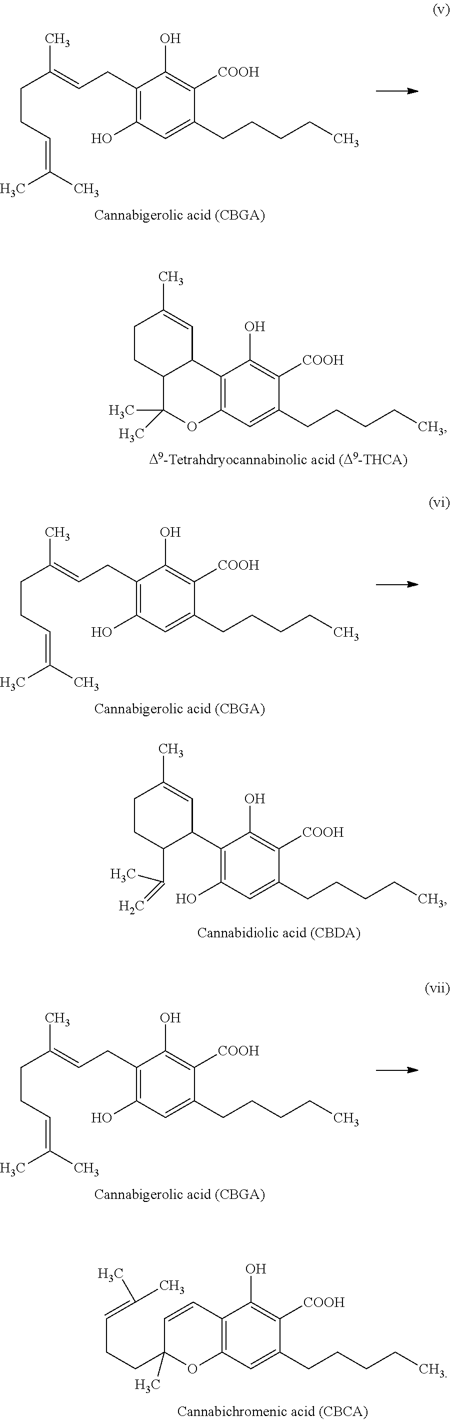

As shown in FIG. 2, exemplary enzymes capable of catalyzing reaction (v)-(vii) are: (v) THCA synthase (THCAS); (vi) CBDA synthase (CBDAS); and (vii) CBCA synthase (CBCAS). The extension of the four enzyme exemplary pathway of FIG. 1 with polynucleotide sequence capable of expressing such a cannabinoid synthase (e.g., CBDAS, THCAS, and/or CBCAS) allows for the biosynthetic production of one or more of the cannabinoids, Δ⁹-THCA, CBDA, and/or CBCA. These cannabinoids can then be decarboxylated to provide the cannabinoids, Δ⁹-THC, CBD, and/or CBC. Accordingly, it is contemplated, that in some embodiments this further decarboxylation reaction can be carried out under in vitro reaction conditions using the cannabinoid acids separated and/or isolated from the recombinant host cells.

Other cannabinoid pathway enzymes useful in the recombinant host cells and associated methods of the present disclosure are known in the art, and can include naturally occurring enzymes obtained or derived from cannabis plants, or non-naturally occurring enzymes that have been engineered based on the naturally occurring cannabis plant sequences. It is also contemplated that enzymes obtained or derived from other organisms (e.g., microorganisms) having a catalytic activity related to a desired conversion activity useful in a cannabinoid pathway can be engineered for use in a recombinant host cell of the present disclosure.

A wide range of cannabinoid compounds can be produced biosynthetically by a recombinant host cell integrated with such a cannabinoid pathway. The cannabinoid pathways of FIGS. 1-2 depict the production of the more common naturally occurring cannabinoids, CBGA, Δ⁹-THCA, CBDA, and CBCA. It is also contemplated, however, that the engineered genes, recombinant polypeptides, cannabinoid pathways, recombinant host cells, and associated methods of the present disclosure can also be used to biosynthesize a range of additional rarely occurring, and/or synthetic cannabinoid compounds. Table 1 (above) lists the names and depicts the chemical structures of a wide range of exemplary rarely occurring, and/or synthetic cannabinoid compounds (e.g., CBGVA, CBDVA, THCVA) that are contemplated for production using the recombinant polypeptides, host cells, compositions, and methods of the present disclosure.

Similarly, Table 2 (above) depicts additional rarely occurring, and/or synthetic cannabinoid precursor compounds (e.g., DA) that could be produced by such recombinant host cells in the pathway for production of certain rarely occurring, and/or synthetic cannabinoid compounds of Table 1. Accordingly, in at least one embodiment, a recombinant host cell that includes a pathway to a cannabinoid precursor and that expresses a recombinant polypeptide having OAC activity of the present disclosure (e.g., as in Tables 3, 7, 8, 9, 10, 13, 14, and 15) can be used for the biosynthetic production of a rarely occurring, and/or synthetic cannabinoid compound, or a composition comprising such a cannabinoid compound. It is contemplated that the produced rarely occurring, and/or synthetic cannabinoid precursors and cannabinoids can include, but is not limited to, the compounds listed in Tables 1 and 2. Accordingly, in at least embodiment, a recombinant host cell of the present disclosure can be used for production of a cannabinoid compound selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

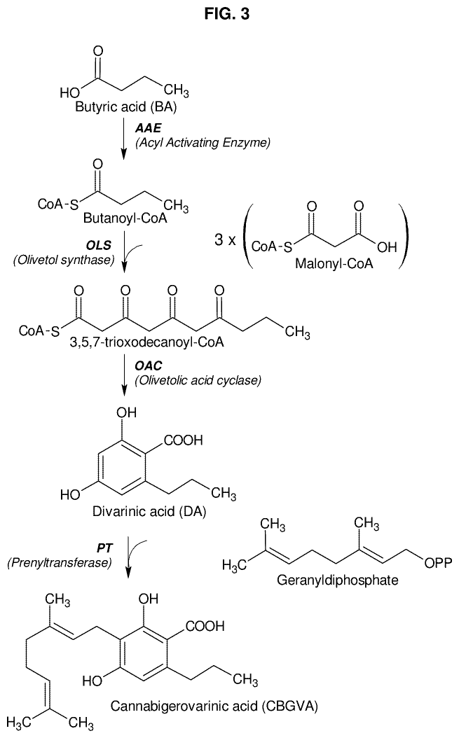

In at least one embodiment, the compositions and methods of the present disclosure can be used for the production of the rare varin series of cannabinoids, CBGVA, Δ⁹-THCVA, CBDVA, and CBCVA, and cannabinoid precursor, DA. As shown in Table 1, the varin cannabinoids feature a 3 carbon propyl side-chain rather than the 5 carbon pentyl side chain found in the common cannabinoids, CBGA, Δ⁹-THCA, CBDA, and CBCA. An exemplary cannabinoid pathway capable of producing the rare naturally occurring cannabinoid, cannabigerovarinic acid (CBGVA), is depicted in FIG. 3. Instead of starting with hexanoic acid, the pathway of FIG. 3 is fed butyric acid (BA) which is converted to cannabinoid precursor, divarinic acid (DA) via the same three enzyme pathway of AAE, OLS, and OAC. The cannabinoid precursor DA is then converted by an prenyltransferase to the rare cannabinoid, CBGVA. In at least one embodiment of the present disclosure, the OAC of the pathway of the recombinant host cell is a recombinant polypeptide having OAC activity of the present disclosure, such as an exemplary recombinant polypeptide as disclosed in Tables 3, 7, 8, 9, 10, 13, 14, and 15. Accordingly, in at least one embodiment of the recombinant host cell, the pathway capable of producing a cannabinoid comprises enzymes capable of catalyzing reactions (i)-(iv):

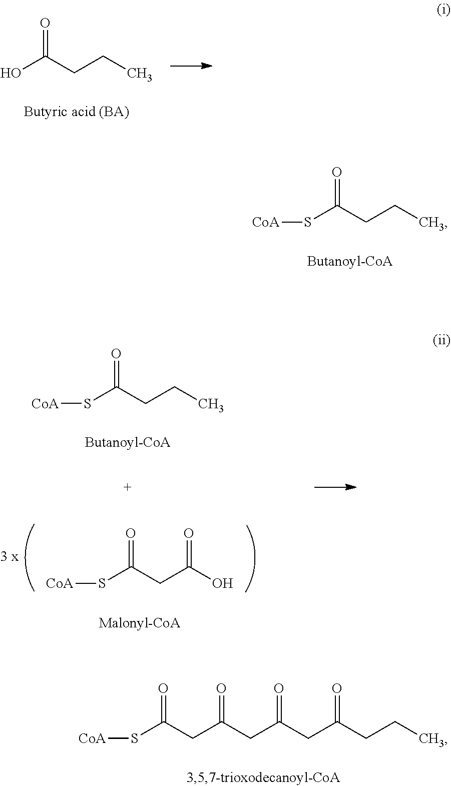

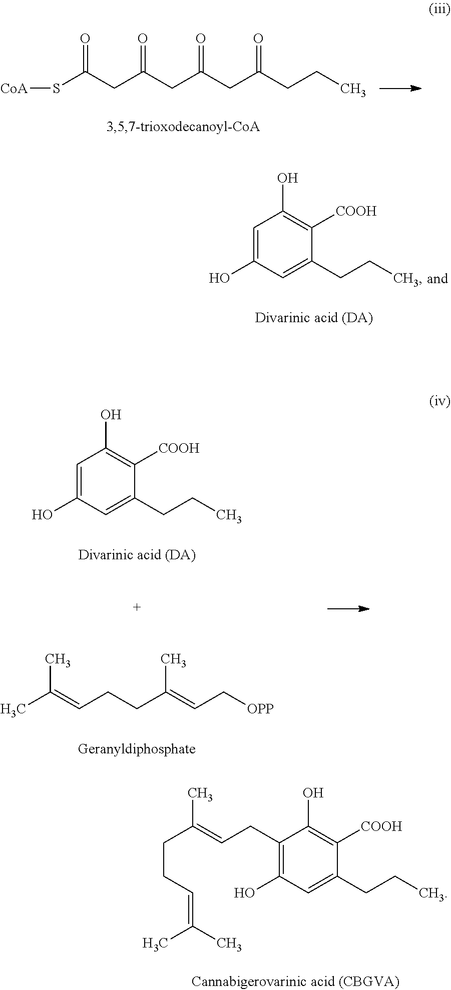

Exemplary enzymes capable of catalyzing reactions (i), (ii), (iii) and (iv) are: (i) acyl activating enzyme (AAE) or fatty acyl-CoA ligase (FACL); (ii) olivetol synthase (OLS); (iii) a recombinant polypeptide having OAC activity as disclosed herein (e.g., a polypeptide of Tables 3, 7, 8, 9, 10, 13, 14, and 15); and (iv) prenyltransferase (PT4). Exemplary enzymes, AAE1,OLS, and PT4, derived from C. sativa are known in the art and also provided in Table 1, and the accompanying Sequence Listing. In at least one embodiment, it is contemplated that FAA1 from S. cerevisiae (UniProt entry: P30624) or FAA4 from S. cerevisiae (Uniprot entry: P47912) can be used to catalyze reaction (i) rather than an AAE enzyme in a pathway with OLS, and PT4.

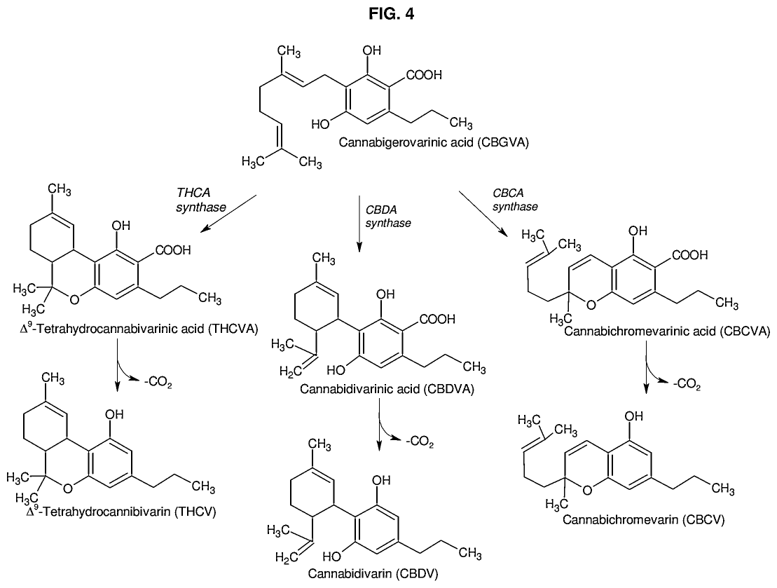

As further illustrated in FIG. 4, the heterologous pathway depicted in FIG. 3 which is capable of producing a rare cannabinoid, such as CBGVA, can be further modified to include one or more cannabinoid synthase enzymes (e.g., CBDAS, THCAS, CBCAS). As shown by the exemplary pathway of FIG. 4, with the incorporation of one or more synthase enzymes, the rare varin cannabinoid, CBGVA, can be converted to the rare varin cannabinoids, cannabidivarinic acid (CBDVA), Δ⁹-tetrahydrocannabivarinic acid (Δ⁹-THCVA), and cannabichromevarinic acid (CBCVA). Enzymes capable of carrying out these conversions include the C. sativa CBDA synthase, THCA synthase, and CBCA synthase, respectively. Accordingly, in at least one embodiment, the present disclosure provides a recombinant host cell comprising a pathway capable of converting BA to CBGVA and further comprising an enzyme capable of catalyzing the conversion of (v) CBGVA to Δ⁹-THCVA; (vi) CBGVA to CBDVA; and/or (vii) CBGVA to CBCVA. Thus, in at least one embodiment, the recombinant host cell comprises pathway capable of converting BA to CBGVA further comprises further comprises enzymes capable of catalyzing a reaction (v), (vi), and/or (vii):

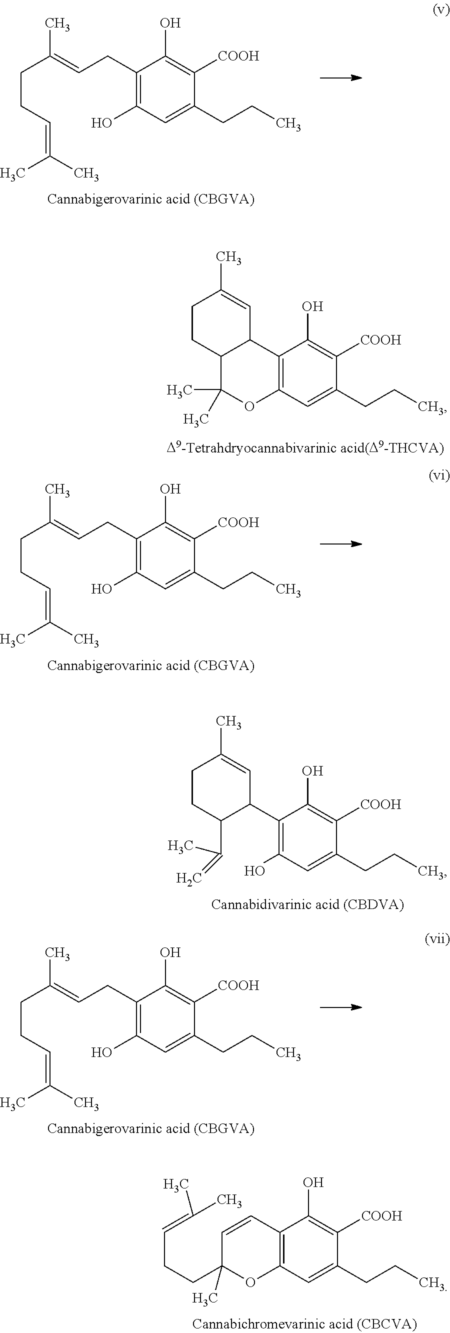

Exemplary enzymes capable of catalyzing reaction (v)-(vii) as shown above are: (v) THCA synthase (THCAS); (vi) CBDA synthase (CBDAS); and (vii) CBCA synthase (CBCAS). Exemplary THCAS, CBDAS, and CBCAS enzymes are provided in Table 1.

Furthermore, as shown in FIG. 4, the rare cannabinoid acids, CBDVA, Δ⁹-THCVA, and CBCVA, can undergo a further decarboxylation reaction to provide the varin cannabinoid products, cannabidivarin (CBDV). Δ⁹-tetrahydrocannabivarin (Δ⁹-THCV), and cannabichromevarin (CBCV), respectively. In some embodiments, this further decarboxylation can be carried out under in vitro reaction conditions using the cannabinoid acids isolated from the recombinant host cells.

Similarly, as shown in FIGS. 1 and 3, a heterologous cannabinoid pathway comprising the sequence of at least the four enzymes AAE, OLS, OAC, and PT (wherein, the OAC is a recombinant polypeptide having OAC activity of the present disclosure) is capable of converting a precursor substrate compound, such as hexanoic acid (HA) to an initial cannabinoid compound, such as CBGA or CBGVA. These initial cannabinoid product compounds can themselves be used as a substrate for the in vitro biosynthesis of a range of further cannabinoid product compounds, such as THCA and THCVA, as shown in FIGS. 2 and 4. A wide range of cannabinoid compounds, such as those shown in Table 1, are contemplated for in vivo biosynthetic production in a recombinant host cell of the present disclosure or via a partial or full in vitro biosynthesis process using recombinant polypeptides of the present disclosure.

As described herein, the heterologous cannabinoid pathways of the present disclosure can be incorporated into a range of host cells to provide a system for biosynthetic production of cannabinoids (e.g., CBGA, CBGVA, CBDA, CBDVA, THCA, THCVA). Methods and techniques for integrating polynucleotides into recombinant host cells, such as yeast, so that they express functional pathways of enzymes are well known in the art and described elsewhere herein including the Examples. Generally, the host cell used in the recombinant host cells of the present disclosure can be any cell that can be recombinantly modified with nucleic acids and cultured to express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure.

The recombinant host cells of the present disclosure comprise heterologous nucleic acids encoding a pathway of enzymes capable of producing a tetraketide-CoA precursor compound (e.g., 3,5,7-trioxododecanoyl-CoA or 3,5,7-trioxodecanoyl-CoA), and a heterologous nucleic acid comprising a sequence encoding a recombinant polypeptide having OAC activity capable of cyclizing this tetraketide-CoA to form a cannabinoid precursor product (e.g., OA or DA). As described elsewhere herein, nucleic acid sequences encoding the cannabinoid pathway enzymes, are known in the art, and provided herein, and can readily be used in accordance with the present disclosure. Typically, the nucleic acid sequence encoding enzymes which form a part of a cannabinoid pathway, further include one or more additional nucleic acid sequences, for example, a nucleic acid sequence controlling expression of the enzymes which form a part of a cannabinoid biosynthetic enzyme pathway, and these one or more additional nucleic acid sequences together with the nucleic acid sequence encoding the enzyme can be considered a heterologous nucleic acid sequence. A variety of techniques and methodologies are available and well known in the art for introducing heterologous nucleic acid sequences, such as nucleic acid sequences encoding the cannabinoid pathway enzymes (e.g., AAE, OLS, OAC, and PT), into a host cell so as to attain expression the host cell. Such techniques are well known to the skilled artisan and can, for example, be found in Sambrook et al., Molecular Cloning, a Laboratory Manual, Cold Spring Harbor Laboratory Press, 2012, Fourth Ed.

One of ordinary skill will recognize that the heterologous nucleic acids encoding the recombinant olivetolic acid cyclase enzymes and/or other pathway enzymes will further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on. Promoters useful in the recombinant host cells of the present disclosure may be constitutive or inducible, provided such promoters are operable in the host cells. Promoters that may be used to control expression in fungal host cells, such as Saccharomyces cerevisiae, are well known in the art and include, but are not limited to: inducible promoters, such as a Gal1 promoter or Gal10 promoter, a constitutive promoter, such as an alcohol dehydrogenase (ADH) promoter, a glyceraldehyde-3-phosphate dehydrogenase (GPD) promoter, or an S. pombe Nmt, or ADH promoter. Exemplary promoters that may be used to control expression in bacterial cells can include the Escherichia coli promoters lac, tac, trc, trp or the 77 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers, and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding cannabinoid pathway genes. Such techniques are described in e.g., Sambrook et al., Molecular Cloning, a Laboratory Manual, Cold Spring Harbor Laboratory Press, 2012, Fourth Ed. Accordingly, in at least one embodiment, the heterologous nucleic acid sequences of the present disclosure comprise a promoter capable of controlling expression in a host cell, wherein the promoter is linked to a nucleic acid sequence encoding a recombinant polypeptide having OAC activity of the present disclosure, and as necessary, other enzymes constituting a cannabinoid pathway (e.g., AAE, OLS, OAC). This heterologous nucleic acid sequence can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell's genome.

It is also contemplated that in some embodiments an expression vector comprising a heterologous nucleic acid of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14:403).

In at least one embodiment, the present disclosure also provides of a method for producing a cannabinoid, wherein a heterologous nucleic acid encoding a recombinant polypeptide having OAC activity (e.g., an exemplary engineered polypeptide of Tables 3, 7, 8, 9, 10, 13, 14, and 15) can be introduced into a recombinant host cell. The recombinant host cell can then be used for production of the polypeptide, or incorporated in a biocatalytic process that utilized the OAC activity of the recombinant polypeptide expressed by the host cell for the catalytic cyclization of a tetraketide-CoA substrate, e.g., the cyclization of 3,5,7-trioxododecanoyl-CoA to produce OA. In at least one embodiment, the recombinant host cell can further comprise a pathway of enzymes capable of producing a tetraketide-CoA precursor (e.g., 3,5,7-trioxododecanoyl-CoA) which can act as a substrate for the recombinant polypeptide with OAC activity. It is contemplated that a recombinant host cell comprising a heterologous nucleic acid encoding a recombinant polypeptide having OAC activity of the present disclosure can provide improved biosynthesis of a desired cannabinoid precursor (e.g., OA) or a cannabinoid (e.g., CBGA) product in terms of titer, yield, and production rate, due to the improved characteristics of the expressed OAC activity in the cell associated with the amino acid and codon differences engineered in the gene.

Accordingly, in at least one embodiment, the present disclosure provides a method of producing a cannabinoid derivative, wherein the method comprises: (a) culturing in a suitable medium a recombinant host cell of the present disclosure; and (b) recovering the produced cannabinoid derivative. In at least one embodiment, the method of producing a cannabinoid derivative further contacting a cell-free extract of the culture containing the produced cannabinoid with a biocatalytic reagent or chemical reagent capable of converting the cannabinoid to a cannabinoid derivative. In at least one embodiment, the biocatalytic reagent is an enzyme capable of converting the produced cannabinoid to a different cannabinoid or a cannabinoid derivative compound. In at least one embodiment, the chemical reagent is capable of chemically modifying the produced cannabinoid to produce a different cannabinoid or a cannabinoid derivative compound. In at least one embodiment of the method for producing a cannabinoid, the method can further comprise contacting a cell-free extract of the culture containing the produced cannabinoid with a biocatalytic reagent or chemical reagent.

It is contemplated that the cannabinoid, or cannabinoid derivative produced using the methods of the present disclosure can be produced and/or recovered from the reaction in the form of a salt. In at least one embodiment, the recovered salt of the cannabinoid, cannabinoid precursor, cannabinoid precursor derivative, or cannabinoid derivative is a pharmaceutically acceptable salt. Such pharmaceutically acceptable salts retain the biological effectiveness and properties of the free base compound.

It also is contemplated the recombinant polypeptides with OAC activity of the present disclosure can be incorporated in any biosynthesis method requiring a OAC catalyzed biocatalytic step. Thus, in at least one embodiment, the recombinant polypeptides having OAC activity (e.g., exemplary polypeptides of Tables 3, 7, 8, 9, 10, 13, 14, and 15) can be used in a method for preparing a cannabinoid precursor compound of structural formula (I)

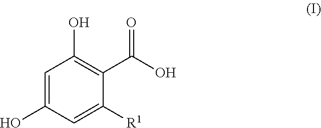

wherein, R¹ is C1-C7 alkyl, wherein the method comprises contacting an recombinant polypeptide having OAC activity of the present disclosure (e.g., an exemplary recombinant of Table 3) under suitable reactions conditions, with tetraketide-CoA cannabinoid precursor compound of structural formula (II)

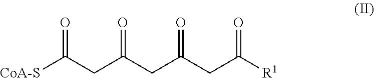

wherein, R¹ is C1-C7 alkyl.

Exemplary conversions of cannabinoid precursor compounds of structural formula (II) to cannabinoid compounds of structural formula (I) that are catalyzed by the recombinant polypeptides having OAC activity of the present disclosure include: (1) conversion of 3,5,7-trioxododecanoyl-CoA to olivetolic acid (OA); and (2) conversion 3,5,7-trioxodecanoyl-CoA to divarinic acid (DA).

It is contemplated that the recombinant polypeptides having OAC activity of the present disclosure (e.g., polypeptides disclosed in Tables 3, 7, 8, 9, 10, 13, 14, and 15) can catalyze the cyclization of other cannabinoid precursor compounds that are structural analogs of the tetraketide-CoA, 3,5,7-trioxododecanoyl-CoA. Accordingly, in at least one embodiment of the biosynthesis method for conversion a cannabinoid precursor compound of structural formula (II) to a cannabinoid compound of structural formula (I), the compound of structure formula (I) is olivetolic acid (OA) and the compound of structural formula (II) is 3,5,7-trioxododecanoyl-CoA. In at least one embodiment, the compound of structure formula (I) is divarinic acid (DA) and the compound of structural formula (II) is 3,5,7-trioxodecanoyl-CoA acid.

Suitable reaction conditions for the biosynthesis of cannabinoid precursors and cannabinoids are known in the art, and can be used with the recombinant polypeptides having OAC activity of the present disclosure. Additionally, suitable reaction conditions for the exemplary polypeptides of the present disclosure can be determined using routine techniques known in the art for optimizing biocatalytic reactions. It is contemplated that various ranges of suitable reaction conditions with the recombinant polypeptides of the present disclosure, including but not limited to ranges of pH, temperature, buffer, solvent system, substrate loading, polypeptide loading, co-substrate or co-factor loading, atmosphere, and reaction time. Suitable reaction conditions can be readily determined and optimized for particular reactions by routine experimentation that includes, but is not limited to, contacting the recombinant polypeptide and substrate under experimental reaction conditions of concentration, pH, temperature, solvent conditions, and detecting the production of the desired compound of structural formula (I). In at least one embodiment, the suitable reaction conditions comprise a reaction solution of ˜pH 7-8, a temperature of 25 C to 37 C; optionally, the reaction conditions comprise a reaction solution of ˜PH 7 and a temperature of ˜30C. In at least one embodiment, the reaction solution is allowed to incubate at a temperature of 25 C to 37 C for a reaction time of at least 1, 6, 12, 24, or 48 hours, before the amount of reaction product is determined.

The present disclosure also contemplates that the methods for biocatalytic conversion of a cannabinoid precursor compound of structural formula (II) to a cannabinoid compound of structural formula (I) using an recombinant polypeptide having OAC activity of the present disclosure can comprise additional chemical or biocatalytic steps carried out on the product compound of structural formula (II), including steps of product compound work-up, extraction, isolation, purification, and/or crystallization, each of which can be carried out under a range of conditions.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Preparation and Screening of Engineered Genes Encoding Recombinant Polypeptides with Olivetolic Acid Cyclase Activity

This example illustrates preparation and screening of libraries of engineered genes expressing OAC activity when integrated in a yeast strain already engineered with the C. sativa genes, AAE1 (SEQ ID NO: 1) and OLS (SEQ ID NO: 3). An initial library was generated by site saturation mutagenesis (SSM) of an OAC parent gene with SEQ ID NO: 5, a synthesized yeast codon-optimized variant of CsOAC. A further codon optimization library was designed for increased expression in yeast on SEQ ID NO 5, from which SEQ ID NO 19 was identified to have 1.6-fold improvement in gene expression and or transcript stability. Codon usage can affect secondary structure of mRNA and translation efficiency. SEQ ID NO: 5 and SEQ ID NO 19 encode the wild-type CsOAC polypeptide sequence of SEQ ID NO: 6 or 20. Both the SSM and codon optimization libraries based on SEQ ID NO. 5 were integrated in yeast strains already engineered with the C. sativa genes, AAE1 (SEQ ID NO: 1) and OLS (SEQ ID NO: 3) and screened for OA production indicating expression of a recombinant polypeptide having OAC activity.

### Materials and Methods

### A. Site Saturation Mutagenesis Library Build:

A yeast codon-optimized gene encoding the wild-type C. sativa OAC polypeptide of SEQ ID NO: 6 or 20 was synthesized as the polynucleotide of SEQ ID NO: 5. Further codon optimization of SEQ NO ID NO: 5 identified an alternative yeast codon-optimized version of the gene encoding the wild-type C. sativa OAC as polynucleotide of SEQ ID NO: 19. It was found that yeast strains integrated with this alternative codon-optimized gene of SEQ ID NO: 19 exhibited increased OA titer (˜1.6-fold) likely due to enhanced expression or transcript stability.

The synthetic gene of SEQ ID NO: 5 was integrated into a parent yeast strain as a knock-in using CRISPR-Cas9 at the XI-2 locus. The integrated OAC gene was expressed under the bidirectional Gal1/10 promoter and the PGK1 terminator sequences. The parent strain was previously engineered to include the two C. sativa genes, AAE1 (SEQ ID NO: 1) and OLS (SEQ ID NO: 3), which form a pathway capable of converting hexanoic acid to 3,5,7-trioxododecanoyl-CoA, the precursor for olivetolic acid (OA) synthesis. The resulting control strain (MV005), integrated with the OAC gene thus included a pathway of the genes AAE1, OLS, and OAC capable of converting hexanoic acid to OA. The MV005 control strain was further modified to build a screening strain for integration of the saturation mutagenesis and codon optimization libraries. A screening strain (EVO002), was built by integrating the m-Venus cassette at the XI-2 site under control of the bidirectional Gal1/10 promoter and PGK1 terminator, thereby replacing the previous integrated OAC gene. The EVO002 strain was no longer capable of converting hexanoic acid to OA.

Genomic DNA from the control strain MV005, was used as the template to generate two overlapping PCR products: (1) a first PCR product (Fragment A), which does not harbor any degenerate codons, and (2) a second PCR product (Fragment B), which has sequence overlap with the Fragment A, and is amplified harboring one NNK degenerate codon only. Primers used for amplification of Fragments A and B and overlap extension were designed according to standard site-saturation mutagenesis protocols. Fragment B was amplified with a series of forward primers that included the single NNK degenerate codon scanned across the various desired positions and a single reverse primer: 5′-CTAAGTCTAGCCACGAAAACTGCAA-3′ (SEQ ID NO: 423). Fragment A was amplified using a single forward primer: 5′-GGTTATGAAGAGGAAAAATTGGCAGTAACC-3′ (SEQ ID NO: 424) and a series of reverse primers designed according to the location of the mutagenesis site. The two fragments A and B were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426).

The assembled OE-PCR products were then pooled together, and gel purified to provide a saturation mutagenesis library for integration as linear donor DNA.

### B. Codon Optimization Library Build:

A total of 96 codon optimized variants were designed based on SEQ ID NO 5 using an AFIAK python script developed in house using the preferred Saccharomyces cerevisiae codon usage table and optimization optimal GC count. To facilitate efficient integration, a 50-nucleotide 5′ flanking sequence, 5′-CAAAAAATTGTTAATATACCTCTATACTTTAACGTCAAGGAGAAAAAACC-3′ (SEQ ID NO: 427), and a 50-nucleotide 3′ flanking sequence, 5′-TAAATTGAATTGAATTGAAATCGATAGATCAATTTTTTTCTTTTCTCTTT-3′ (SEQ ID NO: 428), were introduced into each codon variant. This sequence provided the overlap homology to build longer DNA donors including the pGal1 promoter: 5′-TTTTCAAAAATTCTTACTTTTTTTTTGGATGGACGCAAAGAAGTTTAATAATCATATTACATG GCATTACCACCATATACATATCCATATACATATCCATATCTAATCTTACTTATATGTTGTGGA AATGTAAAGAGCCCCATTATCTTAGCCTAAAAAAACCTTCTCTTTGGAACTTTCAGTAATAC GCTTAACTGCTCATTGCTATATTGAAGTACGGATTAGAAGCCGCCGAGCGGGTGACAGCC CTCCGAAGGAAGACTCTCCTCCGTGCGTCCTCGTCTTCACCGGTCGCGTTCCTGAAACGC AGATGTGCCTCGCGCCGCACTGCTCCGAACAATAAAGATTCTACAATACTAGCTTTTATGG TTATGAAGAGGAAAAATTGGCAGTAACCTGGCCCCACAAACCTTCAAATGAACGAATCAAA TTAACAACCATAGGATGATAATGCGATTAGTTTTTTAGCCTTATTTCTGGGGTAATTAATCA GCGAAGCGATGATTTTTGATCTATTAACAGATATATAAATGCAAAAACTGCATAACCACTTT AACTAATACTTTCAACATTTTCGGTTTGTATTACTTCTTATTCAAATGTAATAAAAGTATCAAC AAAAAATTGTTAATATACCTCTATACTTTAACGTCAAGGAGAAAAAACC-3′ (SEQ ID NO: 429) and the PGK1t terminator: 5′-ATTGAATTGAATTGAAATCGATAGATCAATTTTTTTCTTTTCTCTTTCCCCATCCTTTACGCT AAAATAATAGTTTATTTTATTTTTTGAATATTTTTTATTTATATACGTATATATAGACTATTATT TATCTTTTAATGATTATTAAGATTTTTATTAAAAAAAAATTCGCTCCTCTTTTAATGCCTTTAT GCAGTTTTTTTTTCCCATTCGATATTTCTATGT-3′ (SEQ ID NO: 430). The codon optimized variants including the flanking sequences were synthesized by Twist. The individual codon optimized variants, promoter and terminator DNA pieces were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426).

The assembled OE-PCR products were then pooled together, and gel purified to provide a codon optimization library for integration as linear donor DNA.

Both the saturation mutagenesis and codon optimization libraries consisting of linear donor DNA were transformed separately into the screening strain (EVO002), along with sequence specific guide to integrate the library in place of the m-Venus cassette using CRISPR-Cas9 at the XI-2 site in a yeast strain that already had integrated genes encoding the C. sativa enzymes, AAE1, and OLS. The resulting libraries would contain integrated OAC mutants integrated at the XI-2 site under control of the bidirectional Gal1/10 promoter and PGK1 terminator, in place of the m-Venus cassette and therefore restoring ability to convert hexanoic acid to OA. The resulting libraries integrated into the EVO002 strain were plated on selective YPD agar to select for strains with positive integration events.

### C. Screening of Site Saturation Mutagenesis and Codon Optimization Libraries for Olivetolic Acid Biosynthesis:

Individual colonies from the saturation mutagenesis and codon optimization libraries integrated in EVO002 and the respective MV005 control strain were grown in 0.3 mL YPD in 96-well microtiter plates. The microtiter plates were incubated in shaking incubators for 48 h at 30 C. 85% humidity, and 250 rpm. The resulting liquid cultures were then sub-cultured into additional 96 well plates with 0.27 mL fresh YPD and hexanoic acid (HA) was added to 2 mM final concentration. Subculture microtiter plates were then incubated in shaking incubators for an additional 48 hours at 30 C, 85% humidity, and 250 rpm. The whole broth from these subculture plates was extracted and analyzed for the presence of the cannabinoid precursor compound, OA, using HPLC, as described below.

1. HPLC sample preparation: The whole broth of each culture within the 96 well plates were extracted and diluted with MeOH for sample preparation. The extracted samples were loaded onto RapidFire365 coupled with a triple quadrupole mass spectrometry detector. The cannabinoid precursor compound, OA, was detected using MRM mode. A calibration curve of OA was generated by running serial dilutions of standards, and then used to calculate the concentration of the cultures within the library plates.

2. HPLC instrumentation and parameters: HPLC system: Agilent RapidFire 365; Column: Agilent Cartridge C18 (12 μl, type C): Mobile phase: Pump 1 uses 95:5 H2O: acetonitrile with 0.1% formic acid at 1 mL/min”. Pump 2 uses 20:80 acetonitrile: H₂O at 0.8 mL/min; Pump 3 uses MeOH with 0.1% formic acid, at 0.8 mL/min.; Aqueous wash uses H₂O; Organic wash uses acetonitrile; RapidFire cycle time: Aspiration 600 ms; Load/wash 3000 ms; Extra wash 2000 ms; Elute 4000 ms; Re-equilibration 500 ms.

### D. Sequencing

Those clones from the saturation mutagenesis and codon optimization libraries determined by screening to exhibit an OA titer were re-tested and sequenced using Sanger sequencing technology to determine the specific codon differences (relative to SEQ ID NO 5) and amino acid differences (relative to SEQ ID NO 6).

### E. Results

Results for relative OA titer and corresponding amino acid changes of the SSM library strains, and nucleotide changes of the codon optimized library generated from the parent codon-optimized gene of SEQ ID NO: 5 are summarized in Table 7 (below).

TABLE 7

| NT | AA | | |
|---|---|---|---|
| SEQ ID | SEQ ID | aa substitution(s) | Relative OA |
| NO: | NO: | relative to CsOAC | titer¹ |
| 5 | 6 | n/a | 1 |
| 19 | 20 | n/a | 1.58 |
| 21 | 22 | T16Q | 1.6 |
| 23 | 24 | P76V | 1.11 |
| 25 | 26 | L9V | 1.07 |
| 27 | 28 | K44P | 1.01 |
| 29 | 30 | I74S | 1.00 |
| 31 | 32 | H57G | 0.93 |
| 33 | 34 | K49R | 0.90 |
| 35 | 36 | E14G | 0.90 |
| 37 | 38 | D71G | 0.90 |
| 39 | 40 | K91E | 0.89 |
| 41 | 42 | L9V, E21V | 0.74 |
| 45 | 46 | K12L | 0.49 |
| 47 | 48 | A18S | 0.67 |
| 51 | 52 | E17G, K44P | 0.44 |
| 53 | 54 | I94K | 0.44 |
| 55 | 56 | E64K | 0.40 |
| 61 | 62 | A77E | 0.29 |
| 65 | 66 | T62G | 0.27 |
| 67 | 68 | F11L | 0.26 |
| 69 | 70 | S87K | 0.29 |
| 71 | 72 | D83R | 0.26 |
| 75 | 76 | G82R; | 0.25 |

¹The OA titer for the MV005 control strain integrated with codon optimized OAC gene of SEQ ID NO: 5 was set as 1, and the values for “Relative OA titer” for each library strain clone were determined by comparison to this MV005 control strain.

### Example 2: Preparation and Screening of Engineered Genes Encoding Recombinant Polypeptides with Olivetolic Acid Cyclase Activity

This example illustrates preparation and screening of additional libraries of engineered genes expressing OAC activity when integrated in a yeast strain already engineered with the C. sativa genes, AAE1 (SEQ ID NO: 1) and OLS (SEQ ID NO: 3). A combinatorial library of 56variant sequences was synthesized using diversity identified in Table 5. 26 combinatorial variants were synthesized based on SEQ ID NO. 5 and 26 variants were synthesized based on the codon optimized SEQ ID NO. 19. One combinatorial variant synthesized based on SEQ ID NO 19 was further used as a parent for SSM to introduce additional genetic diversity (SEQ ID NO 85).

### Materials and Methods

### A. Combinatorial Library Build:

The synthetic codon-optimized genes of SEQ ID NO: 5 and SEQ ID NO. 19 described in Example 1 were used as parent backbones to design and synthesize 56 variants incorporating combinations of amino acid changes selected from Table 1. As in Example 1, to facilitate efficient integration, the 50-mer 5′ and 3′ flanking sequences of SEQ ID NO: 427 and SEQ ID NO: 428 were introduced into each combinatorial variant. As in Example 1, these flanking sequences provided the overlap homology to build longer DNA donors including the pGal1 promoter and the PGK1t terminator. The combinatorial variants including the flanking sequence were synthesized by Twist. The individual combinatorial variants, promoter and terminator DNA pieces were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426). The integrated synthesized genes were expressed under the bidirectional Gal1/10 promoter and the PGK1 terminator sequences. As in Example 1, The parent strain was previously engineered to include the two C. sativa genes, AAE1 (SEQ ID NO: 1) and OLS (SEQ ID NO: 3), which form a pathway capable of converting hexanoic acid to 3,5,7-trioxododecanoyl-CoA, the precursor for olivetolic acid (OA) synthesis. The control strain was (MV005) thus included a pathway of the genes AAE1, OLS, and OAC capable of converting hexanoic acid to OA. As in Example 1, the MV005 control strain was further modified to build a screening strain for integration of the combinatorial and additional site saturation mutagenesis libraries. The screening strain (EVO002), was built by integrating the m-Venus cassette at the XI-2 site under control of the bidirectional Gal1/10 promoter and PGK1 terminator, thereby replacing the previous integrated OAC gene. The EVO002 strain was no longer capable of converting hexanoic acid to OA.

The pooled combinatorial library of linear donor DNA was transformed into the screening strain (EVO002), along with sequence specific guide to integrate the library in place of the m-Venus cassette using CRISPR-Cas9 at the XI-2 site in a yeast strain that already had integrated genes encoding the C. sativa enzymes, AAE1, and OLS. The resulting libraries would contain integrated OAC mutants from the combinatorial library integrated at the XI-2 site under control of the bidirectional Gal1/10 promoter and PGK1 terminator, in place of the m-Venus cassette and therefore restoring ability to convert hexanoic acid to OA. The resulting combinatorial libraries integrated into the EVO002 strain were plated on selective YPD agar to select for strains with positive integration events.

### B. Additional SSM Library Build

The combinatorial variant, SEQ ID NO. 85 (derived from the codon-optimized parent gene of SEQ ID NO 19) was used as a parent gene to build an additional SSM library introducing additional genetic diversity along with the 5 amino acid changes L9V, E14G, K49R, D71G, K91E already encoded by SEQ ID NO: 85 in the variant polypeptide sequence of SEQ ID NO: 86.

Genomic DNA from the combinatorial variant with SEQ ID NO. 85 was used as the template to generate two overlapping PCR products: (1) a first PCR product (Fragment A), which does not harbor any degenerate codons, and (2) a second PCR product (Fragment B), which has sequence overlap with the Fragment A, and is amplified harboring one NNK degenerate codon only. Primers used for amplification of Fragments A and B and overlap extension were designed according to standard site-saturation mutagenesis protocols. Fragment B was amplified with a series of forward primers that included the single NNK degenerate codon scanned across the various desired positions and a single reverse primer: 5′-CTAAGTCTAGCCACGAAAACTGCAA-3′ (SEQ ID NO: 423). Fragment A was amplified using a single forward primer: 5′-GGTTATGAAGAGGAAAAATTGGCAGTAACC-3′ (SEQ ID NO: 424) and a series of reverse primers designed according to the location of the mutagenesis site. The two fragments A and B were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426). The assembled OE-PCR products were then pooled together, and gel purified to provide a saturation mutagenesis library for integration as linear donor DNA.

### C. Screening of Combinatorial and Additional SSM Libraries for Olivetolic Acid Biosynthesis:

Individual colonies from the combinatorial libraries in EVO002 (section A above) and additional SSM libraries in EVO002 (section B above) and the respective MV005 control strain were grown in 0.3 mL YPD in 96-well microtiter plates and screened as described in Example 1. Whole broth culture samples were extracted and screened using HPLC as described in Example 1.

### D. Sequencing

Clones determined by screening to exhibit an OA titer were re-tested and sequenced using Sanger sequencing technology to determine their nucleotide and amino acid differences compared to SEQ ID NO 5 and SEQ ID NO 6, respectively.

### E. Results

Results for relative OA titer and corresponding amino acid changes of the combinatorial and additional SSM libraries is summarized in Table 8 (below).

TABLE 8

| NT | AA | | |
|---|---|---|---|
| SEQ ID | SEQ ID | | Relative OA |
| NO: | NO: | aa substitution(s) relative to CsOAC | titer¹ |
| 5 | 6 | n/a | 1 |
| 19 | 20 | n/a | 1.58 |
| 43 | 44 | T26N, K49R, D71G, K91E | 0.5 |
| 49 | 50 | L9V, E14G, T16Q, V40G, K49R, D71G, I74M, K91E | 0.44 |
| 57 | 58 | I33E, K49R, D71G, K91E | 0.3 |
| 59 | 60 | L9V, E21V, G82A | 0.3 |
| 73 | 74 | L9V, E14G, T16Q, K49R, D71G, V84M, K91E | 0.26 |
| 77 | 78 | K25R, K49R, D71G, K91E | 0.23 |
| 79 | 80 | L9V, E14G, T16Q, A36E, K49R, D71G, K91E | 0.23 |
| 81 | 82 | K49R, D71G, K91E | 0.23 |
| 85 | 86 | L9V, E14G, K49R, D71G, K91E | 0.54 |
| 87 | 88 | L9V, K49R, E14G, D71G, H57G, K91E | 0.24 |
| 89 | 90 | K25S, K49R, D71G, K91E | 0.26 |
| 91 | 92 | L9V, E14G, T16Q, K49R, D71G, K91E | 0.2 |
| 93 | 94 | L9V, E14G, K49R, D71G, K91E, Y97F | 0.34 |
| 95 | 96 | L9V, E14G, K49R, D71G, K91E, Y55W | 0.37 |
| 97 | 98 | L9I, E14G, K49R, D71G, K91E | 0.47 |
| 99 | 100 | L9V, E14G, K49R, D71G, K91E, V66L | 0.38 |
| 101 | 102 | L9V, E14G, K49R, D71G, K91E, V31S | 0.38 |
| 103 | 104 | L9V, E14G, K49R, D71G, K91E, V31M | 0.34 |
| 105 | 106 | L9V, E14G, K49R, D71G, K91E, V31E | 0.31 |
| 107 | 108 | L9V, E14G, K49R, D71G, K91E, V28C | 0.34 |
| 109 | 110 | L9V, E14G, K49R, D71G, K91E, T68S | 0.4 |
| 111 | 112 | L9V, E14G, K49R, D71G, K91E, T68Q | 0.32 |
| 113 | 114 | L9V, E14G, K49R, D71G, K91E, T68M | 0.45 |
| 115 | 116 | L9V, E14G, K49R, D71G, K91E, T68G | 0.4 |
| 117 | 118 | L9V, E14G, K49R, D71G, K91E, T68E | 0.42 |
| 119 | 120 | L9V, E14G, K49R, D71G, K91E, T68A | 0.38 |
| 121 | 122 | L9V, E14G, K49R, D71G, K91E, N29G | 0.31 |
| 123 | 124 | L9V, E14G, K49R, D71G, K91E, L6F | 0.35 |
| 125 | 126 | L9V, E14G, K49R, D71G, K91E, K25G, H78P | 0.53 |
| 127 | 128 | L9V, E14G, K49R, D71G, K91E, I74V | 0.36 |
| 129 | 130 | L9V, E14G, K49R, D71G, K91E, I74T | 0.42 |
| 131 | 132 | L9V, E14G, K49R, D71G, K91E, I74M | 0.41 |
| 133 | 134 | L9V, E14G, K49R, D71G, K91E, I74L | 0.4 |
| 135 | 136 | L9V, E14G, K49R, D71G, K91E, I74G | 0.32 |
| 137 | 138 | L9V, E14G, K49R, D71G, K91E, I33V | 0.43 |
| 139 | 140 | L9V, E14G, K49R, D71G, K91E, I33D | 0.41 |
| 141 | 142 | L9V, E14G, K49R, D71G, K91E, E64D | 0.38 |
| 143 | 144 | L9V, E14G, K49R, D71G, K91E, E53S | 0.37 |
| 145 | 146 | L9V, E14G, K49R, D71G, K91E, E53R, V84I | 0.26 |
| 147 | 148 | L9V, E14G, K49R, D71G, K91E, E53R | 0.35 |
| 149 | 150 | L9V, E14G, K49R, D71G, K91E, E53L | 0.37 |
| 151 | 152 | L9V, E14G, K49R, D71G, K91E, E53H | 0.36 |
| 153 | 154 | L9V, E14G, K49R, D71G, K91E, E53F | 0.27 |
| 155 | 156 | L9V, E14G, K49R, D71G, K91E, E53A | 0.38 |
| 157 | 158 | L9V, E14G, K49R, D71G, K91E, E52R | 0.34 |
| 159 | 160 | L9V, E14G, K49R, D71G, K91E, E52Q | 0.43 |
| 161 | 162 | L9V, E14G, K49R, D71G, K91E, D45V | 0.36 |
| 163 | 164 | L9V, E14G, K49R, D71G, K91E, A2G, I74N | 0.42 |

¹The OA titer for the MV005 control strain integrated with codon optimized OAC gene of SEQ ID NO: 5 was set as 1, and the values for “Relative OA titer” for each library strain clone (including parent of SEQ ID NO 85), were determined by comparison to this MV005 control strain.

### Example 3: Preparation and Screening of Engineered Genes Encoding Recombinant Polypeptides with Olivetolic Acid Cyclase Activity

This example illustrates preparation and screening of libraries of engineered genes expressing OAC activity when integrated in a yeast strain already engineered with the C. sativa genes, AAE1 (SEQ ID NO: 1). OLS (SEQ ID NO: 3) and d82PT4 (SEQ ID NO: 9). The libraries were generated by site saturation mutagenesis (SSM) of two parent OAC variant genes from Example 2, SEQ ID NO. 85 and SEQ ID. NO 143 which encode the polypeptides of SEQ ID NO: 86 and 143, each of which has either 5 or 6 amino acid changes relative to the wild-type OAC polypeptide sequence of SEQ ID NO: 6. These SSM libraries were integrated in a yeast strain similar to EVO002 but engineered to include the C. sativa genes, AAE1 (SEQ ID NO: 1), OLS (SEQ ID NO: 3). and d82PT4 (SEQ ID NO: 9) and screened for OA production indicating expression of a recombinant polypeptide having OAC activity.

### Materials and Methods

### A. Site Saturation Mutagenesis Library Builds:

Each of the two OAC parent genes, SEQ ID NO: 143 and SEQ ID. NO: 85 was integrated into a parent yeast strain using CRISPR-Cas9 at the XI-2 site to create the control strains EVO038 and EVO039, respectively. The integrated OAC genes were expressed under the bidirectional Gal1/10 promoter and the PGK1 terminator sequences. The parent strain was previously engineered to include the genes, AAE1 (SEQ ID NO: 1), OLS (SEQ ID NO: 3), and d82PT4 (SEQ ID NO: 9). The resulting control strains (EVO038 and EVO039) integrated with the variant OAC genes of SEQ ID NO. 143 and 85, respectively, thus included a pathway of the genes AAE1, OLS, and OAC capable of converting hexanoic acid to OA, as well as PT4capable of converting OA to CBGA. As in Examples 1 and 2, the control strain was further modified to build a screening strain for integration of the site saturation mutagenesis libraries. This screening strain (EVO029), was built by integrating the m-Venus cassette at the XI-2 site under control of the bidirectional Gal1/10 promoter and PGK1 terminator, thereby replacing the previous integrated OAC gene. The EVO029 strain was no longer capable of converting hexanoic acid to OA or further to CBGA.

The EVO038 and EVO039 control strains were used to calculate fold-improvement in OA titer in each respective library as described below.

Genomic DNA from the control strains were used as the template to generate two PCR products: (1) a first PCR product (Fragment A), which does not harbor any degenerate codons, and (2) a second PCR product (Fragment B), which has sequence overlap with the Fragment A, and is amplified harboring one NNK degenerate codon only. Primers used for amplification of Fragments A and B and overlap extension were designed according to standard site-saturation mutagenesis protocols. Fragment B was amplified with a series of forward primers that included the single NNK degenerate codon scanned across the various desired positions and a single reverse primer: 5′-CTAAGTCTAGCCACGAAAACTGCAA-3′ (SEQ ID NO: 423). Fragment A was amplified using a single forward primer: 5′-GGTTATGAAGAGGAAAAATTGGCAGTAACC-3′ (SEQ ID NO: 424) and a series of reverse primers designed according to the location of the mutagenesis site. The two fragments A and B were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426).

The assembled OE-PCR products were then pooled together per template and gel purified to provide two saturation mutagenesis libraries of linear donor DNA.

The two pooled saturation mutagenesis libraries of linear donor DNA were transformed and integrated as a knock-in using CRISPR-Cas9 into an m-Venus cassette located at the XI-2 site in a yeast strain that already had integrated genes encoding the C. sativa enzymes, AAE1, OLS and PT4 (EVO029). The m-Venus cassette was integrated at the XI-2 site under control of the bidirectional Gal1/10 promoter and PGK1 terminator.

### B. Screening of Site Saturation Mutagenesis Libraries for Olivetolic Acid Biosynthesis:

Individual clones from the saturation mutagenesis libraries integrated in EVO029 and the respective EVO038 or EVO039 control strains were grown in 0.3 mL YPD in 96-well microtiter plates and screened as described in Example 1. Whole broth culture samples were extracted and screened using HPLC as described in Example 1.

### C. Sequencing

Clones determined by screening to exhibit an OA titer were re-tested and sequenced using Sanger sequencing technology to determine their nucleotide and amino acid differences compared to SEQ ID NO 5 and SEQ ID NO 6, respectively.

### D. Results

Results for relative OA titer and corresponding amino acid changes of the SSM libraries generated from the parent codon-optimized genes of SEQ ID NO: 85 and 143 is summarized in Tables 9 and 10 respectively (below).

TABLE 9

| | | | aa | |
|---|---|---|---|---|
| | | | substitution | |
| NT | AA | | relative to | |
| SEQ ID | SEQ ID | | SEQ ID NO: | Relative |
| NO: | NO: | aa substitution(s) relative to CsOAC | 144 | OA titer¹ |
| 143 | 144 | L9V, E14G, K49R, E53S, D71G, K91E | n/a | 1 |
| 165 | 166 | L9V, E14G, K49R, E53S, D71G, G80K, K91E | G80K | 1.12 |
| 167 | 168 | L9V, E14G, K49R, E53S, T68H, D71G, K91E | T68H | 1.15 |
| 169 | 170 | L9V, E14G, K49R, E53S, D71G, I74S, K91E | I74S | 1.08 |
| 171 | 172 | L9V, E14G, K25D, K49R, E53S, D71G, K91E | K25D | 1.02 |
| 173 | 174 | L9V, E14G, K49R, E53S, D71G, S87H, K91E | S87H | 1 |
| 175 | 176 | L9V, E14G, K49R, E53S, T68C, D71G, K91E | T68C | 0.96 |
| 177 | 178 | L9V, E14G, K49R, E53S, D71G, F88Y, K91E | F88Y | 1.05 |
| 179 | 180 | L9V, E14G, K49R, E53S, D71G, I74K, K91E | I74K | 1.12 |
| 181 | 182 | L9V, E14G, Y41Q, K49R, E53S, D71G, K91E | Y41Q | 0.96 |
| 183 | 184 | L9V, E14G, Y41T, K49R, E53S, D71G, K91E | Y41T | 1.05 |
| 185 | 186 | L9V, E14G, K49R, E53S, T68G, D71G, K91E | T68G | 1.05 |
| 187 | 188 | L9V, E14G, K49R, E53S, D71G, D83R, K91E | D83R | 0.92 |
| 189 | 190 | L9V, E14G, Y41S, K49R, E53S, D71G, K91E | Y41S | 1.08 |
| 231 | 232 | L9V, E14G, V46L, K49R, E53S, D71G, K91E | V46L | 0.9 |

¹The OA titer for the EVO038 control strain integrated with codon optimized OAC gene of SEQ ID NO: 143 was set as 1, and the values for “Relative OA titer” for each library strain clone was determined by comparison to this control strain.

TABLE 10

| | | | aa | |
|---|---|---|---|---|
| | | | substitution | |
| NT | AA | | (s) relative | Relative |
| SEQ ID | SEQ ID | | to SEQ ID | OA |
| NO: | NO: | aa substitution(s) relative to CsOAC | NO: 86 | titer¹ |
| 85 | 86 | L9V, E14G, K49R, D71G, K91E | n/a | 1 |
| 191 | 192 | L9V, E14G, E21L, K49R, D71G, K91E | E21L | 0.58 |
| 193 | 194 | L9V, E14G, K49R, E52S, D71G, K91E | E52S | 0.84 |
| 195 | 196 | L9V, E14G, K49R, D71G, D83K, K91E | D83K | 0.69 |
| 197 | 198 | L9V, E14G, K49R, D71G, K91E, T98V | T98V | 1.21 |
| 199 | 200 | L9V, E14G, K49R, D71G, I74R, K91E | I74R | 1.17 |
| 201 | 202 | A2V, L9V, E14G, K49R, D71G, K91E | A2V | 1.24 |
| 203 | 204 | L9V, E14G, K49R, T68C, D71G, K91E | T68C | 1.16 |
| 205 | 206 | L9V, E14G, K49R, D71G, G82A, K91E | G82A | 0.99 |
| 207 | 208 | L9V, E14G, K49R, T68A, D71G, K91E | T68A | 1.43 |
| 209 | 210 | L9V, E14G, T47S, K49R, D71G, I74Q, K91E | T47S, I74Q | 1.49 |
| 211 | 212 | L9V, E14G, T47G, K49R, D71G, K91E | T47G | 1.26 |
| 213 | 214 | L9V, E14G, K49R, D71G, F88W, K91E | F88W | 0.86 |
| 215 | 216 | L9V, E14G, V31A, K49R, D71G, K91E | V31A | 1.29 |
| 217 | 218 | A2S, L9V, E14G, K49R, D71G, K91E | A2S | 1.01 |
| 219 | 220 | L9V, E14G, A18E, K49R, D71G, K91E | A18E | 0.99 |
| 221 | 222 | L9V, E14G, K49R, D71G, R86S, K91E | R86S | 0.9 |
| 223 | 224 | L9V, E14G, K49R, D71G, I74H, K91E | I74H | 1.19 |
| 225 | 226 | L9V, E14G, K49R, D71G, I74Q, K91E | I74Q | 1.28 |
| 227 | 228 | L9V, E14G, T47S, K49R, D71G, K91E | T47S | 1.38 |
| 229 | 230 | L9V, E14G, K49R, D71G, I74N, K91E | I74N | 1.49 |

¹The OA titer for the EVO039 control strain integrated with codon optimized OAC gene of SEQ ID NO: 85 was set as 1, and the values for “Relative OA titer” for each library strain clone was determined by comparison to this control strain.

### Example 4: Preparation and Screening of Engineered Genes Encoding Recombinant Polypeptides with Olivetolic Acid Cyclase Activity

This example illustrates preparation and screening of libraries of engineered genes expressing OAC activity when integrated in a yeast strain already engineered with the C. sativa genes, AAE1 (SEQ ID NO: 1), OLS (SEQ ID NO: 3), and d82PT4 (SEQ ID NO: 9). Combinatorial libraries were generated by two methods: (1) a semi-synthetic method of introducing mutations into a codon-optimized OAC parent gene of SEQ ID NO. 5, using oligonucleotides harboring specific amino acid mutations; and (2) synthesis of 576 combinatorial variants of SEQ ID NO: 5 that contain encode six amino acid mutations relative to the parent sequence. These semi-synthetic and synthesized combinatorial libraries were integrated into yeast strains engineered with the C. sativa genes, AAE1, OLS, and PT4 and screened for OA production indicating expression of a recombinant polypeptide having OAC activity.

### Materials and Methods

### A. Semi Synthetic Combinatorial Library Builds:

The yeast codon-optimized gene of SEQ ID NO: 5 encoding the wild-type C. sativa OAC polypeptide of SEQ ID NO: 6 was synthesized. Oligonucleotides were synthesized to randomly introduce mutations into the parent gene of SEQ ID NO: 5 via PCR amplification. The OAC parent gene of SEQ ID NO: 5 was integrated into a parent yeast strain using CRISPR-Cas9 at the XI-2 site to create the control strain EVO033. The integrated OAC gene was expressed under the bidirectional Gal1/10 promoter and the PGK1 terminator sequences. The parent strain was previously engineered to include the three C. sativa genes, AAE1 (SEQ ID NO: 1), OLS (SEQ ID NO: 3), and d82PT4 (SEQ ID NO: 9), which form a pathway capable of converting hexanoic acid to 3,5,7-trioxododecanoyl-CoA, the precursor for olivetolic acid (OA) synthesis and further to CBGA. The resulting control strain (EVO033) integrated with the OAC gene of SEQ ID NO. 5 thus included a pathway of the genes AAE1, OLS, and OAC capable of converting hexanoic acid to OA, as well as PT4 capable of converting OA to CBGA. As in Example 3, the screening strain for integration of the semi synthetic combinatorial libraries was EVO029, a strain was built by integrating the m-Venus cassette at the XI-2 site under control of the bidirectional Gal1/10 promoter and PGK1 terminator, thereby replacing the previous integrated OAC gene. The EVO029 strain was no longer capable of converting hexanoic acid to OA or further to CBGA. The EVO033 control strain was used to calculate fold-improvement in OA titer in each respective library as described below.

Genomic DNA from the control strain EVO033 was used as the template to generate a PCR product of SEQ ID NO. 5 including 5′ and 3′ flanking sequences using forward primer: 5′-GGTTATGAAGAGGAAAAATTGGCAGTAACC-3′ (SEQ ID NO: 424) and reverse primer: 5′-CTAAGTCTAGCCACGAAAACTGCAA-3′ (SEQ ID NO: 423). The PCR product was then digested into smaller fragments according to standard DNA shuffling protocols. The smaller fragments were gel purified, mixed with a 2 mM pool of oligonucleotides encoding combinations of mutations, and re-assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426). The combinations of mutations introduced per library are listed in Table 11 below:

TABLE 11

| | | | | | | | | |
|---|---|---|---|---|---|---|---|---|
| Combi 1 | A2G | L9V/I | T16Q/P | K25S/G/R | A36E | E53F/S/R/ | E64D/K | I94K |
| | | | | | | L/H/A | | |
| Combi 2 | A2G | L9V/I | Y27F | E52R/Q | V66L | P76V | V84/I/M | |
| Combi 3 | L9V/I | E14G | N29G | K44P | T62G | T68/S/G/A/ | I94k | |
| | | | | | | E/Q/M/ | | |
| Combi 4 | A2G | K12L | T16Q/P | V31M/E/S | K49R | S87K | K91E | |
| Combi 5 | L9V/I | E14G | T16Q | Y27F | K49R | H57G | D71G | K91E |
| Combi 6 | A2G | K12L | Y27F | K49R | E64D/K | 174/V/T/N/ | | |
| | | | | | | M/L/G/S | | |

The oligonucleotide primers used to incorporate mutations are listed in Table 12 below.

TABLE 12

| | | SEQ ID |
|---|---|---|
| Mutation | Primer | NO: |
| A2G | TACGATAAGGTGCTTGACACCCATGGTTTTTTCTCCTTGACG | 431 |
| T16Q | TTCTTCTTTCTGGGCTTCTTGAATTTCGTCCTTGAATTTCAG | 432 |
| E53R | GACGATGTGAGTGTAACCTCTTTCTTTGTTTTTTTGGGTGAC | 433 |
| T62G | CGTCTCTACTGACTCGAAACCGACTTCGACGATGTGAGTGTA | 434 |
| T68E | TATTATGTAATCTTGGATTTCCTCTACTGACTCGAAAGTGAC | 435 |
| I74M | TCCCACATGGGCCGGGTGCATTATGTAATCTTGGATCGTCTC | 436 |
| V84I | TTCCCAGAAGCTACGGTAAATGTCTCCGAATCCCACATGGGC | 437 |
| A36E | CCAATAAACATCTTTCATTTCAGGGATGATATTCACCAGGTT | 438 |
| E53F | GACGATGTGAGTGTAACCAAATTCTTTGTTTTTTTGGGTGAC | 439 |
| E64K | TTGGATCGTCTCTACTGATTTGAAAGTGACTTCGACGATGTG | 440 |
| T68S | TATTATGTAATCTTGGATAGACTCTACTGACTCGAAAGTGAC | 441 |
| I74G | TCCCACATGGGCCGGGTGACCTATGTAATCTTGGATCGTCTC | 442 |
| V84M | TTCCCAGAAGCTACGGTACATGTCTCCGAATCCCACATGGGC | 443 |
| L9V | AATTTCGTCCTTGAATTTAACTACGATAAGGTGCTTGACAGC | 444 |
| N29G | AGGGATGATATTCACCAGACCTACATAGGTCTTGAAAAATTC | 445 |
| E53S | GACGATGTGAGTGTAACCAGATTCTTTGTTTTTTTGGGTGAC | 446 |
| E64D | TTGGATCGTCTCTACTGAATCGAAAGTGACTTCGACGATGTG | 447 |
| D71G | GGCCGGGTGTATTATGTAACCTTGGATCGTCTCTACTGACTC | 448 |
| S87K | CAAAAGTTTTTCCCAGAATTTACGGTAAACGTCTCCGAATCC | 449 |
| L9I | AATTTCGTCCTTGAATTTAATTACGATAAGGTGCTTGACAGC | 450 |
| V31S | CATCGCAGGGATGATATTAGACAGGTTTACATAGGTCTTGAA | 451 |
| K44P | GTTTTTTTGGGTGACGTCTGGGCCCCAATAAACATCTTTCAT | 452 |
| E53L | GACGATGTGAGTGTAACCCAATTCTTTGTTTTTTTGGGTGAC | 453 |
| V66L | GTAATCTTGGATCGTCTCCAATGACTCGAAAGTGACTTCGAC | 454 |
| I74N | TCCCACATGGGCCGGGTGATTTATGTAATCTTGGATCGTCTC | 455 |
| K91E | ATAGTCGAAAATCAAAAGTTCTTCCCAGAAGCTACGGTAAAC | 456 |
| K25S | CACCAGGTTTACATAGGTAGAGAAAAATTCTTCTTTCTGGGC | 457 |
| V31M | CATCGCAGGGATGATATTCATCAGGTTTACATAGGTCTTGAA | 458 |
| E53H | GACGATGTGAGTGTAACCATGTTCTTTGTTTTTTTGGGTGAC | 459 |
| T68Q | TATTATGTAATCTTGGATTTGCTCTACTGACTCGAAAGTGAC | 460 |
| I74V | TCCCACATGGGCCGGGTGAACTATGTAATCTTGGATCGTCTC | 461 |
| I94K | TCTCGGGGTATAGTCGAATTTCAAAAGTTTTTCCCAGAAGCT | 462 |
| K12L | GGCTTCTGTAATTTCGTCCAAGAATTTCAGTACGATAAGGTG | 463 |
| K25G | CACCAGGTTTACATAGGTACCGAAAAATTCTTCTTTCTGGGC | 464 |
| V31E | CATCGCAGGGATGATATTTTCCAGGTTTACATAGGTCTTGAA | 465 |
| K49R | GTAACCTTCTTCTTTGTTTCTTTGGGTGACGTCTTTGCCCCA | 466 |
| E53A | GACGATGTGAGTGTAACCAGCTTCTTTGTTTTTTTGGGTGAC | 467 |
| T68A | TATTATGTAATCTTGGATAGCCTCTACTGACTCGAAAGTGAC | 468 |
| I74T | TCCCACATGGGCCGGGTGAGTTATGTAATCTTGGATCGTCTC | 469 |
| E14G | TTTCTGGGCTTCTGTAATACCGTCCTTGAATTTCAGTACGAT | 470 |
| K25R | CACCAGGTTTACATAGGTTCTGAAAAATTCTTCTTTCTGGGC | 471 |
| E52Q | GATGTGAGTGTAACCTTCTTGTTTGTTTTTTTGGGTGACGTC | 472 |
| T68G | TATTATGTAATCTTGGATACCCTCTACTGACTCGAAAGTGAC | 473 |
| I74S | TCCCACATGGGCCGGGTGAGATATGTAATCTTGGATCGTCTC | 474 |
| T16P | TTCTTCTTTCTGGGCTTCTGGAATTTCGTCCTTGAATTTCAG | 475 |
| E52R | GATGTGAGTGTAACCTTCTCTTTTGTTTTTTTGGGTGACGTC | 476 |
| H57G | GAAAGTGACTTCGACGATACCAGTGTAACCTTCTTCTTTGTT | 477 |
| T68M | TATTATGTAATCTTGGATCATCTCTACTGACTCGAAAGTGAC | 478 |
| I74L | TACGATAAGGTGCTTGACACCCATGGTTTTTTCTCCTTGACG | 479 |

The final libraries were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426). The assembled OE-PCR products were then pooled together per library and gel purified to provide six semi-synthetic combinatorial libraries of linear donor DNA. The six pooled semi-synthetic combinatorial libraries of linear donor DNA were transformed and integrated as a knock-in using CRISPR-Cas9 into an m-Venus cassette located at the XI-2 site in a yeast strain that already had integrated genes encoding the C. sativa enzymes, AAE1, OLS and PT4 (EVO029). The m-Venus cassette was integrated at the XI-2 site under control of the bidirectional Gal1/10 promoter and PGK1 terminator. B. Synthesis of 576 Combinatorial Variants

The synthetic gene of SEQ ID NO: 5 was used to design and synthesize 576 variants each with six amino acid changes with respect to SEQ ID NO: 6. As in Example 1, to facilitate efficient integration, the 50-mer 5′ and 3′ flanking sequences of SEQ ID NO: 427 and SEQ ID NO: 428 were introduced into each combinatorial variant. As in Example 1, these flanking sequences provided the overlap homology to build longer DNA donors including the pGal1 promoter and the PGK1t terminator. The combinatorial variants including the flanking sequence were synthesized by Twist.

A first PCR product (Fragment A) that has sequence overlap with the 5′ end of each of the OAC variants was amplified from EVO029 genomic DNA using the following primers 5′-GGTTATGAAGAGGAAAAATTGGCAGTAACC-3′ (SEQ ID NO: 480), and 5′-CTTTAACACTATCAAGTGCTTTACAGCCATGGTTTTTTCTCCTTGACGTTAAAGTATAGA-3′ ( SEQ ID NO: 481). A second PCR product (Fragment B) that has sequence overlap with the 3′ end of each of the OAC was amplified from EVO029 genomic DNA using the following primers 5′-GGAAACCTCTACACATAGAAATATCGAATGGG-3′ (SEQ ID NO: 482) and 5′-TTGTTAATTTTTGATTACACTCCAAGGAAGTAAATTGAATTGAATTGAAATCGATAGATC-3′ (SEQ ID NO: 483). The third product, OAC Variants (Fragment C) was synthesized as described above with between 47 and 50 base pair sequence overlap with the 3′ end of Fragment A (amplified from gDNA template) and overlap with the 5′ end of Fragment B (amplified from gDNA template). The three Fragments A, B, and C were assembled by overlap extension PCR using the forward primer 5′-GAACGAATCAAATTAACAACCATAGGATGA-3′ (SEQ ID NO: 425) and reverse primer 5′-GAGGAGCGAATTTTTTTTTAATAAAAATCT-3′ (SEQ ID NO: 484). Thus, the integrated synthesized genes were expressed under the bidirectional Gal1/10 promoter and the PGK1 terminator sequences. The parent strain was previously engineered to include the three C. sativa genes, AAE1 (SEQ ID NO: 1), OLS (SEQ ID NO: 3), and d82PT4 (SEQ ID NO: 9), which form a pathway capable of converting hexanoic acid to 3,5,7-trioxododecanoyl-CoA, the precursor for olivetolic acid (OA) synthesis and further to CBGA. The resulting control strain (EVO033) integrated with the OAC gene of SEQ ID NO. 5, thus included a pathway of the genes AAE1, OLS, and OAC capable of converting hexanoic acid to OA, as well as PT4 capable of converting OA to CBGA. As in Example 3, the screening strain for integration of the semi synthetic combinatorial libraries was EVO029, a strain was built by integrating the m-Venus cassette at the XI-2 site under control of the bidirectional Gal1/10 promoter and PGK1 terminator, thereby replacing the previous integrated OAC gene. The EVO029 strain was no longer capable of converting hexanoic acid to OA or further to CBGA. The EVO033 control strain was used to calculate fold-improvement in OA titer in each respective library as described below.

### C. Screening of the Semi-Synthetic and Synthesized Combinatorial Libraries for Olivetolic Acid Biosynthesis:

Individual clones from the semi-synthetic and synthesized combinatorial libraries were integrated in EVO029 and the respective EVO033 control strains were grown in 0.3 mL YPD in 96-well microtiter plates and screened as described in Example 1. Whole broth culture samples were extracted and screened using HPLC as described in Example 1.

### C. Sequencing

Clones determined by screening to exhibit an OA titer were re-tested and sequenced using Sanger sequencing technology to determine their nucleotide and amino acid differences compared to SEQ ID NO 5 and SEQ ID NO 6, respectively.

### D. Results

Results for relative OA titer and corresponding amino acid changes of the semi-synthetic combinatorial libraries generated from the parent codon-optimized genes of SEQ ID NO: 5 are summarized in Table 13 (below).

TABLE 13

| NT | AA | | |
|---|---|---|---|
| SEQ ID | SEQ ID | | Relative |
| NO: | NO: | aa substitution(s) relative to CsOAC | OA titer¹ |
| 233 | 234 | A2G, L9I, K25R | 0.87 |
| 235 | 236 | L9I, E53S, I94K | 1.16 |
| 237 | 238 | A36E | 1.15 |
| 239 | 240 | E53H, E64D | 0.97 |
| 241 | 242 | L9I, K25R, A36E, E53H, I94K | 1.32 |
| 243 | 244 | A2G, L9I, K25R, E53S, E64D, I94K | 0.9 |
| 245 | 246 | K25S, A36E | 1.23 |
| 247 | 248 | L9I, T16Q, K25R, E53S, E64D | 1.17 |
| 249 | 250 | K25S, I94K | 1.04 |
| 251 | 252 | A2G, L9I, K25G | 1.04 |
| 253 | 254 | E53A | 0.93 |
| 255 | 256 | A2G, L9V, T16P | 0.23 |
| 257 | 258 | E53A, E64D, I94K | 0.9 |
| 259 | 260 | L9I, K25S, A36E, E64D, I94K | 1.39 |
| 26 | 262 | L9I, K25S, A36E, E53R, I94K | 1.11 |
| 263 | 264 | K25E, A36E, E64D, I94K | 1.22 |
| 265 | 266 | K25R, E64D | 1.1 |
| 267 | 268 | K25R, A36E, E53S, E64D, I94K | 1.2 |
| 269 | 270 | A2G, L9I, A36E, E53S, E64D, I94K | 1.09 |
| 271 | 272 | A2G, L9V, Y27F, E52R, V66L, V84M | 1.36 |
| 273 | 274 | L9V, V66L | 1.27 |
| 275 | 276 | L9V, Y27F, V84M | 1.18 |
| 277 | 278 | A2G, L9V | 1.06 |
| 279 | 280 | L9I, Y27F, E53S, V66L, V84M | 0.71 |
| 281 | 282 | V66L, V84M | 1.05 |
| 283 | 284 | L9V, E52Q, V66L, P76V | 0.55 |
| 285 | 286 | L9I, Y27F, V66L, V84I | 0.94 |
| 287 | 288 | A2G, L9V, Y27F, V66L | 0.85 |
| 289 | 290 | Y27F | 0.82 |
| 291 | 292 | A2G, L9V, V66L, V84M | 1.01 |
| 293 | 294 | L9V, T26A, E52Q, P76V | 1.05 |
| 295 | 296 | L9V, E52R, V84M | 1.18 |
| 297 | 298 | L9V, Y27F, V66L, V84M | 1.14 |
| 299 | 300 | L9I, N29G, T62G, T68E, I94K | 0.86 |
| 301 | 302 | L9I, E14G, K44P, T68E, I94K | 1 |
| 303 | 304 | E14G, T68S, I94K | 1.19 |
| 305 | 306 | E14G, T68A, I94K | 1.24 |
| 307 | 308 | T62G, T68G, I94K | 0.49 |
| 309 | 310 | L9V, E14G, T68E, I94K | 1.14 |
| 311 | 312 | L9V, I94K | 1 |
| 313 | 314 | L9V, N29G, T68E, I94K | 1.16 |
| 315 | 316 | A2G, T16P, V31M, K49R, S87K | 0.23 |
| 317 | 318 | V31E, F63L, S87K | 1.16 |
| 319 | 320 | A2G, K49R | 1.09 |
| 321 | 322 | V31M, K49R, S87K | 1.1 |
| 323 | 324 | A2G, V31S, K49R, K91E | 0.5 |
| 325 | 326 | V31E, K49R, S87K | 0.84 |
| 327 | 328 | T16P, V31M, K49R | 1.05 |
| 329 | 330 | L9V, E14G, V31S, K49R, K91E | 1.01 |
| 331 | 332 | K12L, V31M, K49R | 0.98 |
| 333 | 334 | L9V, K91E | 0.72 |
| 335 | 336 | L9I, E14G, V40A, K49R | 1.01 |
| 337 | 338 | L9V, K49R, D71G, K91E | 0.49 |
| 339 | 340 | L9I, E14G, Y27F, D71G, K91E | 0.28 |
| 341 | 342 | E14G, K49R, D71G, K91E | 0.37 |
| 343 | 344 | L9I, Y27F, K91E | 0.74 |
| 345 | 346 | K49R, Y85F, K91E | 0.86 |
| 347 | 348 | L9I, E14G, D71G, K91E | 0.65 |
| 349 | 350 | L9V, K49R, K91E | 1.05 |
| 351 | 352 | L9I, E14G, K49R, D71G | 1.21 |
| 353 | 354 | E14G, Y27F, K49R | 0.93 |
| 355 | 356 | L9V, T16Q, K49R, K91E | 0.85 |
| 357 | 358 | L9I, K49R, K91E | 1.1 |
| 359 | 360 | L9V, E14G, Y27F, K49R, K91E | 0.75 |
| 361 | 362 | K49R, K91E | 0.85 |
| 363 | 364 | L91, E14G, D71G | 1.15 |
| 365 | 366 | E14G, K49R, K91E | 0.72 |
| 367 | 368 | K49R, E64D, I74T | 1.15 |
| 369 | 370 | K12L, Y27F, K49R, I74M | 0.94 |
| 371 | 372 | A2G, Y27F, E64K, I74V | 0.3 |
| 373 | 374 | Y27F, K49R, E64D, I74V | 1 |
| 375 | 376 | Y27F, K49R, E64D, I74T | 0.9 |
| 377 | 378 | A2G, K49R, E64D, I74M | 1.11 |
| 379 | 380 | A2G, K49R, E64D, I74N | 1 |
| 381 | 382 | K49R, E64K, I74N | 1.1 |
| 383 | 384 | L9V, E14G, K25A, Q48P, I74N | 0.99 |
| 385 | 386 | K12L, K49R, I74V | 1.19 |
| 387 | 388 | Y27F, K49R, I74V | 1.02 |
| 389 | 390 | K49R, I74S | 1.16 |
| 391 | 392 | A2G, K12L, K49R, I74N | 0.68 |
| 393 | 394 | L9V, E14G, T47A, E64D, I74V | 1.37 |
| 395 | 396 | K12L, K49R, E64D, I74S | 1.08 |
| 397 | 398 | Y27F, K49R, I74N | 0.97 |
| 399 | 400 | A2G, E64K, I74M | 0.54 |
| 401 | 402 | V8I, L9I, I33V, K49R, T56S, D71T | 1.35 |
| 403 | 404 | V8I, L9I, I33D, K49R, T56S, I58V | 1.27 |
| 405 | 406 | V8I, L9I, K12N, K49R, Y55W, T56S | 1 |
| 407 | 408 | V8I, L9I, K12V, I33V, T56S, E64D | 1.29 |
| 409 | 410 | L9I, K12V, I33V, T56S, I58V, E64D | 1.24 |
| 411 | 412 | A2P, V8I, L9I, E64D, T68S, R100G | 1.33 |
| 413 | 414 | V8I, K12V, T56S, I58V, E64D, Y97F | 0.86 |
| 415 | 416 | V31M, K49R, I58V, T62C, E64D, T68A | 1.08 |
| 417 | 418 | V8I, K49R, I58V, E64D, T68M, I74M | 1.25 |
| 419 | 420 | A36P, I58V, E64D, T68G, Q70K, I74M | 1.14 |
| 421 | 422 | E14G, A36Q, K49R, I58V, E64D, Q70K | 1.28 |
| 485 | 486 | A2P, V8I, L91, I33V, E64D, I94K | 1.51 |
| 487 | 488 | V8I, L9I, A36S, Y55W, E64D, I94K | 1.48 |
| 489 | 490 | V8I, L9I, I33V, K49R, I58V, T62C | 1.46 |
| 491 | 492 | V8I, L9I, I33V, K49R, I58V, T62C | 1.45 |
| 493 | 494 | V8I, L9I, I33D, K49R, T56S, D71T | 1.44 |
| 495 | 496 | V8I, L9I, K12N, I33V, K49R, D71T | 1.42 |
| 497 | 498 | V8I, L9I, I33V, K49R, I58V, R100G | 1.39 |
| 499 | 500 | V8I, L9I, K12N, I33V, K49R, D71T | 1.38 |
| 501 | 502 | V8I, L9I, K49R, Y55W, I58V, R100G | 1.38 |
| 503 | 504 | V8I, L9I, I33V, K49R, T56S, R100G | 1.38 |
| 505 | 506 | V8I, L9I, I33V, A36L, K49R, I58V | 1.37 |
| 507 | 508 | L9I, I33V, A36Q, I58V, E64D, I94K | 1.36 |
| 509 | 510 | V8I, L9I, K10A, I33V, T56S, I58V | 1.35 |
| 511 | 512 | L9I, I33V, K49R, T56S, I58V, R100G | 1.34 |
| 513 | 514 | V8I, L9I, K12N, I33V, K49R, D71T | 1.33 |
| 515 | 516 | L9I, I33V, A36Q, I58V, E64D, I94K | 1.32 |
| 517 | 518 | V8I, L9I, K10A, N29D, I33V, I58V | 1.32 |
| 519 | 520 | L9I, I33V, K49R, T56S, I58V, R100G | 1.32 |
| 521 | 522 | V8I, L9I, I33D, T56S, I58V, E64D | 1.30 |
| 523 | 524 | V8I, L9I, I33D, K49R, T56S, D71T | 1.30 |
| 525 | 526 | V8I, L9I, I33D, K49R, T56S, D71T | 1.28 |
| 527 | 528 | L9I, A36Q, Y55W, I58V, E64D, I94K | 1.27 |
| 529 | 530 | V8I, L9I, I33V, T56S, I58V, Q70K | 1.27 |
| 531 | 532 | V8I, L9I, K49R, Y55W, T56S, R100G | 1.26 |
| 533 | 534 | V8I, L9I, I33D, K49R, T56S, D71T | 1.25 |
| 535 | 536 | V8I, L9I, K10A, N29D, I33V, T56S | 1.25 |
| 537 | 538 | V8I, L9I, K10A, N29D, I33V, T56S | 1.25 |
| 539 | 540 | A2P, V8I, L9I, Y55W, E64D, I94K | 1.24 |
| 541 | 542 | L9I, A36Q, Y55W, I58V, E64D, I94K | 1.24 |
| 543 | 544 | V8I, L9I, I33V, T56S, I58V, R100G | 1.23 |
| 545 | 546 | V8I, L9I, Y55W, T56S, I58V, R100G | 1.22 |
| 547 | 548 | L9I, A36Q, Y55W, I58V, E64D, I94K | 1.22 |
| 549 | 550 | V8I, L9I, K12N, T16Q, K49R, T56S | 1.21 |
| 551 | 552 | V8I, L9I, K49R, Y55W, I58V, T62C | 1.21 |
| 553 | 554 | A2P, V8I, L9I, T16Q, E64D, I94K | 1.20 |
| 555 | 556 | V8I, L9I, I33D, T56S, I58V, E64D | 1.19 |
| 557 | 558 | V8I, L9I, T16Q, A36F, K49R, I58V | 1.19 |
| 559 | 560 | V8I, I33V, K49R, T56S, I58V, R100G | 1.19 |
| 561 | 562 | V8I, L9I, T16Q, T56S, I58V, Q70K | 1.19 |
| 563 | 564 | V8I, L9I, T16Q, T56S, I58V, R100G | 1.18 |
| 565 | 566 | V8I, L9I, K10A, N29D, I33V, T56S | 1.17 |
| 567 | 568 | V8I, L9I, K10A, N29D, I33V, T56S | 1.17 |
| 569 | 570 | L9I, I33V, K49R, T56S, I58V, R100G | 1.17 |
| 571 | 572 | V8I, L9I, Y55W, T56S, I58V, R100G | 1.16 |
| 573 | 574 | L9I, A36Q, Y55W, I58V, E64D, I94K | 1.16 |
| 575 | 576 | V8I, L9I, K12V, I33V, T56S, I58V | 1.15 |
| 577 | 578 | V8I, L9I, T16Q, A36F, K49R, I58V | 1.14 |
| 579 | 580 | V8I, L9I, K12N, I33V, T56S, D71T | 1.14 |
| 581 | 582 | V8I, L9I, K49R, Y55W, T56S, R100G | 1.13 |
| 583 | 584 | V8I, L9I, I33V, A36S, T62C, E64D | 1.13 |
| 585 | 586 | V8I, K49R, Y55W, T56S, I58V, R100G | 1.13 |
| 587 | 588 | L9I, A36Q, I58V, E64D, T68S, I94K | 1.64 |
| 589 | 590 | V8I, L9I, T56S, I58V, T68S, R100G | 1.52 |
| 591 | 592 | V8I, L9I, K12V, T56S, E64D, T68S | 1.47 |
| 593 | 594 | L9I, A36Q, I58V, E64D, I94K, Y97F | 1.46 |
| 595 | 596 | V8I, L9I, V28C, K49R, T56S, I58V | 1.44 |
| 597 | 598 | V8I, L9I, K12V, I58V, E64D, T68G | 1.44 |
| 599 | 600 | V8I, L9I, A36S, E64D, I94K, Y97F | 1.44 |
| 601 | 602 | V8I, L9I, K12N, K49R, T68G, D71T | 1.39 |
| 603 | 604 | V8I, L9I, K49R, I58V, T62C, Y97F | 1.38 |
| 605 | 606 | V8I, L9I, V28C, K49R, T56S, D71T | 1.36 |
| 607 | 608 | V8I, L9I, K49R, I58V, T62C, Y97F | 1.36 |
| 609 | 610 | V8I, L9I, K10A, N29D, I58V, T68G | 1.35 |
| 611 | 612 | V8I, L9I, K49R, I58V, T68S, R100G | 1.35 |
| 613 | 614 | L9I, K49R, T56S, I58V, T68G, R100G | 1.32 |
| 615 | 616 | V8I, L9I, K12V, T56S, E64D, T68S | 1.31 |
| 617 | 618 | V8I, L9I, K10A, N29D, I58V, T68G | 1.30 |
| 619 | 620 | V8I, L9I, A36S, E64D, I94K, Y97F | 1.29 |
| 621 | 622 | V8I, L9I, K12N, K49R, T56S, Y97F | 1.28 |
| 623 | 624 | V8I, L9I, K12V, I58V, E64D, T68G | 1.28 |
| 625 | 626 | V8I, L9I, K12V, I58V, E64D, Y97F | 1.27 |
| 627 | 628 | V8I, L9I, T56S, I58V, T68S, R100G | 1.27 |
| 629 | 630 | V8I, L9I, K49R, I58V, T62C, T68G | 1.27 |
| 631 | 632 | V8I, L9I, K12V, T56S, E64D, T68S | 1.27 |
| 633 | 634 | V8I, K49R, T56S, I58V, T68S, R100G | 1.25 |
| 635 | 636 | V8I, L9I, T56S, I58V, T68G, Q70K | 1.25 |
| 637 | 638 | V8I, L9I, K49R, I58V, T62C, T68G | 1.24 |
| 639 | 640 | V8I, L9I, K12V, T56S, I58V, T68G | 1.24 |
| 641 | 642 | V8I, L9I, K12V, T56S, I58V, T68S | 1.24 |
| 643 | 644 | V8I, L9I, A36F, K49R, I58V, T68S | 1.23 |
| 645 | 646 | V8I, L9I, K49R, I58V, T62C, Y97F | 1.23 |
| 647 | 648 | V8I, L9I, A36F, K49R, I58V, Y97F | 1.23 |
| 649 | 650 | V8I, L9I, K12N, K49R, T68G, D71T | 1.23 |
| 651 | 652 | V8I, L9I, K12V, T56S, E64D, T68S | 1.22 |
| 653 | 654 | V8I, L9I, K49R, I58V, Y97F, R100G | 1.22 |
| 655 | 656 | V8I, L9I, A36F, K49R, I58V, Y97F | 1.22 |
| 657 | 658 | V8I, L9I, K12V, T56S, E64D, T68S | 1.21 |
| 659 | 660 | V8I, L9I, K10A, N29D, T56S, T68S | 1.21 |
| 661 | 662 | V8I, L9I, V28C, K49R, T56S, I58V | 1.21 |
| 663 | 664 | V8I, L9I, K10A, T56S, I58V, Y97F | 1.19 |
| 665 | 666 | V8I, L9I, K12V, E14G, K49R, E67S | 1.60 |
| 667 | 668 | L9I, A36S, K49R, E64D, T68M, I74M | 1.57 |
| 669 | 670 | L9I, Y41E, K49R, I58V, V66I, T68E | 1.54 |
| 671 | 672 | V8I, L9I, K12V, E14G, K49R, E67S | 1.53 |
| 673 | 674 | V8I, L9I, K12N, T16Q, K49R, I58V | 1.49 |
| 675 | 676 | V8I, L9I, K12V, E14G, K49R, E67S | 1.47 |
| 677 | 678 | V8I, L9I, K25N, K49R, I58V, T68S | 1.46 |
| 679 | 680 | V8I, L9I, K49R, T56S, V66I, T68M | 1.44 |
| 681 | 682 | V8I, L9I, K12V, E14G, K49R, E67S | 1.43 |
| 683 | 684 | V8I, L9I, K25N, A36P, T68G, I74M | 1.43 |
| 685 | 686 | L9I, Y41E, K49R, I58V, V66I, T68E | 1.42 |
| 687 | 688 | V8I, L9I, K49R, E64D, Y97F, R100G | 1.41 |
| 689 | 690 | V8I, L9I, K12V, E14G, K49R, E67S | 1.40 |
| 691 | 692 | V8I, L9I, I33V, 158V, E64D, R100G | 1.40 |
| 693 | 694 | L9I, A36S, K49R, E64D, T68M, I74M | 1.40 |
| 695 | 696 | V8I, L9I, K49R, E64D, Y97F, R100G | 1.37 |
| 697 | 698 | V8I, L9I, Y41E, I58V, E64D, T68A | 1.36 |
| 699 | 700 | V8I, K12Q, V31M, V66I, T68S, I74M | 1.35 |
| 701 | 702 | V8I, L9I, Y41E, I58V, E64D, T68A | 1.35 |
| 703 | 704 | L9I, E14G, I58V, T62C, E64D, V66I | 1.35 |
| 705 | 706 | V8I, L9I, K49R, T56S, V66I, T68M | 1.35 |
| 707 | 708 | V8I, L9I, K25N, A36P, T68G, I74M | 1.34 |
| 709 | 710 | V8I, L9I, K25N, K49R, I58V, T68S | 1.34 |
| 711 | 712 | L9I, T16Q, K49R, I58V, E64D, R100G | 1.34 |
| 713 | 714 | V8I, K12Q, V31M, V661, T68S, I74M | 1.33 |
| 715 | 716 | V8I, K12Q, I33V, K49R, I58V, I74M | 1.33 |
| 717 | 718 | V8I, L9I, K49R, E64D, Y97F, R100G | 1.33 |
| 719 | 720 | L9I, T16Q, K49R, I58V, E64D, R100G | 1.31 |
| 721 | 722 | L9I, E14G, I58V, T62C, E64D, V66I | 1.29 |
| 723 | 724 | V8I, L9I, K49R, E64D, Y97F, R100G | 1.29 |
| 725 | 726 | V8I, K12N, I33V, I58V, E64D, V66I | 1.29 |
| 727 | 728 | A2P, L9I, K25N, V31M, K49R, Y55W | 1.29 |
| 729 | 730 | V8I, L9I, K12N, T16Q, K49R, I58V | 1.28 |
| 731 | 732 | L9I, A36S, K49R, E64D, T68M, I74M | 1.28 |
| 733 | 734 | L9I, K49R, I58V, T68A, D71T, I94K | 1.27 |
| 735 | 736 | L9I, E14G, I58V, T62C, E64D, V66I | 1.27 |
| 737 | 738 | V8I, E14G, K49R, I58V, E64D, R100A | 1.26 |
| 739 | 740 | L9V, E14G, K49R, E53S, D71G, K91E | 1.26 |
| 741 | 742 | A2S, V8I, K49R, E53V, T56S, T68S | 1.26 |
| 743 | 744 | V8I, K12Q, V31M, V66I, T68S, I74M | 1.26 |
| 745 | 746 | V8I, K12Q, V31M, V66I, T68S, I74M | 1.25 |
| 747 | 748 | A2P, V8I, K49R, T62C, T68E, I74M | 1.25 |
| 749 | 750 | V8I, L9I, K12N, T16Q, K49R, I58V | 1.25 |
| 751 | 752 | V8I, L9I, I33V, K49R, T62C, E64D | 1.23 |
| 753 | 754 | V8I, K12Q, V31M, V66I, T68S, I74M | 1.23 |
| 755 | 756 | L9I, E53V, I58V, E64D, T68S, I74M | 1.22 |
| 757 | 758 | V8I, V28C, A36S, K49R, T56S, V66I | 1.22 |
| 759 | 760 | V8I, K12Q, V31M, V66I, T68S, I74M | 1.22 |
| 761 | 762 | V8I, L9I, K49R, E64D, Y97F, R100G | 1.21 |
| 763 | 764 | V8I, L9I, I33V, I58V, E64D, R100G | 1.21 |
| 765 | 766 | V8I, E14G, K49R, I58V, E64D, R100A | 1.20 |
| 767 | 768 | V8I, L9I, K25N, A36P, T68G, I74M | 1.19 |
| 769 | 770 | V8I, V31M, E64D, T68Q, Q70K, D71T | 1.19 |
| 771 | 772 | L9I, K25N, A36Q, K49R, V66I, T68Q | 1.50 |
| 773 | 774 | L9I, K25N, A36Q, K49R, V66I, T68Q | 1.44 |
| 775 | 776 | V8I, K49R, E64D, T68S, E90D, I94K | 1.39 |
| 777 | 778 | V8I, A36S, T56S, T68S, E90D, I94K | 1.35 |
| 779 | 780 | L9I, K49R, T62C, E64D, T68G, I74M | 1.31 |
| 781 | 782 | K12N, T56S, I58V, V66I, T68A, D71T | 1.29 |
| 783 | 784 | L9I, I33V, Q48M, I58V, E64D, I74M | 1.28 |
| 785 | 786 | L9I, V31M, K49R, N50Y, T68S, I74M | 1.27 |
| 787 | 788 | V8I, K25N, I33V, K49R, E64D, I74M | 1.26 |
| 789 | 790 | L9I, A18S, K25N, K49R, I58V, S87P | 1.24 |
| 791 | 792 | L9I, E22L, K49R, E64D, I74M, Y97F | 1.21 |
| 793 | 794 | V8I, K49R, E64D, T68S, E90D, I94K | 1.19 |
| 795 | 796 | L9I, E22L, V46I, E64D, I74M, Y97F | 1.17 |
| 797 | 798 | V8I, A18S, K49R, E64D, V66I, I94K | 1.15 |
| 799 | 800 | L9I, E22L, K49R, E64D, I74M, Y97F | 1.14 |
| 801 | 802 | A2S, V8I, T56S, T62C, T68S, I74M | 1.10 |
| 803 | 804 | Y41E, K49R, T56S, I58V, V66I, T68A | 1.09 |
| 805 | 806 | K10A, K49R, I58V, E64D, V66I, T68E | 1.45 |
| 807 | 808 | K12N, A18S, K49R, I58V, V66I, I74M | 1.31 |
| 809 | 810 | K49R, 158V, T62C, T68E, D71T, I74M | 1.21 |
| 811 | 812 | I33V, K49R, T62C, E64D, V66I, I74M | 1.20 |
| 813 | 814 | T16Q, K25N, A36Q, K49R, V66I, I74M | 1.16 |
| 815 | 816 | A2P, I58V, E64D, E67S, T68Q, R100G | 1.14 |
| 817 | 818 | I33V, K49R, T62C, E64D, V66I, I74M | 1.14 |
| 819 | 820 | K49R, I58V, T68A, D71T, I74M, R100G | 1.14 |
| 821 | 822 | K25N, K49R, I58V, E64D, V66I, T68S | 1.13 |
| 823 | 824 | A36Q, K49R, I58V, E64D, V66I, K91E | 1.10 |
| 825 | 826 | K12N, A18S, K49R, I58V, V66I, I74M | 1.07 |
| 827 | 828 | E21V, A36Q, I58V, V66I, E67S, I74M | 1.06 |
| 829 | 830 | A2P, E14G, Q48C, I58V, E64D, V66I | 1.05 |
| 831 | 832 | A2P, K12V, T16Q, K49R, I58V, V66I | 1.03 |
| 833 | 834 | A2P, K12V, T16Q, K49R, I58V, V66I | 1.02 |
| 835 | 836 | I33V, K49R, T62C, E64D, V66I, I74M | 1.01 |
| 837 | 838 | A2P, V31M, I58V, T62C, E64D, T68S | 0.91 |
| 839 | 840 | A36P, I58V, E67S, T68S, I74M, R100G | 0.90 |
| 841 | 842 | F23I, K49R, V66I, T68Q, Q70A, I74M | 0.90 |
| 843 | 844 | E22L, V31M, A36S, V66I, T68S, I74M | 0.85 |
| 845 | 846 | A36S, K49R, 158C, E64D, V66I, T68S | 1.23 |
| 847 | 848 | T16Q, A36Q, Q48H, I58V, E64D, I74M | 1.16 |
| 849 | 850 | K25N, K49R, Y55W, 158V, E64D, I74M | 1.12 |
| 851 | 852 | A36S, Q48C, E64D, T68E, I74M, S87P | 1.05 |
| 853 | 854 | K49R, V66I, T68Q, D71T, I74M, 194K | 0.92 |

¹The OA titer for the EVO033 control strain integrated with codon optimized OAC gene of SEQ ID NO: 5 was set as 1, and the values for “Relative OA titer” for each library strain clone was determined by comparison to this control strain.

### Example 5: Preparation and Screening of Engineered Genes Encoding Recombinant Polypeptides with Olivetolic Acid Cyclase Activity

This example illustrates preparation and screening of libraries of engineered genes expressing OAC activity when integrated in a yeast strain already engineered with the C. sativa genes, AAE1 (SEQ ID NO: 1), OLS (SEQ ID NO: 3) and d82PT4 (SEQ ID NO: 9). The libraries were generated by site saturation mutagenesis (SSM) of two parent OAC variant genes from Example 5, SEQ ID NO. 411 and SEQ ID. NO 417 which encode the polypeptides of SEQ ID NO: 412 and 418, each of which has 6 amino acid changes relative to the wild-type OAC polypeptide sequence of SEQ ID NO: 6. These SSM libraries were integrated in a yeast strain similar to EVO002 but engineered to include the C. sativa genes, AAE1 (SEQ ID NO: 1), OLS (SEQ ID NO: 3), and d82PT4 (SEQ ID NO: 9) and screened for OA production indicating expression of a recombinant polypeptide having OAC activity.

### Materials and Methods

### A. Site Saturation Mutagenesis Library Builds:

Each of the two OAC parent genes, SEQ ID NO: 411 and SEQ ID. NO: 417 was integrated into a parent yeast strain using CRISPR-Cas9 at the XI-2 site to create the control strains EVO067 and EVO068, respectively. The integrated OAC genes were expressed under the bidirectional Gal1/10 promoter and the PGK1 terminator sequences. The parent strain was previously engineered to include the genes, AAE1 (SEQ ID NO: 1), OLS (SEQ ID NO: 3), and d82PT4 (SEQ ID NO: 9). The resulting control strains (EVO067 and EVO068) integrated with the variant OAC genes of SEQ ID NO. 411 and 417, respectively, thus included a pathway of the genes AAE1, OLS, and OAC capable of converting hexanoic acid to OA, as well as PT4 capable of converting OA to CBGA. As in Examples 1 and 2, the screening strain EVO029, was built by integrating the m-Venus cassette at the XI-2 site under control of the bidirectional Gal1/10 promoter and PGK1 terminator, thereby replacing the previous integrated OAC gene. The EVO029 strain was no longer capable of converting hexanoic acid to OA or further to CBGA.

The EVO067 and EVO068 control strains were used to calculate fold-improvement in OA titer in each respective library as described below.

Genomic DNA from the control strains (EVO067 and EVO068) were used as the template to generate two PCR products: (1) a first PCR product (Fragment A), which does not harbor any degenerate codons, and (2) a second PCR product (Fragment B), which has sequence overlap with the Fragment A, and is amplified harboring one NNK degenerate codon only. Primers used for amplification of Fragments A and B and overlap extension were designed according to standard site-saturation mutagenesis protocols. Fragment B was amplified with a single forward primer that included the single NNK degenerate codon at either position 9 or position 49 respectively and a single reverse primer: 5′-CTAAGTCTAGCCACGAAAACTGCAA-3′ (SEQ ID NO: 423). Fragment A was amplified using a single forward primer: 5′-GGTTATGAAGAGGAAAAATTGGCAGTAACC-3′ (SEQ ID NO: 424) and a single primer designed according to the location of the mutagenesis site, in this case position 9 or 49 respectively. The two fragments A and B were assembled by overlap extension PCR using the forward primer: 5′-GAACGAATCAAATTAACAACCATAGGATGA-3′ (SEQ ID NO: 425) and reverse primer: 5′-GCACCAAAAGTAAGAAACGACAAAGTTT-3′ (SEQ ID NO: 426).

The assembled OE-PCR products were then pooled together per template and gel purified to provide two saturation mutagenesis libraries of linear donor DNA.

The two pooled saturation mutagenesis libraries of linear donor DNA were transformed and integrated as a knock-in using CRISPR-Cas9 into an m-Venus cassette located at the XI-2 site in a yeast strain that already had integrated genes encoding the C. sativa enzymes, AAE1, OLS and PT4 (EVO029). The m-Venus cassette was integrated at the XI-2 site under control of the bidirectional Gal1/10 promoter and PGK1 terminator.

### B. Screening of Site Saturation Mutagenesis Libraries for Olivetolic Acid Biosynthesis:

Individual clones from the saturation mutagenesis libraries integrated in EVO029 and the respective EVO067 or EVO068 control strains were grown in 0.3 mL YPD in 96-well microtiter plates and screened as described in Example 1. Whole broth culture samples were extracted and screened using HPLC as described in Example 1.

### C. Sequencing

Clones determined by screening to exhibit an OA titer were re-tested and sequenced using Sanger sequencing technology to determine their nucleotide and amino acid differences compared to SEQ ID NO 5 and SEQ ID NO 6, respectively.

### D. Results

Results for relative OA titer and corresponding amino acid changes of the SSM libraries generated from the parent codon-optimized genes of SEQ ID NO: 411 and 417 is summarized in Tables 14 and 15 respectively (below).

TABLE 14

| | | | aa | |
|---|---|---|---|---|
| NT | AA | | substitution | Rela- |
| SEQ | SEQ | | (s) relative | tive |
| ID | ID | | to SEQ ID | OA |
| NO: | NO: | aa substitution(s) relative to CsOAC | NO: 412 | titer¹ |
| 411 | 412 | A2P, V8I, L9I, E64D, T68S, R100G | n/a | 1 |
| 855 | 856 | A2P, V8I, L9V, E64D, T68S, R100G | L9V | 1.171 |
| 857 | 858 | A2P, V8I, L9T, E64D, T68S, R100G | L9T | 0.774 |
| 859 | 860 | A2P, V8I, L9C, E64D, T68S, R100G | L9C | 1.046 |
| 861 | 862 | A2P, V8I, L9G, E64D, T68S, R100G | L9G | 0.185 |
| 863 | 864 | A2P, V8I, L9A, E64D, T68S, R100G | L9A | 0.916 |
| 865 | 866 | A2P, V8I, L9M, E64D, T68S, R100G | L9M | 0.951 |
| 867 | 868 | A2P, V8I, L9F, E64D, T68S, R100G | L9F | 0.951 |
| 869 | 870 | A2P, V8I, L9S, E64D, T68S, R100G | L9S | 0.191 |

¹The OA titer for the EVO067 control strain integrated with codon optimized OAC gene of SEQ ID NO: 411 was set as 1, and the values for “Relative OA titer” for each library strain clone was determined by comparison to this control strain.

TABLE 15

| | | | aa | |
|---|---|---|---|---|
| NT | AA | | substitution | Rela- |
| SEQ | SEQ | | (s) relative | tive |
| ID | ID | | to SEQ ID | OA |
| NO: | NO: | aa substitution(s) relative to CsOAC | NO: 418 | titer¹ |
| 417 | 418 | V8I, K49R, I58V, E64D, T68M, I74M | n/a | 1 |
| 871 | 872 | V8I, K49G, I58V, E64D, T68M, I74M | K49G | 0.83 |
| 873 | 874 | V8I, K49A, I58V, E64D, T68M, I74M | K49A | 0.74 |
| 875 | 876 | V8I, K49H, I58V, E64D, T68M, I74M | K49H | 1.07 |
| 877 | 878 | V8I, K49C, I58V, E64D, T68M, I74M | K49C | 0.78 |
| 879 | 880 | V8I, K49T, I58V, E64D, T68M, I74M | K49T | 0.76 |
| 881 | 882 | V8I, K49V, I58V, E64D, T68M, I74M | K49V | 0.78 |
| 883 | 884 | V8I, K49S, I58V, E64D, T68M, I74M | K49S | 0.86 |
| 885 | 886 | V8I, K49N, I58V, E64D, T68M, I74M | K49N | 0.75 |
| 887 | 888 | V8I, K49P, I58V, E64D, T68M, I74M | K49P | 0.78 |
| 889 | 890 | V8I, K49L, I58V, E64D, T68M, I74M | K49L | 0.74 |

¹The OA titer for the EVO068 control strain integrated with codon optimized OAC gene of SEQ ID NO: 417 was set as 1, and the values for “Relative OA titer” for each library strain clone was determined by comparison to this control strain.

As shown by the results in Tables 3, 7, 8, 9, 10, 13, 14, and 15, the presence of the following amino acid differences in the recombinant polypeptides having OAC activity expressed in the strains from the various SSM, combinatorial and codon optimization libraries resulted in substantial OA titer produced by the yeast strain: A2G, A2S, A2P, A2V, L6F, V8I, L9A, L9F, L9G, L9I, L9M, L9S, L9V, K10A, F11L, K12L, K12N, K12Q, K12V, E14G, T16P, T16Q, E17G, A18E, A18S, E21L, E21V, E22L, F23I, K25D, K25G, K25E, K25N, K25R, K25S, T26A, T26N, Y27F, V28C, N29D, N29G, V31A, V31E, V31M, V31S, I33D, I33E, I33V, A36E, A36F, A36L, A36Q, A36S, V40A, V40G, Y41E, Y41Q, Y41S, Y41T, K44P, D45V, V46I, V46L, T47A, T47G, T47S, T47S, Q48C, Q48H, Q48M, Q48P, K49A, K49C, K49G, K49H, K49L, K49N, K49P, K49R, K49S, K49T, K49V, N50Y, E52Q, E52R, E52S, E53A, E53F, E53H, E53L, E53R, E53S, E53V, Y55W, T56S, H57G, 158C, 158V, T62C, T62G, E64D, E64K, V66I, V66L, E67S, T68A, T68C, T68E, T68G, T68H, T68M, T68Q, T68S, Q70A, Q70K, D71G, I74G, I74H, I74K, I74L, I74M, I74N, I74Q, I74R, I74S, I74T, I74V, P76V, A77E, H78P, G80K, G82A, G82R, D83K, D83R, V84I, V84M, Y85F, R86S, S87H, S87K, S87P, F88W, F88Y, E90D, K91E, I94K, Y97F, T98V, R100A, and R100G. Additionally, at least the combinations of two, three, four, five, six, seven, or more amino acid residue differences listed for the specific variant polypeptides listed in Tables 7, 8, 9, 10, 13, 14, and 15 when engineered in the gene encoding the OAC polypeptides result in substantial OA titer produced by the yeast strain.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. A recombinant polypeptide having olivetolic acid cyclase activity, wherein the polypeptide comprises an amino acid sequence of at least 80% identity to SEQ ID NO: 6, and an amino acid residue difference as compared to SEQ ID NO: 6 at each of a combination of six positions, wherein the combination of six positions are selected from V8, K49, 158, E64, T68, and I74.

2. The polypeptide of claim 1 in which the amino acid residue difference at each of the combination of six positions is selected from: V8I, K49A, K49C, K49G, K49H, K49L, K49N, K49P, K49R, K49S, K49T, K49V, 158C, 158V, E64D, E64K, T68A, T68C, T68E, T68G, T68H, T68M, T68Q, T68S, I74G, I74H, I74K, I74L, I74M, I74N, I74Q, I74R, I74S, I74T, and I74V.

3. The polypeptide of claim 2, wherein the combination of six amino acid differences selected from the following:
V8I, K49R, I58V, E64D, T68M, I74M
V8I, K49G, I58V, E64D, T68M, I74M
V8I, K49A, I58V, E64D, T68M, I74M
V8I, K49H, I58V, E64D, T68M, I74M
V8I, K49C, I58V, E64D, T68M, I74M
V8I, K49T, I58V, E64D, T68M, I74M
V8I, K49V, I58V, E64D, T68M, I74M
V8I, K49S, I58V, E64D, T68M, I74M
V8I, K49N, I58V, E64D, T68M, I74M
V8I, K49P, I58V, E64D, T68M, I74M
V8I, K49L, I58V, E64D, T68M, I74M.

4. The polypeptide of claim 1 in which the polypeptide comprises an amino acid sequence of at least 90% identity to a sequence selected from the group consisting of even-numbered SEQ ID NOs: 22 to 890.

5. The polypeptide of claim 1 in which the olivetolic acid cyclase activity of the polypeptide as compared to a polypeptide consisting of SEQ ID NO: 20 is at least 0.2-fold.

6. A polynucleotide encoding the polypeptide of claim 1.

7. The polynucleotide of claim 6 in which the polynucleotide sequence comprises:
(a) a sequence of at least 80% identity to a sequence selected from the group consisting of odd-numbered SEQ ID NOs: 21 to 889; or
(b) a codon degenerate sequence of a sequence selected from the group consisting of odd-numbered SEQ ID NOs: 21 to 889.

8. An expression vector comprising the polynucleotide of claim 6.

9. An isolated host cell comprising the polynucleotide of claim 6 or the expression vector of claim 8.

10. An isolated host cell comprising a nucleic acid encoding a recombinant polypeptide having olivetolic acid cyclase activity of claim 1.

11. The host cell of claim 10, wherein the host cell further comprises a pathway of enzymes capable of producing a tetraketide cannabinoid precursor; optionally, wherein the tetraketide cannabinoid precursor is 3,5,7-trioxododecanoyl-CoA.

12. The host cell of claim 10, wherein the cell further comprises a nucleic acid encoding an enzyme capable of catalyzing the conversion of OA to CBGA.

13. The host cell of claim 10, wherein the cell further comprises a nucleic acid encoding an enzyme capable of catalyzing the conversion of CBGA to Δ⁹-THCA, CBDA, and/or CBCA.

14. The host cell of claim 12, wherein the cell produces a cannabinoid selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁹-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

15. The host cell of claim 10, wherein recombinant host cell source is selected from Saccharomyces cerevisiae , Yarrowia lipolytica, Pichia pastoris, and Escherichia coli.

16. A method for producing a cannabinoid or cannabinoid precursor comprising:
(a) culturing in a suitable medium a recombinant host cell of claim 10; and
(b) recovering the produced cannabinoid or cannabinoid precursor.

17. The method of claim 16, wherein the method further comprises contacting a cell-free extract of the culture with a biocatalytic reagent or chemical reagent.

18. A method for preparing a compound of structural formula (I) 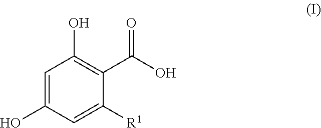
wherein, R¹ is C1-C7 alkyl,
comprising contacting under suitable reactions conditions a compound of structural formula (II) 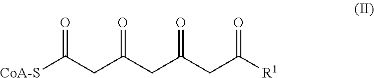
wherein, R¹ is C1-C7 alkyl,
and a recombinant polypeptide of claim 1.

19. The method of claim 18, wherein:
(a) the compound of structure formula (I) is olivetolic acid (OA) and the compound of structural formula (II) is 3,5,7-trioxododecanoyl-CoA; or
(b) the compound of structure formula (I) is divarinic acid (DA) and the compound of structural formula (II) is 3,5,7-trioxodecanoyl-CoA acid.


SEQUENCE LISTING (890 sequences)

SEQ ID NO: 1 - DNA, 2163 bp, synthetic construct
  misc_feature 1..2163: note = Synthetic biopolymer
  source 1..2163: mol_type = other DNA
atgggaaaaaattataagtcacttgacagtgtggttgctagtgatttcatagccttgggtatcacatctgaggtagcggagactcttcatgggcgtttagctgagattgtgtgtaattacggggcggccacccctcagacttggatcaatatagcaaaccacatcttatctcctgatctacccttttctctacaccaaatgctgttctatgggtgttacaaagatttcgggcccgcaccgcccgcatggataccggatccagagaaagtcaaatccaccaacttaggcgcgttgcttgaaaaaagaggcaaggaattcctgggcgtcaagtacaaggaccccatctcatctttctcccacttccaagagttctctgtacgtaatccggaggtttactggcgtaccgtccttatggatgagatgaagatatcattctccaaggacccggaatgtatactaagaagggatgacattaacaacccagggggcagtgagtggcttccgggtggataccttaatagtgcaaaaaactgtcttaacgtgaactcaaacaaaaagttaaacgatacaatgatcgtgtggcgtgatgagggcaatgatgaccttccgctaaataagttgacgcttgatcagctaagaaagcgtgtctggcttgtcggatatgccctagaggagatgggccttgagaagggatgcgcgatcgccatagatatgcctatgcatgtcgatgctgtggttatatatctggccatcgttctagcgggctatgttgttgtgagtattgccgatagcttcagtgcacccgaaatatcaacgagactgcgtttgtccaaagccaaagcaatattcacacaggatcatatcattagaggaaagaagcgtattccgctatattcaagggtggtggaggccaaaagcccgatggctatagtgataccctgttcaggcagcaacatcggagctgaattaagagatggagatatatcctgggactattttttagagagggcgaaagaatttaagaactgcgagttcaccgcaagggaacagccagtggatgcgtatacaaacatactattcagttcaggtactacgggagaaccaaaggctataccctggacgcaggcaaccccgctaaaagctgccgctgacgggtggtctcatttggatatcaggaagggtgatgttatcgtatggccaacaaatcttggatggatgatgggaccgtggcttgtgtacgctagcttgttaaatggagcttcaatcgctctatataatggttcccctcttgtcagcggttttgcgaagttcgtacaggatgccaaggtaactatgctgggagtcgttcctagcatcgtcaggagttggaaatcaactaactgtgtgtctggttacgactggtccactatcagatgttttagctccagtggtgaggcctccaacgtagatgagtacctttggcttatgggacgtgccaactataaaccggtaatagagatgtgtgggggtactgaaataggaggagcgttttcagctggcagttttcttcaggctcaaagcttgtctagtttctcttcacagtgtatgggctgtacgttatacattcttgataagaacggttatccaatgcccaaaaacaaaccaggtataggagaattggcgctgggtccagtcatgtttggcgctagtaaaactctactgaacggaaatcatcacgacgtttactttaaaggtatgcctactcttaatggagaagtacttaggcgtcatggcgatatctttgagttgacatccaatggctactatcacgcccatggcagggcggatgacaccatgaacatcggggggatcaaaatctccagtatagagatcgagagagtgtgtaacgaggtagacgaccgtgtattcgagacaacagccattggggttccacccctaggtggtggccccgaacaacttgttatcttttttgtgctgaaggactcaaatgacactaccattgatttaaatcaactacgtttgtcattcaatctgggattacaaaagaagttgaatcctttattcaaggtcacaagagtagtaccccttagttccctgccaagaactgcgacaaacaagataatgcgtagagtgctaaggcagcagtttagtcattttgagtaa

SEQ ID NO: 2 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 3 - DNA, 1158 bp, synthetic construct
  misc_feature 1..1158: note = Synthetic biopolymer
  source 1..1158: mol_type = other DNA
atgaaccacttgagagcagaagggccggccagtgtactggctatagggacagccaaccccgaaaatatactgttgcaagatgagttcccagattattactttagagtgactaaatccgagcacatgacgcaacttaaggagaaattcaggaaaatatgcgacaaatctatgattagaaaaagaaattgtttcttaaatgaggagcatctaaagcagaatccccgtctggtagaacatgaaatgcaaactttggacgcgcgtcaagacatgctagttgtcgaagtgcctaaattaggcaaagacgcgtgtgcaaaggctataaaagagtggggccaaccgaagtccaaaattacacacctaatattcacttctgcgtccaccaccgacatgcccggagccgactaccactgtgcgaaacttctaggcctatccccttcagtcaagcgtgtaatgatgtatcaactggggtgctacggaggaggcaccgttttgaggattgcaaaggatatcgctgaaaataacaagggggctcgtgtacttgctgtgtgctgtgatatcatggcctgccttttcagaggcccctcagagtcagatcttgaactgttagtaggtcaggctatcttcggagatggcgctgcagccgtcatagttggggcggagcctgacgaatcagttggggagaggcccattttcgagctggtcagtacgggacagaccatcttgccaaatagcgagggcacgatcggaggccacataagggaggcgggtttgatatttgaccttcataaggatgtaccgatgttgatctccaataatattgagaagtgtcttattgaagcatttacccctattggtatttcagactggaacagtatcttctggattacgcatccgggaggtaaggcgattcttgataaagtcgaagaaaagctacacctgaagtcagacaagttcgttgactccagacacgttctttcagagcacggcaacatgagttcttccaccgtccttttcgtaatggacgagctgaggaaacgtagccttgaggaaggtaaaagtacgacaggagatgggtttgagtggggagtgttgtttggcttcggcccagggttaacagttgaacgtgtagtcgttagatctgtccctattaaatactaa

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 5 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 6 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 7 - DNA, 1517 bp, Cannabis sativa
  source 1..1517: mol_type = genomic DNA
atcaataataatcttcatgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataagaccataatataacatatatatgtttattacataaaattgggacacaaaaacgtcaattatttggacaaaagtactcagaaagacctctttcactacaaggggaggccatttagttatacttgggtttcaatcaacaaatttataaatttttaagattttatttacaaaacattttcatgtgtaattaaatcgatcgtcatttattttttggatacaacttggttcaacttattttaattagagtgcttcgtaatttaactacaattatagaagggcattttataaaaatactggatttggggt

SEQ ID NO: 8 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 9 - DNA, 954 bp, synthetic construct
  misc_feature 1..954: note = Synthetic biopolymer
  source 1..954: mol_type = other DNA
atgatcgaaggttcacctcatcatgaaagtgataacagcatagccacgaagattttgaatttcggccatacttgttggaagctacagaggccgtacgtcgttaaggggatgatttccattgcgtgcggtctgtttggcagggaattatttaacaacagacacttattcagttggggcctgatgtggaaggccttcttcgctcttgtacccattctgtccttcaacttttttgcagcgatcatgaatcaaatatacgatgtagacatcgatagaataaacaagcccgatttacctctggtatcaggcgaaatgagcatcgaaactgcgtggattttatcaatcatcgttgcattgactgggctgatagtgaccataaagttaaagtcagccccgttgtttgtcttcatatacatcttcggcattttcgcgggctttgcgtatagtgtacctcccattagatggaagcagtacccgtttactaactttcttattacaattagcagccatgtcggtcttgcattcacgtcctactcagccaccacatccgcactggggctaccgtttgtgtggcgtccagccttcagcttcatcatcgcattcatgacagtaatgggtatgacgatagcttttgcaaaggatataagtgatatcgagggtgacgctaagtatggagtgtctactgtggccacgaagctgggggcccgtaatatgactttcgtggtatcaggtgtactattgcttaattaccttgtttctatatcaatcggaattatttggccacaagttttcaaatccaatataatgatcctatcacacgctattttagcgttttgtttgatatttcagactagagagcttgcactagcgaattacgcgagtgccccgagtaggcagtttttcgagttcatatggctattatactatgctgagtactttgtttacgtatttatttaa

SEQ ID NO: 10 - protein, 317 aa, synthetic construct
  REGION 1..317: note = Synthetic biopolymer
MIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - DNA, 1635 bp, Cannabis sativa
  source 1..1635: mol_type = genomic DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 12 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 13 - DNA, 1554 bp, synthetic construct
  misc_feature 1..1554: note = Synthetic biopolymer
  source 1..1554: mol_type = other DNA
atgaacccccgtgaaaattttttgaaatgtttctctcaatacatacccaacaatgcaaccaacttaaagctggtatatactcaaaacaaccccctatatatgtctgttctaaatagtactatccataacttacgtttcacctcagataccacccctaaaccgctggtcatcgtgactccgtctcatgtttcacacatacagggcacgatattgtgctcaaaaaaggtcgggttacagattcgtacccgttcaggaggtcatgatagtgagggaatgtcttacatctcccaggtcccttttgtaattgtcgaccttcgtaatatgagatccataaagatcgacgttcattcacagacggcgtgggtagaggctggtgcaaccctaggtgaagtctactactgggtcaacgaaaaaaacgagaacttatcattagctgcggggtattgccctacagtttgtgccggaggtcattttggaggtggaggctacgggccactgatgaggaactacggtctggcagcagacaacattatagatgcacacctagtgaacgtgcatggtaaagttttagatagaaagtccatgggagaagatttgttttgggcactacgtggaggaggggctgagtcattcgggattattgtagcgtggaagatccgtctggtcgcagtccctaaatctacgatgttttccgtgaaaaagattatggaaattcacgagctagtgaaacttgtcaataagtggcagaatatagcatacaaatatgacaaggatctattgttgatgacgcatttcatcacaagaaacattacggacaatcaaggtaagaataagacggctattcacacttacttcagctccgtttttctaggaggggtagattccctagttgacctgatgaataagagttttcccgagttgggtattaaaaaaactgattgtagacagctgtcttggatcgacacaatcatattctactctggtgtggtaaactatgacaccgataatttcaacaaagaaatcttactggatagatcagccggtcaaaacggcgcgtttaaaatcaagctggattacgtaaagaagcctatacccgaatccgtatttgtacagattctggaaaagttatacgaggaagacattggggcgggtatgtacgctctttacccttacggcgggatcatggatgagatttccgaaagtgctatcccgttccctcatcgtgctggcattctgtacgagttatggtatatttgcagttgggagaagcaggaggataatgaaaagcacctaaattggattcgtaatatttataatttcatgactccctatgttagtaagaaccccagactggcctaccttaattatagagacctggacatcgggataaatgatccgaagaacccaaataactatacgcaggccaggatttggggggaaaagtatttcggaaagaactttgacagactggtgaaagttaagaccctggtggatccaaataattttttcaggaacgagcagagtattcccccgcttccacgtcacaggcattaa

SEQ ID NO: 14 - protein, 517 aa, synthetic construct
  REGION 1..517: note = Synthetic biopolymer
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - DNA, 1638 bp, Cannabis sativa
  source 1..1638: mol_type = genomic DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 16 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 17 - DNA, 1557 bp, synthetic construct
  misc_feature 1..1557: note = Synthetic biopolymer
  source 1..1557: mol_type = other DNA
atgaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 18 - protein, 518 aa, synthetic construct
  REGION 1..518: note = Synthetic biopolymer
MNPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 19 - DNA, 303 bp, synthetic construct
  misc_feature 1..303: note = Synthetic biopolymer
  source 1..303: mol_type = other DNA
atggccgtcaagcatttaatcgtcttaaaatttaaggacgaaatcacagaagctcaaaaggaggaattcttcaaaacctatgtaaacttagtgaacataattcctgctatgaaagacgtttattggggtaaggatgttacgcaaaaaaataaggaagaaggatacacccacattgtagaagtcacttttgaatcagtggaaaccatacaagattatatcattcaccctgcacatgttgggttcggtgatgtctacagatcattttgggaaaagttattgatctttgattacacacctagaaaa

SEQ ID NO: 20 - protein, 101 aa, Cannabis sativa; identical to SEQ ID NO: 6

SEQ ID NO: 21 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattcaggaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 22 - protein, 101 aa, synthetic construct
  REGION 1..101: note = Synthetic biopolymer
MAVKHLIVLKFKDEIQEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 23 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacgtggcccatgtgggattcggagacgtttaccgtagcttctgggaaaaacttttgattttcgactataccccgagaaaa

SEQ ID NO: 24 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHVAHVGFGDVYRSFWEKLLIFDYTPRK

SEQ ID NO: 25 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 26 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 27 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcccggacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 28 - protein, 101 aa, synthetic construct
  REGION 1..101: note = Synthetic biopolymer
MAVKHLIVLKFKDEITEAQKEEFFKTYVNLVNIIPAMKDVYWGPDVTQKNKEEGYTHIVEVTFESVETIQDYIIHPAHVGFGDVYRSFWEKLLIFDYTPRK

SEQ ID NO: 29 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacatatctcacccggcccatgtgggattcggagacgtttaccgtagcttctgggaaaaacttttgattttcgactataccccgagaaaa

SEQ ID NO: 30 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYISHPAHVGFGDVYRSFWEKLLIFDYTPRK

SEQ ID NO: 31 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactggtatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 32 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTGIVEVTFESVETIQDYIIHPAHVGFGDVYRSFWEKLLIFDYTPRK

SEQ ID NO: 33 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 34 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFESVETIQDYIIHPAHVGFGDVYRSFWEKLLIFDYTPRK

SEQ ID NO: 35 - DNA, 303 bp, synthetic construct
  misc_feature 1..303: note = Synthetic biopolymer
  source 1..303: mol_type = other DNA
atggctgtcaagcaccttatcgtactgaaattcaaggacgggattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 36 - protein, 101 aa, synthetic construct
  REGION 1..101: note = Synthetic biopolymer
MAVKHLIVLKFKDGITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 37 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagggtacataatacacccggcccatgtgggattcggagacgtttaccgtagcttctgggaaaaacttttgattttcgactataccccgagaaaa

SEQ ID NO: 38 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQGYIIHPAHVGFGDVYRSFWEKLLIFDYTPRK

SEQ ID NO: 39 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaagagcttttgattttcgactataccccgagaaaa

SEQ ID NO: 40 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPAHVGFGDVYRSFWEELLIFDYTPRK

SEQ ID NO: 41 - DNA, 303 bp, synthetic construct
  misc_feature 1..303: note = Synthetic biopolymer
  source 1..303: mol_type = other DNA
atggctgtcaagcaccttatcgtagtgaaattcaaggacgaaattacagaagcccagaaagttgaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 42 - protein, 101 aa, synthetic construct
  REGION 1..101: note = Synthetic biopolymer
MAVKHLIVVKFKDEITEAQKVEFFKTYVNLVNIIPAMKDVYWGKDVTQKNKEEGYTHIVEVTFESVETIQDYIIHPAHVGFGDVYRSFWEKLLIFDYTPRK

SEQ ID NO: 43 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagaattatgtaaacctggtgaatatcatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 44 - protein, 101 aa, synthetic construct
  REGION 1..101: note = Synthetic biopolymer
MAVKHLIVLKFKDEITEAQKEEFFKNYVNLVNIIPAMKDVYWGKDVTQRNKEEGYTHIVEVTFESVETIQGYIIHPAHVGFGDVYRSFWEELLIFDYTPRK

SEQ ID NO: 45 - DNA, 303 bp, synthetic construct
  misc_feature 1..303: note = Synthetic biopolymer
  source 1..303: mol_type = other DNA
atggctgtcaagcaccttatcgtactgaaattcttggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 46 - protein, 101 aa, synthetic construct
  REGION 1..101: note = Synthetic biopolymer
MAVKHLIVLKFLDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 47 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaatcgcagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 48 - protein, 101 aa, synthetic construct
  REGION 1..101: note = Synthetic biopolymer
MAVKHLIVLKFKDEITESQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 49 - DNA, 303 bp, synthetic construct
  misc_feature 1..303: note = Synthetic biopolymer
  source 1..303: mol_type = other DNA
atggctgtaaagcaccttatcgtagtcaaattcaaggacggcattcaggaagcccagaaagaagaatttttcaagacctatgtaaacctggtgaatatcatccctgcgatgaaagatgggtattggggcaaagacgtcacccaacgtaacaaagaagaaggttacactcacatcgtcgaagtcactttcgagtcagtagagacgatccaaggctacataatgcacccggcccatgtgggattcggagacgtttaccgtagcttctgggaagaacttttgattttcgactataccccgagaaaa

SEQ ID NO: 50 - protein, 101 aa, synthetic construct
  REGION 1..101: note = Synthetic biopolymer
MAVKHLIVVKFKDGIQEAQKEEFFKTYVNLVNIIPAMKDGYWGKDVTQRNKEEGYTHIVEVTFESVETIQGYIMHPAHVGFGDVYRSFWEELLIFDYTPRK

SEQ ID NO: 51 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacaggagcccagaaagaagaatttttcaagacctatgtaaacctggtgaatatcatccctgcgatgaaagatgtttattggggcccggacgtcacccaaaaaaacaaagaagaaggttacacccacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 52 - protein, 101 aa, synthetic construct
  REGION 1..101: note = Synthetic biopolymer
MAVKHLIVLKFKDEITGAQKEEFFKTYVNLVNIIPAMKDVYWGPDVTQKNKEEGYTHIVEVTFESVETIQDYIIHPAHVGFGDVYRSFWEKLLIFDYTPRK

SEQ ID NO: 53 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgaagttcgactataccccgagaaaa

SEQ ID NO: 54 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLKFDYTPRK

SEQ ID NO: 55 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcaagtcagtagagacgatccaggattacataatacacccggcccatgtgggattcggagacgtttaccgtagcttctgggaaaaacttttgattttcgactataccccgagaaaa

SEQ ID NO: 56 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFKSVETIQDYIIHPAHVGFGDVYRSFWEKLLIFDYTPRK

SEQ ID NO: 57 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatgagatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 58 - protein, 101 aa, synthetic construct
  REGION 1..101: note = Synthetic biopolymer
MAVKHLIVLKFKDEITEAQKEEFFKTYVNLVNEIPAMKDVYWGKDVTQRNKEEGYTHIVEVTFESVETIQGYIIHPAHVGFGDVYRSFWEELLIFDYTPRK

SEQ ID NO: 59 - DNA, 303 bp, synthetic construct
  misc_feature 1..303: note = Synthetic biopolymer
  source 1..303: mol_type = other DNA
atggctgtcaagcaccttatcgtagtgaaattcaaggacgaaattacagaagcccagaaagttgaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcgctgacgtttaccgtagcttctgggaaaaacttttgattttcgactataccccgagaaaa

SEQ ID NO: 60 - protein, 101 aa, synthetic construct
  REGION 1..101: note = Synthetic biopolymer
MAVKHLIVVKFKDEITEAQKVEFFKTYVNLVNIIPAMKDVYWGKDVTQKNKEEGYTHIVEVTFESVETIQDYIIHPAHVGFADVYRSFWEKLLIFDYTPRK

SEQ ID NO: 61 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggagcatgtgggattcggagacgtttaccgtagcttctgggaaaaacttttgattttcgactataccccgagaaaa

SEQ ID NO: 62 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPEHVGFGDVYRSFWEKLLIFDYTPRK

SEQ ID NO: 63 - DNA, 303 bp, synthetic construct
  misc_feature 1..303: note = Synthetic biopolymer
  source 1..303: mol_type = other DNA
atggctgtcaagcaccttatcgtagtcaaattcaaggacggcattcaggaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 64 - protein, 101 aa, synthetic construct
  REGION 1..101: note = Synthetic biopolymer
MAVKHLIVVKFKDGIQEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 65 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcgggttcgagtcagtagagacgatccaagattacataatacacccggcccatgtgggattcggagacgtttaccgtagcttctgggaaaaacttttgattttcgactataccccgagaaaa

SEQ ID NO: 66 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVGFESVETIQDYIIHPAHVGFGDVYRSFWEKLLIFDYTPRK

SEQ ID NO: 67 - DNA, 303 bp, synthetic construct
  misc_feature 1..303: note = Synthetic biopolymer
  source 1..303: mol_type = other DNA
atggctgtcaagcaccttatcgtactgaaattgaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 68 - protein, 101 aa, synthetic construct
  REGION 1..101: note = Synthetic biopolymer
MAVKHLIVLKLKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 69 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtaagttctgggaaaaacttttgattttcgactataccccgagaaaa

SEQ ID NO: 70 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPAHVGFGDVYRKFWEKLLIFDYTPRK

SEQ ID NO: 71 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggacgggtttaccgtagcttctgggaaaaacttttgattttcgactataccccgagaaaa

SEQ ID NO: 72 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPAHVGFGRVYRSFWEKLLIFDYTPRK

SEQ ID NO: 73 - DNA, 303 bp, synthetic construct
  misc_feature 1..303: note = Synthetic biopolymer
  source 1..303: mol_type = other DNA
atggctgtcaagcaccttatcgtagtcaaattcaaggacggcattcaggaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacatgtaccgtagcttctgggaagaacttttgattttcgactataccccgagaaaa

SEQ ID NO: 74 - protein, 101 aa, synthetic construct
  REGION 1..101: note = Synthetic biopolymer
MAVKHLIVVKFKDGIQEAQKEEFFKTYVNLVNIIPAMKDVYWGKDVTQRNKEEGYTHIVEVTFESVETIQGYIIHPAHVGFGDMYRSFWEELLIFDYTPRK

SEQ ID NO: 75 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattccgggacgtttaccgtagcttctgggaaaaacttttgattttcgactataccccgagaaaa

SEQ ID NO: 76 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESVETIQDYIIHPAHVGFRDVYRSFWEKLLIFDYTPRK

SEQ ID NO: 77 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttccggacctatgtaaacctggtgaatatcatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 78 - protein, 101 aa, synthetic construct
  REGION 1..101: note = Synthetic biopolymer
MAVKHLIVLKFKDEITEAQKEEFFRTYVNLVNIIPAMKDVYWGKDVTQRNKEEGYTHIVEVTFESVETIQGYIIHPAHVGFGDVYRSFWEELLIFDYTPRK

SEQ ID NO: 79 - DNA, 303 bp, synthetic construct
  misc_feature 1..303: note = Synthetic biopolymer
  source 1..303: mol_type = other DNA
atggctgtaaagcaccttatcgtagtcaaattcaaggacggcattcaggaagcccagaaagaagaatttttcaagacctatgtaaacctggtgaatatcatccctgagatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 80 - protein, 101 aa, synthetic construct
  REGION 1..101: note = Synthetic biopolymer
MAVKHLIVVKFKDGIQEAQKEEFFKTYVNLVNIIPEMKDVYWGKDVTQRNKEEGYTHIVEVTFESVETIQGYIIHPAHVGFGDVYRSFWEELLIFDYTPRK

SEQ ID NO: 81 - DNA, 303 bp, synthetic construct
  misc_feature 1..303: note = Synthetic biopolymer
  source 1..303: mol_type = other DNA
atggctgtcaagcaccttatcgtagtgaaattcaaggacgaaattccagaagcccagaaagaagaatttttcaaggcttatgtaaacctggtgaatatcatccctgcgatgaaagatgtttattggggcaaagacgtcacccaacgtaacaaagaagaaggttacactcacatcgtcgaagtcactttcgagtcagtagagacgatccaaggctacataatacacccggcccatgtgggattcggagacgtttaccgtagcttctgggaagaacttttgattttcgactataccccgagaaaa

SEQ ID NO: 82 - protein, 101 aa, synthetic construct
  REGION 1..101: note = Synthetic biopolymer
MAVKHLIVVKFKDEIPEAQKEEFFKAYVNLVNIIPAMKDVYWGKDVTQRNKEEGYTHIVEVTFESVETIQGYIIHPAHVGFGDVYRSFWEELLIFDYTPRK

SEQ ID NO: 83 - DNA, 303 bp, synthetic construct; identical to SEQ ID NO: 63
  misc_feature 1..303: note = Synthetic biopolymer
  source 1..303: mol_type = other DNA

SEQ ID NO: 84 - protein, 101 aa, synthetic construct; identical to SEQ ID NO: 64
  REGION 1..101: note = Synthetic biopolymer

SEQ ID NO: 85 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 86 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 87 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacaccggtattgtagaagtcacttttgaatcagtggaaaccatacaaggttatatcattcaccctgcacatgttgggttcggtgatgtctacagatcattttgggaagaattattgatctttgattacacacctagaaaa

SEQ ID NO: 88 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTGIVEVTFESVETIQGYIIHPAHVGFGDVYRSFWEELLIFDYTPRK

SEQ ID NO: 89 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttctcgacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 90 - protein, 101 aa, synthetic construct
  REGION 1..101: note = Synthetic biopolymer
MAVKHLIVLKFKDEITEAQKEEFFSTYVNLVNIIPAMKDVYWGKDVTQRNKEEGYTHIVEVTFESVETIQGYIIHPAHVGFGDVYRSFWEELLIFDYTPRK

SEQ ID NO: 91 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatccaagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacacacctagaaaa

SEQ ID NO: 92 - protein, 101 aa, synthetic construct; identical to SEQ ID NO: 64
  REGION 1..101: note = Synthetic biopolymer

SEQ ID NO: 93 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattttactccaaggaag

SEQ ID NO: 94 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGDVYRSFWEELLIFDFTPRK

SEQ ID NO: 95 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatggacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 96 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGWTHIVEVTFESVETIQGYIIHPAHVGFGDVYRSFWEELLIFDYTPRK

SEQ ID NO: 97 - DNA, 303 bp, synthetic construct
  misc_feature 1..303: note = Synthetic biopolymer
  source 1..303: mol_type = other DNA
atggccgtcaagcatttaatcgtcatcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 98 - protein, 101 aa, synthetic construct
  REGION 1..101: note = Synthetic biopolymer
MAVKHLIVIKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 99 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcactggaaaccatacaaggttatatcattcaccctgcacatgttgggttcggtgatgtctacagatcattttgggaagaattattgatctttgattacactccaaggaag

SEQ ID NO: 100 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESLETIQGYIIHPAHVGFGDVYRSFWEELLIFDYTPRK

SEQ ID NO: 101 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttatcgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 102 - protein, 101 aa, synthetic construct
  REGION 1..101: note = Synthetic biopolymer
MAVKHLIVVKFKDGITEAQKEEFFKTYVNLSNIIPAMKDVYWGKDVTQRNKEEGYTHIVEVTFESVETIQGYIIHPAHVGFGDVYRSFWEELLIFDYTPRK

SEQ ID NO: 103 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttaatgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 104 - protein, 101 aa, synthetic construct
  REGION 1..101: note = Synthetic biopolymer
MAVKHLIVVKFKDGITEAQKEEFFKTYVNLMNIIPAMKDVYWGKDVTQRNKEEGYTHIVEVTFESVETIQGYIIHPAHVGFGDVYRSFWEELLIFDYTPRK

SEQ ID NO: 105 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagagaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 106 - protein, 101 aa, synthetic construct
  REGION 1..101: note = Synthetic biopolymer
MAVKHLIVVKFKDGITEAQKEEFFKTYVNLENIIPAMKDVYWGKDVTQRNKEEGYTHIVEVTFESVETIQGYIIHPAHVGFGDVYRSFWEELLIFDYTPRK

SEQ ID NO: 107 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctattgtaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 108 - protein, 101 aa, synthetic construct
  REGION 1..101: note = Synthetic biopolymer
MAVKHLIVVKFKDGITEAQKEEFFKTYCNLVNIIPAMKDVYWGKDVTQRNKEEGYTHIVEVTFESVETIQGYIIHPAHVGFGDVYRSFWEELLIFDYTPRK

SEQ ID NO: 109 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaagtatacaaggttatatcatacacccggcccatgtgggattcggtgatgtctacagatcattttgggaagaattattgatctttgattacactccaaggaag

SEQ ID NO: 110 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVESIQGYIIHPAHVGFGDVYRSFWEELLIFDYTPRK

SEQ ID NO: 111 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaacagatacaaggttatatcattcaccctgcacatgttgggttcggtgatgtctacagatcattttgggaagaattattgatctttgattacactccaaggaag

SEQ ID NO: 112 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVEQIQGYIIHPAHVGFGDVYRSFWEELLIFDYTPRK

SEQ ID NO: 113 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaatgatacaaggttatatcattcaccctgcacatgttgggttcggtgatgtctacagatcattttgggaagaattattgatctttgattacactccaaggaag

SEQ ID NO: 114 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVEMIQGYIIHPAHVGFGDVYRSFWEELLIFDYTPRK

SEQ ID NO: 115 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaggtatacaaggttatatcattcaccctgcacatgttgggttcggtgatgtctacagatcattttgggaagaattattgatctttgattacactccaaggaag

SEQ ID NO: 116 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVEGIQGYIIHPAHVGFGDVYRSFWEELLIFDYTPRK

SEQ ID NO: 117 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaagagatacaaggttatatcattcaccctgcacatgttgggttcggtgatgtctacagatcattttgggaagaattattgatctttgattacactccaaggaag

SEQ ID NO: 118 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVEEIQGYIIHPAHVGFGDVYRSFWEELLIFDYTPRK

SEQ ID NO: 119 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaagcgatacaaggttatatcattcaccctgcacatgttgggttcggtgatgtctacagatcattttgggaagaattattgatctttgattacactccaaggaag

SEQ ID NO: 120 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVEAIQGYIIHPAHVGFGDVYRSFWEELLIFDYTPRK

SEQ ID NO: 121 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtagggttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 122 - protein, 101 aa, synthetic construct
  REGION 1..101: note = Synthetic biopolymer
MAVKHLIVVKFKDGITEAQKEEFFKTYVGLVNIIPAMKDVYWGKDVTQRNKEEGYTHIVEVTFESVETIQGYIIHPAHVGFGDVYRSFWEELLIFDYTPRK

SEQ ID NO: 123 - DNA, 303 bp, synthetic construct
  misc_feature 1..303: note = Synthetic biopolymer
  source 1..303: mol_type = other DNA
atggccgtcaagcattttattgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 124 - protein, 101 aa, synthetic construct
  REGION 1..101: note = Synthetic biopolymer
MAVKHFIVVKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 125 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcgggacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcaccggttgggttcggtgatgtctacagatcattttgggaagaattattgatctttgattacactccaaggaag

SEQ ID NO: 126 - protein, 101 aa, synthetic construct
  REGION 1..101: note = Synthetic biopolymer
MAVKHLIVVKFKDGITEAQKEEFFGTYVNLVNIIPAMKDVYWGKDVTQRNKEEGYTHIVEVTFESVETIQGYIIHPAPVGFGDVYRSFWEELLIFDYTPRK

SEQ ID NO: 127 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcgtgcaccctgcacatgttgggttcggtgatgtctacagatcattttgggaagaattattgatctttgattacactccaaggaag

SEQ ID NO: 128 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIVHPAHVGFGDVYRSFWEELLIFDYTPRK

SEQ ID NO: 129 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcacgcaccctgcacatgttgggttcggtgatgtctacagatcattttgggaagaattattgatctttgattacactccaaggaag

SEQ ID NO: 130 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYITHPAHVGFGDVYRSFWEELLIFDYTPRK

SEQ ID NO: 131 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcatgcaccctgcacatgttgggttcggtgatgtctacagatcattttgggaagaattattgatctttgattacactccaaggaag

SEQ ID NO: 132 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIMHPAHVGFGDVYRSFWEELLIFDYTPRK

SEQ ID NO: 133 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcttgcaccctgcacatgttgggttcggtgatgtctacagatcattttgggaagaattattgatctttgattacactccacggaag

SEQ ID NO: 134 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYILHPAHVGFGDVYRSFWEELLIFDYTPRK

SEQ ID NO: 135 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcgggcaccctgcacatgttgggttcggtgatgtctacagatcattttgggaagaattattgatctttgattacactccaaggaag

SEQ ID NO: 136 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIGHPAHVGFGDVYRSFWEELLIFDYTPRK

SEQ ID NO: 137 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacgtgattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 138 - protein, 101 aa, synthetic construct
  REGION 1..101: note = Synthetic biopolymer
MAVKHLIVVKFKDGITEAQKEEFFKTYVNLVNVIPAMKDVYWGKDVTQRNKEEGYTHIVEVTFESVETIQGYIIHPAHVGFGDVYRSFWEELLIFDYTPRK

SEQ ID NO: 139 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacgatattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 140 - protein, 101 aa, synthetic construct
  REGION 1..101: note = Synthetic biopolymer
MAVKHLIVVKFKDGITEAQKEEFFKTYVNLVNDIPAMKDVYWGKDVTQRNKEEGYTHIVEVTFESVETIQGYIIHPAHVGFGDVYRSFWEELLIFDYTPRK

SEQ ID NO: 141 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgattcagtggaaaccatacaaggttatatcattcaccctgcacatgttgggttcggtgatgtctacagatcattttgggaagaattattgatctttgattacactccaaggaag

SEQ ID NO: 142 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFDSVETIQGYIIHPAHVGFGDVYRSFWEELLIFDYTPRK

SEQ ID NO: 143 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 144 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIIHPAHVGFGDVYRSFWEELLIFDYTPRK

SEQ ID NO: 145 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaacggggatacacccacattgtagaagtcacttttgaatcagtggaaaccatacaaggttatatcatacaccctgcacatgttgggttcggtgatatctacagatcattttgggaagaattattgatctttgattacactccaaggaag

SEQ ID NO: 146 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKERGYTHIVEVTFESVETIQGYIIHPAHVGFGDIYRSFWEELLIFDYTPRK

SEQ ID NO: 147 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaacgtggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 148 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKERGYTHIVEVTFESVETIQGYIIHPAHVGFGDVYRSFWEELLIFDYTPRK

SEQ ID NO: 149 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaactgggatacacccacattgtagaagtcacttttgaatcagtggaaaccatacaaggttatatcatacacccggcccatgtgggattcggtgatgtctacagatcattttgggaagaattattgatctttgattacactccaaggaag

SEQ ID NO: 150 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKELGYTHIVEVTFESVETIQGYIIHPAHVGFGDVYRSFWEELLIFDYTPRK

SEQ ID NO: 151 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaacatggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 152 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEHGYTHIVEVTFESVETIQGYIIHPAHVGFGDVYRSFWEELLIFDYTPRK

SEQ ID NO: 153 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatttggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 154 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEFGYTHIVEVTFESVETIQGYIIHPAHVGFGDVYRSFWEELLIFDYTPRK

SEQ ID NO: 155 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagcgggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 156 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEAGYTHIVEVTFESVETIQGYIIHPAHVGFGDVYRSFWEELLIFDYTPRK

SEQ ID NO: 157 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataagagggaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 158 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKREGYTHIVEVTFESVETIQGYIIHPAHVGFGDVYRSFWEELLIFDYTPRK

SEQ ID NO: 159 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataagcaggaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 160 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKQEGYTHIVEVTFESVETIQGYIIHPAHVGFGDVYRSFWEELLIFDYTPRK

SEQ ID NO: 161 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggtggttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 162 - protein, 101 aa, synthetic construct
  REGION 1..101: note = Synthetic biopolymer
MAVKHLIVVKFKDGITEAQKEEFFKTYVNLVNIIPAMKDVYWGKVVTQRNKEEGYTHIVEVTFESVETIQGYIIHPAHVGFGDVYRSFWEELLIFDYTPRK

SEQ ID NO: 163 - DNA, 303 bp, synthetic construct
  misc_feature 1..303: note = Synthetic biopolymer
  source 1..303: mol_type = other DNA
atgggcgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcaatcaccctgcacatgttgggttcggtgatgtctacagatcattttgggaagaattattgatctttgattacactccaaggaag

SEQ ID NO: 164 - protein, 101 aa, synthetic construct
  REGION 1..101: note = Synthetic biopolymer
MGVKHLIVVKFKDGITEAQKEEFFKTYVNLVNIIPAMKDVYWGKDVTQRNKEEGYTHIVEVTFESVETIQGYINHPAHVGFGDVYRSFWEELLIFDYTPRK

SEQ ID NO: 165 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcattcaccctgcacatgttaagttcggtgatgtctacagatcattttgggaagaattattgatctttgattacactccaaggaag

SEQ ID NO: 166 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIIHPAHVKFGDVYRSFWEELLIFDYTPRK

SEQ ID NO: 167 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaacatatacaaggttatatcatacaccctgcacatgttgggttcggtgatgtctacagatcattttgggaagaattattgatctttgattacactccaaggaag

SEQ ID NO: 168 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVEHIQGYIIHPAHVGFGDVYRSFWEELLIFDYTPRK

SEQ ID NO: 169 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatctctcaccctgcacatgttgggttcggtgatgtctacagatcattttgggaagaattattgatctttgattacactccaaggaag

SEQ ID NO: 170 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYISHPAHVGFGDVYRSFWEELLIFDYTPRK

SEQ ID NO: 171 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcgatacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 172 - protein, 101 aa, synthetic construct
  REGION 1..101: note = Synthetic biopolymer
MAVKHLIVVKFKDGITEAQKEEFFDTYVNLVNIIPAMKDVYWGKDVTQRNKESGYTHIVEVTFESVETIQGYIIHPAHVGFGDVYRSFWEELLIFDYTPRK

SEQ ID NO: 173 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcatacaccctgcacatgttgggttcggtgatgtctacagacatttttgggaagaattattgatctttgattacactccaaggaag

SEQ ID NO: 174 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIIHPAHVGFGDVYRHFWEELLIFDYTPRK

SEQ ID NO: 175 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaatgtatacaaggttatatcatacaccctgcacatgttgggttcggtgatgtctacagatcattttgggaagaattattgatctttgattacactccaaggaag

SEQ ID NO: 176 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVECIQGYIIHPAHVGFGDVYRSFWEELLIFDYTPRK

SEQ ID NO: 177 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcatacaccctgcacatgttgggttcggtgatgtctacagatcatattgggaagaattattgatctttgattacactccaaggaag

SEQ ID NO: 178 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIIHPAHVGFGDVYRSYWEELLIFDYTPRK

SEQ ID NO: 179 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcaagcaccctgcacatgttgggttcggtgatgtctacagatcattttgggaagaattattgatctttgattacactccaaggaag

SEQ ID NO: 180 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIKHPAHVGFGDVYRSFWEELLIFDYTPRK

SEQ ID NO: 181 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgttcagtggggtaaggatgttacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 182 - protein, 101 aa, synthetic construct
  REGION 1..101: note = Synthetic biopolymer
MAVKHLIVVKFKDGITEAQKEEFFKTYVNLVNIIPAMKDVQWGKDVTQRNKESGYTHIVEVTFESVETIQGYIIHPAHVGFGDVYRSFWEELLIFDYTPRK

SEQ ID NO: 183 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgttacgtggggtaaggatgttacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 184 - protein, 101 aa, synthetic construct
  REGION 1..101: note = Synthetic biopolymer
MAVKHLIVVKFKDGITEAQKEEFFKTYVNLVNIIPAMKDVTWGKDVTQRNKESGYTHIVEVTFESVETIQGYIIHPAHVGFGDVYRSFWEELLIFDYTPRK

SEQ ID NO: 185 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaagggatacaaggttatatcatacaccctgcacatgttgggttcggtgatgtctacagatcattttgggaagaattattgatctttgattacactccaaggaag

SEQ ID NO: 186 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVEGIQGYIIHPAHVGFGDVYRSFWEELLIFDYTPRK

SEQ ID NO: 187 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaatcgggatacacccacattgtagaagtcacttttgaatcagtggaaaccatacaaggttatatcattcaccctgcacatgttgggttcggtagggtctacagatcattttgggaagaattattgatctttgattacactccaaggaag

SEQ ID NO: 188 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKESGYTHIVEVTFESVETIQGYIIHPAHVGFGRVYRSFWEELLIFDYTPRK

SEQ ID NO: 189 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttcttggggtaaggatgttacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 190 - protein, 101 aa, synthetic construct
  REGION 1..101: note = Synthetic biopolymer
MAVKHLIVVKFKDGITEAQKEEFFKTYVNLVNIIPAMKDVSWGKDVTQRNKESGYTHIVEVTFESVETIQGYIIHPAHVGFGDVYRSFWEELLIFDYTPRK

SEQ ID NO: 191 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaagttggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 192 - protein, 101 aa, synthetic construct
  REGION 1..101: note = Synthetic biopolymer
MAVKHLIVVKFKDGITEAQKLEFFKTYVNLVNIIPAMKDVYWGKDVTQRNKEEGYTHIVEVTFESVETIQGYIIHPAHVGFGDVYRSFWEELLIFDYTPRK

SEQ ID NO: 193 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataagtcggaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 194 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKSEGYTHIVEVTFESVETIQGYIIHPAHVGFGDVYRSFWEELLIFDYTPRK

SEQ ID NO: 195 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtaaggtctacagatcattttgggaagaattattgatctttgattacactccaaggaag

SEQ ID NO: 196 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGKVYRSFWEELLIFDYTPRK

SEQ ID NO: 197 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacgttccaaggaag

SEQ ID NO: 198 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGDVYRSFWEELLIFDYVPRK

SEQ ID NO: 199 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcaggcaccctgcacatgttgggttcggtgatgtctacagatcattttgggaagaattattgatctttgattacactccaaggaag

SEQ ID NO: 200 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIRHPAHVGFGDVYRSFWEELLIFDYTPRK

SEQ ID NO: 201 - DNA, 303 bp, synthetic construct
  misc_feature 1..303: note = Synthetic biopolymer
  source 1..303: mol_type = other DNA
atggtggtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 202 - protein, 101 aa, synthetic construct
  REGION 1..101: note = Synthetic biopolymer
MVVKHLIVVKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 203 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaatgtatacaaggttatatcattcaccctgcacatgttgggttcggtgatgtctacagatcattttgggaagaattattgatctttgattacactccaaggaag

SEQ ID NO: 204 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVECIQGYIIHPAHVGFGDVYRSFWEELLIFDYTPRK

SEQ ID NO: 205 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcgctgatgtctacagatcattttgggaagaattattgatctttgattacactccaaggaag

SEQ ID NO: 206 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFADVYRSFWEELLIFDYTPRK

SEQ ID NO: 207 - DNA, 303 bp, synthetic construct; identical to SEQ ID NO: 119
  misc_feature 1..303: note = Synthetic biopolymer
  source 1..303: mol_type = other DNA

SEQ ID NO: 208 - protein, 101 aa, synthetic construct; identical to SEQ ID NO: 120
  REGION 1..101: note = Synthetic biopolymer

SEQ ID NO: 209 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgtttctcaaagaaataaggaagaaggatacacccacattgtagaagtcacttttgaatcagtggaaaccatacaaggttatatccagcaccctgcacatgttgggttcggtgatgtctacagatcattttgggaagaattattgatctttgattacactccaaggaag

SEQ ID NO: 210 - protein, 101 aa, synthetic construct
  REGION 1..101: note = Synthetic biopolymer
MAVKHLIVVKFKDGITEAQKEEFFKTYVNLVNIIPAMKDVYWGKDVSQRNKEEGYTHIVEVTFESVETIQGYIQHPAHVGFGDVYRSFWEELLIFDYTPRK

SEQ ID NO: 211 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttgggcaaagaaataaggaagaaggatacacccacattgtagaagtcacttttgaatcagtggaaaccatacaaggttatatcattcaccctgcacatgttgggttcggtgatgtctaccgttcattttgggaagaattattgatctttgattacactccaaggaag

SEQ ID NO: 212 - protein, 101 aa, synthetic construct
  REGION 1..101: note = Synthetic biopolymer
MAVKHLIVVKFKDGITEAQKEEFFKTYVNLVNIIPAMKDVYWGKDVGQRNKEEGYTHIVEVTFESVETIQGYIIHPAHVGFGDVYRSFWEELLIFDYTPRK

SEQ ID NO: 213 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcatggtgggaagaattattgatctttgattacactccaaggaag

SEQ ID NO: 214 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGDVYRSWWEELLIFDYTPRK

SEQ ID NO: 215 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagctaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 216 - protein, 101 aa, synthetic construct
  REGION 1..101: note = Synthetic biopolymer
MAVKHLIVVKFKDGITEAQKEEFFKTYVNLANIIPAMKDVYWGKDVTQRNKEEGYTHIVEVTFESVETIQGYIIHPAHVGFGDVYRSFWEELLIFDYTPRK

SEQ ID NO: 217 - DNA, 303 bp, synthetic construct
  misc_feature 1..303: note = Synthetic biopolymer
  source 1..303: mol_type = other DNA
atgtcggtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 218 - protein, 101 aa, synthetic construct
  REGION 1..101: note = Synthetic biopolymer
MSVKHLIVVKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 219 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagagcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 220 - protein, 101 aa, synthetic construct
  REGION 1..101: note = Synthetic biopolymer
MAVKHLIVVKFKDGITEEQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 221 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcattcaccctgcacatgttgggttcggtgatgtctacagttcattttgggaagaattattgatctttgattacactccaaggaag

SEQ ID NO: 222 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIIHPAHVGFGDVYSSFWEELLIFDYTPRK

SEQ ID NO: 223 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatccatcaccctgcacatgttgggttcggtgatgtctacagatcattttgggaagaattattgatctttgattacactccaaggaag

SEQ ID NO: 224 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIHHPAHVGFGDVYRSFWEELLIFDYTPRK

SEQ ID NO: 225 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatccagcaccctgcacatgttgggttcggtgatgtctacagatcattttgggaagaattattgatctttgattacactccaaggaag

SEQ ID NO: 226 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYIQHPAHVGFGDVYRSFWEELLIFDYTPRK

SEQ ID NO: 227 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgtttctcaaagaaataaggaagaaggatacacccacattgtagaagtcacttttgaatcagtggaaaccatacaaggttatatcattcaccctgcacatgttgggttcggtgatgtctacagatcattttgggaagaattattgatctttgattacactccaaggaag

SEQ ID NO: 228 - protein, 101 aa, synthetic construct
  REGION 1..101: note = Synthetic biopolymer
MAVKHLIVVKFKDGITEAQKEEFFKTYVNLVNIIPAMKDVYWGKDVSQRNKEEGYTHIVEVTFESVETIQGYIIHPAHVGFGDVYRSFWEELLIFDYTPRK

SEQ ID NO: 229 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatgttacgcaaagaaataaggaagaaggatacacccacattgtagaagtcacttttgaatcagtggaaaccatacaaggttatatcaatcaccctgcacatgttgggttcggtgatgtctacagatcattttgggaagaattattgatctttgattacactccaaggaag

SEQ ID NO: 230 - protein, 101 aa, synthetic construct
  REGION 1..101: note = Synthetic biopolymer
MAVKHLIVVKFKDGITEAQKEEFFKTYVNLVNIIPAMKDVYWGKDVTQRNKEEGYTHIVEVTFESVETIQGYINHPAHVGFGDVYRSFWEELLIFDYTPRK

SEQ ID NO: 231 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagacgtttattggggtaaggatttgacgcaaagaaataaggaatcgggatacacccacattgtagaagtcacttttgaatcagtggaaaccatacaaggttatatcatacaccctgcacatgttgggttcggtgatgtctacagatcattttgggaagaattattgatctttgattacactccaaggaag

SEQ ID NO: 232 - protein, 101 aa, synthetic construct
  REGION 1..101: note = Synthetic biopolymer
MAVKHLIVVKFKDGITEAQKEEFFKTYVNLVNIIPAMKDVYWGKDLTQRNKESGYTHIVEVTFESVETIQGYIIHPAHVGFGDVYRSFWEELLIFDYTPRK

SEQ ID NO: 233 - DNA, 303 bp, synthetic construct
  misc_feature 1..303: note = Synthetic biopolymer
  source 1..303: mol_type = other DNA
atgggtgtcaagcaccttatcgtaattaaattcaaggacgaaattacagaagcccagaaagaagaatttttcagaacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 234 - protein, 101 aa, synthetic construct
  REGION 1..101: note = Synthetic biopolymer
MGVKHLIVIKFKDEITEAQKEEFFRTYVNLVNIIPAMKDVYWGKDVTQKNKEEGYTHIVEVTFESVETIQDYIIHPAHVGFGDVYRSFWEKLLIFDYTPRK

SEQ ID NO: 235 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctatgtaaacctggtgaatatcatccctgcgatgaaagatgtttattggggcaaagacgtcacccaaaaaaacaaagaatctggttacactcacatcgtcgaagtcactttcgagtcagtagagacgatccaagattacataatacacccggcccatgtgggattcggagacgtttaccgtagcttctgggaaaaacttttgaaattcgactataccccgagaaaa

SEQ ID NO: 236 - protein, 101 aa, synthetic construct
  REGION 1..101: note = Synthetic biopolymer
MAVKHLIVIKFKDEITEAQKEEFFKTYVNLVNIIPAMKDVYWGKDVTQKNKESGYTHIVEVTFESVETIQDYIIHPAHVGFGDVYRSFWEKLLKFDYTPRK

SEQ ID NO: 237 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgaaatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 238 - protein, 101 aa, synthetic construct
  REGION 1..101: note = Synthetic biopolymer
MAVKHLIVLKFKDEITEAQKEEFFKTYVNLVNIIPEMKDVYWGKDVTQKNKEEGYTHIVEVTFESVETIQDYIIHPAHVGFGDVYRSFWEKLLIFDYTPRK

SEQ ID NO: 239 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaacatggttacactcacatcgtcgaagtcactttcgattcagtagagacgatccaagattacataatacacccggcccatgtgggattcggagacgtttaccgtagcttctgggaaaaacttttgattttcgactataccccgagaaaa

SEQ ID NO: 240 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEHGYTHIVEVTFDSVETIQDYIIHPAHVGFGDVYRSFWEKLLIFDYTPRK

SEQ ID NO: 241 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcagaacctatgtaaacctggtgaatatcatccctgaaatgaaagatgtttattggggcaaagacgtcacccaaaaaaacaaagaacatggttacactcacatcgtcgaagtcactttcgagtcagtagagacgatccaagattacataatacacccggcccatgtgggattcggagacgtttaccgtagcttctgggaaaaacttttgaaattcgactataccccgagaaaa

SEQ ID NO: 242 - protein, 101 aa, synthetic construct
  REGION 1..101: note = Synthetic biopolymer
MAVKHLIVIKFKDEITEAQKEEFFRTYVNLVNIIPEMKDVYWGKDVTQKNKEHGYTHIVEVTFESVETIQDYIIHPAHVGFGDVYRSFWEKLLKFDYTPRK

SEQ ID NO: 243 - DNA, 303 bp, synthetic construct
  misc_feature 1..303: note = Synthetic biopolymer
  source 1..303: mol_type = other DNA
atgggtgtcaagcaccttatcgtaattaaattcaaggacgaaattacagaagcccagaaagaagaatttttcagaacctatgtaaacctggtgaatatcatccctgcgatgaaagatgtttattggggcaaagacgtcacccaaaaaaacaaagaatctggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 244 - protein, 101 aa, synthetic construct
  REGION 1..101: note = Synthetic biopolymer
MGVKHLIVIKFKDEITEAQKEEFFRTYVNLVNIIPAMKDVYWGKDVTQKNKESGYTHIVEVTFDSVETIQDYIIHPAHVGFGDVYRSFWEKLLKFDYTPRK

SEQ ID NO: 245 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttctctacctatgtaaacctggtgaatatcatccctgaaatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 246 - protein, 101 aa, synthetic construct
  REGION 1..101: note = Synthetic biopolymer
MAVKHLIVLKFKDEITEAQKEEFFSTYVNLVNIIPEMKDVYWGKDVTQKNKEEGYTHIVEVTFESVETIQDYIIHPAHVGFGDVYRSFWEKLLIFDYTPRK

SEQ ID NO: 247 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattcaagaagcccagaaagaagaatttttcagaacctatgtaaacctggtgaatatcatccctgcgatgaaagatgtttattggggcaaagacgtcacccaaaaaaacaaagaatctggttacactcacatcgtcgaagtcactttcgattcagtagagacgatccaagattacataatacacccggcccatgtgggattcggagacgtttaccgtagcttctgggaaaaacttttgattttcgactataccccgagaaaa

SEQ ID NO: 248 - protein, 101 aa, synthetic construct
  REGION 1..101: note = Synthetic biopolymer
MAVKHLIVIKFKDEIQEAQKEEFFRTYVNLVNIIPAMKDVYWGKDVTQKNKESGYTHIVEVTFDSVETIQDYIIHPAHVGFGDVYRSFWEKLLIFDYTPRK

SEQ ID NO: 249 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttctctacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgaaattcgactataccccgagaaaa

SEQ ID NO: 250 - protein, 101 aa, synthetic construct
  REGION 1..101: note = Synthetic biopolymer
MAVKHLIVLKFKDEITEAQKEEFFSTYVNLVNIIPAMKDVYWGKDVTQKNKEEGYTHIVEVTFESVETIQDYIIHPAHVGFGDVYRSFWEKLLKFDYTPRK

SEQ ID NO: 251 - DNA, 303 bp, synthetic construct
  misc_feature 1..303: note = Synthetic biopolymer
  source 1..303: mol_type = other DNA
atgggtgtcaagcaccttatcgtaattaaattcaaggacgaaattacagaagcccagaaagaagaatttttcggtacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 252 - protein, 101 aa, synthetic construct
  REGION 1..101: note = Synthetic biopolymer
MGVKHLIVIKFKDEITEAQKEEFFGTYVNLVNIIPAMKDVYWGKDVTQKNKEEGYTHIVEVTFESVETIQDYIIHPAHVGFGDVYRSFWEKLLIFDYTPRK

SEQ ID NO: 253 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagctggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 254 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEAGYTHIVEVTFESVETIQDYIIHPAHVGFGDVYRSFWEKLLIFDYTPRK

SEQ ID NO: 255 - DNA, 303 bp, synthetic construct
  misc_feature 1..303: note = Synthetic biopolymer
  source 1..303: mol_type = other DNA
atgggtgtcaagcaccttatcgtagttaaattcaaggacgaaattccagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 256 - protein, 101 aa, synthetic construct
  REGION 1..101: note = Synthetic biopolymer
MGVKHLIVVKFKDEIPEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 257 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagctggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 258 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEAGYTHIVEVTFDSVETIQDYIIHPAHVGFGDVYRSFWEKLLKFDYTPRK

SEQ ID NO: 259 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttctctacctatgtaaacctggtgaatatcatccctgaaatgaaagatgtttattggggcaaagacgtcacccaaaaaaacaaagaagaaggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 260 - protein, 101 aa, synthetic construct
  REGION 1..101: note = Synthetic biopolymer
MAVKHLIVIKFKDEITEAQKEEFFSTYVNLVNIIPEMKDVYWGKDVTQKNKEEGYTHIVEVTFDSVETIQDYIIHPAHVGFGDVYRSFWEKLLKFDYTPRK

SEQ ID NO: 261 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttctctacctatgtaaacctggtgaatatcatccctgaaatgaaagatgtttattggggcaaagacgtcacccaaaaaaacaaagaaagaggttacactcacatcgtcgaagtcactttcgagtcagtagagacgatccaagattacataatacacccggcccatgtgggattcggagacgtttaccgtagcttctgggaaaaacttttgaaattcgactataccccgagaaaa

SEQ ID NO: 262 - protein, 101 aa, synthetic construct
  REGION 1..101: note = Synthetic biopolymer
MAVKHLIVIKFKDEITEAQKEEFFSTYVNLVNIIPEMKDVYWGKDVTQKNKERGYTHIVEVTFESVETIQDYIIHPAHVGFGDVYRSFWEKLLKFDYTPRK

SEQ ID NO: 263 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcgagacctatgtaaacctggtgaatatcatccctgaaatgaaagatgtttattggggcaaagacgtcacccaaaaaaacaaagaagaaggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 264 - protein, 101 aa, synthetic construct
  REGION 1..101: note = Synthetic biopolymer
MAVKHLIVLKFKDEITEAQKEEFFETYVNLVNIIPEMKDVYWGKDVTQKNKEEGYTHIVEVTFDSVETIQDYIIHPAHVGFGDVYRSFWEKLLKFDYTPRK

SEQ ID NO: 265 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcagaacctatgtaaacctggtgaatatcatccctgcgatgaaagatgtttattggggcaaagacgtcacccaaaaaaacaaagaagaaggttacactcacatcgtcgaagtcactttcgattcagtagagacgatccaagattacataatacacccggcccatgtgggattcggagacgtttaccgtagcttctgggaaaaacttttgattttcgactataccccgagaaaa

SEQ ID NO: 266 - protein, 101 aa, synthetic construct
  REGION 1..101: note = Synthetic biopolymer
MAVKHLIVLKFKDEITEAQKEEFFRTYVNLVNIIPAMKDVYWGKDVTQKNKEEGYTHIVEVTFDSVETIQDYIIHPAHVGFGDVYRSFWEKLLIFDYTPRK

SEQ ID NO: 267 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcagaacctatgtaaacctggtgaatatcatccctgaaatgaaagatgtttattggggcaaagacgtcacccaaaaaaacaaagaatctggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 268 - protein, 101 aa, synthetic construct
  REGION 1..101: note = Synthetic biopolymer
MAVKHLIVLKFKDEITEAQKEEFFRTYVNLVNIIPEMKDVYWGKDVTQKNKESGYTHIVEVTFDSVETIQDYIIHPAHVGFGDVYRSFWEKLLKFDYTPRK

SEQ ID NO: 269 - DNA, 303 bp, synthetic construct
  misc_feature 1..303: note = Synthetic biopolymer
  source 1..303: mol_type = other DNA
atgggtgtcaagcaccttatcgtaattaaattcaaggacgaaattacagaagcccagaaagaagaatttttcaagacctatgtaaacctggtgaatatcatccctgaaatgaaagatgtttattggggcaaagacgtcacccaaaaaaacaaagaatctggttacactcacatcgtcgaagtcactttcgattcagtagagacgatccaagattacataatacacccggcccatgtgggattcggagacgtttaccgtagcttctgggaaaaacttttgaaattcgactataccccgagaaaa

SEQ ID NO: 270 - protein, 101 aa, synthetic construct
  REGION 1..101: note = Synthetic biopolymer
MGVKHLIVIKFKDEITEAQKEEFFKTYVNLVNIIPEMKDVYWGKDVTQKNKESGYTHIVEVTFDSVETIQDYIIHPAHVGFGDVYRSFWEKLLKFDYTPRK

SEQ ID NO: 271 - DNA, 303 bp, synthetic construct
  misc_feature 1..303: note = Synthetic biopolymer
  source 1..303: mol_type = other DNA
atgggtgtcaagcaccttatcgtagttaaattcaaggacgaaattacagaagcccagaaagaagaatttttcaagacctttgtaaacctggtgaatatcatccctgcgatgaaagatgtttattggggcaaagacgtcacccaaaaaaacaaaagagaaggttacactcacatcgtcgaagtcactttcgagtcattggagacgatccaagattacataatacacccggcccatgtgggattcggagacatgtaccgtagcttctgggaaaaacttttgattttcgactataccccgagaaaa

SEQ ID NO: 272 - protein, 101 aa, synthetic construct
  REGION 1..101: note = Synthetic biopolymer
MGVKHLIVVKFKDEITEAQKEEFFKTFVNLVNIIPAMKDVYWGKDVTQKNKREGYTHIVEVTFESLETIQDYIIHPAHVGFGDMYRSFWEKLLIFDYTPRK

SEQ ID NO: 273 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacgtttaccgtagcttctgggaaaaacttttgattttcgactataccccgagaaaa

SEQ ID NO: 274 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKEEGYTHIVEVTFESLETIQDYIIHPAHVGFGDVYRSFWEKLLIFDYTPRK

SEQ ID NO: 275 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacatgtaccgtagcttctgggaaaaacttttgattttcgactataccccgagaaaa

SEQ ID NO: 276 - protein, 101 aa, synthetic construct
  REGION 1..101: note = Synthetic biopolymer
MAVKHLIVVKFKDEITEAQKEEFFKTFVNLVNIIPAMKDVYWGKDVTQKNKEEGYTHIVEVTFESVETIQDYIIHPAHVGFGDMYRSFWEKLLIFDYTPRK

SEQ ID NO: 277 - DNA, 303 bp, synthetic construct
  misc_feature 1..303: note = Synthetic biopolymer
  source 1..303: mol_type = other DNA
atgggtgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 278 - protein, 101 aa, synthetic construct
  REGION 1..101: note = Synthetic biopolymer
MGVKHLIVVKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 279 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctttgtaaacctggtgaatatcatccctgcgatgaaagatgtttattggggcaaagacgtcacccaaaaaaacaaagaatcgggatacactcacatcgtcgaagtcactttcgagtcattggagacgatccaagattacataatacacccggcccatgtgggattcggagacatgtaccgtagcttctgggaaaaacttttgattttcgactataccccgagaaaa

SEQ ID NO: 280 - protein, 101 aa, synthetic construct
  REGION 1..101: note = Synthetic biopolymer
MAVKHLIVIKFKDEITEAQKEEFFKTFVNLVNIIPAMKDVYWGKDVTQKNKESGYTHIVEVTFESLETIQDYIIHPAHVGFGDMYRSFWEKLLIFDYTPRK

SEQ ID NO: 281 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacatgtaccgtagcttctgggaaaaacttttgattttcgactataccccgagaaaa

SEQ ID NO: 282 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVTFESLETIQDYIIHPAHVGFGDMYRSFWEKLLIFDYTPRK

SEQ ID NO: 283 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaacaagaaggttacactcacatcgtcgaagtcactttcgagtcattggagacgatccaagattacataatacacgttgcccatgtgggattcggagacgtttaccgtagcttctgggaaaaacttttgattttcgactataccccgagaaaa

SEQ ID NO: 284 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKQEGYTHIVEVTFESLETIQDYIIHVAHVGFGDVYRSFWEKLLIFDYTPRK

SEQ ID NO: 285 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacatttaccgtagcttctgggaaaaacttttgattttcgactataccccgagaaaa

SEQ ID NO: 286 - protein, 101 aa, synthetic construct
  REGION 1..101: note = Synthetic biopolymer
MAVKHLIVIKFKDEITEAQKEEFFKTFVNLVNIIPAMKDVYWGKDVTQKNKEEGYTHIVEVTFESLETIQDYIIHPAHVGFGDIYRSFWEKLLIFDYTPRK

SEQ ID NO: 287 - DNA, 303 bp, synthetic construct
  misc_feature 1..303: note = Synthetic biopolymer
  source 1..303: mol_type = other DNA
atgggtgtcaagcaccttatcgtagttaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacgtttaccgtagcttctgggaaaaacttttgattttcgactataccccgagaaaa

SEQ ID NO: 288 - protein, 101 aa, synthetic construct
  REGION 1..101: note = Synthetic biopolymer
MGVKHLIVVKFKDEITEAQKEEFFKTFVNLVNIIPAMKDVYWGKDVTQKNKEEGYTHIVEVTFESLETIQDYIIHPAHVGFGDVYRSFWEKLLIFDYTPRK

SEQ ID NO: 289 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 290 - protein, 101 aa, synthetic construct
  REGION 1..101: note = Synthetic biopolymer
MAVKHLIVLKFKDEITEAQKEEFFKTFVNLVNIIPAMKDVYWGKDVTQKNKEEGYTHIVEVTFESVETIQDYIIHPAHVGFGDVYRSFWEKLLIFDYTPRK

SEQ ID NO: 291 - DNA, 303 bp, synthetic construct
  misc_feature 1..303: note = Synthetic biopolymer
  source 1..303: mol_type = other DNA
atgggtgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacatgtaccgtagcttctgggaaaaacttttgattttcgactataccccgagaaaa

SEQ ID NO: 292 - protein, 101 aa, synthetic construct
  REGION 1..101: note = Synthetic biopolymer
MGVKHLIVVKFKDEITEAQKEEFFKTYVNLVNIIPAMKDVYWGKDVTQKNKEEGYTHIVEVTFESLETIQDYIIHPAHVGFGDMYRSFWEKLLIFDYTPRK

SEQ ID NO: 293 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaaggcctatgtaaacctggtgaatatcatccctgcgatgaaagatgtttattggggcaaagacgtcacccaaaaaaacaaacaagaaggttacactcacatcgtcgaagtcactttcgagtcagtagagacgatccaagattacataatacacgttgcccatgtgggattcggagacgtttaccgtagcttctgggaaaaacttttgattttcgactataccccgagaaaa

SEQ ID NO: 294 - protein, 101 aa, synthetic construct
  REGION 1..101: note = Synthetic biopolymer
MAVKHLIVVKFKDEITEAQKEEFFKAYVNLVNIIPAMKDVYWGKDVTQKNKQEGYTHIVEVTFESVETIQDYIIHVAHVGFGDVYRSFWEKLLIFDYTPRK

SEQ ID NO: 295 - DNA, 303 bp, synthetic construct
  misc_feature 1..303: note = Synthetic biopolymer
  source 1..303: mol_type = other DNA
atggctgtcaagcaccttatcgtagttaaatttaaggacgaaattacagaagcccagaaagaagaatttttcaagacctatgtaaacctggtgaatatcatccctgcgatgaaagatgtttattggggcaaagacgtcacccaaaaaaacaaaagagaaggttacactcacatcgtcgaagtcactttcgagtcagtagagacgatccaagattacataatacacccggcccatgtgggattcggagacatgtaccgtagcttctgggaaaaacttttgattttcgactataccccgagaaaa

SEQ ID NO: 296 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKREGYTHIVEVTFESVETIQDYIIHPAHVGFGDMYRSFWEKLLIFDYTPRK

SEQ ID NO: 297 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcattggagacgatccaagattacataatacacccggcccatgtgggattcggagacatgtaccgtagcttctgggaaaaacttttgattttcgactataccccgagaaaa

SEQ ID NO: 298 - protein, 101 aa, synthetic construct
  REGION 1..101: note = Synthetic biopolymer
MAVKHLIVVKFKDEITEAQKEEFFKTFVNLVNIIPAMKDVYWGKDVTQKNKEEGYTHIVEVTFESLETIQDYIIHPAHVGFGDMYRSFWEKLLIFDYTPRK

SEQ ID NO: 299 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctatgtaggtctggtgaatatcatccctgcgatgaaagatgtttattggggcaaagacgtcacccaaaaaaacaaagaagaaggttacactcacatcgtcgaagtcggtttcgagtcagtagaggaaatccaagattacataatacacccggcccatgtgggattcggagacgtttaccgtagcttctgggaaaaacttttgaaattcgactataccccgagaaaa

SEQ ID NO: 300 - protein, 101 aa, synthetic construct
  REGION 1..101: note = Synthetic biopolymer
MAVKHLIVIKFKDEITEAQKEEFFKTYVGLVNIIPAMKDVYWGKDVTQKNKEEGYTHIVEVGFESVEEIQDYIIHPAHVGFGDVYRSFWEKLLKFDYTPRK

SEQ ID NO: 301 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctatgtaaacctggtgaatatcatccctgcgatgaaagatgtttattggggcccagacgtcacccaaaaaaacaaagaagaaggttacactcacatcgtcgaagtcactttcgagtcagtagaggaaatccaagattacataatacacccggcccatgtgggattcggagacgtttaccgtagcttctgggaaaaacttttgaaattcgactataccccgagaaaa

SEQ ID NO: 302 - protein, 101 aa, synthetic construct
  REGION 1..101: note = Synthetic biopolymer
MAVKHLIVIKFKDGITEAQKEEFFKTYVNLVNIIPAMKDVYWGPDVTQKNKEEGYTHIVEVTFESVEEIQDYIIHPAHVGFGDVYRSFWEKLLKFDYTPRK

SEQ ID NO: 303 - DNA, 303 bp, synthetic construct
  misc_feature 1..303: note = Synthetic biopolymer
  source 1..303: mol_type = other DNA
atggctgtcaagcaccttatcgtactgaaattcaaggacggtattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagtctatccaagattacataatacacccggcccatgtgggattcggagacgtttaccgtagcttctgggaaaaacttttgaaattcgactataccccgagaaaa

SEQ ID NO: 304 - protein, 101 aa, synthetic construct
  REGION 1..101: note = Synthetic biopolymer
MAVKHLIVLKFKDGITEAQKEEFFKTYVNLVNIIPAMKDVYWGKDVTQKNKEEGYTHIVEVTFESVESIQDYIIHPAHVGFGDVYRSFWEKLLKFDYTPRK

SEQ ID NO: 305 - DNA, 303 bp, synthetic construct
  misc_feature 1..303: note = Synthetic biopolymer
  source 1..303: mol_type = other DNA
atggctgtcaagcaccttatcgtactgaaattcaaggacggtattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagaggctatccaagattacataatacacccggcccatgtgggattcggagacgtttaccgtagcttctgggaaaaacttttgaaattcgactataccccgagaaaa

SEQ ID NO: 306 - protein, 101 aa, synthetic construct
  REGION 1..101: note = Synthetic biopolymer
MAVKHLIVLKFKDGITEAQKEEFFKTYVNLVNIIPAMKDVYWGKDVTQKNKEEGYTHIVEVTFESVEAIQDYIIHPAHVGFGDVYRSFWEKLLKFDYTPRK

SEQ ID NO: 307 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcggtttcgagtcagtagagggtatccaagattacataatacacccggcccatgtgggattcggagacgtttaccgtagcttctgggaaaaacttttgaaattcgactataccccgagaaaa

SEQ ID NO: 308 - protein, 101 aa, synthetic construct
  REGION 1..101: note = Synthetic biopolymer
MAVKHLIVLKFKDEITEAQKEEFFKTYVNLVNIIPAMKDVYWGKDVTQKNKEEGYTHIVEVGFESVEGIQDYIIHPAHVGFGDVYRSFWEKLLKFDYTPRK

SEQ ID NO: 309 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagatgtttattggggcaaagacgtcacccaaaaaaacaaagaagaaggttacactcacattgtagaagtcactttcgagtcagtagaggaaatccaagattacataatacacccggcccatgtgggattcggagacgtttaccgtagcttctgggaaaaacttttgaaattcgactataccccgagaaaa

SEQ ID NO: 310 - protein, 101 aa, synthetic construct
  REGION 1..101: note = Synthetic biopolymer
MAVKHLIVVKFKDGITEAQKEEFFKTYVNLVNIIPAMKDVYWGKDVTQKNKEEGYTHIVEVTFESVEEIQDYIIHPAHVGFGDVYRSFWEKLLKFDYTPRK

SEQ ID NO: 311 - DNA, 303 bp, synthetic construct
  misc_feature 1..303: note = Synthetic biopolymer
  source 1..303: mol_type = other DNA
atggccgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgaaattcgactataccccgagaaaa

SEQ ID NO: 312 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKEEGYTHIVEVTFESVETIQDYIIHPAHVGFGDVYRSFWEKLLKFDYTPRK

SEQ ID NO: 313 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaggtctggtgaatatcatccctgcgatgaaagatgtttattggggcaaagacgtcacccaaaaaaacaaagaagaaggttacactcacatcgtcgaagtcactttcgagtcagtagaggaaatccaagattacataatacacccggcccatgtgggattcggagacgtttaccgtagcttctgggaaaaacttttgaaattcgactataccccgagaaaa

SEQ ID NO: 314 - protein, 101 aa, synthetic construct
  REGION 1..101: note = Synthetic biopolymer
MAVKHLIVVKFKDEITEAQKEEFFKTYVGLVNIIPAMKDVYWGKDVTQKNKEEGYTHIVEVTFESVEEIQDYIIHPAHVGFGDVYRSFWEKLLKFDYTPRK

SEQ ID NO: 315 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattccagaagcccagaaagaagaatttttcaagacctatgtaaacctgatgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtaaattctgggaaaaacttttgattttcgactataccccgagaaaa

SEQ ID NO: 316 - protein, 101 aa, synthetic construct
  REGION 1..101: note = Synthetic biopolymer
MGVKHLIVLKFKDEIPEAQKEEFFKTYVNLMNIIPAMKDVYWGKDVTQRNKEEGYTHIVEVTFESVETIQDYIIHPAHVGFGDVYRKFWEKLLIFDYTPRK

SEQ ID NO: 317 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggaaaatatcatccctgcgatgaaagatgtttattggggcaaagacgtcacccaaaaaaacaaagaagaaggttacactcacatcgtcgaagtcactttggagtcagtagagacgatccaagattacataatccacccggcccatgtgggattcggagacgtttaccgtaaattttgggaaaaacttttgattttcgactataccccgagaaaa

SEQ ID NO: 318 - protein, 101 aa, synthetic construct
  REGION 1..101: note = Synthetic biopolymer
MAVKHLIVLKFKDEITEAQKEEFFKTYVNLENIIPAMKDVYWGKDVTQKNKEEGYTHIVEVTLESVETIQDYIIHPAHVGFGDVYRKFWEKLLIFDYTPRK

SEQ ID NO: 319 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 320 - protein, 101 aa, synthetic construct
  REGION 1..101: note = Synthetic biopolymer
MGVKHLIVLKFKDEITEAQKEEFFKTYVNLVNIIPAMKDVYWGKDVTQRNKEEGYTHIVEVTFESVETIQDYIIHPAHVGFGDVYRSFWEKLLIFDYTPRK

SEQ ID NO: 321 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctgatgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtaaattctgggaaaaacttttgattttcgactataccccgagaaaa

SEQ ID NO: 322 - protein, 101 aa, synthetic construct
  REGION 1..101: note = Synthetic biopolymer
MAVKHLIVLKFKDEITEAQKEEFFKTYVNLMNIIPAMKDVYWGKDVTQRNKEEGYTHIVEVTFESVETIQDYIIHPAHVGFGDVYRKFWEKLLIFDYTPRK

SEQ ID NO: 323 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctatgtaaacctgtctaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 324 - protein, 101 aa, synthetic construct
  REGION 1..101: note = Synthetic biopolymer
MGVKHLIVLKFKDEITEAQKEEFFKTYVNLSNIIPAMKDVYWGKDVTQRNKEEGYTHIVEVTFESVETIQDYIIHPAHVGFGDVYRSFWEELLIFDYTPRK

SEQ ID NO: 325 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggaaaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtaaattctgggaaaaacttttgattttcgactataccccgagaaaa

SEQ ID NO: 326 - protein, 101 aa, synthetic construct
  REGION 1..101: note = Synthetic biopolymer
MAVKHLIVLKFKDEITEAQKEEFFKTYVNLENIIPAMKDVYWGKDVTQRNKEEGYTHIVEVTFESVETIQDYIIHPAHVGFGDVYRKFWEKLLIFDYTPRK

SEQ ID NO: 327 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattccagaagcccagaaagaagaatttttcaagacctatgtaaacctgatgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 328 - protein, 101 aa, synthetic construct
  REGION 1..101: note = Synthetic biopolymer
MAVKHLIVLKFKDEIPEAQKEEFFKTYVNLMNIIPAMKDVYWGKDVTQRNKEEGYTHIVEVTFESVETIQDYIIHPAHVGFGDVYRSFWEKLLIFDYTPRK

SEQ ID NO: 329 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaagacctatgtaaacctgtctaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 330 - protein, 101 aa, synthetic construct
  REGION 1..101: note = Synthetic biopolymer
MAVKHLIVVKFKDGITEAQKEEFFKTYVNLSNIIPAMKDVYWGKDVTQRNKEEGYTHIVEVTFESVETIQDYIIHPAHVGFGDVYRSFWEELLIFDYTPRK

SEQ ID NO: 331 - DNA, 303 bp, synthetic construct
  misc_feature 1..303: note = Synthetic biopolymer
  source 1..303: mol_type = other DNA
atggctgtcaagcaccttatcgtactgaaattcttggacgaaattacagaagcccagaaagaagaatttttcaagacctatgtaaacctgatgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 332 - protein, 101 aa, synthetic construct
  REGION 1..101: note = Synthetic biopolymer
MAVKHLIVLKFLDEITEAQKEEFFKTYVNLMNIIPAMKDVYWGKDVTQRNKEEGYTHIVEVTFESVETIQDYIIHPAHVGFGDVYRSFWEKLLIFDYTPRK

SEQ ID NO: 333 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 334 - protein, 101 aa, synthetic construct
  REGION 1..101: note = Synthetic biopolymer
MAVKHLIVVKFKDEITEAQKEEFFKTYVNLVNIIPAMKDVYWGKDVTQKNKEEGYTHIVEVTFESVETIQDYIIHPAHVGFGDVYRSFWEELLIFDYTPRK

SEQ ID NO: 335 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctatgtaaacctggtgaatatcatccctgcgatgaaagatgcttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 336 - protein, 101 aa, synthetic construct
  REGION 1..101: note = Synthetic biopolymer
MAVKHLIVIKFKDGITEAQKEEFFKTYVNLVNIIPAMKDAYWGKDVTQRNKEEGYTHIVEVTFESVETIQDYIIHPAHVGFGDVYRSFWEKLLIFDYTPRK

SEQ ID NO: 337 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaaggttacataatacacccggcccatgtgggattcggagacgtttaccgtagcttctgggaagaacttttgattttcgactataccccgagaaaa

SEQ ID NO: 338 - protein, 101 aa, synthetic construct
  REGION 1..101: note = Synthetic biopolymer
MAVKHLIVVKFKDEITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 339 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcagtagagacgatccaaggttacataatacacccggcccatgtgggattcggagacgtttaccgtagcttctgggaagaacttttgattttcgactataccccgagaaaa

SEQ ID NO: 340 - protein, 101 aa, synthetic construct
  REGION 1..101: note = Synthetic biopolymer
MAVKHLIVIKFKDGITEAQKEEFFKTFVNLVNIIPAMKDVYWGKDVTQKNKEEGYTHIVEVTFESVETIQGYIIHPAHVGFGDVYRSFWEELLIFDYTPRK

SEQ ID NO: 341 - DNA, 303 bp, synthetic construct
  misc_feature 1..303: note = Synthetic biopolymer
  source 1..303: mol_type = other DNA
atggctgtcaagcaccttatcgtactgaaattcaaggacggtattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaaggttacataatacacccggcccatgtgggattcggagacgtttaccgtagcttctgggaagaacttttgattttcgactataccccgagaaaa

SEQ ID NO: 342 - protein, 101 aa, synthetic construct
  REGION 1..101: note = Synthetic biopolymer
MAVKHLIVLKFKDGITEAQKEEFFKTYVNLVNIIPAMKDVYWGKDVTQRNKEEGYTHIVEVTFESVETIQGYIIHPAHVGFGDVYRSFWEELLIFDYTPRK

SEQ ID NO: 343 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 344 - protein, 101 aa, synthetic construct
  REGION 1..101: note = Synthetic biopolymer
MAVKHLIVIKFKDEITEAQKEEFFKTFVNLVNIIPAMKDVYWGKDVTQKNKEEGYTHIVEVTFESVETIQDYIIHPAHVGFGDVYRSFWEELLIFDYTPRK

SEQ ID NO: 345 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgttttccgtagcttctgggaagaacttttgattttcgactataccccgagaaaa

SEQ ID NO: 346 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFESVETIQDYIIHPAHVGFGDVFRSFWEELLIFDYTPRK

SEQ ID NO: 347 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaaggttacataatacacccggcccatgtgggattcggagacgtttaccgtagcttctgggaagaacttttgattttcgactataccccgagaaaa

SEQ ID NO: 348 - protein, 101 aa, synthetic construct
  REGION 1..101: note = Synthetic biopolymer
MAVKHLIVIKFKDGITEAQKEEFFKTYVNLVNIIPAMKDVYWGKDVTQKNKEEGYTHIVEVTFESVETIQGYIIHPAHVGFGDVYRSFWEELLIFDYTPRK

SEQ ID NO: 349 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 350 - protein, 101 aa, synthetic construct
  REGION 1..101: note = Synthetic biopolymer
MAVKHLIVVKFKDEITEAQKEEFFKTYVNLVNIIPAMKDVYWGKDVTQRNKEEGYTHIVEVTFESVETIQDYIIHPAHVGFGDVYRSFWEELLIFDYTPRK

SEQ ID NO: 351 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaaggttacataatacacccggcccatgtgggattcggagacgtttaccgtagcttctgggaaaaacttttgattttcgactataccccgagaaaa

SEQ ID NO: 352 - protein, 101 aa, synthetic construct
  REGION 1..101: note = Synthetic biopolymer
MAVKHLIVIKFKDGITEAQKEEFFKTYVNLVNIIPAMKDVYWGKDVTQRNKEEGYTHIVEVTFESVETIQGYIIHPAHVGFGDVYRSFWEKLLIFDYTPRK

SEQ ID NO: 353 - DNA, 303 bp, synthetic construct
  misc_feature 1..303: note = Synthetic biopolymer
  source 1..303: mol_type = other DNA
atggctgtcaagcaccttatcgtactgaaattcaaggacggtattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 354 - protein, 101 aa, synthetic construct
  REGION 1..101: note = Synthetic biopolymer
MAVKHLIVLKFKDGITEAQKEEFFKTFVNLVNIIPAMKDVYWGKDVTQRNKEEGYTHIVEVTFESVETIQDYIIHPAHVGFGDVYRSFWEKLLIFDYTPRK

SEQ ID NO: 355 - DNA, 303 bp, synthetic construct
  misc_feature 1..303: note = Synthetic biopolymer
  source 1..303: mol_type = other DNA
atggctgtcaagcaccttatcgtagttaaattcaaggacgaaattcaagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 356 - protein, 101 aa, synthetic construct
  REGION 1..101: note = Synthetic biopolymer
MAVKHLIVVKFKDEIQEAQKEEFFKTYVNLVNIIPAMKDVYWGKDVTQRNKEEGYTHIVEVTFESVETIQDYIIHPAHVGFGDVYRSFWEELLIFDYTPRK

SEQ ID NO: 357 - DNA, 303 bp, synthetic construct
  misc_feature 1..303: note = Synthetic biopolymer
  source 1..303: mol_type = other DNA
atggctgtcaagcaccttatcgtaattaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 358 - protein, 101 aa, synthetic construct
  REGION 1..101: note = Synthetic biopolymer
MAVKHLIVIKFKDEITEAQKEEFFKTYVNLVNIIPAMKDVYWGKDVTQRNKEEGYTHIVEVTFESVETIQDYIIHPAHVGFGDVYRSFWEELLIFDYTPRK

SEQ ID NO: 359 - DNA, 303 bp, synthetic construct
  misc_feature 1..303: note = Synthetic biopolymer
  source 1..303: mol_type = other DNA
atggctgtcaagcaccttatcgtagttaaattcaaggacggtattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 360 - protein, 101 aa, synthetic construct
  REGION 1..101: note = Synthetic biopolymer
MAVKHLIVVKFKDGITEAQKEEFFKTFVNLVNIIPAMKDVYWGKDVTQRNKEEGYTHIVEVTFESVETIQDYIIHPAHVGFGDVYRSFWEELLIFDYTPRK

SEQ ID NO: 361 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 362 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFESVETIQDYIIHPAHVGFGDVYRSFWEELLIFDYTPRK

SEQ ID NO: 363 - DNA, 303 bp, synthetic construct
  misc_feature 1..303: note = Synthetic biopolymer
  source 1..303: mol_type = other DNA
atggctgtcaagcaccttatcgtaattaaattcaaggacggtattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaaggttacataatacacccggcccatgtgggattcggagacgtttaccgtagcttctgggaaaaacttttgattttcgactataccccgagaaaa

SEQ ID NO: 364 - protein, 101 aa, synthetic construct
  REGION 1..101: note = Synthetic biopolymer
MAVKHLIVIKFKDGITEAQKEEFFKTYVNLVNIIPAMKDVYWGKDVTQKNKEEGYTHIVEVTFESVETIQGYIIHPAHVGFGDVYRSFWEKLLIFDYTPRK

SEQ ID NO: 365 - DNA, 303 bp, synthetic construct
  misc_feature 1..303: note = Synthetic biopolymer
  source 1..303: mol_type = other DNA
atggctgtcaagcaccttatcgtactgaaattcaaggacggtattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatacacccggcccatgtgggattcggagacgtttaccgtagcttctgggaagaacttttgattttcgactataccccgagaaaa

SEQ ID NO: 366 - protein, 101 aa, synthetic construct
  REGION 1..101: note = Synthetic biopolymer
MAVKHLIVLKFKDGITEAQKEEFFKTYVNLVNIIPAMKDVYWGKDVTQRNKEEGYTHIVEVTFESVETIQDYIIHPAHVGFGDVYRSFWEELLIFDYTPRK

SEQ ID NO: 367 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgattcagtagagacgatccaagattacataactcacccggcccatgtgggattcggagacgtttaccgtagcttctgggaaaaacttttgattttcgactataccccgagaaaa

SEQ ID NO: 368 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFDSVETIQDYITHPAHVGFGDVYRSFWEKLLIFDYTPRK

SEQ ID NO: 369 - DNA, 303 bp, synthetic construct
  misc_feature 1..303: note = Synthetic biopolymer
  source 1..303: mol_type = other DNA
atggctgtcaagcaccttatcgtactgaaattcttggacgaaattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataatgcacccggcccatgtgggattcggagacgtttaccgtagcttctgggaaaaacttttgattttcgactataccccgagaaaa

SEQ ID NO: 370 - protein, 101 aa, synthetic construct
  REGION 1..101: note = Synthetic biopolymer
MAVKHLIVLKFLDEITEAQKEEFFKTFVNLVNIIPAMKDVYWGKDVTQRNKEEGYTHIVEVTFESVETIQDYIMHPAHVGFGDVYRSFWEKLLIFDYTPRK

SEQ ID NO: 371 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaaaaaacaaagaagaaggttacactcacatcgtcgaagtcactttcaaatcagtagagacgatccaagattacatagttcacccggcccatgtgggattcggagacgtttaccgtagcttctgggaaaaacttttgattttcgactataccccgagaaaa

SEQ ID NO: 372 - protein, 101 aa, synthetic construct
  REGION 1..101: note = Synthetic biopolymer
MGVKHLIVLKFKDEITEAQKEEFFKTFVNLVNIIPAMKDVYWGKDVTQKNKEEGYTHIVEVTFKSVETIQDYIVHPAHVGFGDVYRSFWEKLLIFDYTPRK

SEQ ID NO: 373 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgattcagtagagacgatccaagattacatagttcacccggcccatgtgggattcggagacgtttaccgtagcttctgggaaaaacttttgattttcgactataccccgagaaaa

SEQ ID NO: 374 - protein, 101 aa, synthetic construct
  REGION 1..101: note = Synthetic biopolymer
MAVKHLIVLKFKDEITEAQKEEFFKTFVNLVNIIPAMKDVYWGKDVTQRNKEEGYTHIVEVTFDSVETIQDYIVHPAHVGFGDVYRSFWEKLLIFDYTPRK

SEQ ID NO: 375 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgattcagtagagacgatccaagattacataactcacccggcccatgtgggattcggagacgtttaccgtagcttctgggaaaaacttttgattttcgactataccccgagaaaa

SEQ ID NO: 376 - protein, 101 aa, synthetic construct
  REGION 1..101: note = Synthetic biopolymer
MAVKHLIVLKFKDEITEAQKEEFFKTFVNLVNIIPAMKDVYWGKDVTQRNKEEGYTHIVEVTFDSVETIQDYITHPAHVGFGDVYRSFWEKLLIFDYTPRK

SEQ ID NO: 377 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgattcagtagagacgatccaagattacataatgcacccggcccatgtgggattcggagacgtttaccgtagcttctgggaaaaacttttgattttcgactataccccgagaaaa

SEQ ID NO: 378 - protein, 101 aa, synthetic construct
  REGION 1..101: note = Synthetic biopolymer
MGVKHLIVLKFKDEITEAQKEEFFKTYVNLVNIIPAMKDVYWGKDVTQRNKEEGYTHIVEVTFDSVETIQDYIMHPAHVGFGDVYRSFWEKLLIFDYTPRK

SEQ ID NO: 379 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgattcagtagagacgatccaagattacataaatcacccggcccatgtgggattcggagacgtttaccgtagcttctgggaaaaacttttgattttcgactataccccgagaaaa

SEQ ID NO: 380 - protein, 101 aa, synthetic construct
  REGION 1..101: note = Synthetic biopolymer
MGVKHLIVLKFKDEITEAQKEEFFKTYVNLVNIIPAMKDVYWGKDVTQRNKEEGYTHIVEVTFDSVETIQDYINHPAHVGFGDVYRSFWEKLLIFDYTPRK

SEQ ID NO: 381 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcaaatcagtagagacgatccaagattacataaatcacccggcccatgtgggattcggagacgtttaccgtagcttctgggaaaaacttttgattttcgactataccccgagaaaa

SEQ ID NO: 382 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFKSVETIQDYINHPAHVGFGDVYRSFWEKLLIFDYTPRK

SEQ ID NO: 383 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcgcgacctatgtaaacttagtgaatatcatccctgcgatgaaagatgtttattggggcaaagacgtcaccccaaaaaacaaagaagaaggttacactcacatcgtcgaagtcactttcgagtcagtagagacgatccaagattacataaatcacccggcccatgtgggattcggagacgtttaccgtagcttctgggaaaaacttttgattttcgactataccccgagaaaa

SEQ ID NO: 384 - protein, 101 aa, synthetic construct
  REGION 1..101: note = Synthetic biopolymer
MAVKHLIVVKFKDGITEAQKEEFFATYVNLVNIIPAMKDVYWGKDVTPKNKEEGYTHIVEVTFESVETIQDYINHPAHVGFGDVYRSFWEKLLIFDYTPRK

SEQ ID NO: 385 - DNA, 303 bp, synthetic construct
  misc_feature 1..303: note = Synthetic biopolymer
  source 1..303: mol_type = other DNA
atggctgtcaagcaccttatcgtactgaaattcttggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacatagttcacccggcccatgtgggattcggagacgtttaccgtagcttctgggaaaaacttttgattttcgactataccccgagaaaa

SEQ ID NO: 386 - protein, 101 aa, synthetic construct
  REGION 1..101: note = Synthetic biopolymer
MAVKHLIVLKFLDEITEAQKEEFFKTYVNLVNIIPAMKDVYWGKDVTQRNKEEGYTHIVEVTFESVETIQDYIVHPAHVGFGDVYRSFWEKLLIFDYTPRK

SEQ ID NO: 387 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgagtcagtagagacgatccaagattacatagttcacccggcccatgtgggattcggagacgtttaccgtagcttctgggaaaaacttttgattttcgactataccccgagaaaa

SEQ ID NO: 388 - protein, 101 aa, synthetic construct
  REGION 1..101: note = Synthetic biopolymer
MAVKHLIVLKFKDEITEAQKEEFFKTFVNLVNIIPAMKDVYWGKDVTQRNKEEGYTHIVEVTFESVETIQDYIVHPAHVGFGDVYRSFWEKLLIFDYTPRK

SEQ ID NO: 389 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacatatctcacccggcccatgtgggattcggagacgtttaccgtagcttctgggaaaaacttttgattttcgactataccccgagaaaa

SEQ ID NO: 390 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFESVETIQDYISHPAHVGFGDVYRSFWEKLLIFDYTPRK

SEQ ID NO: 391 - DNA, 303 bp, synthetic construct
  misc_feature 1..303: note = Synthetic biopolymer
  source 1..303: mol_type = other DNA
atgggtgtcaagcaccttatcgtactgaaattcttggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataaatcacccggcccatgtgggattcggagacgtttaccgtagcttctgggaaaaacttttgattttcgactataccccgagaaaa

SEQ ID NO: 392 - protein, 101 aa, synthetic construct
  REGION 1..101: note = Synthetic biopolymer
MGVKHLIVLKFLDEITEAQKEEFFKTYVNLVNIIPAMKDVYWGKDVTQRNKEEGYTHIVEVTFESVETIQDYINHPAHVGFGDVYRSFWEKLLIFDYTPRK

SEQ ID NO: 393 - DNA, 303 bp, synthetic construct
  misc_feature 1..303: note = Synthetic biopolymer
  source 1..303: mol_type = other DNA
atggccgtcaagcatttaatcgtcgtcaaatttaaggacggtatcacagaagctcaaaaggaggaattcttcaaaacctatgtaaacttagtgaacataattcctgctatgaaagatgtttattggggcaaagacgtcgcccaaaaaaacaaagaagaaggttacactcacatcgtcgaagtcactttcgattcagtagagacgatccaagattacatagttcacccggcccatgtgggattcggagacgtttaccgtagcttctgggaaaaacttttgattttcgactataccccgagaaaa

SEQ ID NO: 394 - protein, 101 aa, synthetic construct
  REGION 1..101: note = Synthetic biopolymer
MAVKHLIVVKFKDGITEAQKEEFFKTYVNLVNIIPAMKDVYWGKDVAQKNKEEGYTHIVEVTFDSVETIQDYIVHPAHVGFGDVYRSFWEKLLIFDYTPRK

SEQ ID NO: 395 - DNA, 303 bp, synthetic construct
  misc_feature 1..303: note = Synthetic biopolymer
  source 1..303: mol_type = other DNA
atggctgtcaagcaccttatcgtactgaaattcttggacgaaattacagaagcccagaaagaagaatttttcaagacctatgtaaacctggtgaatatcatccctgcgatgaaagatgtttattggggcaaagacgtcacccaaagaaacaaagaagaaggttacactcacatcgtcgaagtcactttcgattcagtagagacgatccaagattacatatctcacccggcccatgtgggattcggagacgtttaccgtagcttctgggaaaaacttttgattttcgactataccccgagaaaa

SEQ ID NO: 396 - protein, 101 aa, synthetic construct
  REGION 1..101: note = Synthetic biopolymer
MAVKHLIVLKFLDEITEAQKEEFFKTYVNLVNIIPAMKDVYWGKDVTQRNKEEGYTHIVEVTFDSVETIQDYISHPAHVGFGDVYRSFWEKLLIFDYTPRK

SEQ ID NO: 397 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctttgtaaacctggtgaatatcatccctgcgatgaaagatgtttattggggcaaagacgtcacccaaagaaacaaagaagaaggttacactcacatcgtcgaagtcactttcgagtcagtagagacgatccaagattacataaatcacccggcccatgtgggattcggagacgtttaccgtagcttctgggaaaaacttttgattttcgactataccccgagaaaa

SEQ ID NO: 398 - protein, 101 aa, synthetic construct
  REGION 1..101: note = Synthetic biopolymer
MAVKHLIVLKFKDEITEAQKEEFFKTFVNLVNIIPAMKDVYWGKDVTQRNKEEGYTHIVEVTFESVETIQDYINHPAHVGFGDVYRSFWEKLLIFDYTPRK

SEQ ID NO: 399 - DNA, 303 bp, synthetic construct
  misc_feature 1..303: note = Synthetic biopolymer
  source 1..303: mol_type = other DNA
atgggtgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcaaatcagtagagacgatccaagattacataatgcacccggcccatgtgggattcggagacgtttaccgtagcttctgggaaaaacttttgattttcgactataccccgagaaaa

SEQ ID NO: 400 - protein, 101 aa, synthetic construct
  REGION 1..101: note = Synthetic biopolymer
MGVKHLIVLKFKDEITEAQKEEFFKTYVNLVNIIPAMKDVYWGKDVTQKNKEEGYTHIVEVTFKSVETIQDYIMHPAHVGFGDVYRSFWEKLLIFDYTPRK

SEQ ID NO: 401 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttactcacacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 402 - protein, 101 aa, synthetic construct
  REGION 1..101: note = Synthetic biopolymer
MAVKHLIIIKFKDEITEAQKEEFFKTYVNLVNVIPAMKDVYWGKDVTQRNKEEGYSHIVEVTFESVETIQTYIIHPAHVGFGDVYRSFWEKLLIFDYTPRK

SEQ ID NO: 403 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgacatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 404 - protein, 101 aa, synthetic construct
  REGION 1..101: note = Synthetic biopolymer
MAVKHLIIIKFKDEITEAQKEEFFKTYVNLVNDIPAMKDVYWGKDVTQRNKEEGYSHVVEVTFESVETIQDYIIHPAHVGFGDVYRSFWEKLLIFDYTPRK

SEQ ID NO: 405 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattacagaagcccagaaagaagaatttttcaagacctatgtaaacctggtgaatatcatccctgcgatgaaagatgtttattggggcaaagacgtcacccaacgtaacaaagaagaaggttggtcacacatcgtcgaagtcactttcgagtcagtagagacgatccaagattacataatacacccggcccatgtgggattcggagacgtttaccgtagcttctgggaaaaacttttgattttcgactataccccgagaaag

SEQ ID NO: 406 - protein, 101 aa, synthetic construct
  REGION 1..101: note = Synthetic biopolymer
MAVKHLIIIKFNDEITEAQKEEFFKTYVNLVNIIPAMKDVYWGKDVTQRNKEEGWSHIVEVTFESVETIQDYIIHPAHVGFGDVYRSFWEKLLIFDYTPRK

SEQ ID NO: 407 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatgtcatccctgcgatgaaagatgtttattggggcaaagacgtcacccaaaaaaacaaagaagaaggttactcacacatcgtcgaagtcactttcgactcagtagagacgatccaagattacataatacacccggcccatgtgggattcggagacgtttaccgtagcttctgggaaaaacttttgattttcgactataccccgagaaaa

SEQ ID NO: 408 - protein, 101 aa, synthetic construct
  REGION 1..101: note = Synthetic biopolymer
MAVKHLIIIKFVDEITEAQKEEFFKTYVNLVNVIPAMKDVYWGKDVTQKNKEEGYSHIVEVTFDSVETIQDYIIHPAHVGFGDVYRSFWEKLLIFDYTPRK

SEQ ID NO: 409 - DNA, 303 bp, synthetic construct
  misc_feature 1..303: note = Synthetic biopolymer
  source 1..303: mol_type = other DNA
atggctgtaaagcaccttatcgtaatcaaattcgtcgacgaaattacagaagcccagaaagaagaatttttcaagacctatgtaaacctggtgaatgtcatccctgcgatgaaagatgtttattggggcaaagacgtcacccaaaaaaacaaagaagaaggttactcacacgtcgtcgaagtcactttcgactcagtagagacgatccaagattacataatacacccggctcatgtgggattcggagacgtttaccgtagcttctgggaaaaacttttgattttcgactataccccgagaaaa

SEQ ID NO: 410 - protein, 101 aa, synthetic construct
  REGION 1..101: note = Synthetic biopolymer
MAVKHLIVIKFVDEITEAQKEEFFKTYVNLVNVIPAMKDVYWGKDVTQKNKEEGYSHVVEVTFDSVETIQDYIIHPAHVGFGDVYRSFWEKLLIFDYTPRK

SEQ ID NO: 411 - DNA, 303 bp, synthetic construct
  misc_feature 1..303: note = Synthetic biopolymer
  source 1..303: mol_type = other DNA
atgccggtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 412 - protein, 101 aa, synthetic construct
  REGION 1..101: note = Synthetic biopolymer
MPVKHLIIIKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 413 - DNA, 303 bp, synthetic construct
  misc_feature 1..303: note = Synthetic biopolymer
  source 1..303: mol_type = other DNA
atggctgtcaagcaccttatcatcctgaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgactcagtagagacgatccaagattacataatacacccggcccatgtgggattcggagacgtttaccgtagcttctgggaaaaacttttgattttcgacttcaccccgagaaaa

SEQ ID NO: 414 - protein, 101 aa, synthetic construct
  REGION 1..101: note = Synthetic biopolymer
MAVKHLIILKFVDEITEAQKEEFFKTYVNLVNIIPAMKDVYWGKDVTQKNKEEGYSHVVEVTFDSVETIQDYIIHPAHVGFGDVYRSFWEKLLIFDFTPRK

SEQ ID NO: 415 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctgatgaatatcatccctgcgatgaaagatgtttattggggcaaagacgtcacccaacgtaacaaagaagaaggttacactcacgtcgtcgaagtctgtttcgactcagtagaggccatccaagattacataatacacccggcccatgtgggattcggagacgtttaccgtagcttctgggaaaaacttttgattttcgactataccccgagaaaa

SEQ ID NO: 416 - protein, 101 aa, synthetic construct
  REGION 1..101: note = Synthetic biopolymer
MAVKHLIVLKFKDEITEAQKEEFFKTYVNLMNIIPAMKDVYWGKDVTQRNKEEGYTHVVEVCFDSVEAIQDYIIHPAHVGFGDVYRSFWEKLLIFDYTPRK

SEQ ID NO: 417 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 418 - protein, 101 aa, synthetic construct
  REGION 1..101: note = Synthetic biopolymer
MAVKHLIILKFKDEITEAQKEEFFKTYVNLVNIIPAMKDVYWGKDVTQRNKEEGYTHVVEVTFDSVEMIQDYIMHPAHVGFGDVYRSFWEKLLIFDYTPRK

SEQ ID NO: 419 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatatcatccctccgatgaaagatgtttattggggcaaagacgtcacccaaaaaaacaaagaagaaggttacactcacgtcgtcgaagtcactttcgactcagtagagggaatcaaagattacataatgcacccggcccatgtgggattcggagacgtttaccgtagcttctgggaaaaacttttgattttcgactataccccgagaaaa

SEQ ID NO: 420 - protein, 101 aa, synthetic construct
  REGION 1..101: note = Synthetic biopolymer
MAVKHLIVLKFKDEITEAQKEEFFKTYVNLVNIIPPMKDVYWGKDVTQKNKEEGYTHVVEVTFDSVEGIKDYIMHPAHVGFGDVYRSFWEKLLIFDYTPRK

SEQ ID NO: 421 - DNA, 303 bp, synthetic construct
  misc_feature 1..303: note = Synthetic biopolymer
  source 1..303: mol_type = other DNA
atggctgtaaagcaccttatcgtactgaaattcaaggacggaattacagaagcccagaaagaagaatttttcaagacctatgtaaacctggtgaatatcatccctcaaatgaaagatgtttattggggcaaagacgtcacccaacgtaacaaagaagaaggttacactcacgtcgtcgaagtcactttcgactcagtagagacgatcaaagattacataatacacccggcccatgtgggattcggagacgtttaccgtagcttctgggaaaaacttttgattttcgactataccccgagaaaa

SEQ ID NO: 422 - protein, 101 aa, synthetic construct
  REGION 1..101: note = Synthetic biopolymer
MAVKHLIVLKFKDGITEAQKEEFFKTYVNLVNIIPQMKDVYWGKDVTQRNKEEGYTHVVEVTFDSVETIKDYIIHPAHVGFGDVYRSFWEKLLIFDYTPRK

SEQ ID NO: 423 - DNA, 25 bp, synthetic construct
  misc_feature 1..25: note = Synthetic biopolymer
  source 1..25: mol_type = other DNA
ctaagtctagccacgaaaactgcaa

SEQ ID NO: 424 - DNA, 30 bp, synthetic construct
  misc_feature 1..30: note = Synthetic biopolymer
  source 1..30: mol_type = other DNA
ggttatgaagaggaaaaattggcagtaacc

SEQ ID NO: 425 - DNA, 30 bp, synthetic construct
  misc_feature 1..30: note = Synthetic biopolymer
  source 1..30: mol_type = other DNA
gaacgaatcaaattaacaaccataggatga

SEQ ID NO: 426 - DNA, 28 bp, synthetic construct
  misc_feature 1..28: note = Synthetic biopolymer
  source 1..28: mol_type = other DNA
gcaccaaaagtaagaaacgacaaagttt

SEQ ID NO: 427 - DNA, 50 bp, synthetic construct
  misc_feature 1..50: note = Synthetic biopolymer
  source 1..50: mol_type = other DNA
caaaaaattgttaatatacctctatactttaacgtcaaggagaaaaaacc

SEQ ID NO: 428 - DNA, 50 bp, synthetic construct
  misc_feature 1..50: note = Synthetic biopolymer
  source 1..50: mol_type = other DNA
taaattgaattgaattgaaatcgatagatcaatttttttcttttctcttt

SEQ ID NO: 429 - DNA, 667 bp, synthetic construct
  misc_feature 1..667: note = Synthetic biopolymer
  source 1..667: mol_type = other DNA
ttttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaacc

SEQ ID NO: 430 - DNA, 225 bp, synthetic construct
  misc_feature 1..225: note = Synthetic biopolymer
  source 1..225: mol_type = other DNA
attgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgt

SEQ ID NO: 431 - DNA, 42 bp, synthetic construct
  misc_feature 1..42: note = Synthetic biopolymer
  source 1..42: mol_type = other DNA
tacgataaggtgcttgacacccatggttttttctccttgacg

SEQ ID NO: 432 - DNA, 42 bp, synthetic construct
  misc_feature 1..42: note = Synthetic biopolymer
  source 1..42: mol_type = other DNA
ttcttctttctgggcttcttgaatttcgtccttgaatttcag

SEQ ID NO: 433 - DNA, 42 bp, synthetic construct
  misc_feature 1..42: note = Synthetic biopolymer
  source 1..42: mol_type = other DNA
gacgatgtgagtgtaacctctttctttgtttttttgggtgac

SEQ ID NO: 434 - DNA, 42 bp, synthetic construct
  misc_feature 1..42: note = Synthetic biopolymer
  source 1..42: mol_type = other DNA
cgtctctactgactcgaaaccgacttcgacgatgtgagtgta

SEQ ID NO: 435 - DNA, 42 bp, synthetic construct
  misc_feature 1..42: note = Synthetic biopolymer
  source 1..42: mol_type = other DNA
tattatgtaatcttggatttcctctactgactcgaaagtgac

SEQ ID NO: 436 - DNA, 42 bp, synthetic construct
  misc_feature 1..42: note = Synthetic biopolymer
  source 1..42: mol_type = other DNA
tcccacatgggccgggtgcattatgtaatcttggatcgtctc

SEQ ID NO: 437 - DNA, 42 bp, synthetic construct
  misc_feature 1..42: note = Synthetic biopolymer
  source 1..42: mol_type = other DNA
ttcccagaagctacggtaaatgtctccgaatcccacatgggc

SEQ ID NO: 438 - DNA, 42 bp, synthetic construct
  misc_feature 1..42: note = Synthetic biopolymer
  source 1..42: mol_type = other DNA
ccaataaacatctttcatttcagggatgatattcaccaggtt

SEQ ID NO: 439 - DNA, 42 bp, synthetic construct
  misc_feature 1..42: note = Synthetic biopolymer
  source 1..42: mol_type = other DNA
gacgatgtgagtgtaaccaaattctttgtttttttgggtgac

SEQ ID NO: 440 - DNA, 42 bp, synthetic construct
  misc_feature 1..42: note = Synthetic biopolymer
  source 1..42: mol_type = other DNA
ttggatcgtctctactgatttgaaagtgacttcgacgatgtg

SEQ ID NO: 441 - DNA, 42 bp, synthetic construct
  misc_feature 1..42: note = Synthetic biopolymer
  source 1..42: mol_type = other DNA
tattatgtaatcttggatagactctactgactcgaaagtgac

SEQ ID NO: 442 - DNA, 42 bp, synthetic construct
  misc_feature 1..42: note = Synthetic biopolymer
  source 1..42: mol_type = other DNA
tcccacatgggccgggtgacctatgtaatcttggatcgtctc

SEQ ID NO: 443 - DNA, 42 bp, synthetic construct
  misc_feature 1..42: note = Synthetic biopolymer
  source 1..42: mol_type = other DNA
ttcccagaagctacggtacatgtctccgaatcccacatgggc

SEQ ID NO: 444 - DNA, 42 bp, synthetic construct
  misc_feature 1..42: note = Synthetic biopolymer
  source 1..42: mol_type = other DNA
aatttcgtccttgaatttaactacgataaggtgcttgacagc

SEQ ID NO: 445 - DNA, 42 bp, synthetic construct
  misc_feature 1..42: note = Synthetic biopolymer
  source 1..42: mol_type = other DNA
agggatgatattcaccagacctacataggtcttgaaaaattc

SEQ ID NO: 446 - DNA, 42 bp, synthetic construct
  misc_feature 1..42: note = Synthetic biopolymer
  source 1..42: mol_type = other DNA
gacgatgtgagtgtaaccagattctttgtttttttgggtgac

SEQ ID NO: 447 - DNA, 42 bp, synthetic construct
  misc_feature 1..42: note = Synthetic biopolymer
  source 1..42: mol_type = other DNA
ttggatcgtctctactgaatcgaaagtgacttcgacgatgtg

SEQ ID NO: 448 - DNA, 42 bp, synthetic construct
  misc_feature 1..42: note = Synthetic biopolymer
  source 1..42: mol_type = other DNA
ggccgggtgtattatgtaaccttggatcgtctctactgactc

SEQ ID NO: 449 - DNA, 42 bp, synthetic construct
  misc_feature 1..42: note = Synthetic biopolymer
  source 1..42: mol_type = other DNA
caaaagtttttcccagaatttacggtaaacgtctccgaatcc

SEQ ID NO: 450 - DNA, 42 bp, synthetic construct
  misc_feature 1..42: note = Synthetic biopolymer
  source 1..42: mol_type = other DNA
aatttcgtccttgaatttaattacgataaggtgcttgacagc

SEQ ID NO: 451 - DNA, 42 bp, synthetic construct
  misc_feature 1..42: note = Synthetic biopolymer
  source 1..42: mol_type = other DNA
catcgcagggatgatattagacaggtttacataggtcttgaa

SEQ ID NO: 452 - DNA, 42 bp, synthetic construct
  misc_feature 1..42: note = Synthetic biopolymer
  source 1..42: mol_type = other DNA
gtttttttgggtgacgtctgggccccaataaacatctttcat

SEQ ID NO: 453 - DNA, 42 bp, synthetic construct
  misc_feature 1..42: note = Synthetic biopolymer
  source 1..42: mol_type = other DNA
gacgatgtgagtgtaacccaattctttgtttttttgggtgac

SEQ ID NO: 454 - DNA, 42 bp, synthetic construct
  misc_feature 1..42: note = Synthetic biopolymer
  source 1..42: mol_type = other DNA
gtaatcttggatcgtctccaatgactcgaaagtgacttcgac

SEQ ID NO: 455 - DNA, 42 bp, synthetic construct
  misc_feature 1..42: note = Synthetic biopolymer
  source 1..42: mol_type = other DNA
tcccacatgggccgggtgatttatgtaatcttggatcgtctc

SEQ ID NO: 456 - DNA, 42 bp, synthetic construct
  misc_feature 1..42: note = Synthetic biopolymer
  source 1..42: mol_type = other DNA
atagtcgaaaatcaaaagttcttcccagaagctacggtaaac

SEQ ID NO: 457 - DNA, 42 bp, synthetic construct
  misc_feature 1..42: note = Synthetic biopolymer
  source 1..42: mol_type = other DNA
caccaggtttacataggtagagaaaaattcttctttctgggc

SEQ ID NO: 458 - DNA, 42 bp, synthetic construct
  misc_feature 1..42: note = Synthetic biopolymer
  source 1..42: mol_type = other DNA
catcgcagggatgatattcatcaggtttacataggtcttgaa

SEQ ID NO: 459 - DNA, 42 bp, synthetic construct
  misc_feature 1..42: note = Synthetic biopolymer
  source 1..42: mol_type = other DNA
gacgatgtgagtgtaaccatgttctttgtttttttgggtgac

SEQ ID NO: 460 - DNA, 42 bp, synthetic construct
  misc_feature 1..42: note = Synthetic biopolymer
  source 1..42: mol_type = other DNA
tattatgtaatcttggatttgctctactgactcgaaagtgac

SEQ ID NO: 461 - DNA, 42 bp, synthetic construct
  misc_feature 1..42: note = Synthetic biopolymer
  source 1..42: mol_type = other DNA
tcccacatgggccgggtgaactatgtaatcttggatcgtctc

SEQ ID NO: 462 - DNA, 42 bp, synthetic construct
  misc_feature 1..42: note = Synthetic biopolymer
  source 1..42: mol_type = other DNA
tctcggggtatagtcgaatttcaaaagtttttcccagaagct

SEQ ID NO: 463 - DNA, 42 bp, synthetic construct
  misc_feature 1..42: note = Synthetic biopolymer
  source 1..42: mol_type = other DNA
ggcttctgtaatttcgtccaagaatttcagtacgataaggtg

SEQ ID NO: 464 - DNA, 42 bp, synthetic construct
  misc_feature 1..42: note = Synthetic biopolymer
  source 1..42: mol_type = other DNA
caccaggtttacataggtaccgaaaaattcttctttctgggc

SEQ ID NO: 465 - DNA, 42 bp, synthetic construct
  misc_feature 1..42: note = Synthetic biopolymer
  source 1..42: mol_type = other DNA
catcgcagggatgatattttccaggtttacataggtcttgaa

SEQ ID NO: 466 - DNA, 42 bp, synthetic construct
  misc_feature 1..42: note = Synthetic biopolymer
  source 1..42: mol_type = other DNA
gtaaccttcttctttgtttctttgggtgacgtctttgcccca

SEQ ID NO: 467 - DNA, 42 bp, synthetic construct
  misc_feature 1..42: note = Synthetic biopolymer
  source 1..42: mol_type = other DNA
gacgatgtgagtgtaaccagcttctttgtttttttgggtgac

SEQ ID NO: 468 - DNA, 42 bp, synthetic construct
  misc_feature 1..42: note = Synthetic biopolymer
  source 1..42: mol_type = other DNA
tattatgtaatcttggatagcctctactgactcgaaagtgac

SEQ ID NO: 469 - DNA, 42 bp, synthetic construct
  misc_feature 1..42: note = Synthetic biopolymer
  source 1..42: mol_type = other DNA
tcccacatgggccgggtgagttatgtaatcttggatcgtctc

SEQ ID NO: 470 - DNA, 42 bp, synthetic construct
  misc_feature 1..42: note = Synthetic biopolymer
  source 1..42: mol_type = other DNA
tttctgggcttctgtaataccgtccttgaatttcagtacgat

SEQ ID NO: 471 - DNA, 42 bp, synthetic construct
  misc_feature 1..42: note = Synthetic biopolymer
  source 1..42: mol_type = other DNA
caccaggtttacataggttctgaaaaattcttctttctgggc

SEQ ID NO: 472 - DNA, 42 bp, synthetic construct
  misc_feature 1..42: note = Synthetic biopolymer
  source 1..42: mol_type = other DNA
gatgtgagtgtaaccttcttgtttgtttttttgggtgacgtc

SEQ ID NO: 473 - DNA, 42 bp, synthetic construct
  misc_feature 1..42: note = Synthetic biopolymer
  source 1..42: mol_type = other DNA
tattatgtaatcttggataccctctactgactcgaaagtgac

SEQ ID NO: 474 - DNA, 42 bp, synthetic construct
  misc_feature 1..42: note = Synthetic biopolymer
  source 1..42: mol_type = other DNA
tcccacatgggccgggtgagatatgtaatcttggatcgtctc

SEQ ID NO: 475 - DNA, 42 bp, synthetic construct
  misc_feature 1..42: note = Synthetic biopolymer
  source 1..42: mol_type = other DNA
ttcttctttctgggcttctggaatttcgtccttgaatttcag

SEQ ID NO: 476 - DNA, 42 bp, synthetic construct
  misc_feature 1..42: note = Synthetic biopolymer
  source 1..42: mol_type = other DNA
gatgtgagtgtaaccttctcttttgtttttttgggtgacgtc

SEQ ID NO: 477 - DNA, 42 bp, synthetic construct
  misc_feature 1..42: note = Synthetic biopolymer
  source 1..42: mol_type = other DNA
gaaagtgacttcgacgataccagtgtaaccttcttctttgtt

SEQ ID NO: 478 - DNA, 42 bp, synthetic construct
  misc_feature 1..42: note = Synthetic biopolymer
  source 1..42: mol_type = other DNA
tattatgtaatcttggatcatctctactgactcgaaagtgac

SEQ ID NO: 479 - DNA, 42 bp, synthetic construct; identical to SEQ ID NO: 431
  misc_feature 1..42: note = Synthetic biopolymer
  source 1..42: mol_type = other DNA

SEQ ID NO: 480 - DNA, 30 bp, synthetic construct; identical to SEQ ID NO: 424
  misc_feature 1..30: note = Synthetic biopolymer
  source 1..30: mol_type = other DNA

SEQ ID NO: 481 - DNA, 60 bp, synthetic construct
  misc_feature 1..60: note = Synthetic biopolymer
  source 1..60: mol_type = other DNA
ctttaacactatcaagtgctttacagccatggttttttctccttgacgttaaagtataga

SEQ ID NO: 482 - DNA, 32 bp, synthetic construct
  misc_feature 1..32: note = Synthetic biopolymer
  source 1..32: mol_type = other DNA
ggaaacctctacacatagaaatatcgaatggg

SEQ ID NO: 483 - DNA, 60 bp, synthetic construct
  misc_feature 1..60: note = Synthetic biopolymer
  source 1..60: mol_type = other DNA
ttgttaatttttgattacactccaaggaagtaaattgaattgaattgaaatcgatagatc

SEQ ID NO: 484 - DNA, 30 bp, synthetic construct
  misc_feature 1..30: note = Synthetic biopolymer
  source 1..30: mol_type = other DNA
gaggagcgaatttttttttaataaaaatct

SEQ ID NO: 485 - DNA, 303 bp, synthetic construct
  misc_feature 1..303: note = Synthetic biopolymer
  source 1..303: mol_type = other DNA
atgccggtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaaaaaaacaaagaagaaggttacactcacatcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 486 - protein, 101 aa, synthetic construct
  REGION 1..101: note = Synthetic biopolymer
MPVKHLIIIKFKDEITEAQKEEFFKTYVNLVNVIPAMKDVYWGKDVTQKNKEEGYTHIVEVTFDSVETIQDYIIHPAHVGFGDVYRSFWEKLLKFDYTPRK

SEQ ID NO: 487 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatcccttcaatgaaagatgtttattggggcaaagacgtcacccaaaaaaacaaagaagaaggttggactcacatcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 488 - protein, 101 aa, synthetic construct
  REGION 1..101: note = Synthetic biopolymer
MAVKHLIIIKFKDEITEAQKEEFFKTYVNLVNIIPSMKDVYWGKDVTQKNKEEGWTHIVEVTFDSVETIQDYIIHPAHVGFGDVYRSFWEKLLKFDYTPRK

SEQ ID NO: 489 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttacactcacgtcgtcgaagtctgtttcgagtcagtagagacgatccaagattacataatacacccggcccatgtgggattcggagacgtttaccgtagcttctgggaaaaacttttgattttcgactataccccgagaaaa

SEQ ID NO: 490 - protein, 101 aa, synthetic construct
  REGION 1..101: note = Synthetic biopolymer
MAVKHLIIIKFKDEITEAQKEEFFKTYVNLVNVIPAMKDVYWGKDVTQRNKEEGYTHVVEVCFESVETIQDYIIHPAHVGFGDVYRSFWEKLLIFDYTPRK

SEQ ID NO: 491 - DNA, 303 bp, synthetic construct; identical to SEQ ID NO: 489
  misc_feature 1..303: note = Synthetic biopolymer
  source 1..303: mol_type = other DNA

SEQ ID NO: 492 - protein, 101 aa, synthetic construct; identical to SEQ ID NO: 490
  REGION 1..101: note = Synthetic biopolymer

SEQ ID NO: 493 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgacatccctgcgatgaaagatgtttattggggcaaagacgtcacccaacgtaacaaagaagaaggttactcacacatcgtcgaagtcactttcgagtcagtagagacgatccaaacctacataatacacccggcccatgtgggattcggagacgtttaccgtagcttctgggaaaaacttttgatttttgattacactccaaggaag

SEQ ID NO: 494 - protein, 101 aa, synthetic construct
  REGION 1..101: note = Synthetic biopolymer
MAVKHLIIIKFKDEITEAQKEEFFKTYVNLVNDIPAMKDVYWGKDVTQRNKEEGYSHIVEVTFESVETIQTYIIHPAHVGFGDVYRSFWEKLLIFDYTPRK

SEQ ID NO: 495 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 496 - protein, 101 aa, synthetic construct
  REGION 1..101: note = Synthetic biopolymer
MAVKHLIIIKFNDEITEAQKEEFFKTYVNLVNVIPAMKDVYWGKDVTQRNKEEGYTHIVEVTFESVETIQTYIIHPAHVGFGDVYRSFWEKLLIFDYTPRK

SEQ ID NO: 497 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 498 - protein, 101 aa, synthetic construct
  REGION 1..101: note = Synthetic biopolymer
MAVKHLIIIKFKDEITEAQKEEFFKTYVNLVNVIPAMKDVYWGKDVTQRNKEEGYTHVVEVTFESVETIQDYIIHPAHVGFGDVYRSFWEKLLIFDYTPGK

SEQ ID NO: 499 - DNA, 303 bp, synthetic construct
  misc_feature 1..303: note = Synthetic biopolymer
  source 1..303: mol_type = other DNA
atggctgtaaagcaccttatcatcatcaaattcaacgacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtcactttcgagtcagtagagacgatccaaacctacataatacacccggcccatgtgggattcggagacgtttaccgtagcttctgggaaaaacttttgatttttgattacactccaaggaag

SEQ ID NO: 500 - protein, 101 aa, synthetic construct; identical to SEQ ID NO: 496
  REGION 1..101: note = Synthetic biopolymer

SEQ ID NO: 501 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttggactcacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 502 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGWTHVVEVTFESVETIQDYIIHPAHVGFGDVYRSFWEKLLIFDYTPGK

SEQ ID NO: 503 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttactcacacatcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 504 - protein, 101 aa, synthetic construct
  REGION 1..101: note = Synthetic biopolymer
MAVKHLIIIKFKDEITEAQKEEFFKTYVNLVNVIPAMKDVYWGKDVTQRNKEEGYSHIVEVTFESVETIQDYIIHPAHVGFGDVYRSFWEKLLIFDYTPGK

SEQ ID NO: 505 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctttaatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 506 - protein, 101 aa, synthetic construct
  REGION 1..101: note = Synthetic biopolymer
MAVKHLIIIKFKDEITEAQKEEFFKTYVNLVNVIPLMKDVYWGKDVTQRNKEEGYTHVVEVTFESVETIQDYIIHPAHVGFGDVYRSFWEKLLIFDYTPRK

SEQ ID NO: 507 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattacagaagcccagaaagaagaatttttcaagacctatgtaaacctggtgaatgtcatccctcaaatgaaagatgtttattggggcaaagacgtcacccaaaaaaacaaagaagaaggttacactcacgtcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 508 - protein, 101 aa, synthetic construct
  REGION 1..101: note = Synthetic biopolymer
MAVKHLIVIKFKDEITEAQKEEFFKTYVNLVNVIPQMKDVYWGKDVTQKNKEEGYTHVVEVTFDSVETIQDYIIHPAHVGFGDVYRSFWEKLLKFDYTPRK

SEQ ID NO: 509 - DNA, 303 bp, synthetic construct
  misc_feature 1..303: note = Synthetic biopolymer
  source 1..303: mol_type = other DNA
atggctgtcaagcaccttatcatcatcgccttcaaggacgaaattacagaagcccagaaagaagaatttttcaagacctatgtaaacctggtgaatgtcatccctgcgatgaaagatgtttattggggcaaagacgtcacccaaaaaaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 510 - protein, 101 aa, synthetic construct
  REGION 1..101: note = Synthetic biopolymer
MAVKHLIIIAFKDEITEAQKEEFFKTYVNLVNVIPAMKDVYWGKDVTQKNKEEGYSHVVEVTFESVETIQDYIIHPAHVGFGDVYRSFWEKLLIFDYTPRK

SEQ ID NO: 511 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 512 - protein, 101 aa, synthetic construct
  REGION 1..101: note = Synthetic biopolymer
MAVKHLIVIKFKDEITEAQKEEFFKTYVNLVNVIPAMKDVYWGKDVTQRNKEEGYSHVVEVTFESVETIQDYIIHPAHVGFGDVYRSFWEKLLIFDYTPGK

SEQ ID NO: 513 - DNA, 303 bp, synthetic construct
  misc_feature 1..303: note = Synthetic biopolymer
  source 1..303: mol_type = other DNA
atggctgtaaagcaccttatcatcatcaaattcaacgacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 514 - protein, 101 aa, synthetic construct; identical to SEQ ID NO: 496
  REGION 1..101: note = Synthetic biopolymer

SEQ ID NO: 515 - DNA, 303 bp, synthetic construct; identical to SEQ ID NO: 507
  misc_feature 1..303: note = Synthetic biopolymer
  source 1..303: mol_type = other DNA

SEQ ID NO: 516 - protein, 101 aa, synthetic construct; identical to SEQ ID NO: 508
  REGION 1..101: note = Synthetic biopolymer

SEQ ID NO: 517 - DNA, 303 bp, synthetic construct
  misc_feature 1..303: note = Synthetic biopolymer
  source 1..303: mol_type = other DNA
atggctgtcaagcaccttatcatcatcgccttcaaggacgaaattacagaagcccagaaagaagaatttttcaagacctatgtagacctggtgaatgtcatccctgcgatgaaagatgtttattggggcaaagacgtcacccaaaaaaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 518 - protein, 101 aa, synthetic construct
  REGION 1..101: note = Synthetic biopolymer
MAVKHLIIIAFKDEITEAQKEEFFKTYVDLVNVIPAMKDVYWGKDVTQKNKEEGYTHVVEVTFESVETIQDYIIHPAHVGFGDVYRSFWEKLLIFDYTPRK

SEQ ID NO: 519 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 520 - protein, 101 aa, synthetic construct; identical to SEQ ID NO: 512
  REGION 1..101: note = Synthetic biopolymer

SEQ ID NO: 521 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgacatccctgcgatgaaagatgtttattggggcaaagacgtcacccaaaaaaacaaagaagaaggttactcacacgtcgtcgaagtcactttcgactcagtagagacgatccaagattacataatacacccggcccatgtgggattcggagacgtttaccgtagcttctgggaaaaacttttgattttcgactataccccgagaaaa

SEQ ID NO: 522 - protein, 101 aa, synthetic construct
  REGION 1..101: note = Synthetic biopolymer
MAVKHLIIIKFKDEITEAQKEEFFKTYVNLVNDIPAMKDVYWGKDVTQKNKEEGYSHVVEVTFDSVETIQDYIIHPAHVGFGDVYRSFWEKLLIFDYTPRK

SEQ ID NO: 523 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgacatccctgcgatgaaagatgtttattggggcaaagacgtcacccaacgtaacaaagaagaaggttactcacacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 524 - protein, 101 aa, synthetic construct; identical to SEQ ID NO: 494
  REGION 1..101: note = Synthetic biopolymer

SEQ ID NO: 525 - DNA, 303 bp, synthetic construct; identical to SEQ ID NO: 523
  misc_feature 1..303: note = Synthetic biopolymer
  source 1..303: mol_type = other DNA

SEQ ID NO: 526 - protein, 101 aa, synthetic construct; identical to SEQ ID NO: 494
  REGION 1..101: note = Synthetic biopolymer

SEQ ID NO: 527 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctcaaatgaaagatgtttattggggcaaagacgtcacccaaaaaaacaaagaagaaggttggactcacgtcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 528 - protein, 101 aa, synthetic construct
  REGION 1..101: note = Synthetic biopolymer
MAVKHLIVIKFKDEITEAQKEEFFKTYVNLVNIIPQMKDVYWGKDVTQKNKEEGWTHVVEVTFDSVETIQDYIIHPAHVGFGDVYRSFWEKLLKFDYTPRK

SEQ ID NO: 529 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaaaaaaacaaagaagaaggttactcacacgtcgtcgaagtcactttcgagtcagtagagacgatcaaagattacataatacacccggcccatgtgggattcggagacgtttaccgtagcttctgggaaaaacttttgattttcgactataccccgagaaaa

SEQ ID NO: 530 - protein, 101 aa, synthetic construct
  REGION 1..101: note = Synthetic biopolymer
MAVKHLIIIKFKDEITEAQKEEFFKTYVNLVNVIPAMKDVYWGKDVTQKNKEEGYSHVVEVTFESVETIKDYIIHPAHVGFGDVYRSFWEKLLIFDYTPRK

SEQ ID NO: 531 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttggtcacacatcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 532 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGWSHIVEVTFESVETIQDYIIHPAHVGFGDVYRSFWEKLLIFDYTPGK

SEQ ID NO: 533 - DNA, 303 bp, synthetic construct; identical to SEQ ID NO: 523
  misc_feature 1..303: note = Synthetic biopolymer
  source 1..303: mol_type = other DNA

SEQ ID NO: 534 - protein, 101 aa, synthetic construct; identical to SEQ ID NO: 494
  REGION 1..101: note = Synthetic biopolymer

SEQ ID NO: 535 - DNA, 303 bp, synthetic construct
  misc_feature 1..303: note = Synthetic biopolymer
  source 1..303: mol_type = other DNA
atggctgtcaagcaccttatcatcatcgccttcaaggacgaaattacagaagcccagaaagaagaatttttcaagacctatgtagacctggtgaatgtcatccctgcgatgaaagatgtttattggggcaaagacgtcacccaaaaaaacaaagaagaaggttactcacacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 536 - protein, 101 aa, synthetic construct
  REGION 1..101: note = Synthetic biopolymer
MAVKHLIIIAFKDEITEAQKEEFFKTYVDLVNVIPAMKDVYWGKDVTQKNKEEGYSHIVEVTFESVETIQDYIIHPAHVGFGDVYRSFWEKLLIFDYTPRK

SEQ ID NO: 537 - DNA, 303 bp, synthetic construct; identical to SEQ ID NO: 535
  misc_feature 1..303: note = Synthetic biopolymer
  source 1..303: mol_type = other DNA

SEQ ID NO: 538 - protein, 101 aa, synthetic construct; identical to SEQ ID NO: 536
  REGION 1..101: note = Synthetic biopolymer

SEQ ID NO: 539 - DNA, 303 bp, synthetic construct
  misc_feature 1..303: note = Synthetic biopolymer
  source 1..303: mol_type = other DNA
atgccggtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttggactcacatcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 540 - protein, 101 aa, synthetic construct
  REGION 1..101: note = Synthetic biopolymer
MPVKHLIIIKFKDEITEAQKEEFFKTYVNLVNIIPAMKDVYWGKDVTQKNKEEGWTHIVEVTFDSVETIQDYIIHPAHVGFGDVYRSFWEKLLKFDYTPRK

SEQ ID NO: 541 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattacagaagcccagaaagaagaatttttcaagacctatgtaaacctggtgaatatcatccctcaaatgaaagatgtttattggggcaaagacgtcacccaaaaaaacaaagaagaaggttggactcacgtcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 542 - protein, 101 aa, synthetic construct; identical to SEQ ID NO: 528
  REGION 1..101: note = Synthetic biopolymer

SEQ ID NO: 543 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatgtcatccctgcgatgaaagatgtttattggggcaaagacgtcacccaaaaaaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 544 - protein, 101 aa, synthetic construct
  REGION 1..101: note = Synthetic biopolymer
MAVKHLIIIKFKDEITEAQKEEFFKTYVNLVNVIPAMKDVYWGKDVTQKNKEEGYSHVVEVTFESVETIQDYIIHPAHVGFGDVYRSFWEKLLIFDYTPGK

SEQ ID NO: 545 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttggtcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 546 - protein, 101 aa, synthetic construct
  REGION 1..101: note = Synthetic biopolymer
MAVKHLIIIKFKDEITEAQKEEFFKTYVNLVNIIPAMKDVYWGKDVTQKNKEEGWSHVVEVTFESVETIQDYIIHPAHVGFGDVYRSFWEKLLIFDYTPGK

SEQ ID NO: 547 - DNA, 303 bp, synthetic construct; identical to SEQ ID NO: 527
  misc_feature 1..303: note = Synthetic biopolymer
  source 1..303: mol_type = other DNA

SEQ ID NO: 548 - protein, 101 aa, synthetic construct; identical to SEQ ID NO: 528
  REGION 1..101: note = Synthetic biopolymer

SEQ ID NO: 549 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattcaagaagcccagaaagaagaatttttcaagacctatgtaaacctggtgaatatcatccctgcgatgaaagatgtttattggggcaaagacgtcacccaacgtaacaaagaagaaggttactcacacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 550 - protein, 101 aa, synthetic construct
  REGION 1..101: note = Synthetic biopolymer
MAVKHLIIIKFNDEIQEAQKEEFFKTYVNLVNIIPAMKDVYWGKDVTQRNKEEGYSHIVEVTFESVETIQDYIIHPAHVGFGDVYRSFWEKLLIFDYTPRK

SEQ ID NO: 551 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttggactcacgtcgtcgaagtctgtttcgagtcagtagagacgatccaagattacataatacacccggcccatgtgggattcggagacgtttaccgtagcttctgggaaaaacttttgattttcgactataccccgagaaaa

SEQ ID NO: 552 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGWTHVVEVCFESVETIQDYIIHPAHVGFGDVYRSFWEKLLIFDYTPRK

SEQ ID NO: 553 - DNA, 303 bp, synthetic construct
  misc_feature 1..303: note = Synthetic biopolymer
  source 1..303: mol_type = other DNA
atgccggtcaagcaccttatcatcatcaaattcaaggacgaaattcaagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagacgatccaagattacataatacacccggcccatgtgggattcggagacgtttaccgtagcttctgggaaaaacttttgaaattcgactataccccgagaaaa

SEQ ID NO: 554 - protein, 101 aa, synthetic construct
  REGION 1..101: note = Synthetic biopolymer
MPVKHLIIIKFKDEIQEAQKEEFFKTYVNLVNIIPAMKDVYWGKDVTQKNKEEGYTHIVEVTFDSVETIQDYIIHPAHVGFGDVYRSFWEKLLKFDYTPRK

SEQ ID NO: 555 - DNA, 303 bp, synthetic construct; identical to SEQ ID NO: 521
  misc_feature 1..303: note = Synthetic biopolymer
  source 1..303: mol_type = other DNA

SEQ ID NO: 556 - protein, 101 aa, synthetic construct; identical to SEQ ID NO: 522
  REGION 1..101: note = Synthetic biopolymer

SEQ ID NO: 557 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattcaagaagcccagaaagaagaatttttcaagacctatgtaaacctggtgaatatcatccctttcatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 558 - protein, 101 aa, synthetic construct
  REGION 1..101: note = Synthetic biopolymer
MAVKHLIIIKFKDEIQEAQKEEFFKTYVNLVNIIPFMKDVYWGKDVTQRNKEEGYTHVVEVTFESVETIQDYIIHPAHVGFGDVYRSFWEKLLIFDYTPRK

SEQ ID NO: 559 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 560 - protein, 101 aa, synthetic construct
  REGION 1..101: note = Synthetic biopolymer
MAVKHLIILKFKDEITEAQKEEFFKTYVNLVNVIPAMKDVYWGKDVTQRNKEEGYSHVVEVTFESVETIQDYIIHPAHVGFGDVYRSFWEKLLIFDYTPGK

SEQ ID NO: 561 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattcaagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagacgatcaaagattacataatacacccggcccatgtgggattcggagacgtttaccgtagcttctgggaaaaacttttgattttcgactataccccgagaaaa

SEQ ID NO: 562 - protein, 101 aa, synthetic construct
  REGION 1..101: note = Synthetic biopolymer
MAVKHLIIIKFKDEIQEAQKEEFFKTYVNLVNIIPAMKDVYWGKDVTQKNKEEGYSHVVEVTFESVETIKDYIIHPAHVGFGDVYRSFWEKLLIFDYTPRK

SEQ ID NO: 563 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattcaagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 564 - protein, 101 aa, synthetic construct
  REGION 1..101: note = Synthetic biopolymer
MAVKHLIIIKFKDEIQEAQKEEFFKTYVNLVNIIPAMKDVYWGKDVTQKNKEEGYSHVVEVTFESVETIQDYIIHPAHVGFGDVYRSFWEKLLIFDYTPGK

SEQ ID NO: 565 - DNA, 303 bp, synthetic construct; identical to SEQ ID NO: 535
  misc_feature 1..303: note = Synthetic biopolymer
  source 1..303: mol_type = other DNA

SEQ ID NO: 566 - protein, 101 aa, synthetic construct; identical to SEQ ID NO: 536
  REGION 1..101: note = Synthetic biopolymer

SEQ ID NO: 567 - DNA, 303 bp, synthetic construct
  misc_feature 1..303: note = Synthetic biopolymer
  source 1..303: mol_type = other DNA
atggctgtaaagcaccttatcatcatcgccttcaaggacgaaattacagaagcccagaaagaagaatttttcaagacctatgtagacctggtgaatgtcatccctgcgatgaaagatgtttattggggcaaagacgtcacccaaaaaaacaaagaagaaggttactcacacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 568 - protein, 101 aa, synthetic construct; identical to SEQ ID NO: 536
  REGION 1..101: note = Synthetic biopolymer

SEQ ID NO: 569 - DNA, 303 bp, synthetic construct; identical to SEQ ID NO: 511
  misc_feature 1..303: note = Synthetic biopolymer
  source 1..303: mol_type = other DNA

SEQ ID NO: 570 - protein, 101 aa, synthetic construct; identical to SEQ ID NO: 512
  REGION 1..101: note = Synthetic biopolymer

SEQ ID NO: 571 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttggtcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 572 - protein, 101 aa, synthetic construct; identical to SEQ ID NO: 546
  REGION 1..101: note = Synthetic biopolymer

SEQ ID NO: 573 - DNA, 303 bp, synthetic construct; identical to SEQ ID NO: 541
  misc_feature 1..303: note = Synthetic biopolymer
  source 1..303: mol_type = other DNA

SEQ ID NO: 574 - protein, 101 aa, synthetic construct; identical to SEQ ID NO: 528
  REGION 1..101: note = Synthetic biopolymer

SEQ ID NO: 575 - DNA, 303 bp, synthetic construct
  misc_feature 1..303: note = Synthetic biopolymer
  source 1..303: mol_type = other DNA
atggctgtaaagcaccttatcatcatcaaattcgtcgacgaaattacagaagcccagaaagaagaatttttcaagacctatgtaaacctggtgaatgtcatccctgcgatgaaagatgtttattggggcaaagacgtcacccaaaaaaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 576 - protein, 101 aa, synthetic construct
  REGION 1..101: note = Synthetic biopolymer
MAVKHLIIIKFVDEITEAQKEEFFKTYVNLVNVIPAMKDVYWGKDVTQKNKEEGYSHVVEVTFESVETIQDYIIHPAHVGFGDVYRSFWEKLLIFDYTPRK

SEQ ID NO: 577 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattcaagaagcccagaaagaagaatttttcaagacctatgtaaacctggtgaatatcatccctttcatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 578 - protein, 101 aa, synthetic construct; identical to SEQ ID NO: 558
  REGION 1..101: note = Synthetic biopolymer

SEQ ID NO: 579 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattacagaagcccagaaagaagaatttttcaagacctatgtaaacctggtgaatgtcatccctgcgatgaaagatgtttattggggcaaagacgtcacccaaaaaaacaaagaagaaggttactcacacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 580 - protein, 101 aa, synthetic construct
  REGION 1..101: note = Synthetic biopolymer
MAVKHLIIIKFNDEITEAQKEEFFKTYVNLVNVIPAMKDVYWGKDVTQKNKEEGYSHIVEVTFESVETIQTYIIHPAHVGFGDVYRSFWEKLLIFDYTPRK

SEQ ID NO: 581 - DNA, 303 bp, synthetic construct; identical to SEQ ID NO: 531
  misc_feature 1..303: note = Synthetic biopolymer
  source 1..303: mol_type = other DNA

SEQ ID NO: 582 - protein, 101 aa, synthetic construct; identical to SEQ ID NO: 532
  REGION 1..101: note = Synthetic biopolymer

SEQ ID NO: 583 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatcccttcaatgaaagatgtttattggggcaaagacgtcacccaaaaaaacaaagaagaaggttacactcacatcgtcgaagtctgtttcgactcagtagagacgatccaagattacataatacacccggcccatgtgggattcggagacgtttaccgtagcttctgggaaaaacttttgattttcgactataccccgagaaaa

SEQ ID NO: 584 - protein, 101 aa, synthetic construct
  REGION 1..101: note = Synthetic biopolymer
MAVKHLIIIKFKDEITEAQKEEFFKTYVNLVNVIPSMKDVYWGKDVTQKNKEEGYTHIVEVCFDSVETIQDYIIHPAHVGFGDVYRSFWEKLLIFDYTPRK

SEQ ID NO: 585 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttggtcacacgtcgtcgaagtcactttcgagtcagtagagacgatccaagattacataatacacccggcccatgtgggattcggagacgtttaccgtagcttctgggaaaaacttttgattttcgactataccccgggaaaa

SEQ ID NO: 586 - protein, 101 aa, synthetic construct
  REGION 1..101: note = Synthetic biopolymer
MAVKHLIILKFKDEITEAQKEEFFKTYVNLVNIIPAMKDVYWGKDVTQRNKEEGWSHVVEVTFESVETIQDYIIHPAHVGFGDVYRSFWEKLLIFDYTPGK

SEQ ID NO: 587 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctcaaatgaaagatgtttattggggcaaagacgtcacccaaaaaaacaaagaagaaggttacactcacgtcgtcgaagtcactttcgactcagtagagtcaatccaagattacataatacacccggcccatgtgggattcggagacgtttaccgtagcttctgggaaaaacttttgaaattcgactataccccgagaaaa

SEQ ID NO: 588 - protein, 101 aa, synthetic construct
  REGION 1..101: note = Synthetic biopolymer
MAVKHLIVIKFKDEITEAQKEEFFKTYVNLVNIIPQMKDVYWGKDVTQKNKEEGYTHVVEVTFDSVESIQDYIIHPAHVGFGDVYRSFWEKLLKFDYTPRK

SEQ ID NO: 589 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagtcaatccaagattacataatacacccggcccatgtgggattcggagacgtttaccgtagcttctgggaaaaacttttgattttcgactataccccgggaaaa

SEQ ID NO: 590 - protein, 101 aa, synthetic construct
  REGION 1..101: note = Synthetic biopolymer
MAVKHLIIIKFKDEITEAQKEEFFKTYVNLVNIIPAMKDVYWGKDVTQKNKEEGYSHVVEVTFESVESIQDYIIHPAHVGFGDVYRSFWEKLLIFDYTPGK

SEQ ID NO: 591 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttactcacacatcgtcgaagtcactttcgactcagtagagtcaatccaagattacataatacacccggcccatgtgggattcggagacgtttaccgtagcttctgggaaaaacttttgattttcgactataccccgagaaaa

SEQ ID NO: 592 - protein, 101 aa, synthetic construct
  REGION 1..101: note = Synthetic biopolymer
MAVKHLIIIKFVDEITEAQKEEFFKTYVNLVNIIPAMKDVYWGKDVTQKNKEEGYSHIVEVTFDSVESIQDYIIHPAHVGFGDVYRSFWEKLLIFDYTPRK

SEQ ID NO: 593 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctcaaatgaaagatgtttattggggcaaagacgtcacccaaaaaaacaaagaagaaggttacactcacgtcgtcgaagtcactttcgactcagtagagacgatccaagattacataatacacccggcccatgtgggattcggagacgtttaccgtagcttctgggaaaaacttttgaaattcgacttcaccccgagaaaa

SEQ ID NO: 594 - protein, 101 aa, synthetic construct
  REGION 1..101: note = Synthetic biopolymer
MAVKHLIVIKFKDEITEAQKEEFFKTYVNLVNIIPQMKDVYWGKDVTQKNKEEGYTHVVEVTFDSVETIQDYIIHPAHVGFGDVYRSFWEKLLKFDFTPRK

SEQ ID NO: 595 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctattgtaacctggtgaatatcatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgatttttgattacactccaaggaag

SEQ ID NO: 596 - protein, 101 aa, synthetic construct
  REGION 1..101: note = Synthetic biopolymer
MAVKHLIIIKFKDEITEAQKEEFFKTYCNLVNIIPAMKDVYWGKDVTQRNKEEGYSHVVEVTFESVETIQDYIIHPAHVGFGDVYRSFWEKLLIFDYTPRK

SEQ ID NO: 597 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtcactttcgactcagtagagggaatccaagattacataatacacccggcccatgtgggattcggagacgtttaccgtagcttctgggaaaaacttttgattttcgactataccccgagaaaa

SEQ ID NO: 598 - protein, 101 aa, synthetic construct
  REGION 1..101: note = Synthetic biopolymer
MAVKHLIIIKFVDEITEAQKEEFFKTYVNLVNIIPAMKDVYWGKDVTQKNKEEGYTHVVEVTFDSVEGIQDYIIHPAHVGFGDVYRSFWEKLLIFDYTPRK

SEQ ID NO: 599 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatcccttcaatgaaagatgtttattggggcaaagacgtcacccaaaaaaacaaagaagaaggttacactcacatcgtcgaagtcactttcgactcagtagagacgatccaagattacataatacacccggcccatgtgggattcggagacgtttaccgtagcttctgggaaaaacttttgaaattcgacttcaccccgagaaaa

SEQ ID NO: 600 - protein, 101 aa, synthetic construct
  REGION 1..101: note = Synthetic biopolymer
MAVKHLIIIKFKDEITEAQKEEFFKTYVNLVNIIPSMKDVYWGKDVTQKNKEEGYTHIVEVTFDSVETIQDYIIHPAHVGFGDVYRSFWEKLLKFDFTPRK

SEQ ID NO: 601 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcagtagagggaatccaaacctacataatacacccggcccatgtgggattcggagacgtttaccgtagcttctgggaaaaacttttgattttcgactataccccgagaaaa

SEQ ID NO: 602 - protein, 101 aa, synthetic construct
  REGION 1..101: note = Synthetic biopolymer
MAVKHLIIIKFNDEITEAQKEEFFKTYVNLVNIIPAMKDVYWGKDVTQRNKEEGYTHIVEVTFESVEGIQTYIIHPAHVGFGDVYRSFWEKLLIFDYTPRK

SEQ ID NO: 603 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtctgtttcgagtcagtagagacgatccaagattacataatacacccggcccatgtgggattcggagacgtttaccgtagcttctgggaaaaacttttgattttcgacttcaccccgagaaaa

SEQ ID NO: 604 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYTHVVEVCFESVETIQDYIIHPAHVGFGDVYRSFWEKLLIFDFTPRK

SEQ ID NO: 605 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctattgtaacctggtgaatatcatccctgcgatgaaagatgtttattggggcaaagacgtcacccaacgtaacaaagaagaaggttactcacacatcgtcgaagtcactttcgagtcagtagagacgatccaaacctacataatacacccggcccatgtgggattcggagacgtttaccgtagcttctgggaaaaacttttgattttcgactataccccgagaaaa

SEQ ID NO: 606 - protein, 101 aa, synthetic construct
  REGION 1..101: note = Synthetic biopolymer
MAVKHLIIIKFKDEITEAQKEEFFKTYCNLVNIIPAMKDVYWGKDVTQRNKEEGYSHIVEVTFESVETIQTYIIHPAHVGFGDVYRSFWEKLLIFDYTPRK

SEQ ID NO: 607 - DNA, 303 bp, synthetic construct; identical to SEQ ID NO: 603
  misc_feature 1..303: note = Synthetic biopolymer
  source 1..303: mol_type = other DNA

SEQ ID NO: 608 - protein, 101 aa, synthetic construct; identical to SEQ ID NO: 604
  REGION 1..101: note = Synthetic biopolymer

SEQ ID NO: 609 - DNA, 303 bp, synthetic construct
  misc_feature 1..303: note = Synthetic biopolymer
  source 1..303: mol_type = other DNA
atggctgtaaagcaccttatcatcatcgccttcaaggacgaaattacagaagcccagaaagaagaatttttcaagacctatgtagacctggtgaatatcatccctgcgatgaaagatgtttattggggcaaagacgtcacccaaaaaaacaaagaagaaggttacactcacgtcgtcgaagtcactttcgagtcagtagagggaatccaagattacataatacacccggcccatgtgggattcggagacgtttaccgtagcttctgggaaaaacttttgattttcgactataccccgagaaaa

SEQ ID NO: 610 - protein, 101 aa, synthetic construct
  REGION 1..101: note = Synthetic biopolymer
MAVKHLIIIAFKDEITEAQKEEFFKTYVDLVNIIPAMKDVYWGKDVTQKNKEEGYTHVVEVTFESVEGIQDYIIHPAHVGFGDVYRSFWEKLLIFDYTPRK

SEQ ID NO: 611 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagagtcaatccaagattacataatacacccggcccatgtgggattcggagacgtttaccgtagcttctgggaaaaacttttgattttcgactataccccgggaaaa

SEQ ID NO: 612 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYTHVVEVTFESVESIQDYIIHPAHVGFGDVYRSFWEKLLIFDYTPGK

SEQ ID NO: 613 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttactcacacgtcgtcgaagtcactttcgagtcagtagagggaatccaagattacataatacacccggcccatgtgggattcggagacgtttaccgtagcttctgggaaaaacttttgattttcgactataccccgggaaaa

SEQ ID NO: 614 - protein, 101 aa, synthetic construct
  REGION 1..101: note = Synthetic biopolymer
MAVKHLIVIKFKDEITEAQKEEFFKTYVNLVNIIPAMKDVYWGKDVTQRNKEEGYSHVVEVTFESVEGIQDYIIHPAHVGFGDVYRSFWEKLLIFDYTPGK

SEQ ID NO: 615 - DNA, 303 bp, synthetic construct; identical to SEQ ID NO: 591
  misc_feature 1..303: note = Synthetic biopolymer
  source 1..303: mol_type = other DNA

SEQ ID NO: 616 - protein, 101 aa, synthetic construct; identical to SEQ ID NO: 592
  REGION 1..101: note = Synthetic biopolymer

SEQ ID NO: 617 - DNA, 303 bp, synthetic construct
  misc_feature 1..303: note = Synthetic biopolymer
  source 1..303: mol_type = other DNA
atggctgtcaagcaccttatcatcatcgccttcaaggacgaaattacagaagcccagaaagaagaatttttcaagacctatgtagacctggtgaatatcatccctgcgatgaaagatgtttattggggcaaagacgtcacccaaaaaaacaaagaagaaggttacactcacgtcgtcgaagtcactttcgagtcagtagagggaatccaagattacataatacacccggcccatgtgggattcggagacgtttaccgtagcttctgggaaaaacttttgattttcgactataccccgagaaaa

SEQ ID NO: 618 - protein, 101 aa, synthetic construct; identical to SEQ ID NO: 610
  REGION 1..101: note = Synthetic biopolymer

SEQ ID NO: 619 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatcccttcaatgaaagatgtttattggggcaaagacgtcacccaaaaaaacaaagaagaaggttacactcacatcgtcgaagtcactttcgactcagtagagacgatccaagattacataatacacccggcccatgtgggattcggagacgtttaccgtagcttctgggaaaaacttttgaaattcgacttcaccccgagaaaa

SEQ ID NO: 620 - protein, 101 aa, synthetic construct; identical to SEQ ID NO: 600
  REGION 1..101: note = Synthetic biopolymer

SEQ ID NO: 621 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattacagaagcccagaaagaagaatttttcaagacctatgtaaacctggtgaatatcatccctgcgatgaaagatgtttattggggcaaagacgtcacccaacgtaacaaagaagaaggttactcacacatcgtcgaagtcactttcgagtcagtagagacgatccaagattacataatacacccggcccatgtgggattcggagacgtttaccgtagcttctgggaaaaacttttgattttcgacttcaccccgagaaaa

SEQ ID NO: 622 - protein, 101 aa, synthetic construct
  REGION 1..101: note = Synthetic biopolymer
MAVKHLIIIKFNDEITEAQKEEFFKTYVNLVNIIPAMKDVYWGKDVTQRNKEEGYSHIVEVTFESVETIQDYIIHPAHVGFGDVYRSFWEKLLIFDFTPRK

SEQ ID NO: 623 - DNA, 303 bp, synthetic construct
  misc_feature 1..303: note = Synthetic biopolymer
  source 1..303: mol_type = other DNA
atggctgtaaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtcactttcgactcagtagagggaatccaagattacataatacacccggcccatgtgggattcggagacgtttaccgtagcttctgggaaaaacttttgattttcgactataccccgagaaaa

SEQ ID NO: 624 - protein, 101 aa, synthetic construct; identical to SEQ ID NO: 598
  REGION 1..101: note = Synthetic biopolymer

SEQ ID NO: 625 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtcactttcgactcagtagagacgatccaagattacataatacacccggcccatgtgggattcggagacgtttaccgtagcttctgggaaaaacttttgattttcgacttcaccccgagaaaa

SEQ ID NO: 626 - protein, 101 aa, synthetic construct
  REGION 1..101: note = Synthetic biopolymer
MAVKHLIIIKFVDEITEAQKEEFFKTYVNLVNIIPAMKDVYWGKDVTQKNKEEGYTHVVEVTFDSVETIQDYIIHPAHVGFGDVYRSFWEKLLIFDFTPRK

SEQ ID NO: 627 - DNA, 303 bp, synthetic construct; identical to SEQ ID NO: 589
  misc_feature 1..303: note = Synthetic biopolymer
  source 1..303: mol_type = other DNA

SEQ ID NO: 628 - protein, 101 aa, synthetic construct; identical to SEQ ID NO: 590
  REGION 1..101: note = Synthetic biopolymer

SEQ ID NO: 629 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtctgtttcgagtcagtagagggaatccaagattacataatacacccggcccatgtgggattcggagacgtttaccgtagcttctgggaaaaacttttgatttttgattacactccaaggaag

SEQ ID NO: 630 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYTHVVEVCFESVEGIQDYIIHPAHVGFGDVYRSFWEKLLIFDYTPRK

SEQ ID NO: 631 - DNA, 303 bp, synthetic construct; identical to SEQ ID NO: 591
  misc_feature 1..303: note = Synthetic biopolymer
  source 1..303: mol_type = other DNA

SEQ ID NO: 632 - protein, 101 aa, synthetic construct; identical to SEQ ID NO: 592
  REGION 1..101: note = Synthetic biopolymer

SEQ ID NO: 633 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttactcacacgtcgtcgaagtcactttcgagtcagtagagtcaatccaagattacataatacacccggcccatgtgggattcggagacgtttaccgtagcttctgggaaaaacttttgattttcgactataccccgggaaaa

SEQ ID NO: 634 - protein, 101 aa, synthetic construct
  REGION 1..101: note = Synthetic biopolymer
MAVKHLIILKFKDEITEAQKEEFFKTYVNLVNIIPAMKDVYWGKDVTQRNKEEGYSHVVEVTFESVESIQDYIIHPAHVGFGDVYRSFWEKLLIFDYTPGK

SEQ ID NO: 635 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagggaatcaaagattacataatacacccggcccatgtgggattcggagacgtttaccgtagcttctgggaaaaacttttgattttcgactataccccgagaaaa

SEQ ID NO: 636 - protein, 101 aa, synthetic construct
  REGION 1..101: note = Synthetic biopolymer
MAVKHLIIIKFKDEITEAQKEEFFKTYVNLVNIIPAMKDVYWGKDVTQKNKEEGYSHVVEVTFESVEGIKDYIIHPAHVGFGDVYRSFWEKLLIFDYTPRK

SEQ ID NO: 637 - DNA, 303 bp, synthetic construct; identical to SEQ ID NO: 629
  misc_feature 1..303: note = Synthetic biopolymer
  source 1..303: mol_type = other DNA

SEQ ID NO: 638 - protein, 101 aa, synthetic construct; identical to SEQ ID NO: 630
  REGION 1..101: note = Synthetic biopolymer

SEQ ID NO: 639 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagggaatccaagattacataatacacccggcccatgtgggattcggagacgtttaccgtagcttctgggaaaaacttttgattttcgactataccccgagaaaa

SEQ ID NO: 640 - protein, 101 aa, synthetic construct
  REGION 1..101: note = Synthetic biopolymer
MAVKHLIIIKFVDEITEAQKEEFFKTYVNLVNIIPAMKDVYWGKDVTQKNKEEGYSHVVEVTFESVEGIQDYIIHPAHVGFGDVYRSFWEKLLIFDYTPRK

SEQ ID NO: 641 - DNA, 303 bp, synthetic construct
  misc_feature 1..303: note = Synthetic biopolymer
  source 1..303: mol_type = other DNA
atggctgtcaagcaccttatcatcatcaaattcgtcgacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagtcaatccaagattacataatacacccggcccatgtgggattcggagacgtttaccgtagcttctgggaaaaacttttgattttcgactataccccgagaaaa

SEQ ID NO: 642 - protein, 101 aa, synthetic construct
  REGION 1..101: note = Synthetic biopolymer
MAVKHLIIIKFVDEITEAQKEEFFKTYVNLVNIIPAMKDVYWGKDVTQKNKEEGYSHVVEVTFESVESIQDYIIHPAHVGFGDVYRSFWEKLLIFDYTPRK

SEQ ID NO: 643 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctttcatgaaagatgtttattggggcaaagacgtcacccaacgtaacaaagaagaaggttacactcacgtcgtcgaagtcactttcgagtcagtagagtcaatccaagattacataatacacccggcccatgtgggattcggagacgtttaccgtagcttctgggaaaaacttttgattttcgactataccccgagaaaa

SEQ ID NO: 644 - protein, 101 aa, synthetic construct
  REGION 1..101: note = Synthetic biopolymer
MAVKHLIIIKFKDEITEAQKEEFFKTYVNLVNIIPFMKDVYWGKDVTQRNKEEGYTHVVEVTFESVESIQDYIIHPAHVGFGDVYRSFWEKLLIFDYTPRK

SEQ ID NO: 645 - DNA, 303 bp, synthetic construct; identical to SEQ ID NO: 603
  misc_feature 1..303: note = Synthetic biopolymer
  source 1..303: mol_type = other DNA

SEQ ID NO: 646 - protein, 101 aa, synthetic construct; identical to SEQ ID NO: 604
  REGION 1..101: note = Synthetic biopolymer

SEQ ID NO: 647 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctttcatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgacttcaccccgagaaaa

SEQ ID NO: 648 - protein, 101 aa, synthetic construct
  REGION 1..101: note = Synthetic biopolymer
MAVKHLIIIKFKDEITEAQKEEFFKTYVNLVNIIPFMKDVYWGKDVTQRNKEEGYTHVVEVTFESVETIQDYIIHPAHVGFGDVYRSFWEKLLIFDFTPRK

SEQ ID NO: 649 - DNA, 303 bp, synthetic construct; identical to SEQ ID NO: 601
  misc_feature 1..303: note = Synthetic biopolymer
  source 1..303: mol_type = other DNA

SEQ ID NO: 650 - protein, 101 aa, synthetic construct; identical to SEQ ID NO: 602
  REGION 1..101: note = Synthetic biopolymer

SEQ ID NO: 651 - DNA, 303 bp, synthetic construct; identical to SEQ ID NO: 591
  misc_feature 1..303: note = Synthetic biopolymer
  source 1..303: mol_type = other DNA

SEQ ID NO: 652 - protein, 101 aa, synthetic construct; identical to SEQ ID NO: 592
  REGION 1..101: note = Synthetic biopolymer

SEQ ID NO: 653 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgacttcaccccgggaaaa

SEQ ID NO: 654 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYTHVVEVTFESVETIQDYIIHPAHVGFGDVYRSFWEKLLIFDFTPGK

SEQ ID NO: 655 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctttcatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgacttcaccccgagaaaa

SEQ ID NO: 656 - protein, 101 aa, synthetic construct; identical to SEQ ID NO: 648
  REGION 1..101: note = Synthetic biopolymer

SEQ ID NO: 657 - DNA, 303 bp, synthetic construct; identical to SEQ ID NO: 591
  misc_feature 1..303: note = Synthetic biopolymer
  source 1..303: mol_type = other DNA

SEQ ID NO: 658 - protein, 101 aa, synthetic construct; identical to SEQ ID NO: 592
  REGION 1..101: note = Synthetic biopolymer

SEQ ID NO: 659 - DNA, 303 bp, synthetic construct
  misc_feature 1..303: note = Synthetic biopolymer
  source 1..303: mol_type = other DNA
atggctgtcaagcaccttatcatcatcgccttcaaggacgaaattacagaagcccagaaagaagaatttttcaagacctatgtagacctggtgaatatcatccctgcgatgaaagatgtttattggggcaaagacgtcacccaaaaaaacaaagaagaaggttactcacacatcgtcgaagtcactttcgagtcagtagagtcaatccaagattacataatacacccggcccatgtgggattcggagacgtttaccgtagcttctgggaaaaacttttgattttcgactataccccgagaaaa

SEQ ID NO: 660 - protein, 101 aa, synthetic construct
  REGION 1..101: note = Synthetic biopolymer
MAVKHLIIIAFKDEITEAQKEEFFKTYVDLVNIIPAMKDVYWGKDVTQKNKEEGYSHIVEVTFESVESIQDYIIHPAHVGFGDVYRSFWEKLLIFDYTPRK

SEQ ID NO: 661 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctattgtaacctggtgaatatcatccctgcgatgaaagatgtttattggggcaaagacgtcacccaacgtaacaaagaagaaggttactcacacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 662 - protein, 101 aa, synthetic construct; identical to SEQ ID NO: 596
  REGION 1..101: note = Synthetic biopolymer

SEQ ID NO: 663 - DNA, 303 bp, synthetic construct
  misc_feature 1..303: note = Synthetic biopolymer
  source 1..303: mol_type = other DNA
atggctgtaaagcaccttatcatcatcgccttcaaggacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcagtagagacgatccaagattacataatacacccggcccatgtgggattcggagacgtttaccgtagcttctgggaaaaacttttgattttcgacttcaccccgagaaaa

SEQ ID NO: 664 - protein, 101 aa, synthetic construct
  REGION 1..101: note = Synthetic biopolymer
MAVKHLIIIAFKDEITEAQKEEFFKTYVNLVNIIPAMKDVYWGKDVTQKNKEEGYSHVVEVTFESVETIQDYIIHPAHVGFGDVYRSFWEKLLIFDFTPRK

SEQ ID NO: 665 - DNA, 303 bp, synthetic construct
  misc_feature 1..303: note = Synthetic biopolymer
  source 1..303: mol_type = other DNA
atggctgtcaagcaccttatcatcatcaaattcgtcgacggaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcagtatcaacgatccaagattacataatacacccggcccatgtgggattcggagacgtttaccgtagcttctgggaaaaacttttgattttcgactataccccgagaaaa

SEQ ID NO: 666 - protein, 101 aa, synthetic construct
  REGION 1..101: note = Synthetic biopolymer
MAVKHLIIIKFVDGITEAQKEEFFKTYVNLVNIIPAMKDVYWGKDVTQRNKEEGYTHIVEVTFESVSTIQDYIIHPAHVGFGDVYRSFWEKLLIFDYTPRK

SEQ ID NO: 667 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatcccttcaatgaaagatgtttattggggcaaagacgtcacccaacgtaacaaagaagaaggttacactcacatcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 668 - protein, 101 aa, synthetic construct
  REGION 1..101: note = Synthetic biopolymer
MAVKHLIVIKFKDEITEAQKEEFFKTYVNLVNIIPSMKDVYWGKDVTQRNKEEGYTHIVEVTFDSVEMIQDYIMHPAHVGFGDVYRSFWEKLLIFDYTPRK

SEQ ID NO: 669 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctgcgatgaaagatgttgaatggggcaaagacgtcacccaacgtaacaaagaagaaggttacactcacgtcgtcgaagtcactttcgagtcaatcgaggaaatccaagattacataatacacccggcccatgtgggattcggagacgtttaccgtagcttctgggaaaaacttttgattttcgactataccccgagaaaa

SEQ ID NO: 670 - protein, 101 aa, synthetic construct
  REGION 1..101: note = Synthetic biopolymer
MAVKHLIVIKFKDEITEAQKEEFFKTYVNLVNIIPAMKDVEWGKDVTQRNKEEGYTHVVEVTFESIEEIQDYIIHPAHVGFGDVYRSFWEKLLIFDYTPRK

SEQ ID NO: 671 - DNA, 303 bp, synthetic construct; identical to SEQ ID NO: 665
  misc_feature 1..303: note = Synthetic biopolymer
  source 1..303: mol_type = other DNA

SEQ ID NO: 672 - protein, 101 aa, synthetic construct; identical to SEQ ID NO: 666
  REGION 1..101: note = Synthetic biopolymer

SEQ ID NO: 673 - DNA, 303 bp, synthetic construct
  misc_feature 1..303: note = Synthetic biopolymer
  source 1..303: mol_type = other DNA
atggctgtcaagcaccttatcatcatcaaattcaacgacgaaattcaagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 674 - protein, 101 aa, synthetic construct
  REGION 1..101: note = Synthetic biopolymer
MAVKHLIIIKFNDEIQEAQKEEFFKTYVNLVNIIPAMKDVYWGKDVTQRNKEEGYTHVVEVTFESVETIQDYIIHPAHVGFGDVYRSFWEKLLIFDYTPRK

SEQ ID NO: 675 - DNA, 303 bp, synthetic construct; identical to SEQ ID NO: 665
  misc_feature 1..303: note = Synthetic biopolymer
  source 1..303: mol_type = other DNA

SEQ ID NO: 676 - protein, 101 aa, synthetic construct; identical to SEQ ID NO: 666
  REGION 1..101: note = Synthetic biopolymer

SEQ ID NO: 677 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaacacctatgtaaacctggtgaatatcatccctgcgatgaaagatgtttattggggcaaagacgtcacccaacgtaacaaagaagaaggttacactcacgtcgtcgaagtcactttcgagtcagtagagtcaatccaagattacataatacacccggcccatgtgggattcggagacgtttaccgtagcttctgggaaaaacttttgattttcgactataccccgagaaaa

SEQ ID NO: 678 - protein, 101 aa, synthetic construct
  REGION 1..101: note = Synthetic biopolymer
MAVKHLIIIKFKDEITEAQKEEFFNTYVNLVNIIPAMKDVYWGKDVTQRNKEEGYTHVVEVTFESVESIQDYIIHPAHVGFGDVYRSFWEKLLIFDYTPRK

SEQ ID NO: 679 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttactcacacatcgtcgaagtcactttcgagtcaatcgagatgatccaagattacataatacacccggcccatgtgggattcggagacgtttaccgtagcttctgggaaaaacttttgattttcgactataccccgagaaaa

SEQ ID NO: 680 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYSHIVEVTFESIEMIQDYIIHPAHVGFGDVYRSFWEKLLIFDYTPRK

SEQ ID NO: 681 - DNA, 303 bp, synthetic construct; identical to SEQ ID NO: 665
  misc_feature 1..303: note = Synthetic biopolymer
  source 1..303: mol_type = other DNA

SEQ ID NO: 682 - protein, 101 aa, synthetic construct; identical to SEQ ID NO: 666
  REGION 1..101: note = Synthetic biopolymer

SEQ ID NO: 683 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaacacctatgtaaacctggtgaatatcatccctccgatgaaagatgtttattggggcaaagacgtcacccaaaaaaacaaagaagaaggttacactcacatcgtcgaagtcactttcgagtcagtagagggaatccaagattacataatgcacccggcccatgtgggattcggagacgtttaccgtagcttctgggaaaaacttttgattttcgactataccccgagaaaa

SEQ ID NO: 684 - protein, 101 aa, synthetic construct
  REGION 1..101: note = Synthetic biopolymer
MAVKHLIIIKFKDEITEAQKEEFFNTYVNLVNIIPPMKDVYWGKDVTQKNKEEGYTHIVEVTFESVEGIQDYIMHPAHVGFGDVYRSFWEKLLIFDYTPRK

SEQ ID NO: 685 - DNA, 303 bp, synthetic construct; identical to SEQ ID NO: 669
  misc_feature 1..303: note = Synthetic biopolymer
  source 1..303: mol_type = other DNA

SEQ ID NO: 686 - protein, 101 aa, synthetic construct; identical to SEQ ID NO: 670
  REGION 1..101: note = Synthetic biopolymer

SEQ ID NO: 687 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcagtagagacgatccaagattacataatacacccggcccatgtgggattcggagacgtttaccgtagcttctgggaaaaacttttgattttcgacttcaccccgggaaaa

SEQ ID NO: 688 - protein, 101 aa, synthetic construct
  REGION 1..101: note = Synthetic biopolymer
MAVKHLIIIKFKDEITEAQKEEFFKTYVNLVNIIPAMKDVYWGKDVTQRNKEEGYTHIVEVTFDSVETIQDYIIHPAHVGFGDVYRSFWEKLLIFDFTPGK

SEQ ID NO: 689 - DNA, 303 bp, synthetic construct; identical to SEQ ID NO: 665
  misc_feature 1..303: note = Synthetic biopolymer
  source 1..303: mol_type = other DNA

SEQ ID NO: 690 - protein, 101 aa, synthetic construct; identical to SEQ ID NO: 666
  REGION 1..101: note = Synthetic biopolymer

SEQ ID NO: 691 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaaaaaaacaaagaagaaggttacactcacgtcgtcgaagtcactttcgactcagtagagacgatccaagattacataatacacccggcccatgtgggattcggagacgtttaccgtagcttctgggaaaaacttttgattttcgactataccccgggaaaa

SEQ ID NO: 692 - protein, 101 aa, synthetic construct
  REGION 1..101: note = Synthetic biopolymer
MAVKHLIIIKFKDEITEAQKEEFFKTYVNLVNVIPAMKDVYWGKDVTQKNKEEGYTHVVEVTFDSVETIQDYIIHPAHVGFGDVYRSFWEKLLIFDYTPGK

SEQ ID NO: 693 - DNA, 303 bp, synthetic construct; identical to SEQ ID NO: 667
  misc_feature 1..303: note = Synthetic biopolymer
  source 1..303: mol_type = other DNA

SEQ ID NO: 694 - protein, 101 aa, synthetic construct; identical to SEQ ID NO: 668
  REGION 1..101: note = Synthetic biopolymer

SEQ ID NO: 695 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcagtagagacgatccaagattacataatacacccggcccatgtgggattcggagacgtttaccgtagcttctgggaaaaacttttgattttcgacttcaccccgggaaaa

SEQ ID NO: 696 - protein, 101 aa, synthetic construct; identical to SEQ ID NO: 688
  REGION 1..101: note = Synthetic biopolymer

SEQ ID NO: 697 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatatcatccctgcgatgaaagatgttgaatggggcaaagacgtcacccaaaaaaacaaagaagaaggttacactcacgtcgtcgaagtcactttcgactcagtagaggccatccaagattacataatacacccggcccatgtgggattcggagacgtttaccgtagcttctgggaaaaacttttgattttcgactataccccgagaaaa

SEQ ID NO: 698 - protein, 101 aa, synthetic construct
  REGION 1..101: note = Synthetic biopolymer
MAVKHLIIIKFKDEITEAQKEEFFKTYVNLVNIIPAMKDVEWGKDVTQKNKEEGYTHVVEVTFDSVEAIQDYIIHPAHVGFGDVYRSFWEKLLIFDYTPRK

SEQ ID NO: 699 - DNA, 303 bp, synthetic construct
  misc_feature 1..303: note = Synthetic biopolymer
  source 1..303: mol_type = other DNA
atggctgtcaagcaccttatcatcctgaaattccaagacgaaattacagaagcccagaaagaagaatttttcaagacctatgtaaacctgatgaatatcatccctgcgatgaaagatgtttattggggcaaagacgtcacccaaaaaaacaaagaagaaggttacactcacatcgtcgaagtcactttcgagtcaatcgagtcaatccaagattacataatgcacccggcccatgtgggattcggagacgtttaccgtagcttctgggaaaaacttttgattttcgactataccccgagaaaa

SEQ ID NO: 700 - protein, 101 aa, synthetic construct
  REGION 1..101: note = Synthetic biopolymer
MAVKHLIILKFQDEITEAQKEEFFKTYVNLMNIIPAMKDVYWGKDVTQKNKEEGYTHIVEVTFESIESIQDYIMHPAHVGFGDVYRSFWEKLLIFDYTPRK

SEQ ID NO: 701 - DNA, 303 bp, synthetic construct; identical to SEQ ID NO: 697
  misc_feature 1..303: note = Synthetic biopolymer
  source 1..303: mol_type = other DNA

SEQ ID NO: 702 - protein, 101 aa, synthetic construct; identical to SEQ ID NO: 698
  REGION 1..101: note = Synthetic biopolymer

SEQ ID NO: 703 - DNA, 303 bp, synthetic construct
  misc_feature 1..303: note = Synthetic biopolymer
  source 1..303: mol_type = other DNA
atggctgtaaagcaccttatcgtaatcaaattcaaggacggaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtctgtttcgactcaatcgagacgatccaagattacataatacacccggcccatgtgggattcggagacgtttaccgtagcttctgggaaaaacttttgattttcgactataccccgagaaaa

SEQ ID NO: 704 - protein, 101 aa, synthetic construct
  REGION 1..101: note = Synthetic biopolymer
MAVKHLIVIKFKDGITEAQKEEFFKTYVNLVNIIPAMKDVYWGKDVTQKNKEEGYTHVVEVCFDSIETIQDYIIHPAHVGFGDVYRSFWEKLLIFDYTPRK

SEQ ID NO: 705 - DNA, 303 bp, synthetic construct; identical to SEQ ID NO: 679
  misc_feature 1..303: note = Synthetic biopolymer
  source 1..303: mol_type = other DNA

SEQ ID NO: 706 - protein, 101 aa, synthetic construct; identical to SEQ ID NO: 680
  REGION 1..101: note = Synthetic biopolymer

SEQ ID NO: 707 - DNA, 303 bp, synthetic construct; identical to SEQ ID NO: 683
  misc_feature 1..303: note = Synthetic biopolymer
  source 1..303: mol_type = other DNA

SEQ ID NO: 708 - protein, 101 aa, synthetic construct; identical to SEQ ID NO: 684
  REGION 1..101: note = Synthetic biopolymer

SEQ ID NO: 709 - DNA, 303 bp, synthetic construct; identical to SEQ ID NO: 677
  misc_feature 1..303: note = Synthetic biopolymer
  source 1..303: mol_type = other DNA

SEQ ID NO: 710 - protein, 101 aa, synthetic construct; identical to SEQ ID NO: 678
  REGION 1..101: note = Synthetic biopolymer

SEQ ID NO: 711 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattcaagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgactcagtagagacgatccaagattacataatacacccggcccatgtgggattcggagacgtttaccgtagcttctgggaaaaacttttgattttcgactataccccgggaaaa

SEQ ID NO: 712 - protein, 101 aa, synthetic construct
  REGION 1..101: note = Synthetic biopolymer
MAVKHLIVIKFKDEIQEAQKEEFFKTYVNLVNIIPAMKDVYWGKDVTQRNKEEGYTHVVEVTFDSVETIQDYIIHPAHVGFGDVYRSFWEKLLIFDYTPGK

SEQ ID NO: 713 - DNA, 303 bp, synthetic construct; identical to SEQ ID NO: 699
  misc_feature 1..303: note = Synthetic biopolymer
  source 1..303: mol_type = other DNA

SEQ ID NO: 714 - protein, 101 aa, synthetic construct; identical to SEQ ID NO: 700
  REGION 1..101: note = Synthetic biopolymer

SEQ ID NO: 715 - DNA, 303 bp, synthetic construct
  misc_feature 1..303: note = Synthetic biopolymer
  source 1..303: mol_type = other DNA
atggctgtcaagcaccttatcatcctgaaattccaagacgaaattacagaagcccagaaagaagaatttttcaagacctatgtaaacctggtgaatgtcatccctgcgatgaaagatgtttattggggcaaagacgtcacccaacgtaacaaagaagaaggttacactcacgtcgtcgaagtcactttcgagtcagtagagacgatccaagattacataatgcacccggcccatgtgggattcggagacgtttaccgtagcttctgggaaaaacttttgattttcgactataccccgagaaaa

SEQ ID NO: 716 - protein, 101 aa, synthetic construct
  REGION 1..101: note = Synthetic biopolymer
MAVKHLIILKFQDEITEAQKEEFFKTYVNLVNVIPAMKDVYWGKDVTQRNKEEGYTHVVEVTFESVETIQDYIMHPAHVGFGDVYRSFWEKLLIFDYTPRK

SEQ ID NO: 717 - DNA, 303 bp, synthetic construct; identical to SEQ ID NO: 695
  misc_feature 1..303: note = Synthetic biopolymer
  source 1..303: mol_type = other DNA

SEQ ID NO: 718 - protein, 101 aa, synthetic construct; identical to SEQ ID NO: 688
  REGION 1..101: note = Synthetic biopolymer

SEQ ID NO: 719 - DNA, 303 bp, synthetic construct; identical to SEQ ID NO: 711
  misc_feature 1..303: note = Synthetic biopolymer
  source 1..303: mol_type = other DNA

SEQ ID NO: 720 - protein, 101 aa, synthetic construct; identical to SEQ ID NO: 712
  REGION 1..101: note = Synthetic biopolymer

SEQ ID NO: 721 - DNA, 303 bp, synthetic construct
  misc_feature 1..303: note = Synthetic biopolymer
  source 1..303: mol_type = other DNA
atggctgtcaagcaccttatcgtaatcaaattcaaggacggaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtctgtttcgactcaatcgagacgatccaagattacataatacacccggcccatgtgggattcggagacgtttaccgtagcttctgggaaaaacttttgattttcgactataccccgagaaaa

SEQ ID NO: 722 - protein, 101 aa, synthetic construct; identical to SEQ ID NO: 704
  REGION 1..101: note = Synthetic biopolymer

SEQ ID NO: 723 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcagtagagacgatccaagattacataatacacccggcccatgtgggattcggagacgtttaccgtagcttctgggaaaaacttttgattttcgacttcaccccgggaaag

SEQ ID NO: 724 - protein, 101 aa, synthetic construct; identical to SEQ ID NO: 688
  REGION 1..101: note = Synthetic biopolymer

SEQ ID NO: 725 - DNA, 303 bp, synthetic construct
  misc_feature 1..303: note = Synthetic biopolymer
  source 1..303: mol_type = other DNA
atggctgtcaagcaccttatcatcctgaaattcaacgacgaaattacagaagcccagaaagaagaatttttcaagacctatgtaaacctggtgaatgtcatccctgcgatgaaagatgtttattggggcaaagacgtcacccaaaaaaacaaagaagaaggttacactcacgtcgtcgaagtcactttcgactcaatcgagacgatccaagattacataatacacccggcccatgtgggattcggagacgtttaccgtagcttctgggaaaaacttttgattttcgactataccccgagaaaa

SEQ ID NO: 726 - protein, 101 aa, synthetic construct
  REGION 1..101: note = Synthetic biopolymer
MAVKHLIILKFNDEITEAQKEEFFKTYVNLVNVIPAMKDVYWGKDVTQKNKEEGYTHVVEVTFDSIETIQDYIIHPAHVGFGDVYRSFWEKLLIFDYTPRK

SEQ ID NO: 727 - DNA, 303 bp, synthetic construct
  misc_feature 1..303: note = Synthetic biopolymer
  source 1..303: mol_type = other DNA
atgccggtcaagcaccttatcgtaatcaaattcaaggacgaaattacagaagcccagaaagaagaatttttcaacacctatgtaaacctgatgaatatcatccctgcgatgaaagatgtttattggggcaaagacgtcacccaacgtaacaaagaagaaggttggactcacatcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 728 - protein, 101 aa, synthetic construct
  REGION 1..101: note = Synthetic biopolymer
MPVKHLIVIKFKDEITEAQKEEFFNTYVNLMNIIPAMKDVYWGKDVTQRNKEEGWTHIVEVTFESVETIQDYIIHPAHVGFGDVYRSFWEKLLIFDYTPRK

SEQ ID NO: 729 - DNA, 303 bp, synthetic construct
  misc_feature 1..303: note = Synthetic biopolymer
  source 1..303: mol_type = other DNA
atggctgtaaagcaccttatcatcatcaaattcaacgacgaaattcaagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtagcttctgggaaaaacttttgattttcgactataccccgagaaaa

SEQ ID NO: 730 - protein, 101 aa, synthetic construct; identical to SEQ ID NO: 674
  REGION 1..101: note = Synthetic biopolymer

SEQ ID NO: 731 - DNA, 303 bp, synthetic construct; identical to SEQ ID NO: 667
  misc_feature 1..303: note = Synthetic biopolymer
  source 1..303: mol_type = other DNA

SEQ ID NO: 732 - protein, 101 aa, synthetic construct; identical to SEQ ID NO: 668
  REGION 1..101: note = Synthetic biopolymer

SEQ ID NO: 733 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagaggccatccaaacctacataatacacccggcccatgtgggattcggagacgtttaccgtagcttctgggaaaaacttttgaaattcgactataccccgagaaaa

SEQ ID NO: 734 - protein, 101 aa, synthetic construct
  REGION 1..101: note = Synthetic biopolymer
MAVKHLIVIKFKDEITEAQKEEFFKTYVNLVNIIPAMKDVYWGKDVTQRNKEEGYTHVVEVTFESVEAIQTYIIHPAHVGFGDVYRSFWEKLLKFDYTPRK

SEQ ID NO: 735 - DNA, 303 bp, synthetic construct; identical to SEQ ID NO: 721
  misc_feature 1..303: note = Synthetic biopolymer
  source 1..303: mol_type = other DNA

SEQ ID NO: 736 - protein, 101 aa, synthetic construct; identical to SEQ ID NO: 704
  REGION 1..101: note = Synthetic biopolymer

SEQ ID NO: 737 - DNA, 303 bp, synthetic construct
  misc_feature 1..303: note = Synthetic biopolymer
  source 1..303: mol_type = other DNA
atggctgtcaagcaccttatcatcctgaaattcaaggacggaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgactcagtagagacgatccaagattacataatacacccggcccatgtgggattcggagacgtttaccgtagcttctgggaaaaacttttgattttcgactataccccggccaaa

SEQ ID NO: 738 - protein, 101 aa, synthetic construct
  REGION 1..101: note = Synthetic biopolymer
MAVKHLIILKFKDGITEAQKEEFFKTYVNLVNIIPAMKDVYWGKDVTQRNKEEGYTHVVEVTFDSVETIQDYIIHPAHVGFGDVYRSFWEKLLIFDYTPAK

SEQ ID NO: 739 - DNA, 303 bp, synthetic construct; identical to SEQ ID NO: 143
  misc_feature 1..303: note = Synthetic biopolymer
  source 1..303: mol_type = other DNA

SEQ ID NO: 740 - protein, 101 aa, synthetic construct; identical to SEQ ID NO: 144
  REGION 1..101: note = Synthetic biopolymer

SEQ ID NO: 741 - DNA, 303 bp, synthetic construct
  misc_feature 1..303: note = Synthetic biopolymer
  source 1..303: mol_type = other DNA
atgtcagtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagtcggttactcacacatcgtcgaagtcactttcgagtcagtagagtcaatccaagattacataatacacccggcccatgtgggattcggagacgtttaccgtagcttctgggaaaaacttttgattttcgactataccccgagaaaa

SEQ ID NO: 742 - protein, 101 aa, synthetic construct
  REGION 1..101: note = Synthetic biopolymer
MSVKHLIILKFKDEITEAQKEEFFKTYVNLVNIIPAMKDVYWGKDVTQRNKEVGYSHIVEVTFESVESIQDYIIHPAHVGFGDVYRSFWEKLLIFDYTPRK

SEQ ID NO: 743 - DNA, 303 bp, synthetic construct; identical to SEQ ID NO: 699
  misc_feature 1..303: note = Synthetic biopolymer
  source 1..303: mol_type = other DNA

SEQ ID NO: 744 - protein, 101 aa, synthetic construct; identical to SEQ ID NO: 700
  REGION 1..101: note = Synthetic biopolymer

SEQ ID NO: 745 - DNA, 303 bp, synthetic construct
  misc_feature 1..303: note = Synthetic biopolymer
  source 1..303: mol_type = other DNA
atggctgtaaagcaccttatcatcctgaaattccaagacgaaattacagaagcccagaaagaagaatttttcaagacctatgtaaacctgatgaatatcatccctgcgatgaaagatgtttattggggcaaagacgtcacccaaaaaaacaaagaagaaggttacactcacatcgtcgaagtcactttcgagtcaatcgagtcaatccaagattacataatgcacccggcccatgtgggattcggagacgtttaccgtagcttctgggaaaaacttttgattttcgactataccccgagaaaa

SEQ ID NO: 746 - protein, 101 aa, synthetic construct; identical to SEQ ID NO: 700
  REGION 1..101: note = Synthetic biopolymer

SEQ ID NO: 747 - DNA, 303 bp, synthetic construct
  misc_feature 1..303: note = Synthetic biopolymer
  source 1..303: mol_type = other DNA
atgccggtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtctgtttcgagtcagtagaggaaatccaagattacataatgcacccggcccatgtgggattcggagacgtttaccgtagcttctgggaaaaacttttgattttcgactataccccgagaaaa

SEQ ID NO: 748 - protein, 101 aa, synthetic construct
  REGION 1..101: note = Synthetic biopolymer
MPVKHLIILKFKDEITEAQKEEFFKTYVNLVNIIPAMKDVYWGKDVTQRNKEEGYTHIVEVCFESVEEIQDYIMHPAHVGFGDVYRSFWEKLLIFDYTPRK

SEQ ID NO: 749 - DNA, 303 bp, synthetic construct; identical to SEQ ID NO: 729
  misc_feature 1..303: note = Synthetic biopolymer
  source 1..303: mol_type = other DNA

SEQ ID NO: 750 - protein, 101 aa, synthetic construct; identical to SEQ ID NO: 674
  REGION 1..101: note = Synthetic biopolymer

SEQ ID NO: 751 - DNA, 303 bp, synthetic construct
  misc_feature 1..303: note = Synthetic biopolymer
  source 1..303: mol_type = other DNA
atggctgtcaagcaccttatcatcatcaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtctgtttcgactcagtagagacgatccaagattacataatacacccggcccatgtgggattcggagacgtttaccgtagcttctgggaaaaacttttgattttcgactataccccgagaaaa

SEQ ID NO: 752 - protein, 101 aa, synthetic construct
  REGION 1..101: note = Synthetic biopolymer
MAVKHLIIIKFKDEITEAQKEEFFKTYVNLVNVIPAMKDVYWGKDVTQRNKEEGYTHIVEVCFDSVETIQDYIIHPAHVGFGDVYRSFWEKLLIFDYTPRK

SEQ ID NO: 753 - DNA, 303 bp, synthetic construct; identical to SEQ ID NO: 699
  misc_feature 1..303: note = Synthetic biopolymer
  source 1..303: mol_type = other DNA

SEQ ID NO: 754 - protein, 101 aa, synthetic construct; identical to SEQ ID NO: 700
  REGION 1..101: note = Synthetic biopolymer

SEQ ID NO: 755 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctgcgatgaaagatgtttattggggcaaagacgtcacccaaaaaaacaaagaagtcggttacactcacgtcgtcgaagtcactttcgactcagtagagtcaatccaagattacataatgcacccggcccatgtgggattcggagacgtttaccgtagcttctgggaaaaacttttgattttcgactataccccgagaaaa

SEQ ID NO: 756 - protein, 101 aa, synthetic construct
  REGION 1..101: note = Synthetic biopolymer
MAVKHLIVIKFKDEITEAQKEEFFKTYVNLVNIIPAMKDVYWGKDVTQKNKEVGYTHVVEVTFDSVESIQDYIMHPAHVGFGDVYRSFWEKLLIFDYTPRK

SEQ ID NO: 757 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctattgtaacctggtgaatatcatcccttcaatgaaagatgtttattggggcaaagacgtcacccaacgtaacaaagaagaaggttactcacacatcgtcgaagtcactttcgagtcaatcgagacgatccaagattacataatacacccggcccatgtgggattcggagacgtttaccgtagcttctgggaaaaacttttgattttcgactataccccgagaaaa

SEQ ID NO: 758 - protein, 101 aa, synthetic construct
  REGION 1..101: note = Synthetic biopolymer
MAVKHLIILKFKDEITEAQKEEFFKTYCNLVNIIPSMKDVYWGKDVTQRNKEEGYSHIVEVTFESIETIQDYIIHPAHVGFGDVYRSFWEKLLIFDYTPRK

SEQ ID NO: 759 - DNA, 303 bp, synthetic construct; identical to SEQ ID NO: 699
  misc_feature 1..303: note = Synthetic biopolymer
  source 1..303: mol_type = other DNA

SEQ ID NO: 760 - protein, 101 aa, synthetic construct; identical to SEQ ID NO: 700
  REGION 1..101: note = Synthetic biopolymer

SEQ ID NO: 761 - DNA, 303 bp, synthetic construct; identical to SEQ ID NO: 695
  misc_feature 1..303: note = Synthetic biopolymer
  source 1..303: mol_type = other DNA

SEQ ID NO: 762 - protein, 101 aa, synthetic construct; identical to SEQ ID NO: 688
  REGION 1..101: note = Synthetic biopolymer

SEQ ID NO: 763 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaagacctatgtaaacctggtgaatgtcatccctgcgatgaaagatgtttattggggcaaagacgtcacccaaaaaaacaaagaagaaggttacactcacgtcgtcgaagtcactttcgactcagtagagacgatccaagattacataatacacccggcccatgtgggattcggagacgtttaccgtagcttctgggaaaaacttttgattttcgactataccccgggaaaa

SEQ ID NO: 764 - protein, 101 aa, synthetic construct; identical to SEQ ID NO: 692
  REGION 1..101: note = Synthetic biopolymer

SEQ ID NO: 765 - DNA, 303 bp, synthetic construct
  misc_feature 1..303: note = Synthetic biopolymer
  source 1..303: mol_type = other DNA
atggctgtaaagcaccttatcatcctgaaattcaaggacggaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgactcagtagagacgatccaagattacataatacacccggcccatgtgggattcggagacgtttaccgtagcttctgggaaaaacttttgattttcgactataccccggccaaa

SEQ ID NO: 766 - protein, 101 aa, synthetic construct; identical to SEQ ID NO: 738
  REGION 1..101: note = Synthetic biopolymer

SEQ ID NO: 767 - DNA, 303 bp, synthetic construct
  misc_feature 1..303: note = Synthetic biopolymer
  source 1..303: mol_type = other DNA
atggctgtaaagcaccttatcatcatcaaattcaaggacgaaattacagaagcccagaaagaagaatttttcaacacctatgtaaacctggtgaatatcatccctccgatgaaagatgtttattggggcaaagacgtcacccaaaaaaacaaagaagaaggttacactcacatcgtcgaagtcactttcgagtcagtagagggaatccaagattacataatgcacccggcccatgtgggattcggagacgtttaccgtagcttctgggaaaaacttttgattttcgactataccccgagaaaa

SEQ ID NO: 768 - protein, 101 aa, synthetic construct; identical to SEQ ID NO: 684
  REGION 1..101: note = Synthetic biopolymer

SEQ ID NO: 769 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaagcccagaaagaagaatttttcaagacctatgtaaacctgatgaatatcatccctgcgatgaaagatgtttattggggcaaagacgtcacccaaaaaaacaaagaagaaggttacactcacatcgtcgaagtcactttcgactcagtagagcaaatcaaaacctacataatacacccggcccatgtgggattcggagacgtttaccgtagcttctgggaaaaacttttgattttcgactataccccgagaaaa

SEQ ID NO: 770 - protein, 101 aa, synthetic construct
  REGION 1..101: note = Synthetic biopolymer
MAVKHLIILKFKDEITEAQKEEFFKTYVNLMNIIPAMKDVYWGKDVTQKNKEEGYTHIVEVTFDSVEQIKTYIIHPAHVGFGDVYRSFWEKLLIFDYTPRK

SEQ ID NO: 771 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaacacctatgtaaacctggtgaatatcatccctcaaatgaaagatgtttattggggcaaagacgtcacccaacgtaacaaagaagaaggttacactcacatcgtcgaagtcactttcgagtcaatcgagcaaatccaagattacataatacacccggcccatgtgggattcggagacgtttaccgtagcttctgggaaaaacttttgattttcgactataccccgagaaaa

SEQ ID NO: 772 - protein, 101 aa, synthetic construct
  REGION 1..101: note = Synthetic biopolymer
MAVKHLIVIKFKDEITEAQKEEFFNTYVNLVNIIPQMKDVYWGKDVTQRNKEEGYTHIVEVTFESIEQIQDYIIHPAHVGFGDVYRSFWEKLLIFDYTPRK

SEQ ID NO: 773 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattacagaagcccagaaagaagaatttttcaacacctatgtaaacctggtgaatatcatccctcaaatgaaagatgtttattggggcaaagacgtcacccaacgtaacaaagaagaaggttacactcacatcgtcgaagtcactttcgagtcaatcgagcaaatccaagattacataatacacccggcccatgtgggattcggagacgtttaccgtagcttctgggaaaaacttttgattttcgactataccccgagaaaa

SEQ ID NO: 774 - protein, 101 aa, synthetic construct; identical to SEQ ID NO: 772
  REGION 1..101: note = Synthetic biopolymer

SEQ ID NO: 775 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcagtagagtcaatccaagattacataatacacccggcccatgtgggattcggagacgtttaccgtagcttctgggacaaacttttgaaattcgactataccccgagaaaa

SEQ ID NO: 776 - protein, 101 aa, synthetic construct
  REGION 1..101: note = Synthetic biopolymer
MAVKHLIILKFKDEITEAQKEEFFKTYVNLVNIIPAMKDVYWGKDVTQRNKEEGYTHIVEVTFDSVESIQDYIIHPAHVGFGDVYRSFWDKLLKFDYTPRK

SEQ ID NO: 777 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatcccttcaatgaaagatgtttattggggcaaagacgtcacccaaaaaaacaaagaagaaggttactcacacatcgtcgaagtcactttcgagtcagtagagtcaatccaagattacataatacacccggcccatgtgggattcggagacgtttaccgtagcttctgggacaaacttttgaaattcgactataccccgagaaaa

SEQ ID NO: 778 - protein, 101 aa, synthetic construct
  REGION 1..101: note = Synthetic biopolymer
MAVKHLIILKFKDEITEAQKEEFFKTYVNLVNIIPSMKDVYWGKDVTQKNKEEGYSHIVEVTFESVESIQDYIIHPAHVGFGDVYRSFWDKLLKFDYTPRK

SEQ ID NO: 779 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtctgtttcgactcagtagagggaatccaagattacataatgcacccggcccatgtgggattcggagacgtttaccgtagcttctgggaaaaacttttgattttcgactataccccgagaaaa

SEQ ID NO: 780 - protein, 101 aa, synthetic construct
  REGION 1..101: note = Synthetic biopolymer
MAVKHLIVIKFKDEITEAQKEEFFKTYVNLVNIIPAMKDVYWGKDVTQRNKEEGYTHIVEVCFDSVEGIQDYIMHPAHVGFGDVYRSFWEKLLIFDYTPRK

SEQ ID NO: 781 - DNA, 303 bp, synthetic construct
  misc_feature 1..303: note = Synthetic biopolymer
  source 1..303: mol_type = other DNA
atggctgtcaagcaccttatcgtactgaaattcaacgacgaaattacagaagcccagaaagaagaatttttcaagacctatgtaaacctggtgaatatcatccctgcgatgaaagatgtttattggggcaaagacgtcacccaaaaaaacaaagaagaaggttactcacacgtcgtcgaagtcactttcgagtcaatcgaggccatccaaacctacataatacacccggcccatgtgggattcggagacgtttaccgtagcttctgggaaaaacttttgattttcgactataccccgagaaaa

SEQ ID NO: 782 - protein, 101 aa, synthetic construct
  REGION 1..101: note = Synthetic biopolymer
MAVKHLIVLKFNDEITEAQKEEFFKTYVNLVNIIPAMKDVYWGKDVTQKNKEEGYSHVVEVTFESIEAIQTYIIHPAHVGFGDVYRSFWEKLLIFDYTPRK

SEQ ID NO: 783 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctggtgaatgtcatccctgcgatgaaagatgtttattggggcaaagacgtcaccatgaaaaacaaagaagaaggttacactcacgtcgtcgaagtcactttcgactcagtagagacgatccaagattacataatgcacccggcccatgtgggattcggagacgtttaccgtagcttctgggaaaaacttttgattttcgactataccccgagaaaa

SEQ ID NO: 784 - protein, 101 aa, synthetic construct
  REGION 1..101: note = Synthetic biopolymer
MAVKHLIVIKFKDEITEAQKEEFFKTYVNLVNVIPAMKDVYWGKDVTMKNKEEGYTHVVEVTFDSVETIQDYIMHPAHVGFGDVYRSFWEKLLIFDYTPRK

SEQ ID NO: 785 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaagaatttttcaagacctatgtaaacctgatgaatatcatccctgcgatgaaagatgtttattggggcaaagacgtcacccaacgttataaagaagaaggttacactcacatcgtcgaagtcactttcgagtcagtagagtcaatccaagattacataatgcacccggcccatgtgggattcggagacgtttaccgtagcttctgggaaaaacttttgattttcgactataccccgagaaaa

SEQ ID NO: 786 - protein, 101 aa, synthetic construct
  REGION 1..101: note = Synthetic biopolymer
MAVKHLIVIKFKDEITEAQKEEFFKTYVNLMNIIPAMKDVYWGKDVTQRYKEEGYTHIVEVTFESVESIQDYIMHPAHVGFGDVYRSFWEKLLIFDYTPRK

SEQ ID NO: 787 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaagcccagaaagaagaatttttcaacacctatgtaaacctggtgaatgtcatccctgcgatgaaagatgtttattggggcaaagacgtcacccaacgtaacaaagaagaaggttacactcacatcgtcgaagtcactttcgactcagtagagacgatccaagattacataatgcacccggcccatgtgggattcggagacgtttaccgtagcttctgggaaaaacttttgattttcgactataccccgagaaaa

SEQ ID NO: 788 - protein, 101 aa, synthetic construct
  REGION 1..101: note = Synthetic biopolymer
MAVKHLIILKFKDEITEAQKEEFFNTYVNLVNVIPAMKDVYWGKDVTQRNKEEGYTHIVEVTFDSVETIQDYIMHPAHVGFGDVYRSFWEKLLIFDYTPRK

SEQ ID NO: 789 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaatcacagaaagaagaatttttcaacacctatgtaaacctggtgaatatcatccctgcgatgaaagatgtttattggggcaaagacgtcacccaacgtaacaaagaagaaggttacactcacgtcgtcgaagtcactttcgagtcagtagagacgatccaagattacataatacacccggcccatgtgggattcggagacgtttaccgtccgttctgggaaaaacttttgattttcgactataccccgagaaaa

SEQ ID NO: 790 - protein, 101 aa, synthetic construct
  REGION 1..101: note = Synthetic biopolymer
MAVKHLIVIKFKDEITESQKEEFFNTYVNLVNIIPAMKDVYWGKDVTQRNKEEGYTHVVEVTFESVETIQDYIIHPAHVGFGDVYRPFWEKLLIFDYTPRK

SEQ ID NO: 791 - DNA, 303 bp, synthetic construct
  misc_feature 1..303: note = Synthetic biopolymer
  source 1..303: mol_type = other DNA
atggctgtcaagcaccttatcgtaatcaaattcaaggacgaaattacagaagcccagaaagaactttttttcaagacctatgtaaacctggtgaatatcatccctgcgatgaaagatgtttattggggcaaagacgtcacccaacgtaacaaagaagaaggttacactcacatcgtcgaagtcactttcgactcagtagagacgatccaagattacataatgcacccggcccatgtgggattcggagacgtttaccgtagcttctgggaaaaacttttgattttcgacttcaccccgagaaaa

SEQ ID NO: 792 - protein, 101 aa, synthetic construct
  REGION 1..101: note = Synthetic biopolymer
MAVKHLIVIKFKDEITEAQKELFFKTYVNLVNIIPAMKDVYWGKDVTQRNKEEGYTHIVEVTFDSVETIQDYIMHPAHVGFGDVYRSFWEKLLIFDFTPRK

SEQ ID NO: 793 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcagtagagtcaatccaagattacataatacacccggcccatgtgggattcggagacgtttaccgtagcttctgggacaaacttttgaaattcgactataccccgagaaaa

SEQ ID NO: 794 - protein, 101 aa, synthetic construct; identical to SEQ ID NO: 776
  REGION 1..101: note = Synthetic biopolymer

SEQ ID NO: 795 - DNA, 303 bp, synthetic construct
  misc_feature 1..303: note = Synthetic biopolymer
  source 1..303: mol_type = other DNA
atggctgtaaagcaccttatcgtaatcaaattcaaggacgaaattacagaagcccagaaagaactttttttcaagacctatgtaaacctggtgaatatcatccctgcgatgaaagatgtttattggggcaaagacatcacccaaaaaaacaaagaagaaggttacactcacatcgtcgaagtcactttcgactcagtagagacgatccaagattacataatgcacccggcccatgtgggattcggagacgtttaccgtagcttctgggaaaaacttttgattttcgacttcaccccaagaaaa

SEQ ID NO: 796 - protein, 101 aa, synthetic construct
  REGION 1..101: note = Synthetic biopolymer
MAVKHLIVIKFKDEITEAQKELFFKTYVNLVNIIPAMKDVYWGKDITQKNKEEGYTHIVEVTFDSVETIQDYIMHPAHVGFGDVYRSFWEKLLIFDFTPRK

SEQ ID NO: 797 - DNA, 303 bp, synthetic construct
  misc_feature 1..303: note = Synthetic biopolymer
  source 1..303: mol_type = other DNA
atggctgtaaagcaccttatcatcctgaaattcaaggacgaaattacagaatcacagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgactcaatcgagacgatccaagattacataatacacccggcccatgtgggattcggagacgtttaccgtagcttctgggaaaaacttttgaaattcgactataccccgagaaaa

SEQ ID NO: 798 - protein, 101 aa, synthetic construct
  REGION 1..101: note = Synthetic biopolymer
MAVKHLIILKFKDEITESQKEEFFKTYVNLVNIIPAMKDVYWGKDVTQRNKEEGYTHIVEVTFDSIETIQDYIIHPAHVGFGDVYRSFWEKLLKFDYTPRK

SEQ ID NO: 799 - DNA, 303 bp, synthetic construct; identical to SEQ ID NO: 791
  misc_feature 1..303: note = Synthetic biopolymer
  source 1..303: mol_type = other DNA

SEQ ID NO: 800 - protein, 101 aa, synthetic construct; identical to SEQ ID NO: 792
  REGION 1..101: note = Synthetic biopolymer

SEQ ID NO: 801 - DNA, 303 bp, synthetic construct
  misc_feature 1..303: note = Synthetic biopolymer
  source 1..303: mol_type = other DNA
atgtcagtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttactcacacatcgtcgaagtctgtttcgagtcagtagagtcaatccaagattacataatgcacccggcccatgtgggattcggagacgtttaccgtagcttctgggaaaaacttttgattttcgactataccccgagaaaa

SEQ ID NO: 802 - protein, 101 aa, synthetic construct
  REGION 1..101: note = Synthetic biopolymer
MSVKHLIILKFKDEITEAQKEEFFKTYVNLVNIIPAMKDVYWGKDVTQKNKEEGYSHIVEVCFESVESIQDYIMHPAHVGFGDVYRSFWEKLLIFDYTPRK

SEQ ID NO: 803 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctgcgatgaaagatgttgaatggggcaaagacgtcacccaacgtaacaaagaagaaggttactcacacgtcgtcgaagtcactttcgagtcaatcgaggccatccaagattacataatacacccggcccatgtgggattcggagacgtttaccgtagcttctgggaaaaacttttgattttcgactataccccgagaaaa

SEQ ID NO: 804 - protein, 101 aa, synthetic construct
  REGION 1..101: note = Synthetic biopolymer
MAVKHLIVLKFKDEITEAQKEEFFKTYVNLVNIIPAMKDVEWGKDVTQRNKEEGYSHVVEVTFESIEAIQDYIIHPAHVGFGDVYRSFWEKLLIFDYTPRK

SEQ ID NO: 805 - DNA, 303 bp, synthetic construct
  misc_feature 1..303: note = Synthetic biopolymer
  source 1..303: mol_type = other DNA
atggctgtcaagcaccttatcgtactggccttcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgactcaatcgaggaaatccaagattacataatacacccggcccatgtgggattcggagacgtttaccgtagcttctgggaaaaacttttgattttcgactataccccgagaaaa

SEQ ID NO: 806 - protein, 101 aa, synthetic construct
  REGION 1..101: note = Synthetic biopolymer
MAVKHLIVLAFKDEITEAQKEEFFKTYVNLVNIIPAMKDVYWGKDVTQRNKEEGYTHVVEVTFDSIEEIQDYIIHPAHVGFGDVYRSFWEKLLIFDYTPRK

SEQ ID NO: 807 - DNA, 303 bp, synthetic construct
  misc_feature 1..303: note = Synthetic biopolymer
  source 1..303: mol_type = other DNA
atggctgtcaagcaccttatcgtactgaaattcaacgacgaaattacagaatcacagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcaatcgagacgatccaagattacataatgcacccggcccatgtgggattcggagacgtttaccgtagcttctgggaaaaacttttgattttcgactataccccgagaaaa

SEQ ID NO: 808 - protein, 101 aa, synthetic construct
  REGION 1..101: note = Synthetic biopolymer
MAVKHLIVLKFNDEITESQKEEFFKTYVNLVNIIPAMKDVYWGKDVTQRNKEEGYTHVVEVTFESIETIQDYIMHPAHVGFGDVYRSFWEKLLIFDYTPRK

SEQ ID NO: 809 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtctgtttcgagtcagtagaggaaatccaaacctacataatgcacccggcccatgtgggattcggagacgtttaccgtagcttctgggaaaaacttttgattttcgactataccccgagaaaa

SEQ ID NO: 810 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHVVEVCFESVEEIQTYIMHPAHVGFGDVYRSFWEKLLIFDYTPRK

SEQ ID NO: 811 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtctgtttcgactcaatcgagacgatccaagattacataatgcacccggcccatgtgggattcggagacgtttaccgtagcttctgggaaaaacttttgattttcgactataccccgagaaaa

SEQ ID NO: 812 - protein, 101 aa, synthetic construct
  REGION 1..101: note = Synthetic biopolymer
MAVKHLIVLKFKDEITEAQKEEFFKTYVNLVNVIPAMKDVYWGKDVTQRNKEEGYTHIVEVCFDSIETIQDYIMHPAHVGFGDVYRSFWEKLLIFDYTPRK

SEQ ID NO: 813 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattcaagaagcccagaaagaagaatttttcaacacctatgtaaacctggtgaatatcatccctcaaatgaaagatgtttattggggcaaagacgtcacccaacgtaacaaagaagaaggttacactcacatcgtcgaagtcactttcgagtcaatcgagacgatccaagattacataatgcacccggcccatgtgggattcggagacgtttaccgtagcttctgggaaaaacttttgattttcgactataccccgagaaaa

SEQ ID NO: 814 - protein, 101 aa, synthetic construct
  REGION 1..101: note = Synthetic biopolymer
MAVKHLIVLKFKDEIQEAQKEEFFNTYVNLVNIIPQMKDVYWGKDVTQRNKEEGYTHIVEVTFESIETIQDYIMHPAHVGFGDVYRSFWEKLLIFDYTPRK

SEQ ID NO: 815 - DNA, 303 bp, synthetic construct
  misc_feature 1..303: note = Synthetic biopolymer
  source 1..303: mol_type = other DNA
atgccggtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacgtcgtcgaagtcactttcgactcagtatcacaaatccaagattacataatacacccggcccatgtgggattcggagacgtttaccgtagcttctgggaaaaacttttgattttcgactataccccgggaaaa

SEQ ID NO: 816 - protein, 101 aa, synthetic construct
  REGION 1..101: note = Synthetic biopolymer
MPVKHLIVLKFKDEITEAQKEEFFKTYVNLVNIIPAMKDVYWGKDVTQKNKEEGYTHVVEVTFDSVSQIQDYIIHPAHVGFGDVYRSFWEKLLIFDYTPGK

SEQ ID NO: 817 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatgtcatccctgcgatgaaagatgtttattggggcaaagacgtcacccaacgtaacaaagaagaaggttacactcacatcgtcgaagtctgtttcgactcaatcgagacgatccaagattacataatgcacccggcccatgtgggattcggagacgtttaccgtagcttctgggaaaaacttttgattttcgactataccccgagaaaa

SEQ ID NO: 818 - protein, 101 aa, synthetic construct; identical to SEQ ID NO: 812
  REGION 1..101: note = Synthetic biopolymer

SEQ ID NO: 819 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcagtagaggccatccaaacctacataatgcacccggcccatgtgggattcggagacgtttaccgtagcttctgggaaaaacttttgattttcgactataccccgggaaaa

SEQ ID NO: 820 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHVVEVTFESVEAIQTYIMHPAHVGFGDVYRSFWEKLLIFDYTPGK

SEQ ID NO: 821 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaacacctatgtaaacctggtgaatatcatccctgcgatgaaagatgtttattggggcaaagacgtcacccaacgtaacaaagaagaaggttacactcacgtcgtcgaagtcactttcgactcaatcgagtcaatccaagattacataatacacccggcccatgtgggattcggagacgtttaccgtagcttctgggaaaaacttttgattttcgactataccccgagaaaa

SEQ ID NO: 822 - protein, 101 aa, synthetic construct
  REGION 1..101: note = Synthetic biopolymer
MAVKHLIVLKFKDEITEAQKEEFFNTYVNLVNIIPAMKDVYWGKDVTQRNKEEGYTHVVEVTFDSIESIQDYIIHPAHVGFGDVYRSFWEKLLIFDYTPRK

SEQ ID NO: 823 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatccctcaaatgaaagatgtttattggggcaaagacgtcacccaacgtaacaaagaagaaggttacactcacgtcgtcgaagtcactttcgactcaatcgagacgatccaagattacataatacacccggcccatgtgggattcggagacgtttaccgtagcttctgggaagaacttttgattttcgactataccccgagaaaa

SEQ ID NO: 824 - protein, 101 aa, synthetic construct
  REGION 1..101: note = Synthetic biopolymer
MAVKHLIVLKFKDEITEAQKEEFFKTYVNLVNIIPQMKDVYWGKDVTQRNKEEGYTHVVEVTFDSIETIQDYIIHPAHVGFGDVYRSFWEELLIFDYTPRK

SEQ ID NO: 825 - DNA, 303 bp, synthetic construct
  misc_feature 1..303: note = Synthetic biopolymer
  source 1..303: mol_type = other DNA
atggctgtaaagcaccttatcgtactgaaattcaacgacgaaattacagaatcacagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcaatcgagacgatccaagattacataatgcacccggcccatgtgggattcggagacgtttaccgtagcttctgggaaaaacttttgattttcgactataccccgagaaaa

SEQ ID NO: 826 - protein, 101 aa, synthetic construct; identical to SEQ ID NO: 808
  REGION 1..101: note = Synthetic biopolymer

SEQ ID NO: 827 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagtcgaatttttcaagacctatgtaaacctggtgaatatcatccctcaaatgaaagatgtttattggggcaaagacgtcacccaaaaaaacaaagaagaaggttacactcacgtcgtcgaagtcactttcgagtcaatctcaacgatccaagattacataatgcacccggcccatgtgggattcggagacgtttaccgtagcttctgggaaaaacttttgattttcgactataccccgagaaaa

SEQ ID NO: 828 - protein, 101 aa, synthetic construct
  REGION 1..101: note = Synthetic biopolymer
MAVKHLIVLKFKDEITEAQKVEFFKTYVNLVNIIPQMKDVYWGKDVTQKNKEEGYTHVVEVTFESISTIQDYIMHPAHVGFGDVYRSFWEKLLIFDYTPRK

SEQ ID NO: 829 - DNA, 303 bp, synthetic construct
  misc_feature 1..303: note = Synthetic biopolymer
  source 1..303: mol_type = other DNA
atgccggtcaagcaccttatcgtactgaaattcaaggacggaattacagaagcccagaaagaagaatttttcaagacctatgtaaacctggtgaatatcatccctgcgatgaaagatgtttattggggcaaagacgtcacctgtaaaaacaaagaagaaggttacactcacgtcgtcgaagtcactttcgactcaatcgagacgatccaagattacataatacacccggcccatgtgggattcggagacgtttaccgtagcttctgggaaaaacttttgattttcgactataccccgagaaaa

SEQ ID NO: 830 - protein, 101 aa, synthetic construct
  REGION 1..101: note = Synthetic biopolymer
MPVKHLIVLKFKDGITEAQKEEFFKTYVNLVNIIPAMKDVYWGKDVTCKNKEEGYTHVVEVTFDSIETIQDYIIHPAHVGFGDVYRSFWEKLLIFDYTPRK

SEQ ID NO: 831 - DNA, 303 bp, synthetic construct
  misc_feature 1..303: note = Synthetic biopolymer
  source 1..303: mol_type = other DNA
atgccggtcaagcaccttatcgtactgaaattcgtcgacgaaattcaagaagcccagaaagaagaatttttcaagacctatgtaaacctggtgaatatcatccctgcgatgaaagatgtttattggggcaaagacgtcacccaacgtaacaaagaagaaggttacactcacgtcgtcgaagtcactttcgagtcaatcgagacgatccaagattacataatacacccggcccatgtgggattcggagacgtttaccgtagcttctgggaaaaacttttgattttcgactataccccgagaaaa

SEQ ID NO: 832 - protein, 101 aa, synthetic construct
  REGION 1..101: note = Synthetic biopolymer
MPVKHLIVLKFVDEIQEAQKEEFFKTYVNLVNIIPAMKDVYWGKDVTQRNKEEGYTHVVEVTFESIETIQDYIIHPAHVGFGDVYRSFWEKLLIFDYTPRK

SEQ ID NO: 833 - DNA, 303 bp, synthetic construct; identical to SEQ ID NO: 831
  misc_feature 1..303: note = Synthetic biopolymer
  source 1..303: mol_type = other DNA

SEQ ID NO: 834 - protein, 101 aa, synthetic construct; identical to SEQ ID NO: 832
  REGION 1..101: note = Synthetic biopolymer

SEQ ID NO: 835 - DNA, 303 bp, synthetic construct; identical to SEQ ID NO: 817
  misc_feature 1..303: note = Synthetic biopolymer
  source 1..303: mol_type = other DNA

SEQ ID NO: 836 - protein, 101 aa, synthetic construct; identical to SEQ ID NO: 812
  REGION 1..101: note = Synthetic biopolymer

SEQ ID NO: 837 - DNA, 303 bp, synthetic construct
  misc_feature 1..303: note = Synthetic biopolymer
  source 1..303: mol_type = other DNA
atgccggtcaagcaccttatcgtactgaaattcaaggacgaaattacagaagcccagaaagaagaatttttcaagacctatgtaaacctgatgaatatcatccctgcgatgaaagatgtttattggggcaaagacgtcacccaaaaaaacaaagaagaaggttacactcacgtcgtcgaagtctgtttcgactcagtagagtcaatccaagattacataatacacccggcccatgtgggattcggagacgtttaccgtagcttctgggaaaaacttttgattttcgactataccccgagaaaa

SEQ ID NO: 838 - protein, 101 aa, synthetic construct
  REGION 1..101: note = Synthetic biopolymer
MPVKHLIVLKFKDEITEAQKEEFFKTYVNLMNIIPAMKDVYWGKDVTQKNKEEGYTHVVEVCFDSVESIQDYIIHPAHVGFGDVYRSFWEKLLIFDYTPRK

SEQ ID NO: 839 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatatcatccctccgatgaaagatgtttattggggcaaagacgtcacccaaaaaaacaaagaagaaggttacactcacgtcgtcgaagtcactttcgagtcagtatcatcaatccaagattacataatgcacccggcccatgtgggattcggagacgtttaccgtagcttctgggaaaaacttttgattttcgactataccccgggaaaa

SEQ ID NO: 840 - protein, 101 aa, synthetic construct
  REGION 1..101: note = Synthetic biopolymer
MAVKHLIVLKFKDEITEAQKEEFFKTYVNLVNIIPPMKDVYWGKDVTQKNKEEGYTHVVEVTFESVSSIQDYIMHPAHVGFGDVYRSFWEKLLIFDYTPGK

SEQ ID NO: 841 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaaatcttcaagacctatgtaaacctggtgaatatcatccctgcgatgaaagatgtttattggggcaaagacgtcacccaacgtaacaaagaagaaggttacactcacatcgtcgaagtcactttcgagtcaatcgagcaaatcgccgattacataatgcacccggcccatgtgggattcggagacgtttaccgtagcttctgggaaaaacttttgattttcgactataccccgagaaaa

SEQ ID NO: 842 - protein, 101 aa, synthetic construct
  REGION 1..101: note = Synthetic biopolymer
MAVKHLIVLKFKDEITEAQKEEIFKTYVNLVNIIPAMKDVYWGKDVTQRNKEEGYTHIVEVTFESIEQIADYIMHPAHVGFGDVYRSFWEKLLIFDYTPRK

SEQ ID NO: 843 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaactttttttcaagacctatgtaaacctgatgaatatcatcccttcaatgaaagatgtttattggggcaaagacgtcacccaaaaaaacaaagaagaaggttacactcacatcgtcgaagtcactttcgagtcaatcgagtcaatccaagattacataatgcacccggcccatgtgggattcggagacgtttaccgtagcttctgggaaaaacttttgattttcgactataccccgagaaaa

SEQ ID NO: 844 - protein, 101 aa, synthetic construct
  REGION 1..101: note = Synthetic biopolymer
MAVKHLIVLKFKDEITEAQKELFFKTYVNLMNIIPSMKDVYWGKDVTQKNKEEGYTHIVEVTFESIESIQDYIMHPAHVGFGDVYRSFWEKLLIFDYTPRK

SEQ ID NO: 845 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatcccttcaatgaaagatgtttattggggcaaagacgtcacccaacgtaacaaagaagaaggttacactcactgtgtcgaagtcactttcgactcaatcgagtcaatccaagattacataatacacccggcccatgtgggattcggagacgtttaccgtagcttctgggaaaaacttttgattttcgactataccccgagaaaa

SEQ ID NO: 846 - protein, 101 aa, synthetic construct
  REGION 1..101: note = Synthetic biopolymer
MAVKHLIVLKFKDEITEAQKEEFFKTYVNLVNIIPSMKDVYWGKDVTQRNKEEGYTHCVEVTFDSIESIQDYIIHPAHVGFGDVYRSFWEKLLIFDYTPRK

SEQ ID NO: 847 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattcaagaagcccagaaagaagaatttttcaagacctatgtaaacctggtgaatatcatccctcaaatgaaagatgtttattggggcaaagacgtcacccacaaaaacaaagaagaaggttacactcacgtcgtcgaagtcactttcgactcagtagagacgatccaagattacataatgcacccggcccatgtgggattcggagacgtttaccgtagcttctgggaaaaacttttaatttttgattacactccaaggaag

SEQ ID NO: 848 - protein, 101 aa, synthetic construct
  REGION 1..101: note = Synthetic biopolymer
MAVKHLIVLKFKDEIQEAQKEEFFKTYVNLVNIIPQMKDVYWGKDVTHKNKEEGYTHVVEVTFDSVETIQDYIMHPAHVGFGDVYRSFWEKLLIFDYTPRK

SEQ ID NO: 849 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaacacctatgtaaacctggtgaatatcatccctgcgatgaaagatgtttattggggcaaagacgtcacccaacgtaacaaagaagaaggttggactcacgtcgtcgaagtcactttcgactcagtagagacgatccaagattacataatgcacccggcccatgtgggattcggagacgtttaccgtagcttctgggaaaaacttttgattttcgactataccccgagaaaa

SEQ ID NO: 850 - protein, 101 aa, synthetic construct
  REGION 1..101: note = Synthetic biopolymer
MAVKHLIVLKFKDEITEAQKEEFFNTYVNLVNIIPAMKDVYWGKDVTQRNKEEGWTHVVEVTFDSVETIQDYIMHPAHVGFGDVYRSFWEKLLIFDYTPRK

SEQ ID NO: 851 - DNA, 303 bp, synthetic construct
  misc_feature 1..303: note = Synthetic biopolymer
  source 1..303: mol_type = other DNA
atggctgtcaagcaccttatcgtactgaaattcaaggacgaaattacagaagcccagaaagaagaatttttcaagacctatgtaaacctggtgaatatcatcccttcaatgaaagatgtttattggggcaaagacgtcacctgtaaaaacaaagaagaaggttacactcacatcgtcgaagtcactttcgactcagtagaggaaatccaagattacataatgcacccggcccatgtgggattcggagacgtttaccgtccgttctgggaaaaacttttgattttcgactataccccgagaaaa

SEQ ID NO: 852 - protein, 101 aa, synthetic construct
  REGION 1..101: note = Synthetic biopolymer
MAVKHLIVLKFKDEITEAQKEEFFKTYVNLVNIIPSMKDVYWGKDVTCKNKEEGYTHIVEVTFDSVEEIQDYIMHPAHVGFGDVYRPFWEKLLIFDYTPRK

SEQ ID NO: 853 - DNA, 303 bp, synthetic construct
  misc_feature 1..303: note = Synthetic biopolymer
  source 1..303: mol_type = other DNA
atggctgtaaagcaccttatcgtactgaaattcaaggacgaaattacagaagcccagaaagaagaatttttcaagacctatgtaaacctggtgaatatcatccctgcgatgaaagatgtttattggggcaaagacgtcacccaacgtaacaaagaagaaggttacactcacatcgtcgaagtcactttcgagtcaatcgagcaaatccaaacctacataatgcacccggcccatgtgggattcggagacgtttaccgtagcttctgggaaaaacttttgaaattcgactataccccgagaaaa

SEQ ID NO: 854 - protein, 101 aa, synthetic construct
  REGION 1..101: note = Synthetic biopolymer
MAVKHLIVLKFKDEITEAQKEEFFKTYVNLVNIIPAMKDVYWGKDVTQRNKEEGYTHIVEVTFESIEQIQTYIMHPAHVGFGDVYRSFWEKLLKFDYTPRK

SEQ ID NO: 855 - DNA, 303 bp, synthetic construct
  misc_feature 1..303: note = Synthetic biopolymer
  source 1..303: mol_type = other DNA
atgccggtcaagcaccttatcatcgttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 856 - protein, 101 aa, synthetic construct
  REGION 1..101: note = Synthetic biopolymer
MPVKHLIIVKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 857 - DNA, 303 bp, synthetic construct
  misc_feature 1..303: note = Synthetic biopolymer
  source 1..303: mol_type = other DNA
atgccggtcaagcaccttatcatcactaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 858 - protein, 101 aa, synthetic construct
  REGION 1..101: note = Synthetic biopolymer
MPVKHLIITKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 859 - DNA, 303 bp, synthetic construct
  misc_feature 1..303: note = Synthetic biopolymer
  source 1..303: mol_type = other DNA
atgccggtcaagcaccttatcatctgtaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 860 - protein, 101 aa, synthetic construct
  REGION 1..101: note = Synthetic biopolymer
MPVKHLIICKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 861 - DNA, 303 bp, synthetic construct
  misc_feature 1..303: note = Synthetic biopolymer
  source 1..303: mol_type = other DNA
atgccggtcaagcaccttatcatcgggaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 862 - protein, 101 aa, synthetic construct
  REGION 1..101: note = Synthetic biopolymer
MPVKHLIIGKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 863 - DNA, 303 bp, synthetic construct
  misc_feature 1..303: note = Synthetic biopolymer
  source 1..303: mol_type = other DNA
atgccggtcaagcaccttatcatcgcgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 864 - protein, 101 aa, synthetic construct
  REGION 1..101: note = Synthetic biopolymer
MPVKHLIIAKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 865 - DNA, 303 bp, synthetic construct
  misc_feature 1..303: note = Synthetic biopolymer
  source 1..303: mol_type = other DNA
atgccggtcaagcaccttatcatcatgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 866 - protein, 101 aa, synthetic construct
  REGION 1..101: note = Synthetic biopolymer
MPVKHLIIMKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 867 - DNA, 305 bp, synthetic construct
  misc_feature 1..305: note = Synthetic biopolymer
  source 1..305: mol_type = other DNA
atgccggtcaagcaccttatcatctttaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgatataccccgggaaaataa

SEQ ID NO: 868 - protein, 101 aa, synthetic construct
  REGION 1..101: note = Synthetic biopolymer
MPVKHLIIFKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 869 - DNA, 303 bp, synthetic construct
  misc_feature 1..303: note = Synthetic biopolymer
  source 1..303: mol_type = other DNA
atgccggtcaagcaccttatcatctcgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgactcagtagagtcaatccaagattacataatacacccggcccatgtgggattcggagacgtttaccgtagcttctgggaaaaacttttgattttcgactataccccgggaaaa

SEQ ID NO: 870 - protein, 101 aa, synthetic construct
  REGION 1..101: note = Synthetic biopolymer
MPVKHLIISKFKDEITEAQKEEFFKTYVNLVNIIPAMKDVYWGKDVTQKNKEEGYTHIVEVTFDSVESIQDYIIHPAHVGFGDVYRSFWEKLLIFDYTPGK

SEQ ID NO: 871 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaaggtaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 872 - protein, 101 aa, synthetic construct
  REGION 1..101: note = Synthetic biopolymer
MAVKHLIILKFKDEITEAQKEEFFKTYVNLVNIIPAMKDVYWGKDVTQGNKEEGYTHVVEVTFDSVEMIQDYIMHPAHVGFGDVYRSFWEKLLIFDYTPRK

SEQ ID NO: 873 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaagcgaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 874 - protein, 101 aa, synthetic construct
  REGION 1..101: note = Synthetic biopolymer
MAVKHLIILKFKDEITEAQKEEFFKTYVNLVNIIPAMKDVYWGKDVTQANKEEGYTHVVEVTFDSVEMIQDYIMHPAHVGFGDVYRSFWEKLLIFDYTPRK

SEQ ID NO: 875 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaacataacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 876 - protein, 101 aa, synthetic construct
  REGION 1..101: note = Synthetic biopolymer
MAVKHLIILKFKDEITEAQKEEFFKTYVNLVNIIPAMKDVYWGKDVTQHNKEEGYTHVVEVTFDSVEMIQDYIMHPAHVGFGDVYRSFWEKLLIFDYTPRK

SEQ ID NO: 877 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaatgtaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 878 - protein, 101 aa, synthetic construct
  REGION 1..101: note = Synthetic biopolymer
MAVKHLIILKFKDEITEAQKEEFFKTYVNLVNIIPAMKDVYWGKDVTQCNKEEGYTHVVEVTFDSVEMIQDYIMHPAHVGFGDVYRSFWEKLLIFDYTPRK

SEQ ID NO: 879 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaaactaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 880 - protein, 101 aa, synthetic construct
  REGION 1..101: note = Synthetic biopolymer
MAVKHLIILKFKDEITEAQKEEFFKTYVNLVNIIPAMKDVYWGKDVTQTNKEEGYTHVVEVTFDSVEMIQDYIMHPAHVGFGDVYRSFWEKLLIFDYTPRK

SEQ ID NO: 881 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaagtgaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 882 - protein, 101 aa, synthetic construct
  REGION 1..101: note = Synthetic biopolymer
MAVKHLIILKFKDEITEAQKEEFFKTYVNLVNIIPAMKDVYWGKDVTQVNKEEGYTHVVEVTFDSVEMIQDYIMHPAHVGFGDVYRSFWEKLLIFDYTPRK

SEQ ID NO: 883 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaatcgaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 884 - protein, 101 aa, synthetic construct
  REGION 1..101: note = Synthetic biopolymer
MAVKHLIILKFKDEITEAQKEEFFKTYVNLVNIIPAMKDVYWGKDVTQSNKEEGYTHVVEVTFDSVEMIQDYIMHPAHVGFGDVYRSFWEKLLIFDYTPRK

SEQ ID NO: 885 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaaaataacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 886 - protein, 101 aa, synthetic construct
  REGION 1..101: note = Synthetic biopolymer
MAVKHLIILKFKDEITEAQKEEFFKTYVNLVNIIPAMKDVYWGKDVTQNNKEEGYTHVVEVTFDSVEMIQDYIMHPAHVGFGDVYRSFWEKLLIFDYTPRK

SEQ ID NO: 887 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaaccgaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 888 - protein, 101 aa, synthetic construct
  REGION 1..101: note = Synthetic biopolymer
MAVKHLIILKFKDEITEAQKEEFFKTYVNLVNIIPAMKDVYWGKDVTQPNKEEGYTHVVEVTFDSVEMIQDYIMHPAHVGFGDVYRSFWEKLLIFDYTPRK

SEQ ID NO: 889 - DNA, 303 bp, synthetic construct
  misc_feature 1..303: note = Synthetic biopolymer
  source 1..303: mol_type = other DNA
atggctgtcaagcaccttatcatcctgaaattcaaggacgaaattacagaagcccagaaagaagaatttttcaagacctatgtaaacctggtgaatatcatccctgcgatgaaagatgtttattggggcaaagacgtcacccaactgaacaaagaagaaggttacactcacgtcgtcgaagtcactttcgactcagtagagatgatccaagattacataatgcacccggcccatgtgggattcggagacgtttaccgtagcttctgggaaaaacttttgattttcgactataccccgagaaaa

SEQ ID NO: 890 - protein, 101 aa, synthetic construct
  REGION 1..101: note = Synthetic biopolymer
MAVKHLIILKFKDEITEAQKEEFFKTYVNLVNIIPAMKDVYWGKDVTQLNKEEGYTHVVEVTFDSVEMIQDYIMHPAHVGFGDVYRSFWEKLLIFDYTPRK